SEQUENCE LISTING (49 sequences)

SEQ ID NO: 1 - DNA, 21 bp, Artificial Sequence
  synthetic primer
gtggactcttgaaagtactat

SEQ ID NO: 2 - DNA, 21 bp, Artificial Sequence
  synthetic primer
gctgagctgttactaggacaa

SEQ ID NO: 3 - DNA, 21 bp, Artificial Sequence
  synthetic primer
cactggtcacgtttggaaaga

SEQ ID NO: 4 - DNA, 21 bp, Artificial Sequence
  synthetic primer
caacaattgccctagcacttg

SEQ ID NO: 5 - DNA, 21 bp, Artificial Sequence
  synthetic primer
gacaacagcaccttgcagagt

SEQ ID NO: 6 - DNA, 22 bp, Artificial Sequence
  synthetic primer
agtctgactgagccggtaattc

SEQ ID NO: 7 - DNA, 22 bp, Artificial Sequence
  synthetic primer
cacatctccagctcgtgtatgc

SEQ ID NO: 8 - DNA, 22 bp, Artificial Sequence
  synthetic primer
ttgccaatgtccgcttcaatgc

SEQ ID NO: 9 - DNA, 22 bp, Artificial Sequence
  synthetic primer
ctacgctcttctgcatcaatgc

SEQ ID NO: 10 - DNA, 21 bp, Artificial Sequence
  synthetic primer
accggaatgtagtaacgattt

SEQ ID NO: 11 - DNA, 20 bp, Artificial Sequence
  synthetic primer
ggcaaatcaccagatacaaa

SEQ ID NO: 12 - DNA, 21 bp, Artificial Sequence
  synthetic primer
tggaacttgacgatactctaa

SEQ ID NO: 13 - DNA, 21 bp, Artificial Sequence
  synthetic primer
gctgaagctatgttcgccatt

SEQ ID NO: 14 - DNA, 77 bp, Artificial Sequence
  synthetic primer
acgaataagcagaatataacatattagcaggtgcttagattacactcatagagatacatggaggcccagaataccct

SEQ ID NO: 15 - DNA, 77 bp, Artificial Sequence
  synthetic primer
agtgtttttatatcctatgtaagtcttcaaactttttttttatctctggtaacctcagtatagcgaccagcattcac

SEQ ID NO: 16 - DNA, 60 bp, Artificial Sequence
  synthetic primer
aaagaacgcacatatgaacaagcattaaacgagcaaagaacggatccccgggttaattaa

SEQ ID NO: 17 - DNA, 60 bp, Artificial Sequence
  synthetic primer
tcataatgcttgtcatacacatttatatgtttaattaatagaattcgagctcgtttaaac

SEQ ID NO: 18 - DNA, 60 bp, Artificial Sequence
  synthetic primer
acctaagggcagctttagacacaacagctccccagaaaaacggatccccgggttaattaa

SEQ ID NO: 19 - DNA, 60 bp, Artificial Sequence
  synthetic primer
cgtgtatgtatttgtacgtattgttttgtatatttgattggaattcgagctcgtttaaac

SEQ ID NO: 20 - DNA, 60 bp, Artificial Sequence
  synthetic primer
tctcactaacctctcttcaactgctcaataatttcccgctcggatccccgggttaattaa

SEQ ID NO: 21 - DNA, 60 bp, Artificial Sequence
  synthetic primer
gggaaatggaaagagaaaagaaaagagtatgaaaggaactgaattcgagctcgtttaaac

SEQ ID NO: 22 - DNA, 77 bp, Artificial Sequence
  synthetic primer
aagtgaggctggacaacaagaacgacatacaccgcgtaaaggcccacaagactgcacatggaggcccagaataccct

SEQ ID NO: 23 - DNA, 77 bp, Artificial Sequence
  synthetic primer
agtgatggttagatcaagaggaagttcgtctgttgccgaaaatggtggaaagtcgcagtatagcgaccagcattcac

SEQ ID NO: 24 - DNA, 77 bp, Artificial Sequence
  synthetic primer
agctttaaaagagagaatagtgagaaaagatagtgttacacaacatcaactaaaaacatggaggcccagaataccct

SEQ ID NO: 25 - DNA, 77 bp, Artificial Sequence
  synthetic primer
ctaccatcttctctcttaaaaaggggcagcattttctatgggtatttgtccttggcagtatagcgaccagcattcac

SEQ ID NO: 26 - DNA, 60 bp, Artificial Sequence
  synthetic primer
gtatatcataagttgctgtatatgggcagcacgtattactcggatccccgggttaattaa

SEQ ID NO: 27 - DNA, 60 bp, Artificial Sequence
  synthetic primer
tgatcagtgcatcttaacccttcttttgtctccattttttgaattcgagctcgtttaaac

SEQ ID NO: 28 - DNA, 57 bp, Artificial Sequence
  synthetic primer
atttaggcactgcatttatcttttaaaaatcattatacggatccccgggttaattaa

SEQ ID NO: 29 - DNA, 60 bp, Artificial Sequence
  synthetic primer
gtctatgcataatttttgcgcggtttataacggatccttcgaattcgagctcgtttaaac

SEQ ID NO: 30 - DNA, 60 bp, Artificial Sequence
  synthetic primer
aagccttcaaagaaaaagggaaatcagtgtaacatagacgcggatccccgggttaattaa

SEQ ID NO: 31 - DNA, 60 bp, Artificial Sequence
  synthetic primer
tataaacaaaaaaaagaagtataaagcatctgcatagcaagaattcgagctcgtttaaac

SEQ ID NO: 32 - DNA, 59 bp, Artificial Sequence
  synthetic primer
atcataggcacgtgctattttcgaacatccactttcaatcggatccccgggttaattaa

SEQ ID NO: 33 - DNA, 60 bp, Artificial Sequence
  synthetic primer
aaacttcctcacttcttcgagcttgtaatgaattgctgttgaattcgagctcgtttaaac

SEQ ID NO: 34 - DNA, 60 bp, Artificial Sequence
  synthetic primer
catttcttcatttacttaactgcgagaagattataatagccggatccccgggttaattaa

SEQ ID NO: 35 - DNA, 60 bp, Artificial Sequence
  synthetic primer
tgaataaagggtgaaaatgtgacaatgtgaatgttttagtgaattcgagctcgtttaaac

SEQ ID NO: 36 - DNA, 60 bp, Artificial Sequence
  synthetic primer
ttctagtccgaagagggtgtatacgaaaagaaaatatacgcggatccccgggttaattaa

SEQ ID NO: 37 - DNA, 60 bp, Artificial Sequence
  synthetic primer
tgcctttacccagcaattgaacgggaggtatgcaaaaggggaattcgagctcgtttaaac

SEQ ID NO: 38 - DNA, 60 bp, Artificial Sequence
  synthetic primer
caggcactaaaagcggtggtaagctaaaactagtacgaagcggatccccgggttaattaa

SEQ ID NO: 39 - DNA, 60 bp, Artificial Sequence
  synthetic primer
ttttcctaggttgacaattttggacgaggctgaccaataagaattcgagctcgtttaaac

SEQ ID NO: 40 - DNA, 60 bp, Artificial Sequence
  synthetic primer
tccgcataaactagatgataaagagtacaaacaagtcgcccggatccccgggttaattaa

SEQ ID NO: 41 - DNA, 60 bp, Artificial Sequence
  synthetic primer
agcatattaagatcaaattagttgaggctgtaaataaaaagaattcgagctcgtttaaac

SEQ ID NO: 42 - DNA, 60 bp, Artificial Sequence
  synthetic primer
agcaattgctgacgaattggattggttaaaatttggtatacggatccccgggttaattaa

SEQ ID NO: 43 - DNA, 60 bp, Artificial Sequence
  synthetic primer
ttattaacataataagaataatgataatgataatacgcgggaattcgagctcgtttaaac

SEQ ID NO: 44 - DNA, 60 bp, Artificial Sequence
  synthetic primer
aatgaacctaagcgaggccattcacgataataatggctcacggatccccgggttaattaa

SEQ ID NO: 45 - DNA, 60 bp, Artificial Sequence
  synthetic primer
tatgtattttttcaatgcaatcaatatcattaatcataaggaattcgagctcgtttaaac

SEQ ID NO: 46 - DNA, 60 bp, Artificial Sequence
  synthetic primer
tgcaggcctagaaaagaaaaagaaaaagcaaaataatcaacggatccccgggttaattaa

SEQ ID NO: 47 - DNA, 60 bp, Artificial Sequence
  synthetic primer
tacaaatgcttggaaaagaaataaaagtggggacgcttatgaattcgagctcgtttaaac

SEQ ID NO: 48 - DNA, 60 bp, Artificial Sequence
  synthetic primer
ctctaatccagctgactatcttttagaatttggttcggggcggatccccgggttaattaa

SEQ ID NO: 49 - DNA, 60 bp, Artificial Sequence
  synthetic primer
tttttcaaatttaattttttcccgctaataagaccataaagaattcgagctcgtttaaac

(19) United States
(12) Patent Application Publication
(10) Pub. No.: US 2020/0164047 A1
(43) Pub. Date: May 28, 2020

(54) THERAPY AND DIAGNOSIS OF DISEASE CHARACTERIZED BY ALTERATIONS IN THE DNA DAMAGE RESPONSE

(71) Applicants: UNIVERSITÀ DEGLI STUDI DI MILANO, Milano (IT); ISTITUTO EUROPEO DI ONCOLOGIA S.R.L., Milano (IT); IFOM - FONDAZIONE ISTITUTO FIRC DI ONCOLOGIA MOLECOLARE, Milano (IT)

(72) Inventors: Saverio MINUCCI, Milano (IT); Mohamed ELGENDY, Prague (CZ); Riccardo CAZZOLI, Milano (IT); Sebastiano PERI, Milano (IT); Elisa FERRARI, Milano (IT); Marco FOIANI, Milano (IT)

(73) Assignees: UNIVERSITÀ DEGLI STUDI DI MILANO, Milano (IT); ISTITUTO EUROPEO DI ONCOLOGIA S.R.L., Milano (IT); IFOM - FONDAZIONE ISTITUTO FIRC DI ONCOLOGIA MOLECOLARE, Milano (IT)

(21) Appl. No.: 16/615,045

(22) PCT Filed: May 21, 2018
(86) PCT No.: PCT/EP2018/063257

§ 371 Date: Nov. 19, 2019

(30) Foreign Application Priority Data
May 19, 2017 (EP) 17172073.3

(51) Int. Cl. A61K 38/46; A61K 31/164; A61K 31/155; A61K 31/436; A61K 31/454; A61K 31/5415

(52) U.S. Cl. CPC A61K 38/465; A61K 31/164; A61K 31/155; A61K 45/06; A61K 31/454; A61K 31/5415; C12Y 301/03016; A61K 31/436

(57) ABSTRACT

The present invention relates to at least one modulator of PP2A or at least one modulator of PP2A-like phosphatase or at least one modulator of PP2A and PP2A-like phosphatase or a combination of said modulators for use in the treatment of a disease characterized by an alteration in the DNA damage response. The present invention also relates to a method to identify a subject to be treated with a PP2A modulator comprising detecting in the genome of said patient a mutation in PP2A.

### FIELD OF INVENTION

The present invention relates to at least one modulator of PP2A or at least one modulator of PP2A-like phosphatase or at least one modulator of PP2A and PP2A-like phosphatase or a combination of said modulators for use in the treatment of a disease characterized by an alteration in the DNA damage response. The present invention also relates to a method to identify a subject to be treated with a PP2A modulator comprising detecting in the genome of said patient a mutation in PP2A.

### STATE OF THE ART

The Tel1^{ATM} and Mec1^{ATR} PI3K-like kinases (PI3KK) mediate DNA damage checkpoints (for a review see (Harrison and Haber, 2006)) by activating the Chk1^{CHK1} and Rad53^{CHK2} kinases, which transduce the signal to downstream targets. Rad53 controls replication fork integrity and phosphorylates Dun1 to up-regulate dNTP levels (Bashkirov et al., 2003; Sogo et al., 2002). Checkpoint deactivation occurs during recovery or adaptation (Bartek and Lukas, 2007) and is mediated by the PP1, PP2C and PP4 phosphatases, which act in response to specific genotoxic contexts (Bazzi et al., 2010; Keogh et al., 2006; Leroy et al., 2003; O'Neill et al., 2007). Hydroxyurea (HU) inhibits replicative DNA polymerases by limiting dNTP pools, thus causing replication stress and Mec1 activation (Slater, 1973; Sun et al., 1996). PP2A and PP2A-like Ser/Thr phosphatases are ceramide-activated protein phosphatases (CAPP) (Janssens and Goris, 2001; Nickels and Broach, 1996) that contain catalytic, regulatory and scaffolding subunits. PPH21 and PPH22 encode PP2A catalytic subunits (Sneddon et al., 1990), while Tpd3 is a scaffolding subunit (van Zyl et al., 1992). The Cdc55 and Rts1 regulatory subunits are mutually exclusive and direct PP2As to distinct cellular processes (Healy et al., 1991; Ronne et al., 1991; Shu et al., 1997; Sneddon et al., 1990). The Ppm1 methyltransferase methylates and activates PP2A catalytic subunits (Wei et al., 2001). Sit4 is a PP2A-like phosphatase (Sutton et al., 1991) that interacts with four activators known as Saps (Sit4 associating proteins) (Luke et al., 1996). Pph21/Pph22, as well as Sit4, physically interact with Tap42, a target of the TOR complex 1 (TORC1) (Di Como and Arndt, 1996). Rrd2 and Rrd1 are PP2A and PP2A-like activators (Jordens et al., 2006; Rempola et al., 2000; Zheng and Jiang, 2005). Tip41 inhibits Tap42 (Jacinto et al., 2001) and their interaction is influenced by Ptc1 phosphatase (Gonzalez et al., 2009). The PP2A/PP2A-like signaling pathways are extremely complex and presently not fully characterized (Duvel et al., 2003). According to the conventional model, TORC1 phosphorylates Tap42 that inhibits PP2As; PP2A mediates the dephosphorylation of TORC1 effectors (Loewith and Hall, 2011). Gln3, Npr1, Nnk1 and Rtg3 are PP2A targets involved in nitrogen and amino acid metabolism (Hughes Hallett et al., 2014). In humans, PP2A influences the ATM-dependent DDR (Freeman and Monteiro, 2010).

UV-radiation generates thymine dimers that are repaired by Nucleotide Excision Repair (NER) (Marteijn et al., 2014). The ATR-mediated DNA damage response (DDR) is activated by UV, through the accumulation of RPA-single stranded (ss) DNA nucleofilaments originating when NER enzymes process the DNA lesions (Neecke et al., 1999). In yeast, Mec1^{ATR}, once active, phosphorylates the Rad53^{Chk2} kinase (Tvegard et al., 2007). UV-radiation also causes protein damage (Krisko and Radman, 2013).

DDR and NER defective cells undergo premature aging and cancer predisposition (Marteijn et al., 2014). Saccharomyces cerevisiae cells experience replicative and chronological aging (Kaeberlein, 2010). Chronological aging refers to the decreased ability of non-dividing cells to survive and re-enter the cell cycle over time (Longo et al., 2012; Steinkraus et al., 2008) and serves as a more accurate model system for post-mitotic mammalian cell aging than replicative aging.

Genetic and pharmacological interventions extending chronological lifespan (CLS) have been identified (Fontana et al., 2010). Rapamycin, an immunosuppressant with potential in cancer therapy, and metformin, an anti-type II diabetes treatment, represent two well-characterized compounds that prolong lifespan. Both drugs target pathways that are highly implicated in chronological aging: Tor1 is specifically inhibited by rapamycin and AMPK is activated by metformin (Harrison et al., 2009; Martin-Montalvo et al., 2013; Powers et al., 2006). While mutations of TOR1 extend CLS, AMPK in mammals and its yeast ortholog Snf1 are required for longevity (Bonawitz et al., 2007; Lu et al., 2011; Yao et al., 2015).

TORC1 stimulates global translation by phosphorylating its downstream targets Sch9^{S6K/AKT}, a major regulator of ribosome biogenesis and translation initiation, and by regulating Eap1^{4E-BP}, an inhibitor of CAP-dependent translation (Cosentino et al., 2000; Urban et al., 2007). Mutations of SCH9 extend both CLS and RLS (Fabrizio et al., 2001; Selman et al., 2009). TORC1-mediated regulation of protein translation is further reinforced by its crosstalk with the Gcn2-eIF2α-Gcn4 axis, a pathway that, under stress conditions, such as nutrient starvation and UV irradiation, stimulates specialized translation (Hinnebusch, 2005). Following Gcn2-dependent phosphorylation of eIF2α, Gcn4, a transcription factor needed for survival in stress condition, is translated by a mechanism that relies on the presence of short open reading frames (uORFs) in its 5′UTR (Hinnebusch, 2005). uORFs have been identified in the 5′UTRs of genes involved in NER in mammalian cells, leading to their specific translation during UV exposure (Powley et al., 2009). The Gcn2-eIF2α-Gcn4 pathway has been implicated in replicative and chronological aging (Hussain and Ramaiah, 2007; McCormick et al., 2015; Steffen et al., 2008; Vlanti et al., 2013). While Tor1 activates Sch9, it represses the phosphatase PP2A-Tap42 complex, a highly conserved phosphatase involved in metabolic processes, transcription and translation (Zabrocki et al., 2002). Yeast PP2A is positively-regulated by the activator gene RRD1 and by Tip41, a Tap42 interacting protein (Jacinto et al., 2001; Rempola et al., 2000). Some PP2A targets have been identified and include Gln3, Nnk1 and Npr1, all of which are components of the TOR pathway and are dephosphorylated in a PP2A-dependent manner (Hughes Hallett et al., 2014; Loewith and Hall, 2011; Tate et al., 2009). Although PP2A has been mainly studied in the context of TOR pathway, several reports have linked this phosphatase complex with DDR (Bazzi et al., 2010; Keogh et al., 2006; Leroy et al., 2003; O'Neill et al., 2007; Szyjka et al., 2008). In addition to TOR, which primarily responds to nitrogen and amino acid levels, the AMP-activated kinase (AMPK in mammals, Snf1 in yeast) is pivotal for sensing energy status of the cell (Hedbacker and Carlson, 2008). A series of 3 highly conserved kinases, Elm1, Sak1 and Tos3, phosphorylate Snf1 on a single residue, Thr 210, leading to its activation. Adaptation to glucose limitation, transcription of metabolic genes and regulation of key metabolic enzymes are among the diverse roles of Snf1 AMPK Snf1 is implicated in aging in many model organisms (Lu et al., 2011; Weinberger et al., 2010; Yao et al., 2015). Metformin, which is known to activate AMPK, as well as glucose deprivation, both extend lifespan, consistent with the fact that the Snf1-mediated pathway is pro-longevity (Burtner et al., 2009; Martin-Montalvo et al., 2013; Smith et al., 2007). A crosstalk has been reported between Tor1 and AMPK (Hardie, 2014). A recent study has extended the relation between Snf1 and Tor1 to be context- and stress-dependent contingent with the survival requirement of the cell (Hughes Hallett et al., 2014). Common to both TOR and AMPK pathways is their link to eIF2α: while the major downstream target of Tor1, Sch9, antagonizes eIF2a phosphorylation to promote global translation initiation (Urban et al., 2007), Snf1 promotes phospho-eIF2a by its inhibitory action on Glc7, a direct phosphatase of eIF2α (Cherkasova et al., 2010). In addition to the established link between TORC1 and PP2A, Snf1 has also been connected to PP2A (Gimeno-Alcaniz and Sanz, 2003). TORC1 and AMPK regulate protein homeostasis not only through their influence on protein translation, but also since they both impinge on autophagy, a catabolic process involved in protein and organelle degradation in response to stress (Chen and Klionsky, 2011). Intriguingly, an AMPK-TORC1-ATM-mediated cytoplasmic pathway has been identified for survival in mammalian cells exposed to oxidative stress by inducing autophagy (Alexander et al., 2010). Autophagy is not only needed to survive nutritional stress, but also required for survival following DNA damage (Dotiwala et al., 2013; Eapen and Haber, 2013) as well as to promote longevity (Alvers et al., 2009a; Alvers et al., 2009b; Aris et al., 2013). A connection between the autophagy pathway and DDR has been described (Robert et al., 2011). Increase in glycolysis in high oxygen conditions represents the first tumor-specific metabolic alteration which drives cell proliferation through an increase of cell bioenergetics and of biosynthetic pathways. Clinically, impaired metabolism, obesity and hyperglycemia play important roles in cancer development, progression and prognosis, and indeed diabetic and obese patients have an increased risk of developing certain types of cancer (Cohen and LeRoith, 2012; Garg et al., 2014). Furthermore, tumors with high glucose uptake show a worsened prognosis (Gatenby and Gillies, 2004). In the past few years, new strategies aimed to tackle metabolic alterations in cancer are gaining greater attention. Dietary limitation through caloric restriction or intermittent fasting is an emerging approach to target tumor metabolism that has been shown to protect against tumorigenesis and to enhance the response to chemotherapeutics (Lee et al., 2012; Longo and Mattson, 2014). Caloric restriction (CR) has been shown to be a potent inhibitor of tumor growth (Qiu et al., 2010), but its clinical use is complicated by several factors (Lee and Longo, 2011). Conversely, intermittent fasting (IF) which refers to a limited time of exposure to a severely restricted diet, can protect yeast, mammalian cells, mice and potentially patients from the toxic effects of oxidative and chemotherapeutic agents without causing chronic weight loss, making it a clinically safer approach. IF has also been shown to selectively protect normal cells but not tumor cells against oxidants and common chemotherapeutic agents (Lee and Longo, 2011) and to protect tumor-bearing mice from toxicity induced by some chemotherapeutic agents with remarkable improvement in survival (Raffaghello et al., 2008). In cancer patients, IF has been shown to be safe, feasible and effective in reducing common side-effects associated with chemotherapy (Safdie et al., 2009); (Articles, 2007). Another attractive approach to target tumor metabolism has emerged after recent reports showing that metformin, the most widely used anti-diabetic drug for Type 2 diabetes (T2D), exhibits anti-cancer activities (Dougan et al., 2005). Several lines of evidence drawn from in vitro, in vivo and epidemiological studies now support the suggested anti-neoplastic properties of metformin in several types of cancer including breast, colon, ovary, pancreas, lung and prostate cancers (Anisimov, 2014; Gandini et al., 2014; Kasznicki et al., 2014; Pollak, 2012; Suissa and Azoulay, 2012). Given the favorable safety profile of metformin concluded from extensive safety data after decades of use as a diabetic medication, several clinical trials are now under way to further explore its therapeutic potential of use in cancer as an adjuvant together with other therapeutic approaches (Pollak, 2012). A warning against hasty generalizations on metformin as a cancer drug recently came from a preliminary study that showed that metformin accelerates the growth of BRAF-mutant melanoma cells in vivo, through activation of VEGF (Martin et al., 2012) which may represent an adaptive response of cancer cells to metformin. A dual effect of metformin on breast cancer cell proliferation has also been shown in initial clinical studies (Bonanni et al., 2012; DeCensi et al., 2014). This would therefore suggest that metformin treatment can also be associated with tumor promoting effects and its use as a bona fide anti-neoplastic drug may require careful patient stratification. Optimization of the clinical use of metformin in cancer would therefore benefit from a better understanding of how it exerts its anti-neoplastic effects. The molecular mechanisms of anti-proliferative actions of metformin, however, remain poorly understood. Particularly, it is not clear whether metformin mainly acts directly on cancer cells possibly by exploiting specific metabolic vulnerabilities in them or indirectly through mechanisms that involve alterations of the host environment. Several studies that examined direct effects of metformin on cancer cells in vitro suggested that metformin exerts direct anti-proliferative effects. However, in many of those studies anti-proliferative activity of metformin was observed at very high exposure levels raising doubts about the clinical relevance of these findings. Mechanistically, direct effects of metformin on cancer cells have been proposed-according to the most widely accepted model of action- to involve the induction of a transient drop in cellular energy by inhibiting respiratory chain complex I in the mitochondrion leading to activation of the energy sensor AMPK by its kinase, the tumor suppressor LKB 1 (Pollak, 2012). As a consequence, activated AMPK in turn fuels a cascade of anti-proliferative effects among which, the best known is the inhibition of the mTOR pathway (Laplante and Sabatini, 2012). Another potential anti-proliferative effect that can contribute to the anti-neoplastic properties of metformin is its systemic action on blood glucose and insulin levels. Through its activity on hepatocytes, metformin can decrease hepatic glucose secretion and ultimately decreases serum insulin, a known mitogen for cancer cells (Dowling et al., 2012).

Due to the complexity of the above described pathways, there is still the need to identify molecular targets implicated in DDR in order to treat pathologies related to DDR.

### SUMMARY OF THE INVENTION

Mec1^{ATR} mediates the DNA damage response (DDR) integrating chromosomal signals and mechanical stimuli. Inventors show that the PP2A phosphatases, ceramide activated enzymes, couple cell metabolism with DDR. Using genomic screens, metabolic analysis, genetic and pharmacological studies inventors found that PP2A attenuates DDR and that three metabolic circuits influence DDR by modulating PP2A activity. Irc21, a putative cytochrome b5 reductase that promotes the condensation reaction generating dihydroceramides, and Ppm1, a PP2A methyltransferase, counteract DDR by activating PP2A. In particular, inventors herein show that Irc21, a cytochrome b₅-like enzyme influencing genome stability (Alvaro et al., 2007; Gallego et al., 2010; Guenole et al., 2013; Lee et al., 2005) activates PP2A by promoting the synthesis of dihydroceramides (DHC) and that PP2A is a central hub in a regulatory loop that couples three metabolic pathways dependent on Irc21, Ppm1 and TORC1, with the ATR-mediated DNA damage response (DDR). Conversely, the nutrient sensing TORC1-Tap42 axis sustains DDR activation by inhibiting PP2A. Loss of function mutations in IRC21, PPM1 and PP2A and hyperactive tap42 alleles rescue mec1 mutants. Ceramides synergize with rapamycin, a TORC1 inhibitor, in counteracting DDR. Hence, PP2A integrates nutrient sensing and metabolic pathways to attenuate the Mec1^{ATR} response. Present observations imply that metabolic changes impact on genome integrity and may help in exploiting novel therapeutic options and repositioning known drugs. Moreover, a shift towards increased glycolysis is a signature of tumors. Inventors' observations, along with emerging reports however suggest that tumors show increased metabolic plasticity, alternating between dependency on glycolysis or oxidative phosphorylation (OXPHOS) to adapt to microenvironmental changes. Targeting one specific metabolic pathway could thus be ineffective. Here, inventors designed a more efficient approach to target tumor metabolism by combining intermittent fasting as a clinically-feasible approach to reduce glucose availability together with OXPHOS inhibitor metformin. In mice exposed to 24-hour feeding/fasting cycles, metformin impaired tumor growth only when administered during hypoglycemia periods (fasting cycles). Synergistic cytotoxicity between metformin and hypoglycemia was independent of AMPK but was mediated by activation of GSK30 downstream of the tumor-suppressor PP2A leading to decline in the levels of the pro-survival MCL-1 and cell death. Mechanistically, the specific activation of PP2A-GSK3β axis by the combination is the sum of metformin-induced inhibition of CIP2A, a PP2A suppressor, together with low glucose-induced upregulation of PP2A regulatory subunit B56δ. Both events simultaneously result in the formation of an active PP2A-B56δ complex which shows high affinity towards GSK303. Collectively, inventors describe a novel approach for targeting tumor metabolic plasticity by hypoglycemia-metformin combination which may offer a novel therapeutic strategy for immediate clinical testing. With the weak direct anti-proliferative effects of clinically-relevant doses of metformin on cancer cells, approaches to selectively enhance metformin's anti-neoplastic properties are important. In the present study, inventors examined the effect of targeting tumor metabolism by a combination of intermittent fasting and metformin. Survival to UV-induced DNA lesions relies on nucleotide excision repair (NER) and the ATR^{Mec1} DNA damage response (DDR). Inventors studied DDR and NER during chronological aging, following UV and found that old cells fail to efficiently repair DNA and activate ATR. Inventors employed pharmacologic, genetic and mechanistic approaches to rescue DDR and repair efficiency during aging. AMPK^{snf1} activation conditions, such as metformin, low glucose or hyper-active mutants, promote DDR efficiency, but not NER, specifically in old cells by restraining PP2A activity that neutralizes the Gcn2 kinase. Inhibition of the Torc1-S6K^{Sch9} axis by rapamycin or genetic tools, enhances DDR and NER, specifically during aging, by counteracting PP2A and stimulating autophagy. The inability of AMPK activating cells to rescue NER in old cells depends on Torc1-mediated NER inhibition. Hence, DDR and NER are suppressed during aging by metabolic inputs sustaining Torc1 activity and restraining AMPK activation and PP2A is detrimental for old cell survival to UV damage. Inventors herein characterized DDR and NER during aging, following UV radiation and found that old cells fail to efficiently repair DNA and activate Mec1. Using pharmacologic, genetic and mechanistic approaches to rescue DDR and repair efficiency in aging cells inventors found that AMPK^{Snf1} activation conditions promote DDR efficiency, specifically in old cells. Conversely, NER is not improved. AMPK acts by restraining PP2A activity that neutralizes the Gcn2 kinase. On the other hand, inhibition of the Tore 1-S6K^{Sch9} axis enhances both DDR and NER, specifically in old cells, through a process dependent on PP2A and ATG1, the master regulator of autophagy. The inability of AMPK activating cells to rescue NER in old cells is due to Torc1 activity, which suppresses NER efficiency. Altogether our data indicate that metabolic inputs sustaining Torc1 activity and restraining AMPK activation suppress DDR and NER, specifically during aging and pinpoint a detrimental function for PP2A in old cells exposed to UV-induced DNA damage. It was found that old cells experiencing UV radiation fail to repair DNA and to activate Mec1. Further, stimulation of AMPK^{Snf1} pathway promotes DDR, but not NER, by counteracting PP2A activity in old cells. Moreover, inhibition of TORC1-S6K^{Sch9} axis improves DDR and NER in aging by restraining PP2A activity and stimulating autophagy. PP2A mediates the cross-talk between Torc1 and Snf1 and is detrimental for cell survival in aging.

### DETAILED DESCRIPTION OF THE INVENTION

In the present invention the authors gained evidences that diseases characterized by an alteration in the DNA damage response, such as cancer, may be treated with the combination of DNA damaging agents such as hydorxyurea, Gemcitabine, Carboplatin and platin-based drugs, camptotechin, topoisomerase inhibitors, and other chemotherapic drugs+PP2A activation and/or the combination of metabolic/dietary and/or pharmacological approaches+PP2A activation. Said PP2A activation includes the assembly of specific PP2A holoenzyme complexes.

Further it was also found that disease caused by excessive cell death due to altered DDR may be controlled by inhibitors of PP2A: among those, aging, aging-associated diseases, neurodegenerative diseases. Such diseases can be prevented and/or treated by drugs or interventions that counteract TORC1-PP2A. Activation of PP2A induces cell death, thus is beneficial for cancer treatment. PP2A inhibition can protect from diseases caused by excessive cell death/deregulation of DDR (aging, aging-associated diseases, degenerative diseases).

The present invention provides at least one modulator of PP2A or at least one modulator of PP2A-like phosphatase or at least one modulator of PP2A and PP2A-like phosphatase or a combination of said modulators for use in the prevention and/or treatment of a disease characterized by an alteration in the DNA damage response.

Therefore, the object of the present invention is at least one modulator of PP2A and/or of PP2A-like phosphatase or a combination thereof for use in the prevention and/or treatment of a disease characterized by an alteration in the DNA damage response (DDR).

Preferably, the at least one modulator or combination thereof modulates the PP2A-GSK3β-MCL-1 axis. Preferably, said modulator is selected from the group consisting of:

a) a small molecule;

b) a polypeptide;

c) an antibody or a fragment thereof;

d) a polynucleotide coding for said antibody or polypeptide or a functional derivative thereof;

e) a polynucleotide, such as antisense construct, antisense oligonucleotide, RNA interference construct or siRNA,

f) a vector comprising or expressing the polynucleotide as defined in d) or e);

g) a host cell genetically engineered expressing said polypeptide or antibody or comprising the polynucleotide as defined in d) or e).

Preferably, the above defined modulator is selected from the group consisting of: a TORC1 inhibitor, a Ppm1 methyltransferase activator, a TOR inhibitor or wherein said modulator is an intervention and/or an agent that inhibits nutrient uptake (inhibition of nutrient uptake). More preferably, the ceramide is selected from the group consisting of: N-Acetyl-D-sphingosine c2 ceramide, C6-Ceramide, ceramidase inhibitor, such as: D-e-MAPP and D-NMAPPD (B13). Ceramidase inhibitor D-e-MAPP and D-NMAPPD (B13) both increase ceramide levels in cells. N-Acetyl-D-sphingosine is a cell-permeable and biologically active ceramide. Other ceramides may be used for instance as described in: https://www.ncbi.nlm.nih.gov/pubmed/14657198 J Lipid Res. 2004 March; 45(3):496-506. The structural requirements for ceramide activation of serine-threonine protein phosphatases. Chalfant C E1, Szulc Z, Roddy P, Bielawska A, Hannun Y A, incorporated by reference; https://www.ncbi.nlm.nih.gov/pubmed/21062159 Future Oncol. 2010 October; 6(10):1603-24. doi: 10.2217/fon.10.116. Sphingolipids and cancer: ceramide and sphingosine-1-phosphate in the regulation of cell death and drug resistance. Ponnusamy S1, Meyers-Needham M, Senkal C E, Saddoughi S A, Sentelle D, Selvam S P, Salas A, Ogretmen B, incorporated by reference; https://www.ncbi.nlm.nih.gov/pubmed/8393446, J Biol Chem. 1993 Jul. 25; 268(21):15523-30. Ceramide activates heterotrimeric protein phosphatase 2A. Dobrowsky R T1, Kamibayashi C, Mumby M C, Hannun Y A, incorporated by reference.

The TORC1 inhibitor preferably inhibits the TORC1-Tap42 pathway.

In the present invention, the modulator is preferably selected from the group consisting of: metformin (NH₂C(═NH)NHC(═NH)N(CH₃)₂), thioridazine (C₂₁H₂₆N₂S₂), perphenazine (C₂₁H₂₆ClN₃OS), ceramide, Irc21, rapamycin (C₅₁H₇₉NO₁₃), caffeine (C₈H₁₀N₄O₂), wortmannin (C₂₃H₂₄O₈), S-adenosyl methionine (C₁₅H₂₃ClN₆O₅S), FTY-720 (C₁₉H₃₃NO₂), fluphenazine (C₂₂H₂₆F₃N₃OS), thiethylperazine, pimozide (C₂₈H₂₉F₂N₃O), clozapine (C₁₈H₁₉ClN₄), loratadine (C₂₂H₂₃N₂O₂Cl), promethazine (C₁₇H₂₀N₂S), haloperidol (C₂₁H₂₃ClFNO₂), mersalyl acid (HOHgCH₂CH(OCH₃)CH₂NHCOC₆H₄OCH₂CO₂H), myriocin (C₂₁H₃₉NO₆), fumonisin B1 (C₃₄H₅₉NO₁₅), okadaic acid (C₄₄H₆₈O₁₃), cardiolipin (C₆₅H₁₆Na₂O₁₇P₂), thiethylperazine maleate (C₂₂H₂₉N₃S2-2C₄H₄O₄).

In the present invention, the at least one modulator or combination thereof as above defined is preferably used in combination with low glucose and/or with at least one DNA damaging agent.

Preferably, said DNA damaging agent is at least one agent selected from the group consisting of: hydroxyurea, gemcitabine, carboplatin, platin-based drug, camptotechin, topoisomerase inhibitors and other chemoterapic drugs. In these preferred embodiments, the diseases to be preferably treated is cancer.

The at least one modulator or combination thereof as above defined is preferably used in combination with an inhibitor of glycosidase and/or an inhibitor of amylase. In the present invention the combination is preferably of perphenazine and metformin; metformin and thioridazine; metformin and fasting; metformin and intermittent fasting; metformin and fasting mimicking diets; metformin and any form of fasting and at least one compound selected from table 1B such as fluphenazine, thiethylperazine, pimozide, clozapine, loratadine, promethazine, haloperidol, metformin and 2-Deoxy-Glucose; metformin and rapamycin; metformin and amylases and/or glycosidases inhibitors, such as acarbose, quercetin, 5,4′-dihydroxy-3,7-dimethoxyflavone, flavone luteolin, luteolin-7-O-glucoside, eupafolin. In a preferred embodiment, when the modulator (e.g. metformin) is combined with treatments that lead to a decrease in glucose levels, said modulator is used in treating the patient.

Preferably, the disease characterized by an alteration in the DNA damage response is a cancer and the modulator is an activator of PP2A and/or of PP2A-like phosphatase. Still preferably, the activator is used in combination with low glucose and/or with at least one DNA damaging agent.

The PP2A activator is preferably a compound able to form an active PP2A holoenzyme comprising the regulatory subunit B56∂ or an activator that induces a PP2A holoenzyme that includes the B56ζ subunit, such as PPZ and Thioridazine, or an activator that needs low glucose (or fasting) and metformin to achieve the formation of an active PP2A holoenzyme that includes B56∂.

Preferably the activator of PP2A and/or of PP2A-like phosphatase is selected from the group consisting of: metformin, thioridazine, perphenazine, ceramide, Irc21, a Ppm1 methyltransferase activator, TORC1 inhibitor, rapamycin, caffeine, wortmannin, S-adenosyl methionine, FTY-720, fluphenazine, thiethylperazine, pimozide, clozapine, loratadine, promethazine, haloperidol, TOR inhibitors.

Preferably the cancer presents at least one defect in at least one DDR pathways gene e.g. cancer that can be scored for instance by the presence of inactivating mutations in components of the DDR, such as RAD51 and/or BRCA1/2 and/or other DDR factors (ATM; ATR; components of the HR, Homologous Recombination, and of the NHEJ (Non-Homologous End Joining) pathways of DDR).

Preferably the subjects to be treated in the present invention were previously stratified by analysis of DDR markers. For example, the subject is positive for inactivating mutations in e.g. RAD51 and/or BRCA1/2 and/or other DDR factors (examples: mutations in the coding sequence of the DDR factor, or indirect measurements of DDR such as gamma-H2AX).

Preferably, the disease characterized by an alteration in the DNA damage response ageing and/or ageing-associated disease and/or degenerative disease and the modulator is an inhibitor of PP2A and/or of PP2A-like phosphatase. Degenerative diseases include e.g. Parkinson disease, Alzheimer Disease, Pick's Disease, Progressive Supranuclear Palsy, Corticobasal Degeneration etc.

Preferably the inhibitor of PP2A and/or of PP2A-like phosphatase is selected from the group consisting of: mersalyl acid or a salt form thereof, myriocin, fumonisin B1, okadaic acid, cardiolipin.

Another object of the invention is an in vitro method to identify a subject to be treated with a PP2A modulator comprising detecting in the genome of said patient a mutation in PP2A (e.g. W257G of PPP2R1A, E64D of PPP2R1A, D540G ofPPP2R1B, C39R, E164K, Q256R, L257R ofPPP2R5C; note that several additional mutations have been identified in tumor patients, including exon deletions, splicing variants, as described in Ruvolo, P., (2016). The broken “Off” switch in cancer signaling: PP2A as a regulator of tumorigenesis, drug resistance, and immune surveillance. BBA Clin. 2016 December; 6: 87-99, incorporated by reference) or variations in its expression levels.

The invention provides an in vitro method to identify a subject to be treated with at least one modulator or combination thereof as defined above comprising detecting in the genome of said patient a mutation in PP2A and/or a mutation in PP2A-like phosphatase or measuring expression level variation of PP2A and/or PP2A-like phosphatase.

In particular, mutations in PP2A subunits that diminish the assembly or catalytic activities of PP2A holoenzyme may be detected by means know in the art. Any known methods in the art (sequencing etc.), including the ones described in the present invention may be used to detected said mutation in PP2A or in a PP2A-like phosphatase.

Measuring expression level variation of PP2A and/or PP2A-like phosphatase may be performed by any means known in the art. Expression level variation is measured by comparing to a proper control that may be the level present in a health subject, the level measured before initiation of the treatment with the modulator or the levels measured after treatment with a gold standard.

Preferably said patient is resistant to treatment with metformin.

Another object of the invention is an in vitro method to identify a subject to be treated with a modulator of PP2A and/or of PP2A-like phosphatase or combination thereof comprising detecting in the genome of said patient at least one mutation in at least one DDR pathways gene, e.g. in RAD51 and/or BRCA1/2 and/or other DDR factors.

Another object of the invention is a pharmaceutical composition comprising at least one modulator or combination thereof as above defined and at least one pharmaceutically acceptable carrier, preferably further comprising a therapeutic agent, more preferably the therapeutic agent in an anti-tumoral agent or an anti-ageing agent.

Further objects of the invention are the above pharmaceutical composition for use in the prevention and/or treatment of a disease characterized by an alteration in the DNA damage response (DDR); a method for the prevention and/or treatment of a disease characterized by an alteration in the DNA damage response (DDR). comprising administering to a subject at least one modulator or combination thereof as above defined; the at least one modulator or combination thereof as above described, in combination with a therapeutic agent.

In the present invention, cancer can be of any subtype, such as e.g. colon cancer. cervical cancer, breast cancer, ovarian cancer, melanoma, lung cancer, pancreatic cancer, neuroendocrine tumors and acute myeloid leukemia.

In the present invention “an alteration” in the DNA damage response, is an alteration in the ability to remove or correct errors in DNA. Such alteration may be caused by:
Mutations in DNA damage factors that cause a defective response (DNA damage response is inactive/defective but required)
Mutations in DNA damage factors that lead to abnormal activation of the response (DNA damage response is active but not required).

In the present invention a modulator may be an inhibitor or an activator of PP2A and/or of PP2A-like phosphatase.

Preferably said modulator modulates the PP2A-GSK33-MCL-1 axis.

The PP2A-GSK3β-MCL-1 axis includes the following molecular events in mammalian cells: the dephosphorylation of Glycogen synthase kinase 3 beta (GSK3β) by PP2A leading to decline in myeloid leukemia cell differentiation protein (MCL-1).

In the context of the present invention, the inhibition of nutrient uptake may be achieved by agents that reduce availability of intracellular nutrients via inhibiting nutrient influx through transporters, receptors, and micropinocytosis or via inhibition of autophagy. Either deprivation of food or inhibition of complex carbohydrates demolition operated in saliva and pancreatic secretions, results in a lower glucose level in the bloodstream.

The term “glycosidases” indicates a family of carbohydrates digesting enzymes.

Amylases and glycosidases enzymes are responsible for the digestion of oligosaccharides and disaccharides to monosaccharides such as glucose or maltose. Intestinal villi are not able to uptake and delivery into the bloodstream complex saccharides. Inhibition of these enzymes retard the absorption of carbohydrates resulting in a lower postprandial plasma glucose level. (Paloma Michelle de Sales, Paula Monteiro de Souza, Luiz Alberto Simeoni, Pérola de Oliveira Magalhães, Dâmaris Silveira. α-Amylase Inhibitors: A Review of Raw Material and Isolated Compounds from Plant Source. J Pharm Pharmaceut Sci 15(1) 141-183, 2012; Natércia F Brás, Nuno MFSA Cerqueira, Maria J Ramos & Pedro A Fernandes Glycosidase inhibitors: a patent review (2008-2013). Expert Opinion on Therapeutic Patents Volume 24, 2014—Issue 8) In the context of the present invention, low glucose is achieved when reducing glucose availability in the serum or intracellularly markedly below the normal levels (by 40-60%).

Yeast range for low glucose conditions: 0.05%-0.5%.

α-amylases inhibitors isolated from wheat and white bean significantly reduced the peak of postprandial glucose. (Lankisch M, Layer P, Rizza R A, DiMagno E P. Acute postprandial gastrointestinal and metabolic effects of wheat amylase inhibitor (WAI) in normal, obese, and diabetic humans. Pancreas, 1998; 17: 176-181. Boivin M, Flourie B, Rizza R A, Go V L, DiMagno E P. Gastrointestinal and metabolic effects of amylase inhibition in diabetics. Gastroenterology, 1988; 94: 387-394.)

Still preferably the at least one modulator or combination thereof is used in combination with an inhibitor of glucosidase and/or an inhibitor of amylase.

Glycosidase and/or amylase inhibitors, including flavonoids, tannins and terpenoids, share a basic structure of polyphenolic rings that are able to interact with amylases and glycosidases catalytic sites resulting in a strong hydrogen bound. Most of the glycosidase inhibitors are carbohydrate mimics in which one atom in the monosaccharide main scaffold is changed. De facto sequestrating the enzymes and inhibiting their sugar demolition activity.

In the present invention, fasting is equivalent to dietary limitation and includes also caloric restriction in addition to total fasting and intermittent fasting.

Intermittent fasting refers to a limited time of exposure to a severely restricted diet. Caloric restriction refers to a 20-40% restriction of usual calorie intake.

Fasting is a complete deprivation of food but not water for a window time of 24 hrs (or shorter duration in humans).

An activator of PP2A and/or of PP2A-like phosphatase induces activation of PP2A and/or PP2A-like phosphatase. It may: induce the assembly of an active PP2A holoenzyme that modulates the phosphorylation of downstream targets; it may bind to components of the PP2A holoenzyme and activates it; it may bind to inhibitors of PP2A and inhibits them; it may lead to expression of specific subunits of PP2A that modulate its activity. Preferably, said activators would lead to incorporation of the B560 regulatory subunit (or other subunits able to regulate the PP2A-GSK3ß-Mcl1 axis), and downregulation of the CIP negative regulator of PP2A activity (or equivalent molecules such as SET).

Activation of PP2A or PP2A-like phosphatase may be detected or measured according to known methods in the art, including the one described in the present invention.

An inhibitor (which includes also inactivators) of PP2A and/or of PP2A-like phosphatase may bind to the catalytic site and inhibits enzymatic activity; may disrupt the assembly of an active PP2A holoenzyme; it may also bind to activators of PP2A and inhibits them.

Inhibition or inactivation of PP2A or/and PP2A-like phosphatase may be detected or measured according to known methods in the art, including the one described in the present invention.

The expression “molecule able to modulate” and “modulator” are herein interchangeable. By the term “modulator” it is meant a molecule that effects a change in the expression and/or function of at least one marker as above defined.

The change is relative to the normal or baseline level of expression and/or function in the absence of the modulator, but otherwise under similar conditions, and it may represent an increase (e.g. by using an inducer or activator) or a decrease (e.g. by using a suppressor or inhibitor) in the normal/baseline expression and/or function. In the context of the present invention, a “modulator” may be a molecule which suppresses or inhibits the expression and/or function of PP2A and/or of PP2A-like phosphatase for use in the prevention and/or treatment of ageing and/or ageing-associated disease and/or degenerative disease.

By the term “suppressor or inhibitor” or a “molecule which (selectively) suppresses or inhibits” it is meant a molecule that effects a change in the expression and/or function of the target.

In the context of the present invention, a “modulator” may be a molecule which induces or activates the expression and/or function of PP2A and/or of PP2A-like phosphatase for use in the prevention and/or treatment of cancer.

The change is relative to the normal or baseline level of expression and/or function in the absence of the modulator, but otherwise under similar conditions, and it may represent an increase (e.g. by using an inducer or activator) or a decrease (e.g. by using a suppressor or inhibitor) in the normal/baseline expression and/or function.

The suppression or inhibition of the expression and/or function of the target may be assessed by any means known to the skilled in the art. The assessment of the expression level or of the presence of the target is preferably performed using classical molecular biology techniques such as (real time Polymerase Chain Reaction) qPCR, microarrays, bead arrays, RNAse protection analysis or Northern blot analysis or cloning and sequencing.

The assessment of target function is preferably performed by in vitro suppression assay, whole transcriptome analysis, mass spectrometry analysis to identify proteins interacting with the target, assays of biochemical activity against natural/synthetic substrates, on immunoprecipitated PP2A-containing complexes.

The above described molecules also include salts, solvates or prodrugs thereof. The above described molecules may be or not solvated by H₂O.

In the context of the present invention, the target of the modulator or the modulator may be intended as the gene, the mRNA, the cDNA, or the encoded protein thereof, including functional fragments, derivatives, variants, isoforms, etc. Preferably, the target of the modulator or the modulator are characterized by or comprise the sequences identified by their NCBI Accession numbers (see Table 5). In particular, in a preferred embodiment PP2A comprises at least one of the subunits identified by the NCBI Accession numbers of Table 5.

The term gene herein also includes corresponding orthologous or homologous genes, isoforms, variants, allelic variants, functional derivatives, functional fragments thereof. The expression “protein” is intended to include also the corresponding protein encoded from a corresponding orthologous or homologous genes, functional mutants, functional derivatives, functional fragments or analogues, isoforms thereof.

In the context of the present invention, the term “polypeptide” or “protein” includes:

i. the whole protein, allelic variants and orthologs thereof;

ii. any synthetic, recombinant or proteolytic functional fragment;

iii. any functional equivalent, such as, for example, synthetic or recombinant functional analogues.

In the present invention “functional mutants” of the protein are mutants that may be generated by mutating one or more amino acids in their sequences and that maintain their activity. Indeed, the protein of the invention, if required, can be modified in vitro and/or in vivo, for example by glycosylation, myristoylation, amidation, carboxylation or phosphorylation, and may be obtained, for example, by synthetic or recombinant techniques known in the art. The term “derivative” as used herein in relation to a protein means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. As used herein, the term “derivatives” also refers to longer or shorter polypeptides having e.g. a percentage of identity of at least 41%, preferably at least 41.5%, 50%, 54.9%, 60%, 61.2%, 64.1%, 65%, 70% or 75%, more preferably of at least 85%, as an example of at least 90%, and even more preferably of at least 95% with the herein disclosed genes and sequences, or with an amino acid sequence of the correspondent region encoded from orthologous or homologous gene thereof. The term “analogue” as used herein referring to a protein means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A “derivative” may be a nucleic acid molecule, as a DNA molecule, coding the polynucleotide as above defined, or a nucleic acid molecule comprising the polynucleotide as above defined, or a polynucleotide of complementary sequence. In the context of the present invention the term “derivatives” also refers to longer or shorter polynucleotides and/or polynucleotides having e.g. a percentage of identity of at least 41%, 50%, 60%, 65%, 70% or 75%, more preferably of at least 85%, as an example of at least 90%, and even more preferably of at least 95% or 100% with sequences herein mentioned or with their complementary sequence or with their DNA or RNA corresponding sequence. The term “derivatives” and the term “polynucleotide” also include modified synthetic oligonucleotides. The modified synthetic oligonucleotide is preferably LNA (Locked Nucleic Acid), phosphoro-thiolated oligos or methylated oligos, morpholinos, 2′-O-methyl, 2′-O-methoxyethyl oligonucleotides and cholesterol-conjugated 2′-O-methyl modified oligonucleotides (antagomirs). The term “derivative” may also include nucleotide analogues, i.e. a naturally occurring ribonucleotide or deoxyribonucleotide substituted by a non-naturally occurring nucleotide. The term “derivatives” also includes nucleic acids or polypeptides that may be generated by mutating one or more nucleotide or amino acid in their sequences, equivalents or precursor sequences. The term “derivatives” also includes at least one functional fragment of the polynucleotide. In the context of the present invention “functional” is intended for example as “maintaining their activity”. As used herein “fragments” refers to polynucleotides having preferably a length of at least 1000 nucleotides, 1100 nucleotide, 1200 nucleotides, 1300 nucleotides, 1400 nucleotides, 1500 nucleotides or to polypeptide having preferably a length of at least 50 aa, 100 aa, 150 aa, 200 aa, 250 aa, 300 aa . . . . The term “polynucleotide” also refers to modified polynucleotides. As used herein, the term “vector” refers to an expression vector, and may be for example in the form of a plasmid, a viral particle, a phage, etc. Such vectors may include bacterial plasmids, phage DNA, baculovirus, yeast plasmids, vectors derived from combinations of plasmids and phage DNA, viral DNA such as vaccinia, adenovirus, lentivirus, fowl pox virus, and pseudorabies. Large numbers of suitable vectors are known to those of skill in the art and are commercially available. The polynucleotide sequence, preferably the DNA sequence in the vector is operatively linked to an appropriate expression control sequence(s) (promoter) to direct mRNA synthesis. As representative examples of such promoters, one can mention prokaryotic or eukaryotic promoters such as CMV immediate early, HSV thymidine kinase, early and late SV40, LTRs from retrovirus, and mouse metallothionein-I. The expression vector may also contain a ribosome binding site for translation initiation and a transcription vector. The vector may also include appropriate sequences for amplifying expression. In addition, the vectors preferably contain one or more selectable marker genes to provide a phenotypic trait for selection of transformed host cells such as dihydro folate reductase or neomycin resistance for eukaryotic cell culture, or such as tetracycline or ampicillin resistance in E. coli. As used herein, the term “host cell genetically engineered” relates to host cells which have been transduced, transformed or transfected with the polynucleotide or with the vector described previously. As representative examples of appropriate host cells, one can cite bacterial cells, such as E. coli, Streptomyces, Salmonella typhimurium, fungal cells such as yeast, insect cells such as Sf9, animal cells such as CHO or COS, plant cells, etc. The selection of an appropriate host is deemed to be within the scope of those skilled in the art from the teachings herein. Preferably, said host cell is an animal cell, and most preferably a human cell. The introduction of the polynucleotide or of the vector described previously into the host cell can be effected by method well known from one of skill in the art such as calcium phosphate transfection, DEAE-Dextran mediated transfection, electroporation, lipofection, microinjection, viral infection, thermal shock, transformation after chemical permeabilisation of the membrane or cell fusion. The polynucleotide may be a vector such as for example a viral vector. The polynucleotides as above defined can be introduced into the body of the subject to be treated as a nucleic acid within a vector which replicates into the host cells and produces the polynucleotides. Suitable administration routes of the pharmaceutical composition of the invention include, but are not limited to, oral, rectal, transmucosal, intestinal, enteral, topical, suppository, through inhalation, intrathecal, intraventricular, intraperitoneal, intranasal, intraocular, parenteral (e.g., intravenous, intramuscular, intramedullary, and subcutaneous), chemoembolization. Other suitable administration methods include injection, viral transfer, use of liposomes, e.g. cationic liposomes, oral intake and/or dermal application. In certain embodiments, a pharmaceutical composition of the present invention is administered in the form of a dosage unit (e.g., tablet, capsule, bolus, etc.). For pharmaceutical applications, the composition may be in the form of a solution, e.g. an injectable solution, emulsion, suspension or the like. The carrier may be any suitable pharmaceutical carrier. Preferably, a carrier is used which is capable of increasing the efficacy of the molecules to enter the target cells. Suitable examples of such carriers are liposomes. In the pharmaceutical composition according to the invention, the suppressor or inhibitor may be associated with other therapeutic agents. The pharmaceutical composition can be chosen on the basis of the treatment requirements. Such pharmaceutical compositions according to the invention can be administered in the form of tablets, capsules, oral preparations, powders, granules, pills, injectable, or infusible liquid solutions, suspensions, suppositories, preparation for inhalation. A reference for the formulations is the book by Remington (“Remington: The Science and Practice of Pharmacy”, Lippincott Williams & Wilkins, 2000). The expert in the art will select the form of administration and effective dosages by selecting suitable diluents, adjuvants and/or excipients. Pharmaceutical compositions of the present invention may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention. For injection, including, without limitation, intravenous, intramusclular and subcutaneous injection, the compounds of the invention may be formulated in aqueous solutions, preferably in physiologically compatible buffers such as physiological saline buffer or polar solvents including, without limitation, a pyrrolidone or dimethylsulfoxide. The compounds are preferably formulated for parenteral administration, e.g., by bolus injection or continuous infusion. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water-soluble form, such as, without limitation, a salt of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use. For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). For administration by inhalation, the molecules of the present invention can conveniently be delivered in the form of an aerosol spray using a pressurized pack or a nebulizer and a suitable propellant. The molecules may also be formulated in rectal compositions such as suppositories or retention enemas, using, e.g., conventional suppository bases such as cocoa butter or other glycerides. In addition to the formulations described previously, the compounds may also be formulated as depot preparations. Such long acting formulations may be administered by implantation (for example, subcutaneously or intramuscularly) or by intramuscular injection. The compounds of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt. Additionally, the compounds may be delivered using a sustained-release system, such as semi-permeable matrices of solid hydrophobic polymers containing the therapeutic agent. Various sustained-release materials have been established and are well known by those skilled in the art. A therapeutically effective amount refers to an amount of compound effective to prevent, alleviate or ameliorate the protein conformational disease. Determination of a therapeutically effective amount is well within the capability of those skilled in the art, especially in light of the disclosure herein. Generally, the amount used in the treatment methods is that amount which effectively achieves the desired therapeutic result in mammals. In particular, the molecules administration should follow the current clinical guidelines. A suitable daily dosage will range from 0.001 to 10 mg/kg body weight, in particular 0.1 to 5 mg/kg. In the case of polynucleotides a suitable daily dosage may be in the range of 0.001 pg/kg body weight to 10 mg/kg body weight. Typically the patient doses for parenteral administration of the molecules described herein range from about 1 mg/day to about 10,000 mg/day, more typically from about 10 mg/day to about 1,000 mg/day, and most typically from about 50 mg/day to about 500 mg/day. The range set forth above is illustrative and those skilled in the art will determine the optimal dosing of the compound selected based on clinical experience and the treatment indication.

Preferably the at least one modulator or combination thereof or the pharmaceutical composition of the invention further comprises at least another therapeutic agent, preferably the other therapeutic agent is selected from the group of: anti-tumoral agent, anti-pain agent, anti-emetic agent (such as aprepitant, fosaprepitant, Dolasetron, granisetron, ondansetron, palonosetron, tropisetron, or ramosetron, Dexamethasone). Preferably the other therapeutic agent is selected from the group consisting of: chemotherapeutic agent or radioactive agents, ATR inhibitor, HR inhibitor, molecule that specifically target telomeres, preferably G-quadruplexes interacting molecules, molecule that cause DNA damage generation specifically at telomeres.

Additionally, administration of the modulators of the present invention may be administered concurrently with other therapies, e. g., administered in conjunction with a chemotherapy or radiation therapy regimen. In the present invention an ATR inhibitor is a small molecule compound able to inhibit the kinase activity of ATR, comprising but not limited to VE-821 (Vertex Pharmaceuticals), VE-822 (Vertex Pharmaceuticals), AZ20 (AstraZeneca), AZD6738 (AstraZeneca) (as described in Flynn et al, Science, 2015; Weber A M, Pharmacol Ther. 2015, all references are incorporated by reference). A HR inhibitor is any compound or experimental approach able to impair or inhibit the cellular process known as DNA repair by homologous recombination (HR), comprising but not limited to: Iniparib (SAR240550, BSI-201; Sanofi-Aventis), Olaparib (AZD2281, KU-0069436; AstraZeneca), Niraparib (Tesaro), Rucaparib (CO-338, AG-014699, PF-O1367338; Pfizer), Veliparib (ABT-888; Abbott), AZD2461 (AstraZeneca), BMN673 (BioMarin Pharmaceutical), CEP-9722 (Cephalon), E7016 (Esai), INO-1001 (Inotek Pharmaceuticals), MK-4827 (Merck), Methoxyamine (Sigma Aldrich), RI-1, IBR2, B02, Halenaquinone (described in Kelley M R, Future Oncol. 2014, Ward A, Cancer Treat Rev. 2015, Feng F Y, Mol Cell. 2015, all references are incorporated by reference).

A molecule that specifically targets and/or causes DNA damage generation at telomeres is any compound or experimental approach which specifically or preferentially interacts with telomeres, inducing DNA damage within telomeric DNA and/or activation or inhibition of DDR signalling and/or DNA repair, comprising but not limited to: G-quadruplex-binding ligands (e.g. BRACO-19, Telomestatin, RHPS4, Quarfloxin, TMPyP4, AS1410), topoisomerase inhibitors, cisplatin, hydroxyurea, (as described in Lu et al, Front. Med. 2013; Neidle FEBS J, 2010; Müller and Rodriguez, Expert Rev Clin Pharmacol. 2014; Sissi and Palumbo, Curr Pharm Des. 2014, Salvati et al, NAR, 2015, all references are incorporated by reference).

The present invention will be illustrated by means of non-limiting examples and figures as follows.

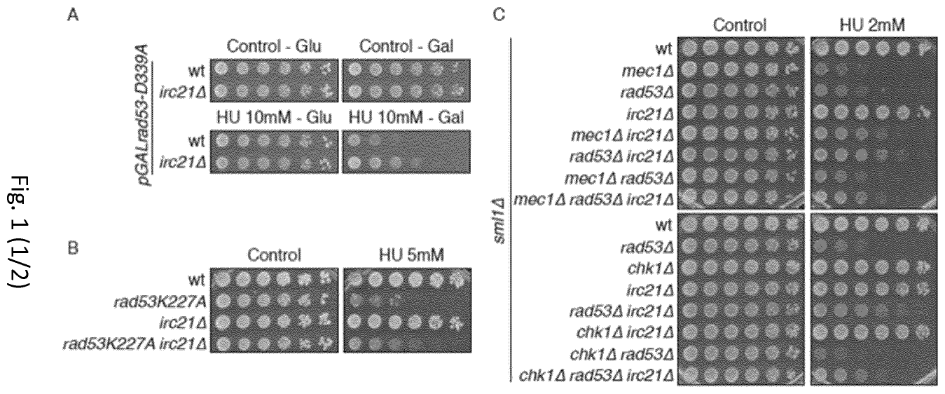

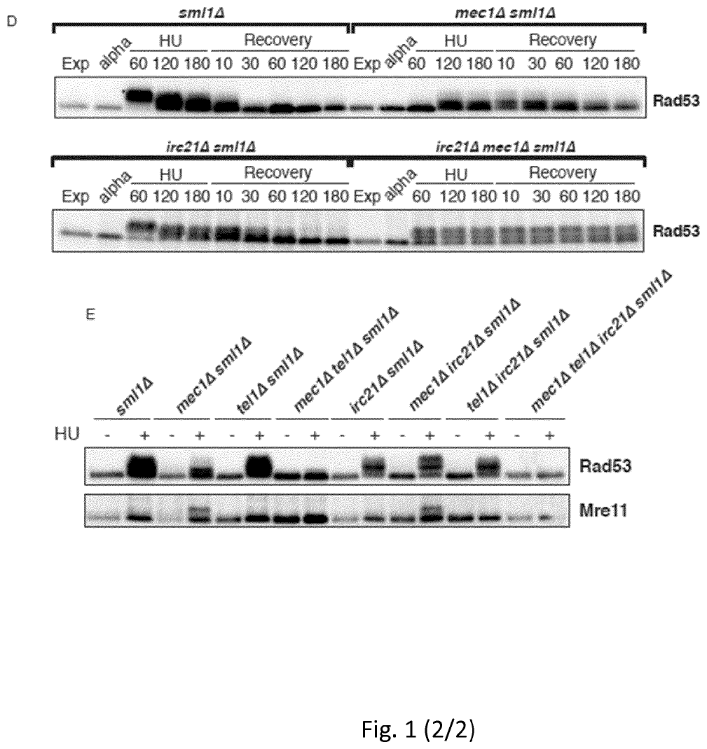

FIG. 1. IRC21 deletion rescue checkpoint mutants.

(A) Cells were grown on SD/-Ura plates containing glucose 2% or galactose 2% with or without hydroxyurea (HU). (B and C) Cells were grown on YPD plates with or without HU. (D) sml1Δ, sml1Δ mec1Δ, sml1Δ irc21Δ and sml1Δ mec1A irc21Δ cells were arrested in G₁ with α-factor (alpha) and released into YPD containing 0.2 M HU. After 3 hours, cells were released into YPD. Samples were collected at the indicated times to detect Rad53 by Western blot analysis. (E) Cells were arrested with α-factor and released in YPD with/without 0.2 M HU. Cells were treated for 3 hours and samples collected to detect Rad53 and Mrel1.

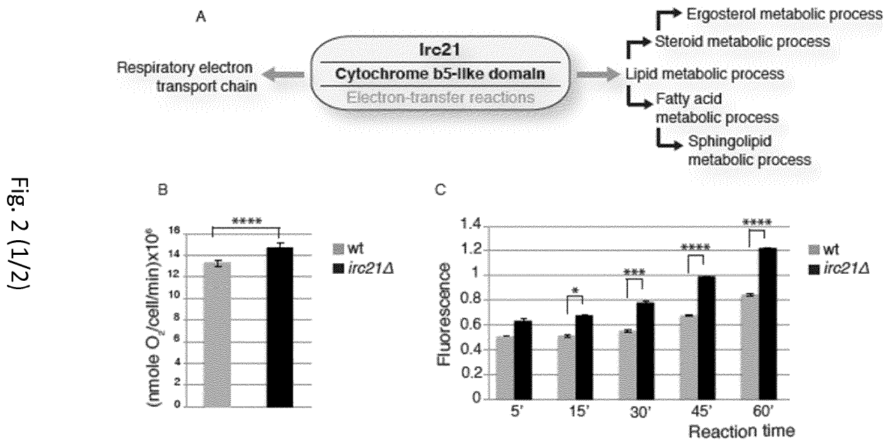

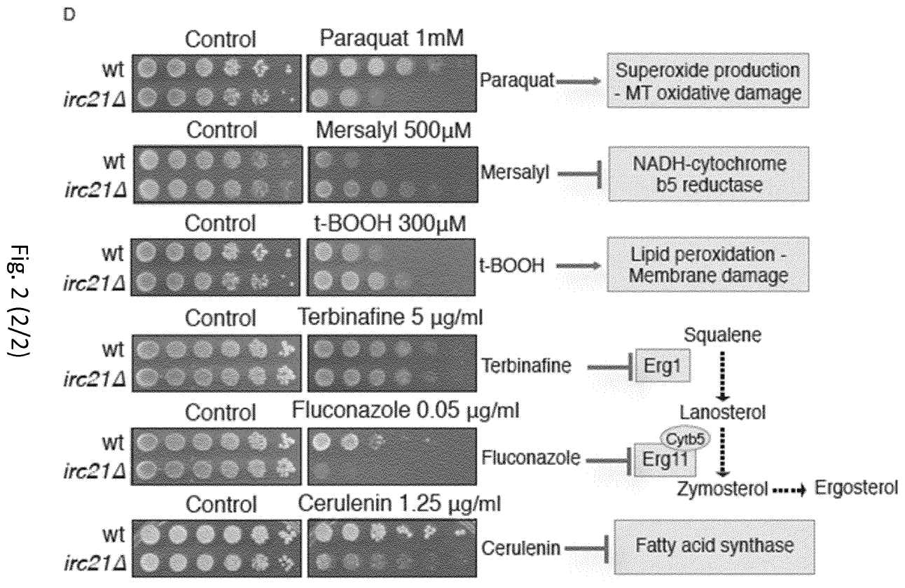

FIG. 2. IRC21 deletion impairs Cytb5-dependent processes.

(A) Classification of putative Irc21 processes based on Gene Ontology—PANTHER classification system. (B) Oxygen consumption rate of exponentially growing wt and irc21Δ cells. The results are shown as means±SD of triplicate. ****<0.0001. (C) Determination of ROS levels using the DCFH-DA assay in wt and irc21Δ cells, depending on incubation period. The results are shown as means±SD of triplicate. P values are indicated. (D) Spot assay of wt and irc21Δ cells on YPD plates with or without Paraquat, Mersalyl, t-BOOH, Terbinafine, Fluconazole, Cerulenin at the indicated concentrations. Drug mechanisms of action are illustrated.

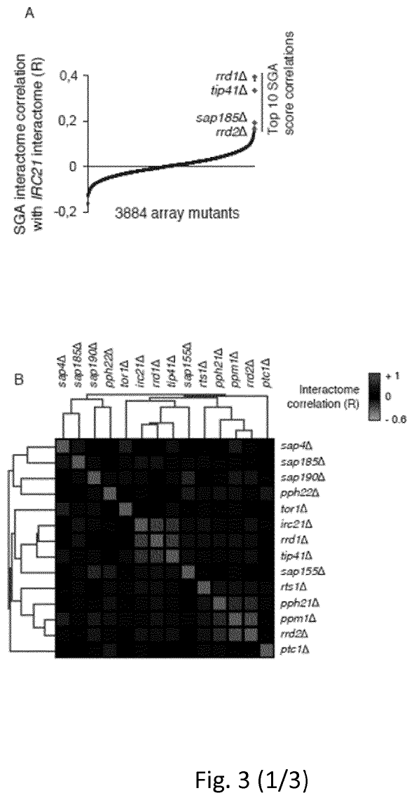

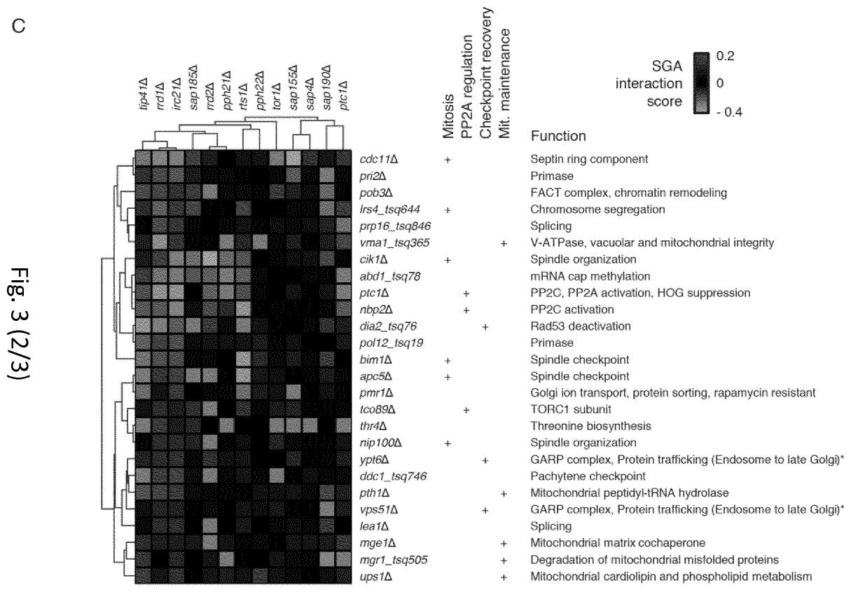

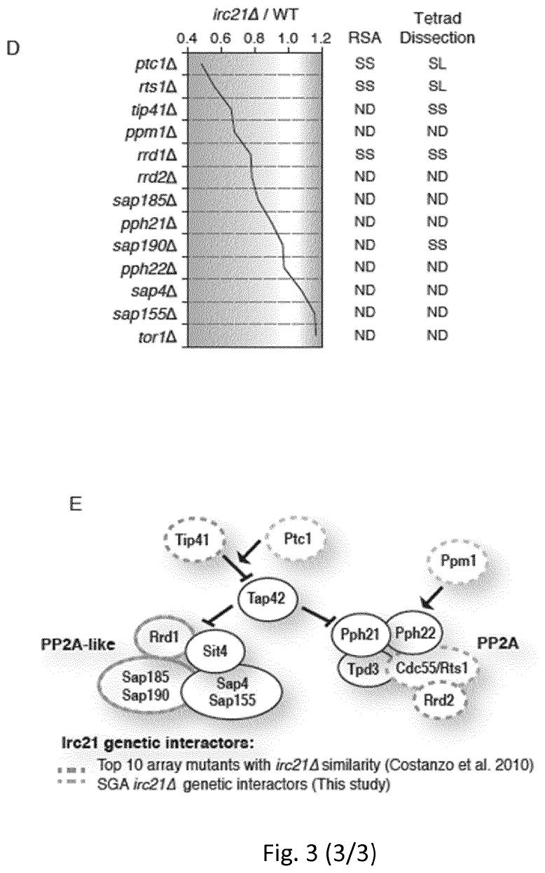

FIG. 3. Irc21 interacts with PP2A and PP2A-like phosphatases.

(A) Comparison between the interactome of the irc21Δ array strain with the interactomes of 3884 mutant array strains by calculating the correlation (R) value of their interaction scores with the 1712 query mutants (datasets from (Costanzo et al., 2010)). (B) Heatmap representing pairwise interactome correlation values (R) of mutants with altered PP2A activity. (C) Heatmap representing SGA interaction scores between query mutants of the irc21A & rrd1A & tip41Δ signature (rows) and PP2A-related array mutants (columns). Gray fields indicate that the interaction score has not been determined. (D) Genetic interactions of IRC21 with PP2A components and regulators assessed by SGA screening. Left panel: Quantitative effect of PP2A component deletions (rows) on the growth of irc21Δ mutants vs. wt. Right panel: Summary of the confirmation of individual genetic interactions. SS=Synthetic sick, SL=synthetic lethal. Interactions with ppm1 and tor1 were confirmed by gene targeting. (E) Representation of PP2A and PP2A-like complex subunits and regulators (see text). Blue and the orange dotted lines indicate irc21Δ mutant genetic interactors found in Costanzo et al. 2010 and in the present study, respectively.

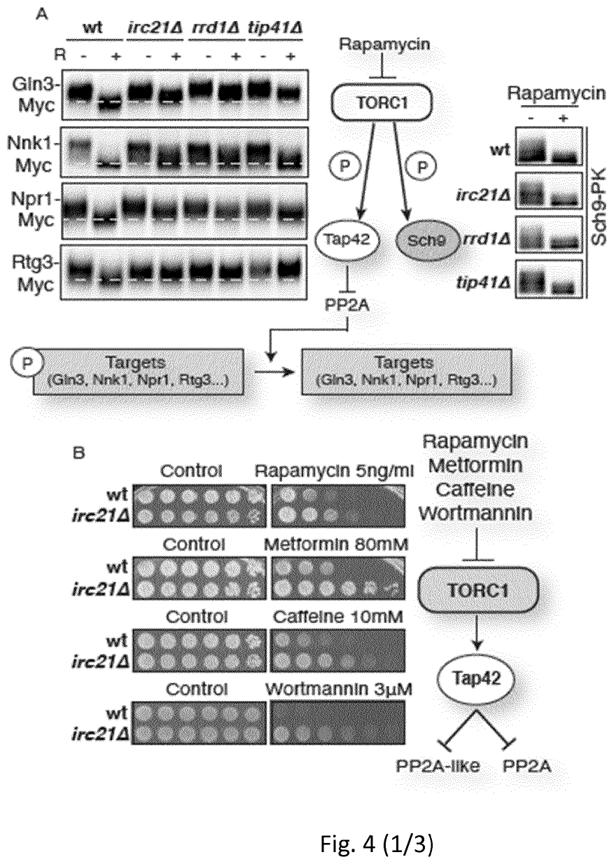

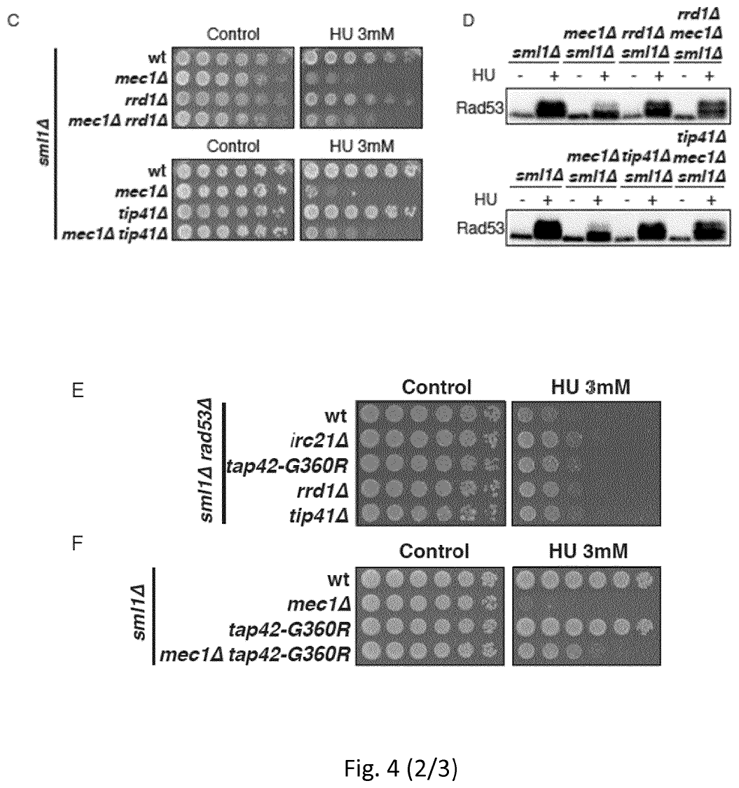

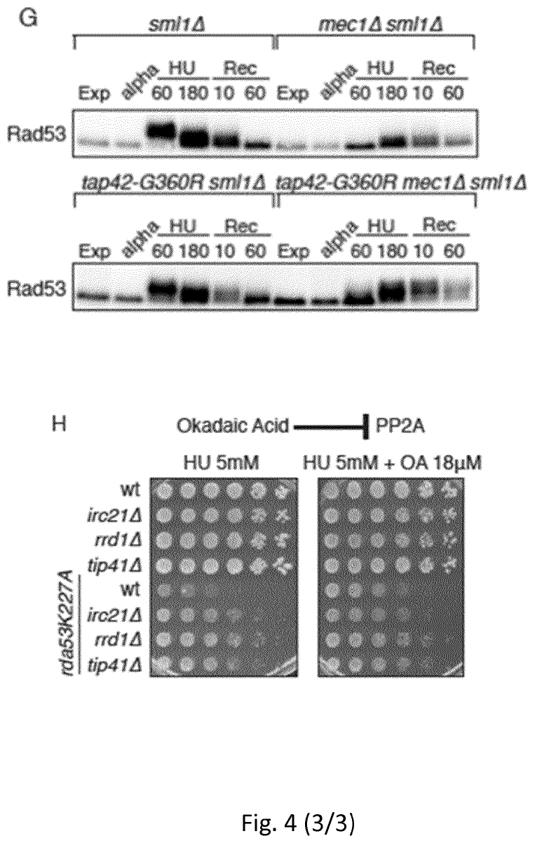

FIG. 4. Irc21 inhibits DDR through PP2A activation.

(A) Cells were treated with 200 ng/ml rapamycin. Bandshift assays following the phosphorylation of PP2A branch proteins Gln3, Nnk1, Npr1 and Rtg3, after 30′ of rapamycin treatment. A horizontal line has been overlaid to assist in determining mobility shifts (left panel). Bandshift assays following the phosphorylation of Sch9, after 2 hours of rapamycin (right panel). (B) wt and irc21Δ cells were grown on YPD plates with or without rapamycin, metformin, caffeine and wortmannin (left panel). All drugs are inhibitors of the Torc11-Tap42 pathway, represented in the right panel. (C, E, F) Cells were grown on YPD plates with or without 3 mM HU. (D) Cells were arrested with α-factor and released in YPD with/without 0.2 M HU. Cells were treated for 3 hours and harvested to detect Rad53. (G) Cells were arrested in G₁ with α-factor and released in YPD-0.2 M HU. After 3 hours, cells were released into YPD. Samples were collected at the indicated times to detect Rad53. (H) Cells were grown on YPD+HU plates with or without Okadaic acid (OA).

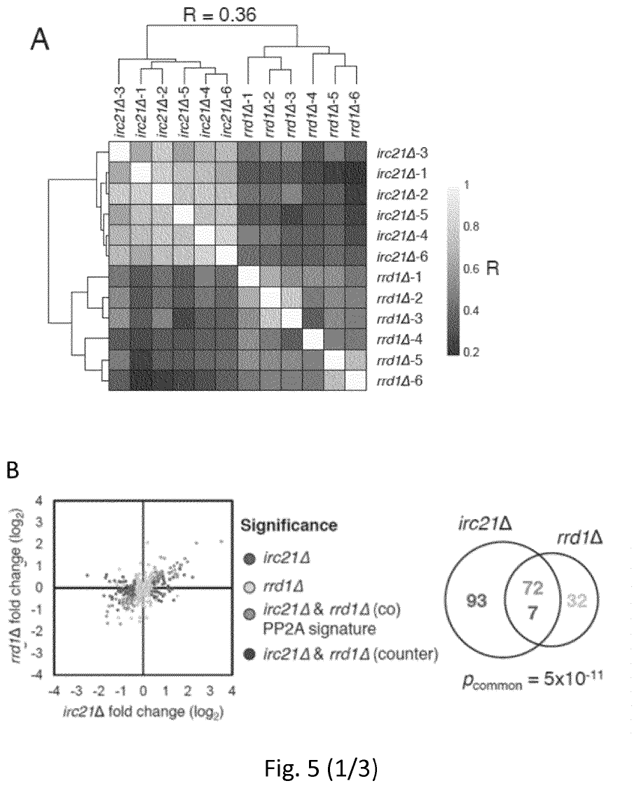

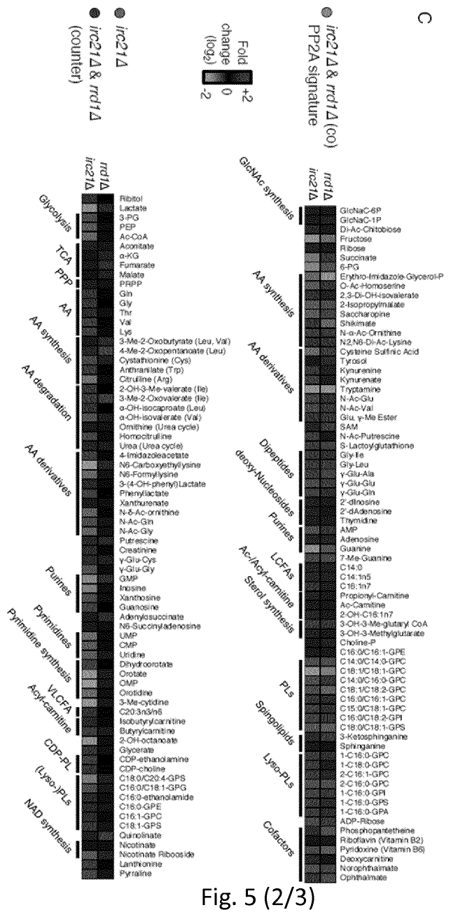

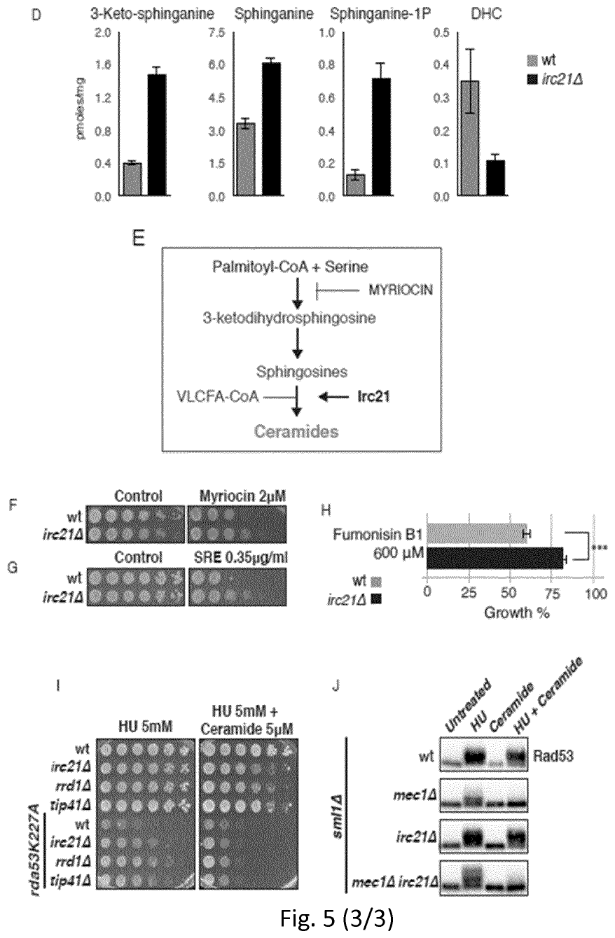

FIG. 5. Irc21 exerts PP2A-dependent and PP2A-activating metabolic regulations.

(A) Unsupervised hierarchical clustering of irc21Δ and rrd1Δ mutants (six replicates each) based on metabolome alterations during logarithmic growth in rich medium.

(B) Summary of metabolome alterations of irc21Δ and rrd1Δ mutants during logarithmic growth in rich medium. Left panel: Scatter plot of quantitative alterations of individual metabolites in irc21A and rrd1Δ mutants compared with a congenic wt identifies irc21A-specific (blue), rrd1A-specific (yellow), common (green, “PP2A signature”) and opposite (red) regulations. Right panel: Venn diagram representation of the intersection of metabolic alterations in irc21A and rrd1Δ mutants, and intersection significance p value determined by chi-squared test.

(C) Heatmap representation of altered metabolites by signature. Top panel: PP2A signature (common alterations in irc21Δ and rrd1A). Bottom panel: Specific regulations in irc21A and opposite regulations in irc21Δ and rrd1A. As indicated, metabolites were grouped by class.

(D) 3-keto-sphinganine, sphinganine, sphinganine-1p and dihydroceramide were quantified in wt and irc21Δ cells. Average values are shown, and error bars represent the standard error of the mean.

(E) Simplified scheme representing ceramide biosynthesis in S. cerevisiae. Colored metabolites indicate an increase (red) or a decrease (green) of their amount in irc21 cells.

(F) wt and irc21Δ cells were grown on YPD plates with or without myriocin.

(G) wt and irc21Δ cells were grown on YPD plates with or without syringomycin E.

(H) wt and irc21Δ cells were grown in SD medium with or without Fumonisin B1.

(I) Cells were grown on YPD+HU with or without ceramide.

(J) Cells were arrested in G₁ with α-factor and released in YPD with/without 0.2M HU, 15 μM ceramide or 0.2M HU in combination with 15 μM ceramide for 3 hours.

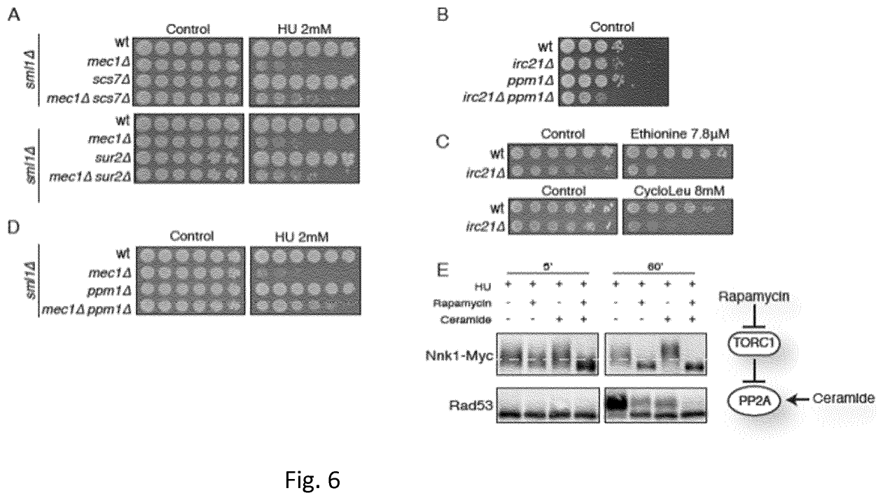

FIG. 6. Ceramides, TORC1, Irc21 and Ppm1 impact on the HU-induced DDR by modulating PP2A activity.

(A and D) Cells were grown on YPD with or without HU.

(B) Cells were grown on YPD. 1:10 dilutions were used to highlight growth rate differences.

(C) wt and irc21Δ cells were grown on YPD plates with or without ethionine or cycloleucine.

(E) Cells were arrested with α-factor and released into YPD containing 0.2M HU alone or in combination with rapamycin 200 ng/ml, ceramide 15 μM, rapamycin 200 ng/ml+ceramide 15 μM. Cells were treated for 1 hour and harvested after 5′ and 60′ in order to detect Nnk1 and Rad53.

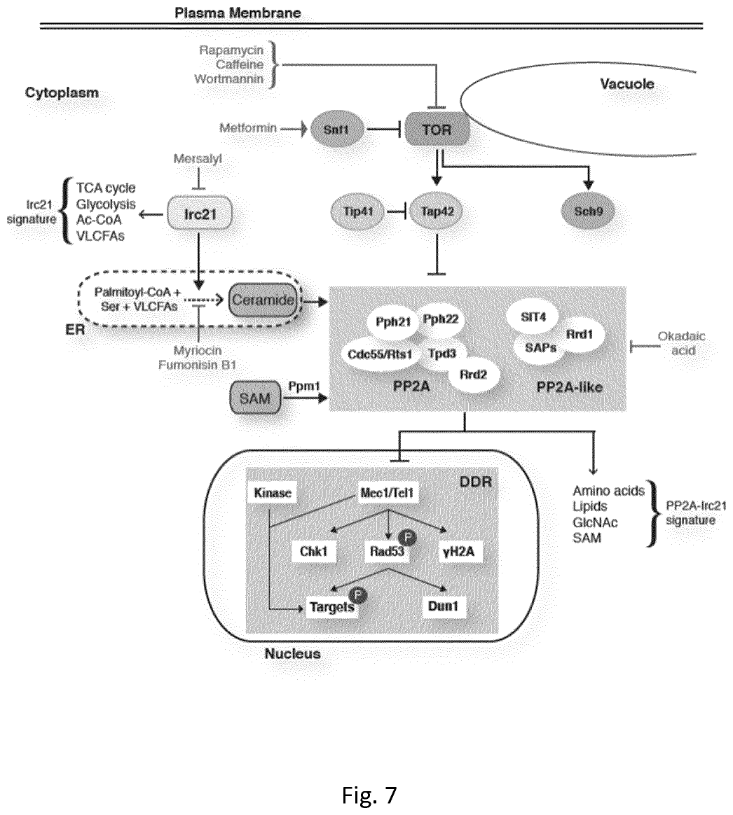

FIG. 7. Model: PP2A links DRR with cell metabolism.

PP2A and PP2A-like phosphatases are regulated by TORC1 (nitrogen availability), Snf1^{AMPK}(carbon availability), ceramide (sphingolipids and fatty acids availability) and SAM (methionine availability). The two PP2A complexes integrate the metabolic input with the control of DDR. Irc21 acts upstream of PP2A: it shares PP2A signature but also display specific metabolic function (Irc21 signature).

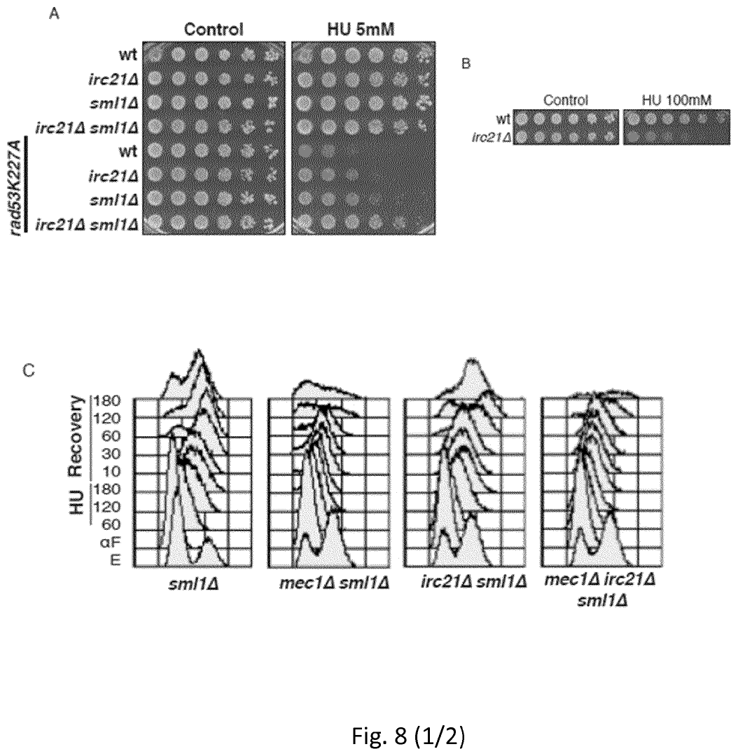

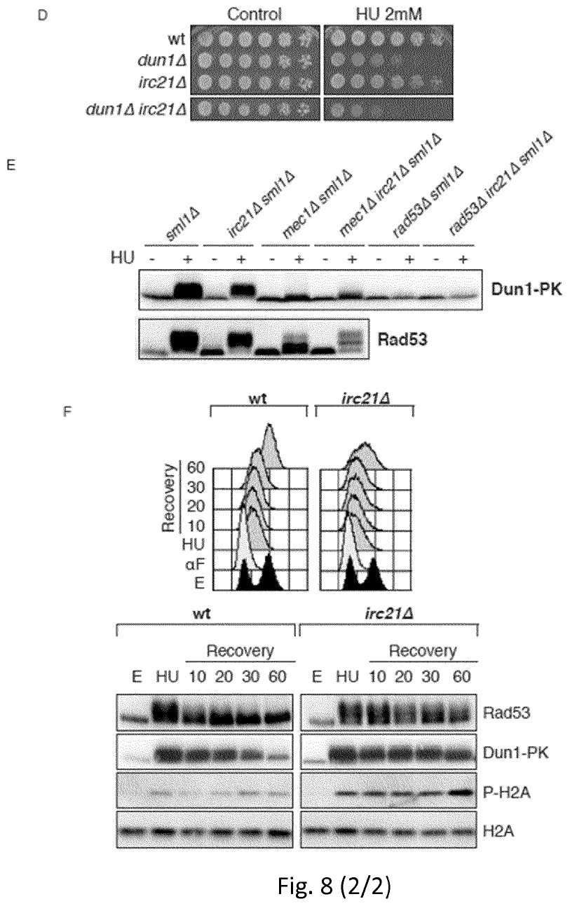

FIG. 8. (A,B,D) Cells were grown on YPD plates with or without HU.

(C) Cells were arrested in G₁ with α-factor (αF) and released into YPD containing 0.2 M HU. After 3 hours, cells were released into YPD. Samples were collected at the indicated times to determine DNA content by fluorescence-activated cell sorting (FACS) analysis (Related to FIG. 1D).

(E) Cells were arrested with α-factor and released in YPD or YPD containing 0.2 M HU. Cells were treated for 3 hours and harvested to detect Dun1 and Rad53.

(F) Cells were arrested in G₁ with α-factor and released into YPD containing 0.2 M HU. After 3 hours, cells were released into YPD. Samples were collected at the indicated times to determine DNA content by fluorescence-activated cell sorting (FACS) analysis (left panel) and to detect Rad53, Dun1, P-H2A and H2A by Western blot analysis (right panel).

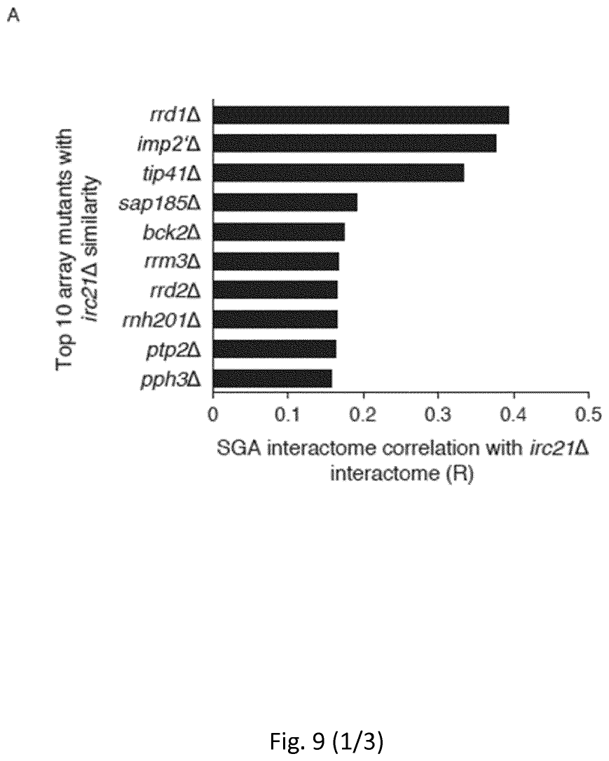

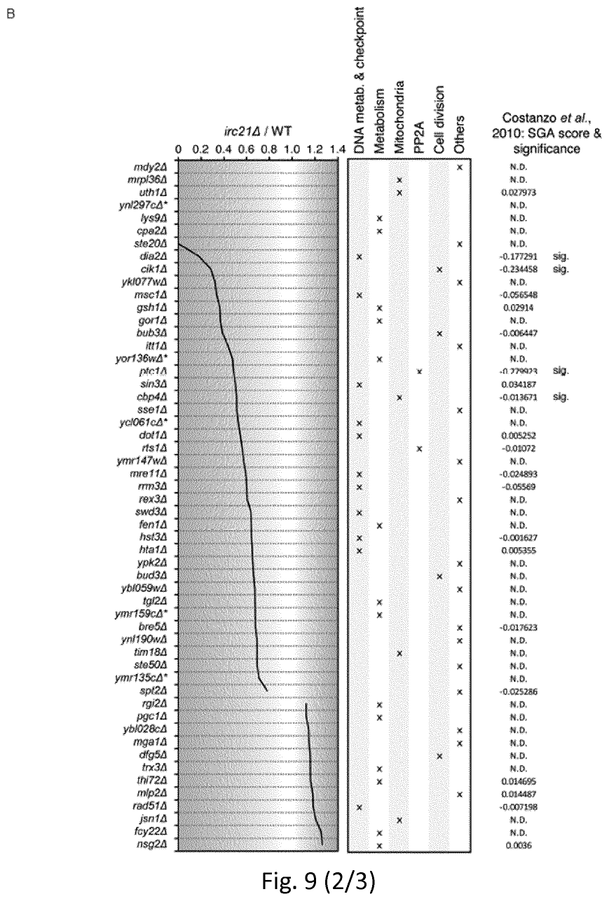

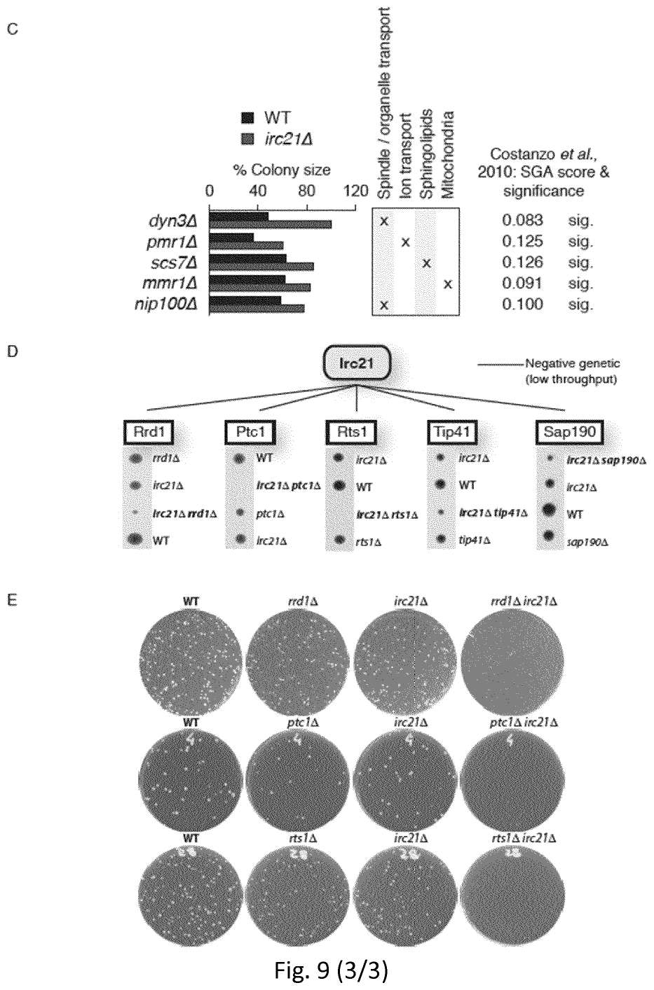

FIG. 9. (A) Top 10 array mutants with highest interactome similarity to irc21Δ. The Pearson correlation (R) value was obtained by comparing the interactome of the irc21Δ array strain with the interactomes of 3884 mutant array strains with the 1712 query mutants (datasets from (Costanzo et al., 2010)). (Related to FIG. 3A).

(B) Negative and rescuing genetic interactions of IRC21 assessed by SGA screening. Left panel: Quantitative effect of array gene deletions (rows) on the growth of irc21Δ mutants vs. wt. Middle panel: Manual functional classification of IRC21 interactors. Right panel: Comparison with published SGA scores (Costanzo et al., 2010).

(C) Epistatic genetic interactions of IRC21 assessed by SGA screening. Left panel: Quantitative effect of array gene deletions (rows) on the growth of irc21Δ mutants vs. wt. Middle panel: Manual functional classification of IRC21 interactors. Right panel: Comparison with published SGA scores (Costanzo et al., 2010).

(D) Tetrad analysis of irc21Δ rrd1Δ, irc21Δ ptc1Δ, irc21Δ rts1Δ, irc21Δ tip41Δ, irc21Δ sap190Δ strains (Related to FIG. 3D).

(E) Confirmation of the genetic interactions between Irc21 and Rrd1, Ptc1 and Rts1 by random spore analysis (spore derived from the SGA screening, performed in S228C genetic background) (Related to FIG. 3D).

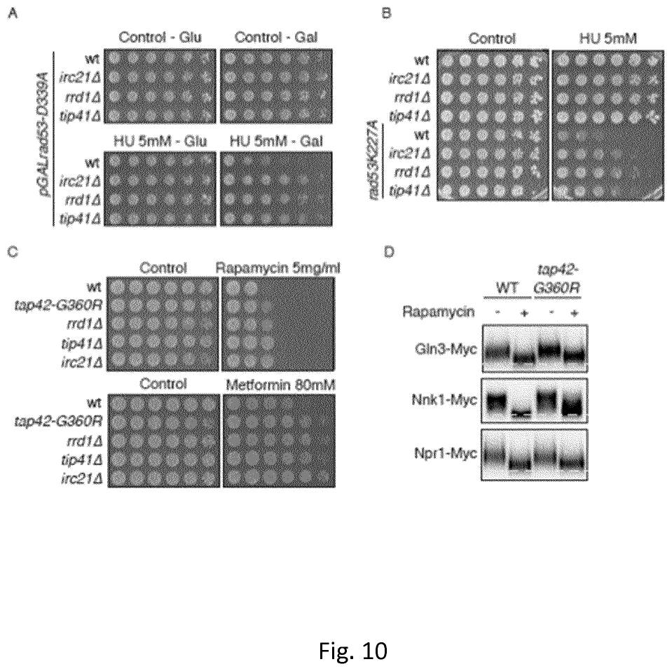

FIG. 10. (A) Cells were grown on SD/-Ura containing glucose 2% or galactose 2% with or without HU.

(B) Cells were grown on YPD plates with or without HU.

(C) Cells were grown on YPD plates with or without rapamycin or metformin.

(D) Cells were treated for 30′ with 200 ng/ml rapamycin. Bandshift assays following the phosphorylation of PP2A branch proteins Gln3, Nnk1, and Npr1.

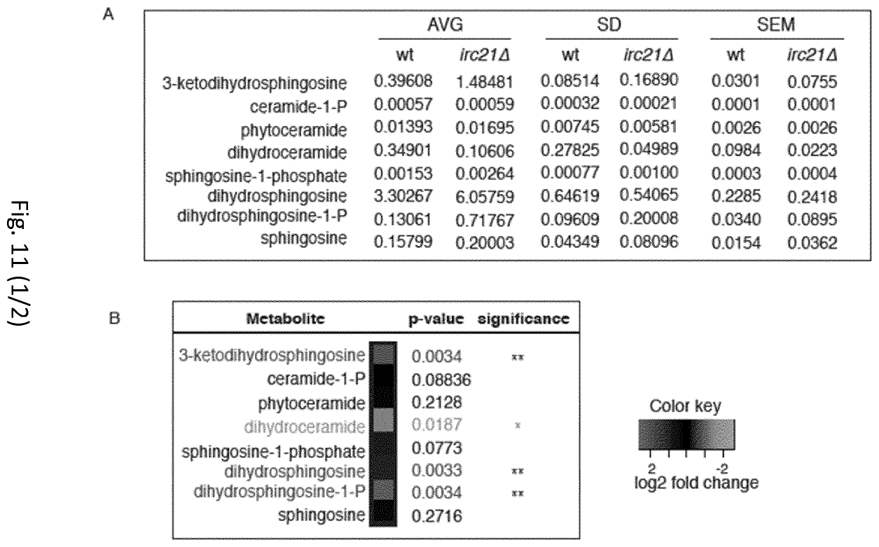

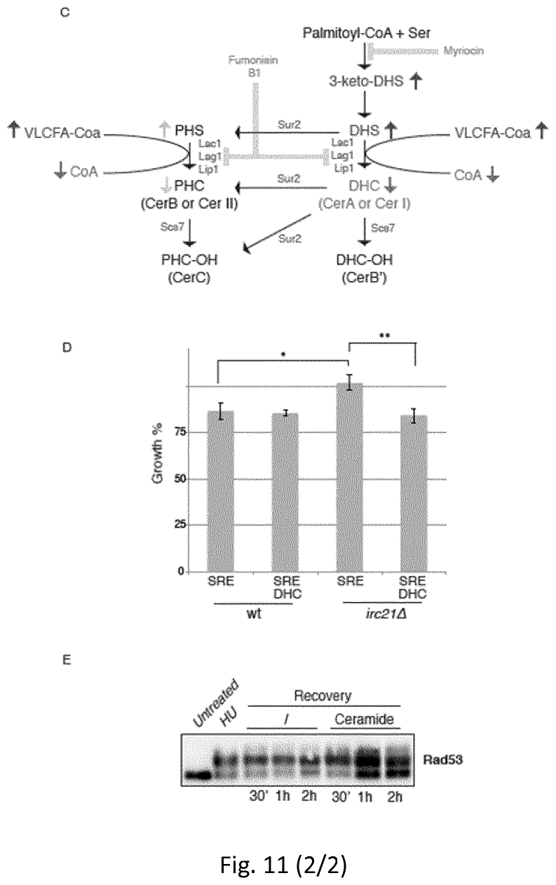

FIG. 11. (A) Quantification (pmol/mg) of the listed metabolites in wt and irc21Δ cells by TrueMass Ceramide analysis. Average values (AVG), standard deviation (SD) and standard error of the mean (SEM) are shown (Related to FIGS. 5D and E).

(B) TrueMass Ceramide panel quantification of the listed metabolites in irc21Δ cells. p-value and statistical significance are shown (Related to FIGS. 5D and E).

(C) Illustration of sphingolipid biosynthesis in S. cerevisiae. Colored metabolites indicate an increase (red) or a decrease (green) of their amount in irc21Δ cells (refer to FIG. 11B). Myriocin inhibits serine palmitoyl-CoA transferase. Fumonisin B1 inhibits ceramide synthase.

(D) Cell sensitivity to syringomycin in presence or absence of dihydroceramide. Cells were grown in SD medium. Average values are shown and error bars represent the standard deviation.

(E) mec1A sml1Δ irc21Δ cells were arrested in G₁ with α-factor and released into YPD containing 0.1 M HU. After 3 hours, cells were released into YPD or YPD with ceramide 15 μM. Samples were collected at the indicated times to detect Rad53, by Western blot analysis.

FIG. 12. (A) Cells were treated with 200 ng/ml rapamycin. Bandshift assays following the phosphorylation of PP2A branch proteins Gln3, Nnk1, Npr1 and Rtg3, after 30′ of rapamycin treatment.

(B) Cells were treated with 0.2M HU alone or in combination with rapamycin 200 ng/ml, ceramide 15 μM, rapamycin 200 ng/ml+ceramide 15 μM. Samples were collected at the indicated times to determine DNA content by FACS analysis, to detect Rad53 and Nnk1 by Western blot analysis, to evaluate the budding index.

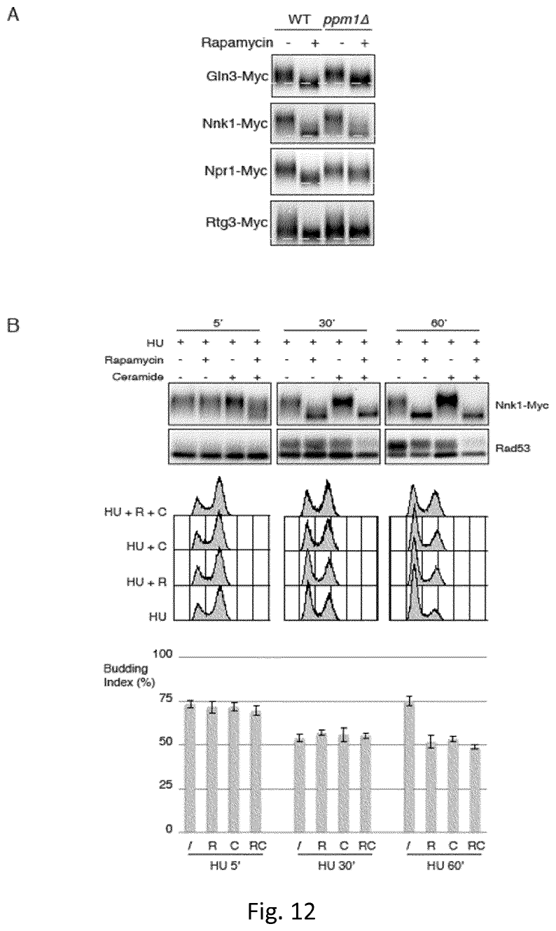

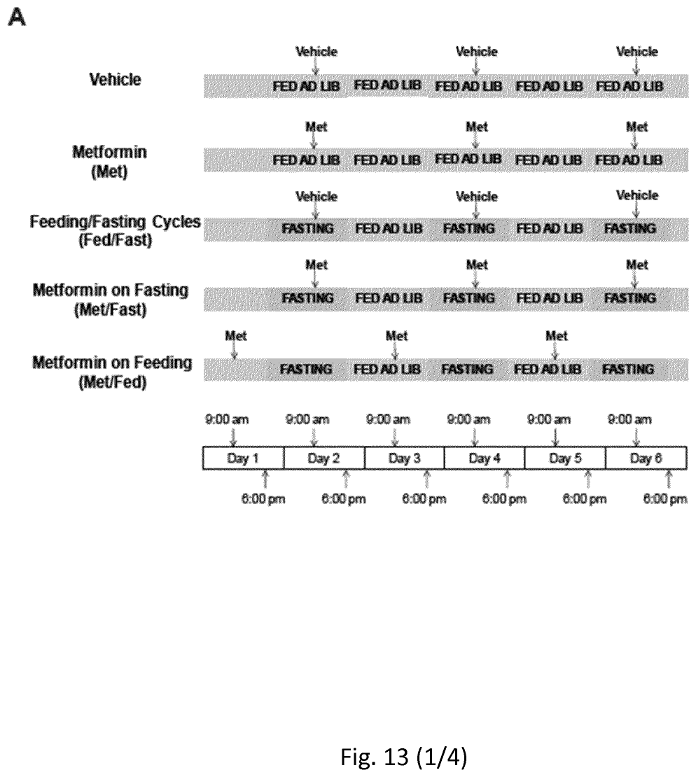

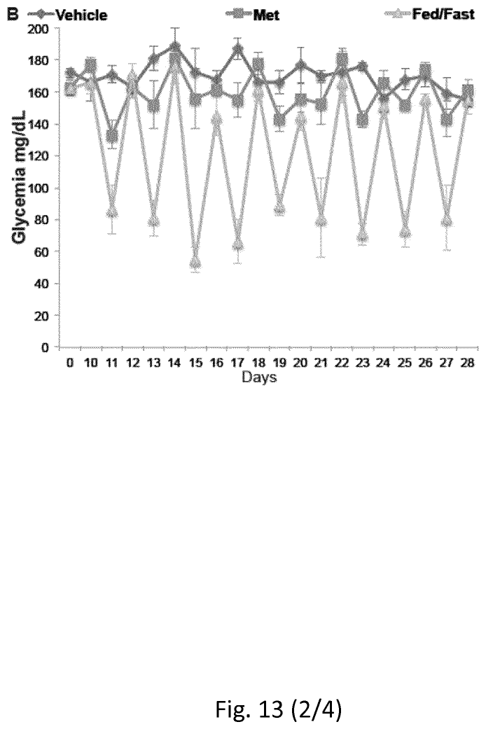

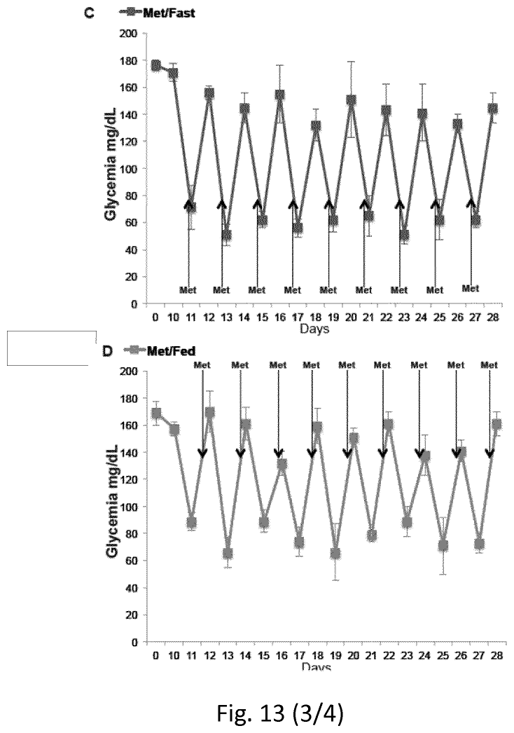

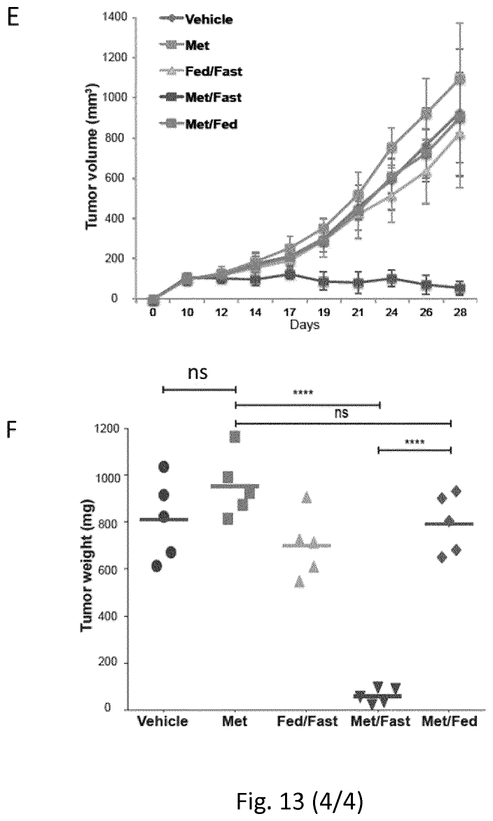

FIG. 13. Intermittent fasting sensitizes tumor-bearing mice to metformin administered during hypoglycemic periods. (A) Schematic representation of the experimental design showing the feeding protocols and timing of metformin administration in different experimental groups. (B-D) The levels of blood glucose measured at the end of each feeding/fasting cycle in different experimental groups. Arrows (C-D) indicate timing of metformin administration. (E) In vivo growth of xenograft tumors as measured by tumor volume (length×width×width/2) in mice inoculated with HCT116 cells. Upon establishment of tumors, mice were kept on 24-hour feeding/fasting cycles as indicated above and treated with either vehicle or metformin (200 mg/kg) administered by oral gavage every 48 hours either during feeding or fasting cycles. Error bars indicate SEM. (n=5 per group). (F) Weight and images of tumors isolated from mice in different groups. student's t test ****: p<0.001, ns: non-significant.

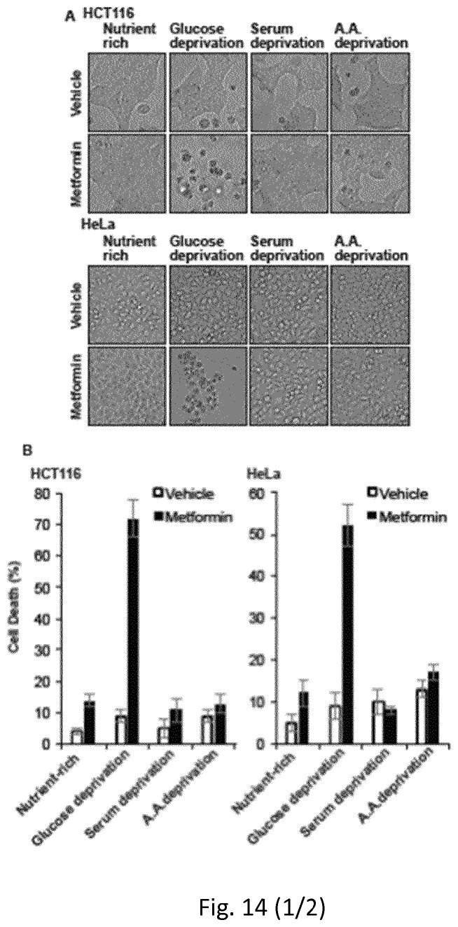

FIG. 14. Glycolysis inhibition sensitizes cancer cells to metformin. (A) Images of HCT116 and HeLa cells cultured for 24 hours in either nutrient-rich DMEM (containing 10% FBS and 10 mM glucose), DMEM containing 2.5 mM glucose (glucose deprivation), DMEM with 0.1% serum (serum deprivation) or DMEM with no glutamine, no methionine and no cysteine (amino acids deprivation). Media were replenished every 6 hours. (B) Quantification of cell death of HCT116 and HeLa cells cultured as in A as measured by propidium iodide uptake using flow cytometry. (C, D) Percentage of cell death measured by propidium iodide uptake using flow cytometry (C) or growth rate as assessed by CellTiter-Glo assay (D) of HCT116 and HeLa cells cultured for 24 hours in DMEM containing the indicated amounts of glucose in the absence or presence of 5 mM metformin (C) or in DMEM containing the indicated concentration of glucose and treated with increasing concentrations of metformin for 24 h (D). (E) Percentage of cell death of HCT116 and HeLa cells cultured for 24 hours in DMEM containing either 10 mM glucose (Normal glucose) or 2.5 mM glucose (Low glucose) in combination with increasing concentrations of metformin for 24 h. (F) Percentage of cell death of HCT116 and HeLa cells cultured for 24 hours in DMEM containing either 10 mM glucose (Normal glucose) or 2.5 mM glucose (Low glucose) in combination with either Metformin (5 mM), SAHA (2.5 μM) or Brefeldin A (10 μM). Media were replenished every 6 hours.

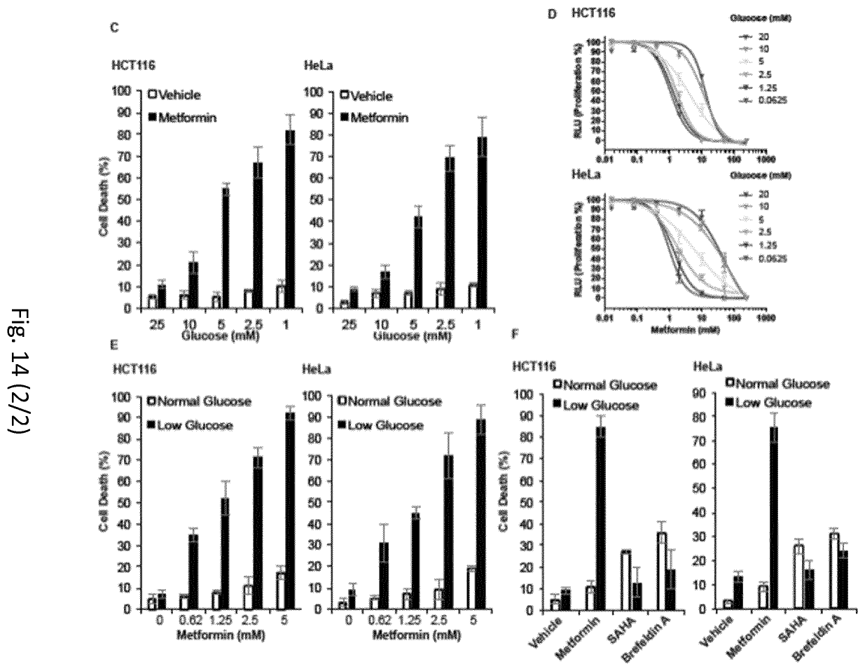

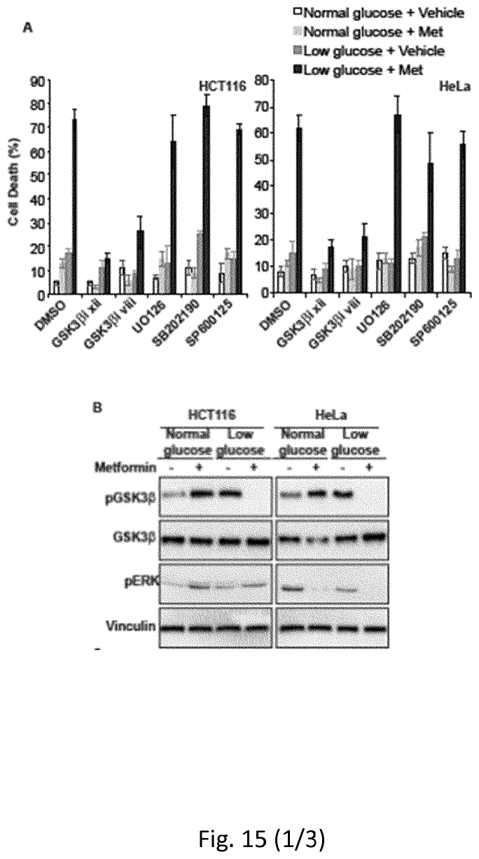

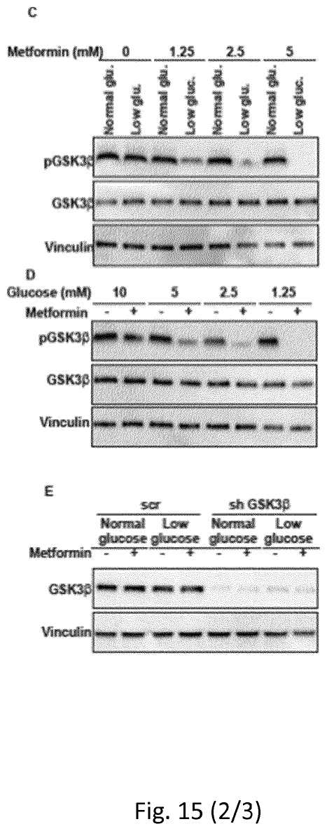

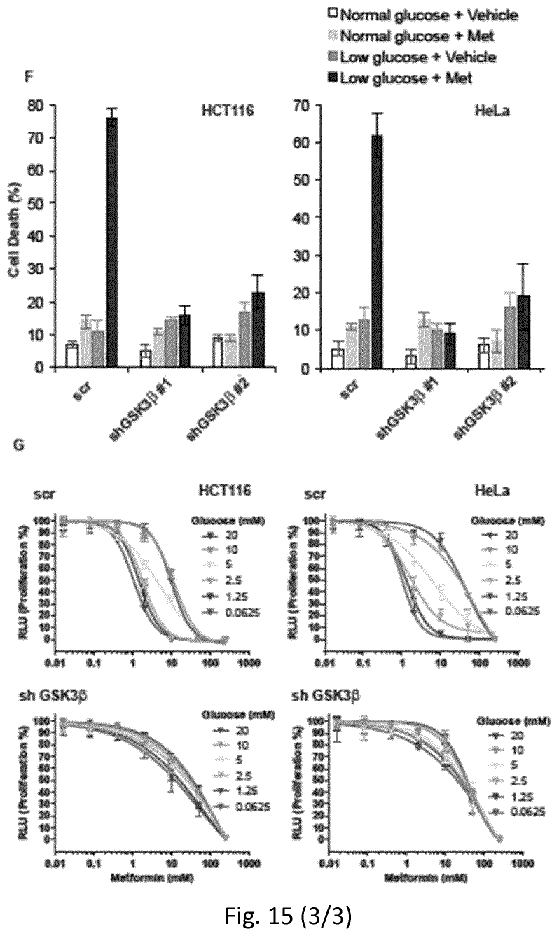

FIG. 15. Synergistic cytotoxicity of low glucose and metformin is mediated by GSK3β. (A) Percentage of cell death of HCT116 and HeLa cells treated with either GSK3P inhibitor xii (20 μM), GSK3 β inhibitor viii (25 μM), ERK inhibitor UO126 (20 μM), p38 inhibitor SB202190 (20 μM) or JNK inhibitor SP600125 (20 μM) and cultured for 24 hours in DMEM (replenished every 6 hours) containing either 10 mM or 2.5 mM glucose (Normal or Low glucose respectively) in the absence or presence of metformin (5 mM). Treatment with the inhibitors started 1 hour before metformin treatment. (B) Immunoblotting analysis of lysates derived from HCT116 and HeLa cells cultured for 24 hours in DMEM (replenished every 6 hours) containing either 10 mM or 2.5 mM glucose (Normal or Low glucose respectively) in the absence or presence of metformin (5 mM). (C) Immunoblotting analysis of lysates derived from HCT116 cells cultured for 24 hours in DMEM (replenished every 6 hours) containing either 10 mM or 2.5 mM glucose (Normal or Low glucose respectively) in the absence or presence of the indicated concentrations of metformin. (D) Immunoblotting analysis of lysates derived from HCT116 cells cultured for 24 hours in DMEM (replenished every 6 hours) containing the indicated concentrations of glucose in the absence or presence of metformin (5 mM). (E) Immunoblotting analysis of lysates derived from HCT116 cells stably expressing either scrambled shRNA or shRNA against GSK3P3 and treated as in B. (F) Percentage of cell death of control or GSK3β-depleted HCT116 and HeLa cells treated as in B. (G) Proliferation assessed by CellTiter-Glo assay of control or GSK3β-depleted HCT116 and HeLa cultured in DMEM containing the indicated concentration of glucose and treated with increasing concentrations of metformin for 24 h

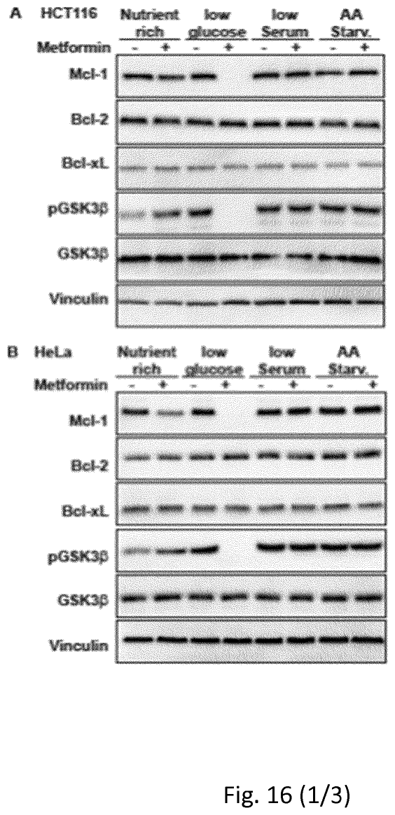

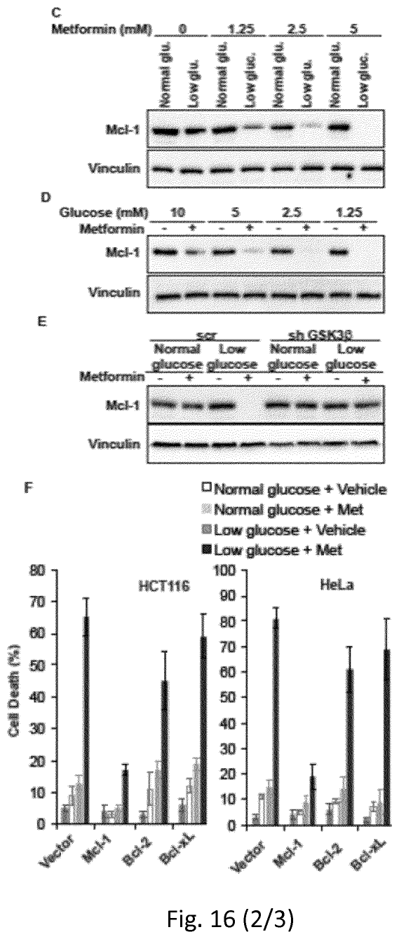

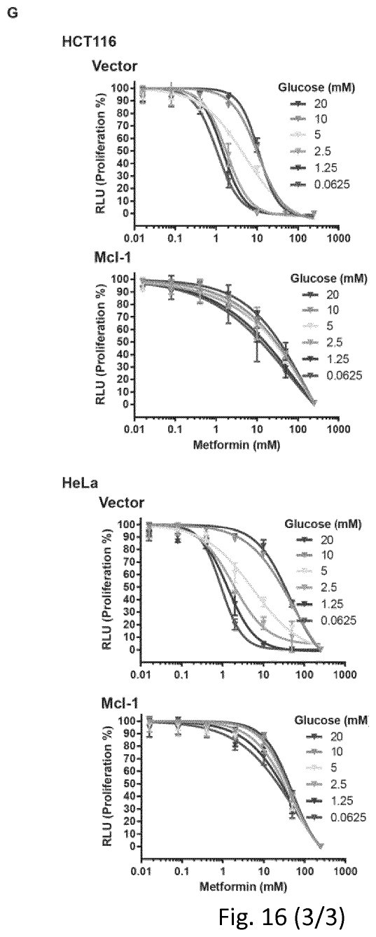

FIG. 16. GSK3β-depleted MCL-1 degradation mediates synergistic cytotoxicity of low glucose and metformin. (A, B) Immunoblotting analysis of lysates derived from HCT116 and HeLa cells cultured for 24 hours in either nutrient-rich DMEM (containing 10% FBS and 10 mM glucose), DMEM containing 2.5 mM glucose (glucose deprivation), DMEM with 0.1% serum (serum deprivation) or DMEM with no glutamine, no methionine and no cysteine (amino acids deprivation). Media were replenished every 6 hours. (C) Immunoblotting analysis of lysates derived from HCT116 cells cultured for 24 hours in DMEM (replenished every 6 hours) containing either 10 mM or 2.5 mM glucose (Normal or Low glucose respectively) in the absence or presence of the indicated concentrations of metformin. (D) Immunoblotting analysis of lysates derived from HCT116 cells cultured for 24 hours in DMEM (replenished every 6 hours) containing the indicated concentrations of glucose in the absence or presence of metformin (5 mM). (E) Immunoblotting analysis of lysates derived from HCT116 cells expressing either scrambled shRNA or shRNA against GSK3β and cultured for 24 hours in DMEM (replenished every 6 hours) containing either 10 mM or 2.5 mM glucose (Normal or Low glucose respectively) in the absence or presence of metformin (5 mM). (F) Percentage of cell death of HCT116 and HeLa cells expressing the indicated constructs and cultured as in D. (G) Proliferation assessed by CellTiter-Glo assay of control or MCL-1 overexpressing HCT116 and HeLa cells cultured in DMEM containing the indicated concentration of glucose and treated with increasing concentrations of metformin for 24 h.

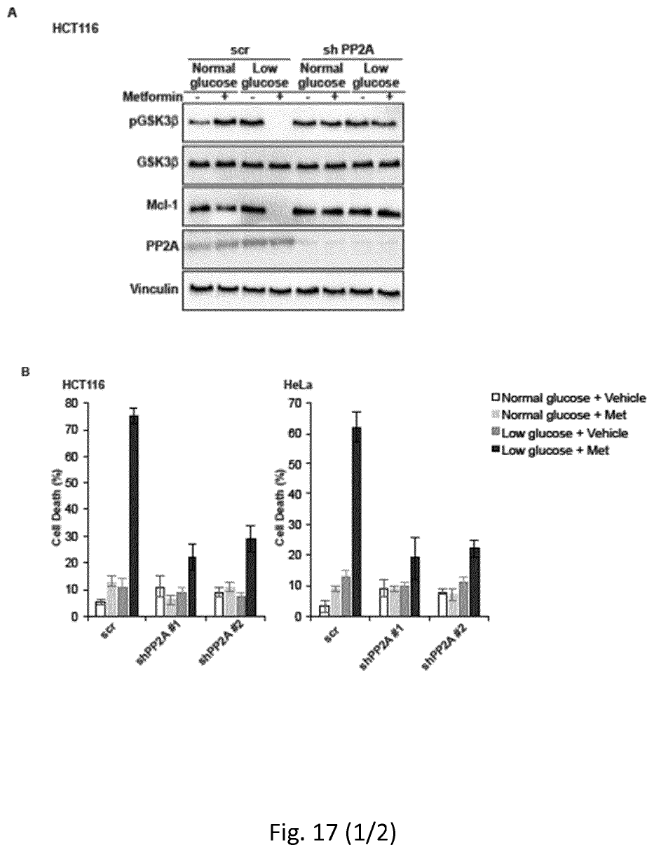

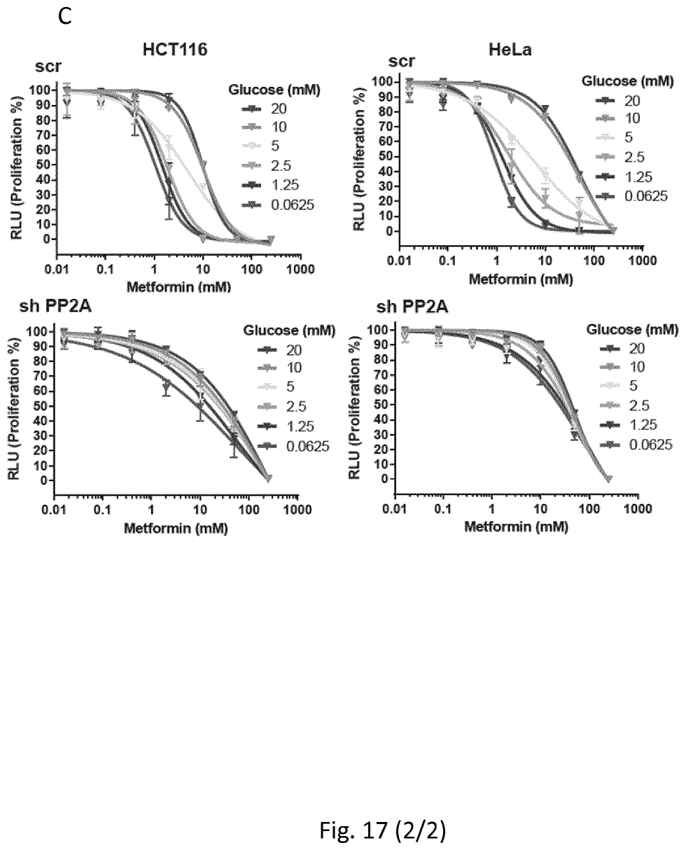

FIG. 17. PP2A-regulated GSK3β dephosphorylation mediates synergistic cytotoxicity of low glucose and metformin. (A) Immunoblotting analysis of lysates derived from HCT116 cells expressing either scrambled shRNA or shRNA against PP2A and cultured for 24 hours in DMEM (replenished every 6 hours) containing either 10 mM or 2.5 mM glucose (Normal or Low glucose respectively) in the absence or presence of metformin (5 mM). (B) Percentage of cell death of control or PP2A-depleted HCT116 and HeLa cells treated as in A. (C) Proliferation assessed by CellTiter-Glo assay of control or PP2A-depleted HCT116 and HeLa cells cultured in DMEM containing the indicated concentration of glucose and treated with increasing concentrations of metformin for 24 h.

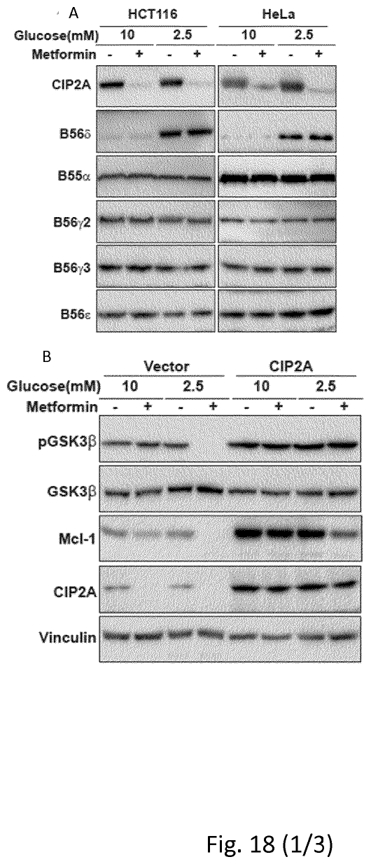

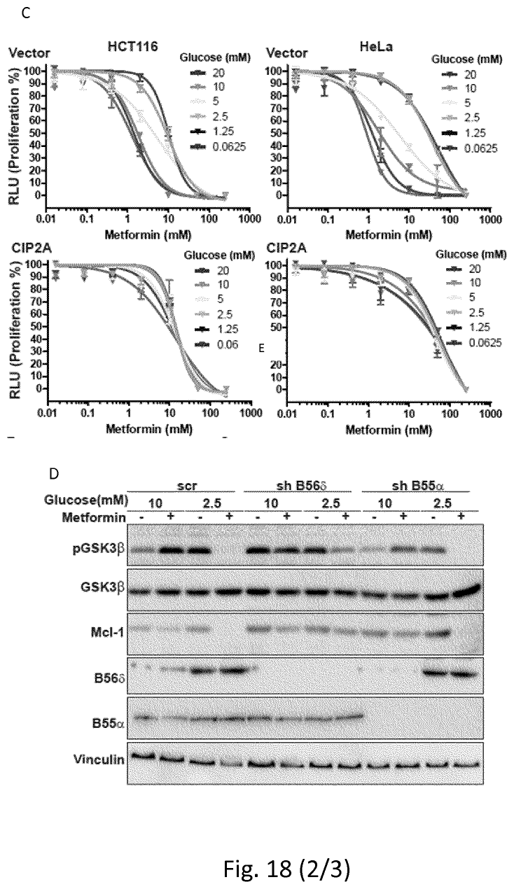

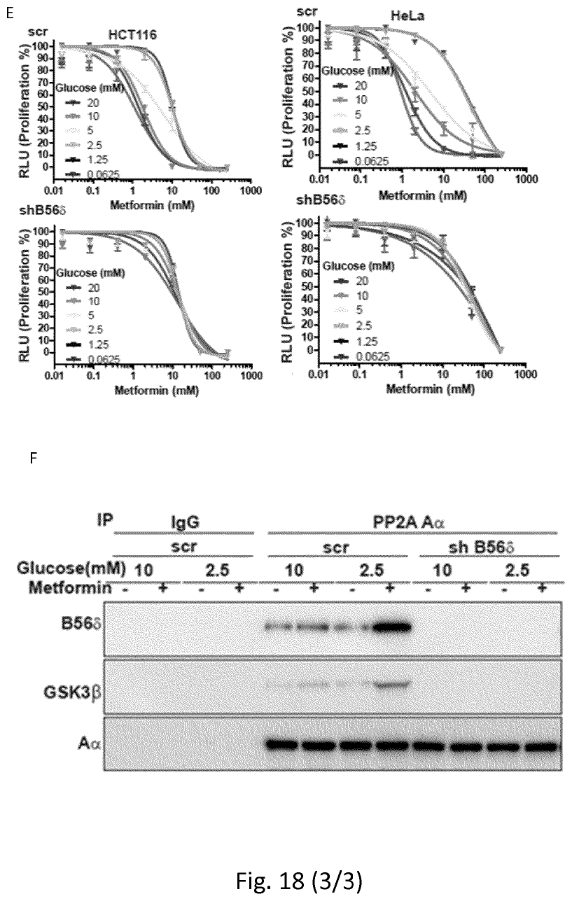

FIG. 18. Simultaneous CIP2A inhibition and B56δ upregulation mediate synergistic cytotoxicity of low glucose/metformin combination. (A) Immunoblotting analysis of lysates derived from HCT116 and HeLa cells cultured for 24 hours in DMEM (replenished every 6 hours) containing either 10 mM or 2.5 mM glucose (Normal or Low glucose respectively) in the absence or presence of metformin (5 mM). (B) Immunoblotting analysis of lysates derived from HCT116 cells stably expressing either scrambled shRNA or shRNA against CIP2A and cultured for 24 hours in DMEM (replenished every 6 hours) containing either 10 mM or 2.5 mM glucose (Normal or Low glucose respectively) in the absence or presence of metformin (5 mM). (C) Proliferation assessed by CellTiter-Glo assay of control or CIP2A-depleted HCT116 and HeLa cells cultured in DMEM containing the indicated concentration of glucose and treated with increasing concentrations of metformin for 24 h. (D) Immunoblotting analysis of lysates derived from HCT116 cells stably expressing scrambled shRNA or shRNAs against wither B56δ or B55α and cultured for 24 hours in DMEM (replenished every 6 hours) containing either 10 mM or 2.5 mM glucose (Normal or Low glucose respectively) in the absence or presence of metformin (5 mM). (E) Proliferation assessed by CellTiter-Glo assay of control or B56δ-depleted HCT116 and HeL cells cultured in DMEM containing the indicated concentration of glucose and treated with increasing concentrations of metformin for 24 h. (F) Immunoprecipitation analysis of PP2A Aα from cell lysates derived from HCT116 cells stably expressing scrambled shRNA or shRNAs against wither B56δ or B55α and cultured for 24 hours in DMEM (replenished every 6 hours) containing either 10 mM or 2.5 mM glucose (Normal or Low glucose respectively) in the absence or presence of metformin (5 mM).

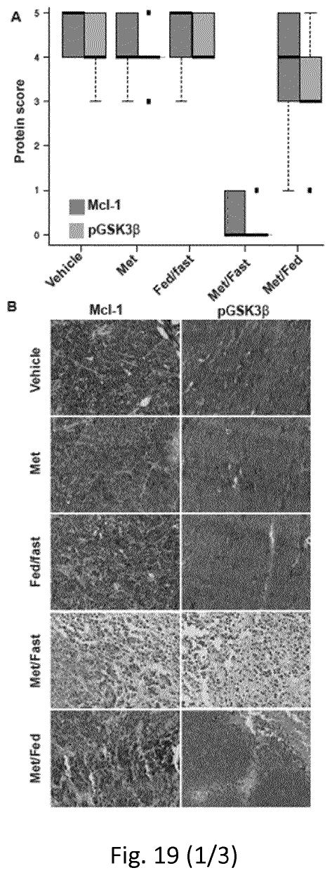

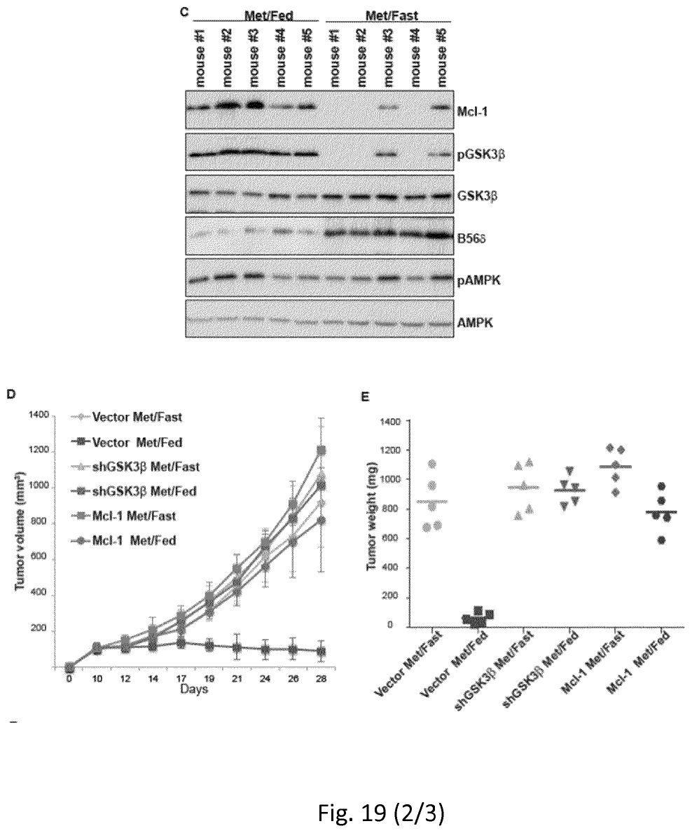

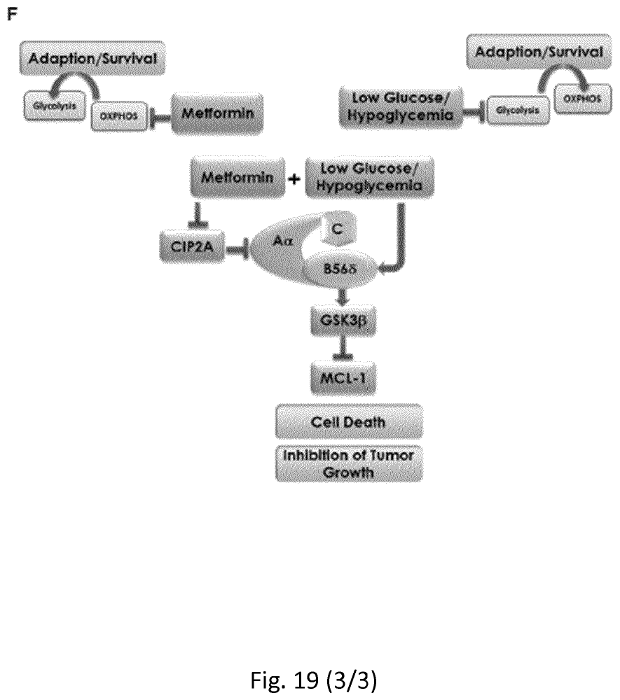

FIG. 19. Modulation of GSK3β-MCL-1 axis mediates tumor sensitization to metformin administered during fasting-induced hypoglycemia. (A,B) Immunohistochemical analysis and representative images (original magnification is 20×) of MCL-1 and phosphorylated GSK3β in tissue samples isolated from mice treated as in FIG. 13. The bars represent the highest and lowest quartiles. (C) Immunoblotting analysis of tumor lysates derived from mice treated as in FIG. 13. (D) In vivo growth of tumors xenografts in mice inoculated with either control, GSk3β-depleted or MCL-1-overexpressing HCT116 cells. Upon establishment of tumors, mice were kept on 24-hour feeding/fasting cycles and treated with metformin (200 mg/kg) administered by oral every 48 hours either during feeding cycles (Met/Fed) or during fasting cycle (Met/fast). Error bars indicate SEM. (n=5 per group). (E) Weight of tumors from D isolated at the end of the treatment. (F) Schematic representation of the molecular mechanism of targeting metabolic plasticity of tumor cells by low glucose-metformin combination.

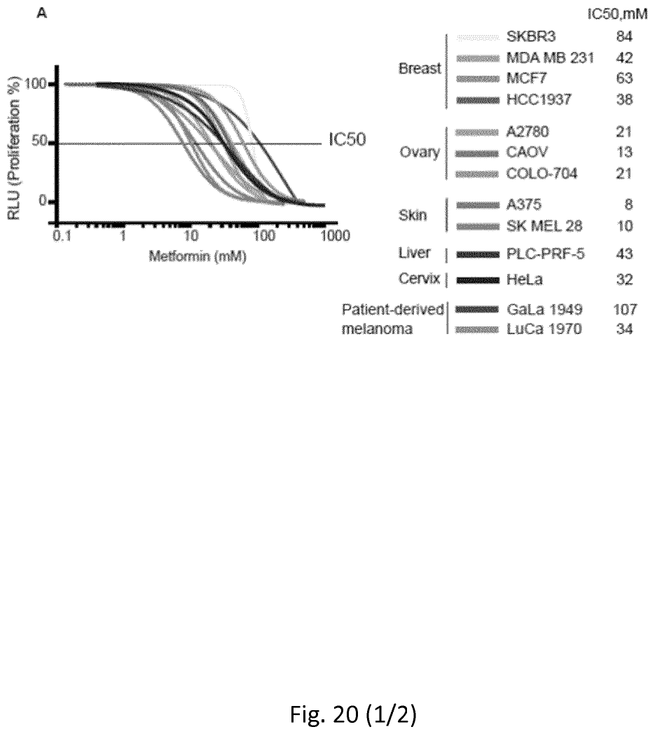

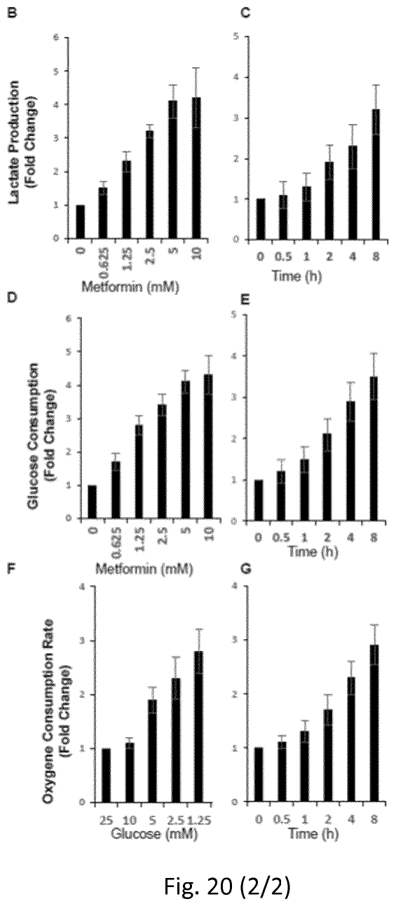

FIG. 20. Targeting metabolic plasticity of cancer cells (A) Proliferation assessed by CellTiter-Glo assay of cell lines representative of different cancer types treated with increasing concentrations of metformin. (B, C) Quantification of lactate production normalized by cell numbers of cells treated with the indicated concentrations of metformin for 12 hours (A) or with 5 mM of metformin for the indicated time points (B). (D, E) Quantification of glucose consumption normalized by cell numbers of cells treated with the indicated concentrations of metformin for 12 hours (D) or with 5 mM of metformin for the indicated time points (E). (F, G) Quantification of oxygen consumption rate normalized by cell numbers of cells cultured in DMEM medium containing the indicated concentrations of glucose for 12 hours (F) or in DMEM medium containing with 2.5 mM glucose for the indicated time points (G).

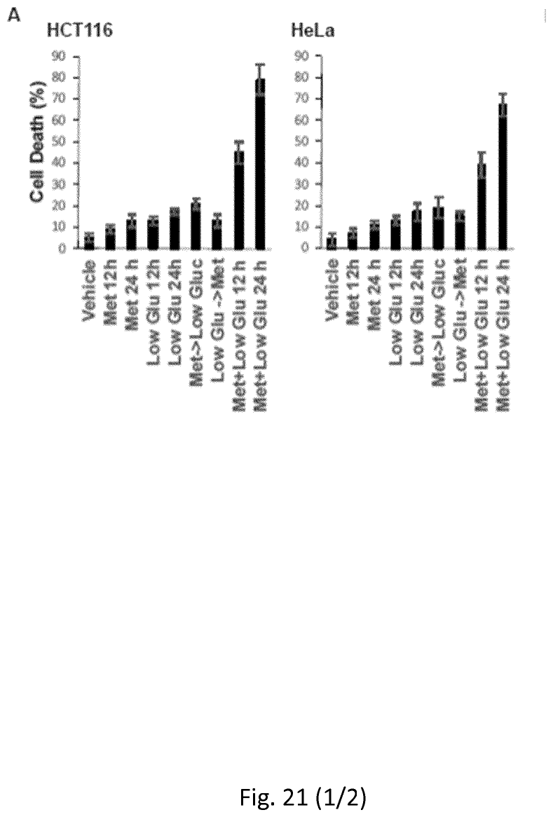

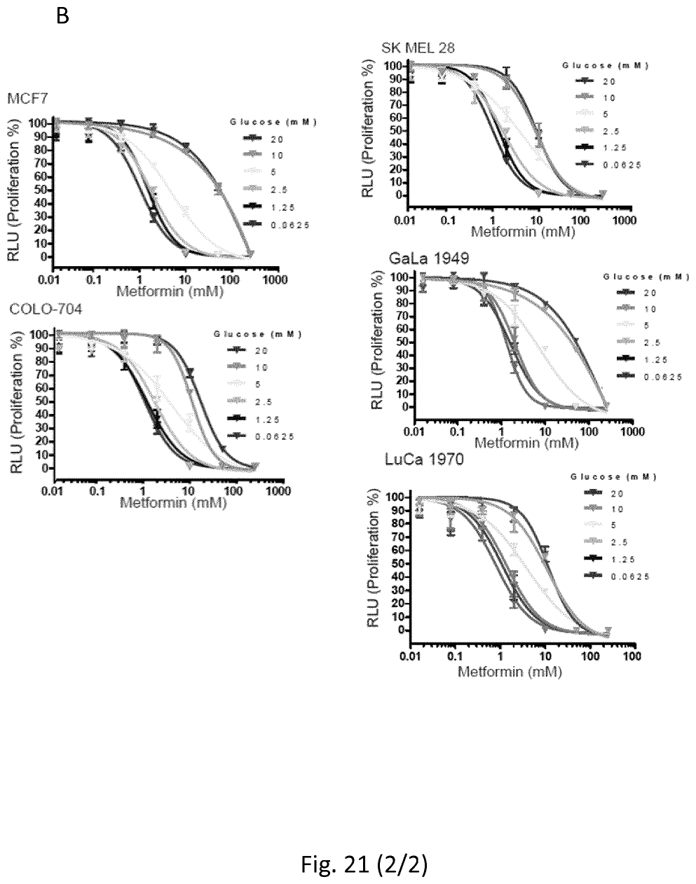

FIG. 21. Synergistic cytotoxicity of simultaneous treatment with metformin and low glucose (A) Percentage of cell death of HCT116 and HeLa cells cultured for 12 or 24 hours in either nutrient-rich DMEM or in DMEM containing 2.5 mM glucose (Low Glu) in the presences or absence of metformin (5 mM). Alternatively, cells were sequentially treated for 12 hours with metformin followed by washing out and plating in low glucose medium or vice versa. (B) Proliferation of the indicated cancer cells cultured in media containing the indicated concentration of glucose and treated with increasing concentrations of metformin for 24 h as assessed by CellTiter-Glo assay.

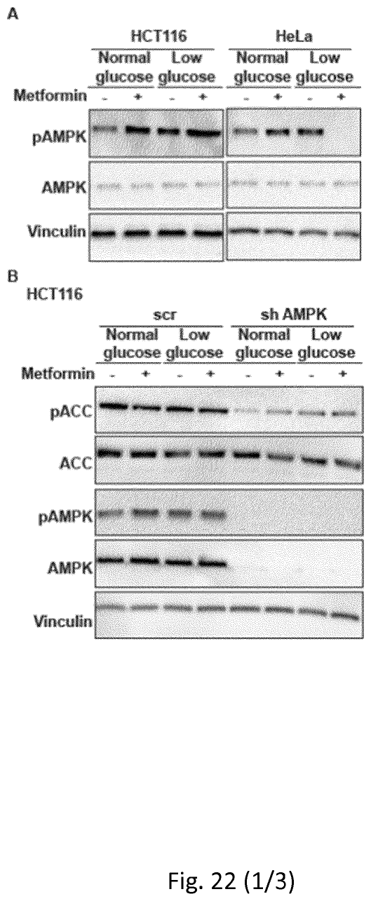

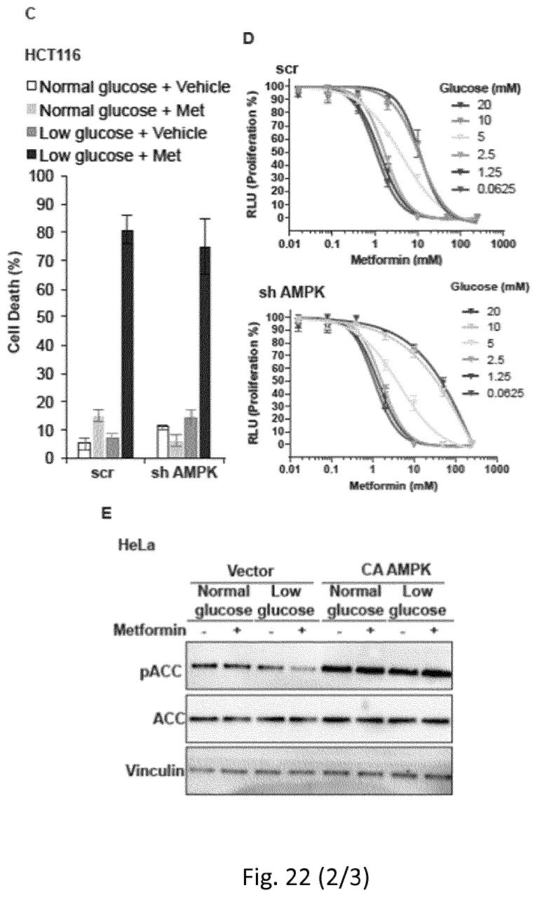

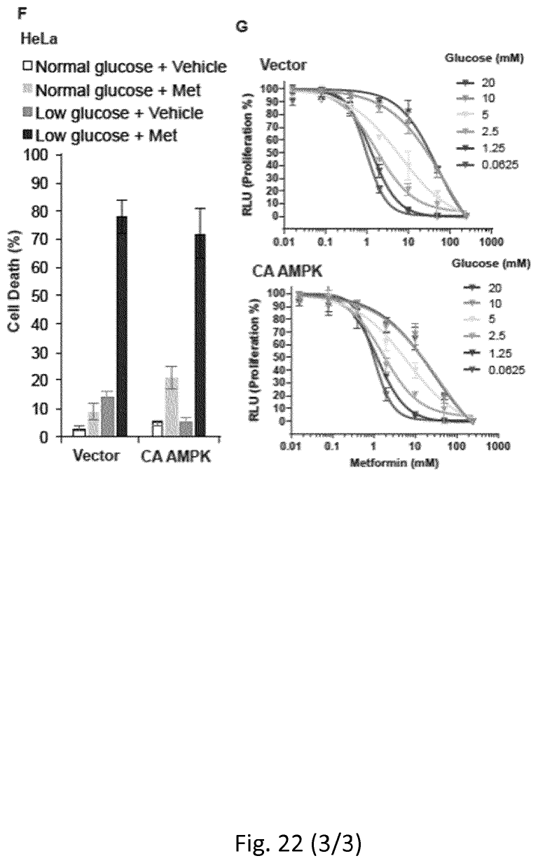

FIG. 22. Synergistic cytotoxicity of low glucose and metformin is AMPK-independent. (A) Immunoblotting analysis of lysates derived from HCT116 and HeLa cells cultured for 24 hours in DMEM (replenished every 6 hours) containing either 10 mM or 2.5 mM glucose (Normal or Low glucose respectively) in the absence or presence of metformin (5 mM). (B) Immunoblotting analysis of lysates derived from HCT116 cells stably expressing either scrambled shRNA or shRNA against AMPK and treated as in A. (C) Percentage of cell death of control or AMPK-depleted HCT116 cells treated as in B. (D) Proliferation assessed by CellTiter-Glo assay of control or AMPK-depleted HCT116 cells cultured in DMEM containing the indicated concentration of glucose and treated with increasing concentrations of metformin for 24 h. (E) Immunoblotting analysis of lysates derived from HeLa stably expressing either vector or constitutively active form of AMPK and treated as in A. (F) Percentage of cell death of HeLa cells treated as in A. (G) Proliferation assessed by CellTiter-Glo assay of control or HeLa cells expressing constitutively active AMPK and cultured in DMEM containing the indicated concentration of glucose and treated with increasing concentrations of metformin for 24 hours.

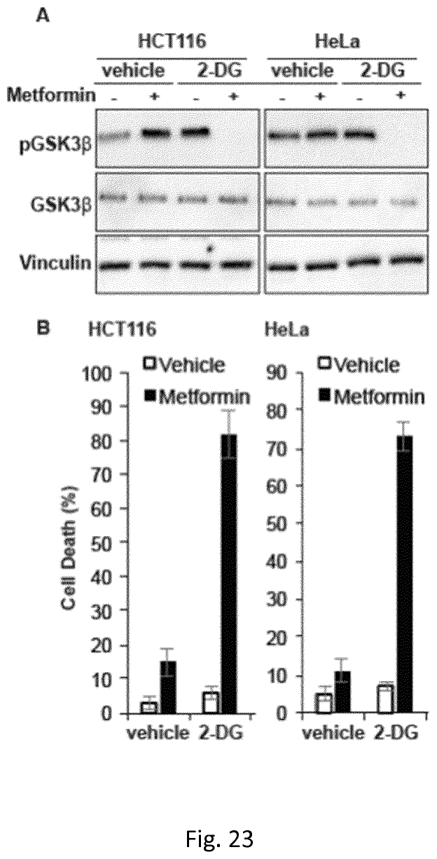

FIG. 23. Pharmacological inhibition of glycolysis by 2-DG synergizes with metformin (A) Immunoblotting analysis of lysates derived from HCT116 and HeLa cells cultured in nutrient-rich DMEM and treated for 24 with either vehicle or metformin (5 mM) in the absence or presence of 2-DG (25 mM). (B) Percentage of cell death of HCT116 and HeLa cells cultured in nutrient-rich DMEM (containing 10% FBS and 10 mM glucose) and treated for 24 with either vehicle or metformin (10 mM) in the absence or presence of 2-DG (25 mM).

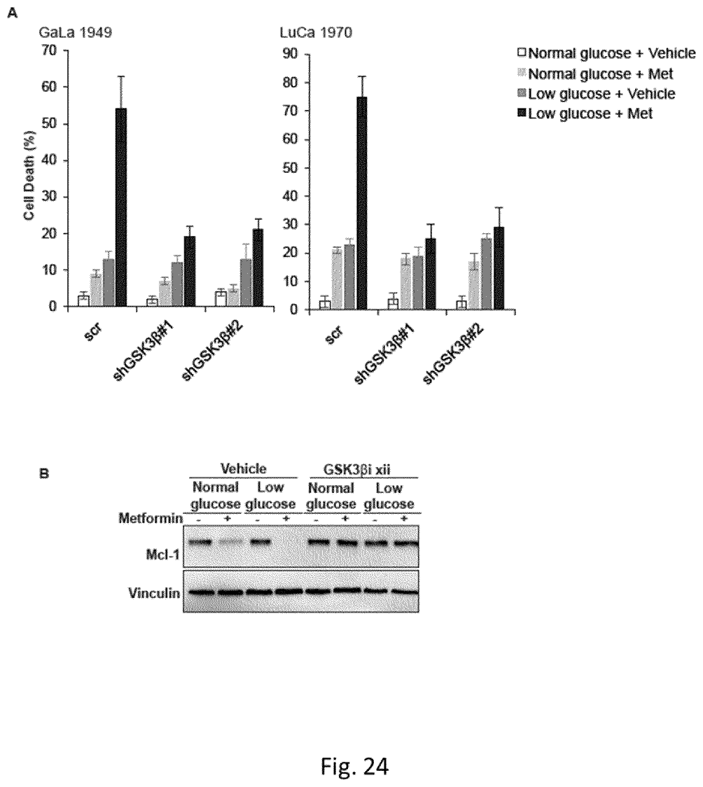

FIG. 24. GSK3β-induced downregulation of MCL-1 mediates the cytotoxicity of metformin-low glucose combination (A) Percentage of cell death of patient-derived melanoma cells GaLa1948 and LuCa1973 expressing either scrambled shRNA or shRNAs against GSK3β and cultured for 72 hours in DMEM (replenished every 6 hours) containing either 10 mM or 2.5 mM glucose (Normal or Low glucose respectively) in the absence or presence of metformin (10 mM). (B) Immunoblotting analysis of lysates derived from HCT116 cells treated with either vehicle or GSK3β inhibitor xii (20 μM) and then cultured as in D. Treatment with GSK3β inhibitor xii started 1 hour before metformin treatment. Media were replenished every 6 hours.

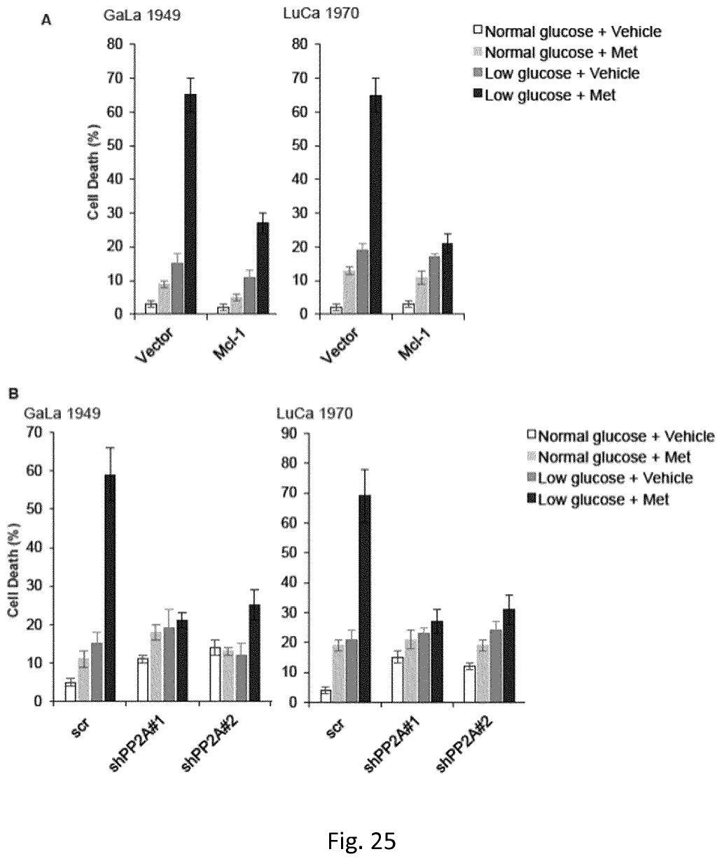

FIG. 25. Validation of the mechanistic model in patient-derived melanoma cells (A) Percentage of cell death of patient-derived melanoma cells GaLa1948 and LuCa1973 expressing either vector or MCL-1 constructs and cultured for 72 hours in DMEM (replenished every 6 hours) containing either 10 mM or 2.5 mM glucose (Normal or Low glucose respectively) in the absence or presence of metformin (10 mM). (B) Percentage of cell death of patient-derived melanoma cells GaLa1948 and LuCa1973 expressing either scrambled shRNA or shRNAs against PP2A and cultured for 72 hours in DMEM (replenished every 6 hours) containing either 10 mM or 2.5 mM glucose (Normal or Low glucose respectively) in the absence or presence of metformin (10 mM).

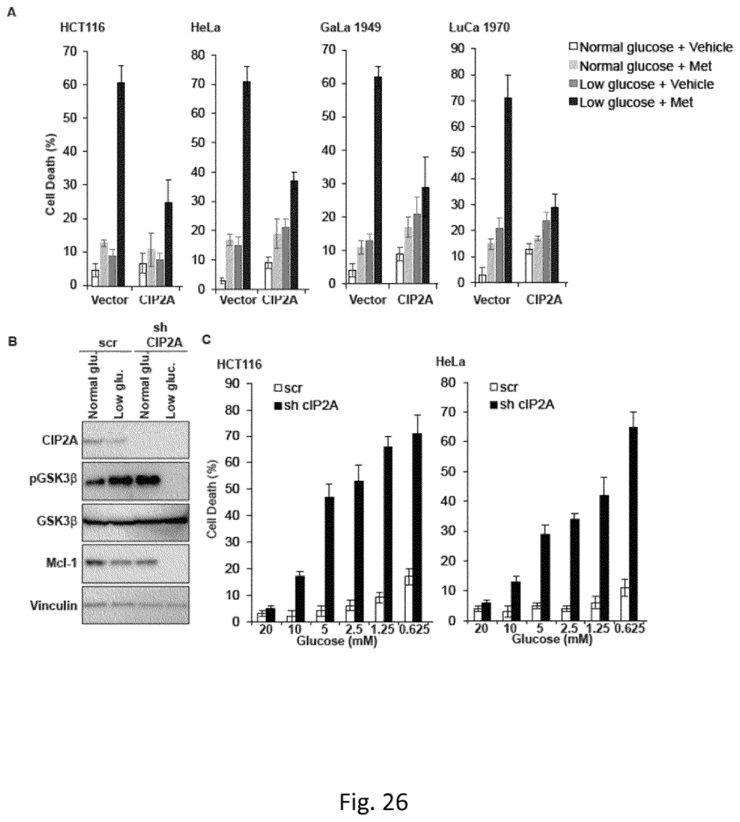

FIG. 26. Modulation of MCL-1 or CIP2A oncoproteins impacts metformin/low glucose cytotoxicity (A) Percentage of cell death of HCT116, HeLa cells and patient-derived melanoma cells GaLa1948 and LuCa1973 expressing either vector or MCL-1 constructs and cultured for 72 hours (GaLa1948 and LuCa1973 cells) or 24 hours (HCT116 and HeLa cells) in DMEM (replenished every 6 hours) containing either 10 mM or 2.5 mM glucose (Normal or Low glucose respectively) in the absence or presence of 10 mM (GaLa1948 and LuCa1973 cells) or 5 mM (HCT116 and HeLa cells) metformin. (B) Immunoblotting analysis of lysates derived from HCT116 cells expressing either scrambled shRNA or shRNA against CIP2A and cultured for 24 hours in DMEM (replenished every 6 hours) containing either 10 mM or 2.5 mM glucose (Normal or Low glucose respectively). (C) Percentage of cell death of HCT116, HeLa cells expressing either scrambled shRNA or shRNA against CIP2A and cultured for 24 hours in DMEM (replenished every 6 hours) containing the indicated concentrations of glucose.

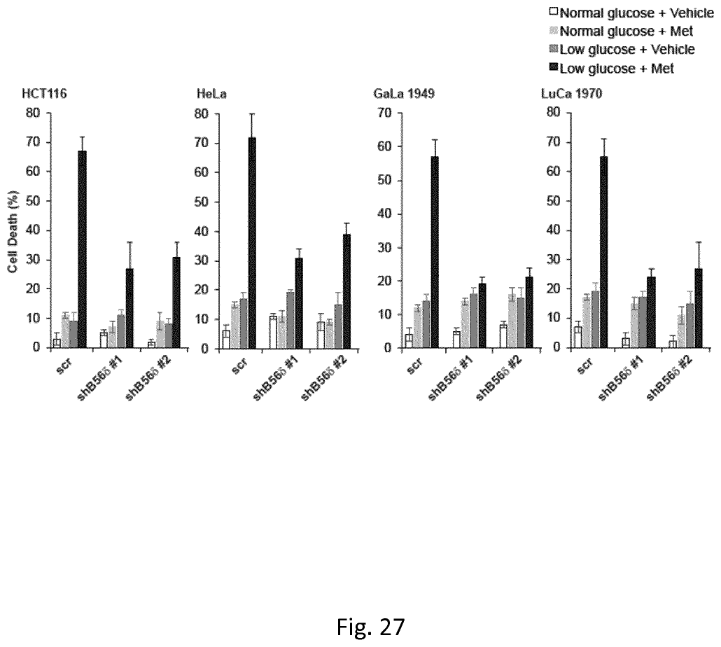

FIG. 27. Depletion of B56δ impedes metformin-low glucose cytotoxicity.

Percentage of cell death of HCT116, HeLa cells and patient-derived melanoma cells GaLa1948 and LuCa1973 expressing either scrambled shRNA or shRNA against B56δ and cultured for 72 hours (GaLa1948 and LuCa1973 cells) or 24 hours (HCT116 and HeLa cells) in DMEM (replenished every 6 hours) containing either 10 mM or 2.5 mM glucose (Normal or Low glucose respectively) in the absence or presence of 10 mM (GaLa1948 and LuCa1973 cells) or 5 mM (HCT116 and HeLa cells) metformin.

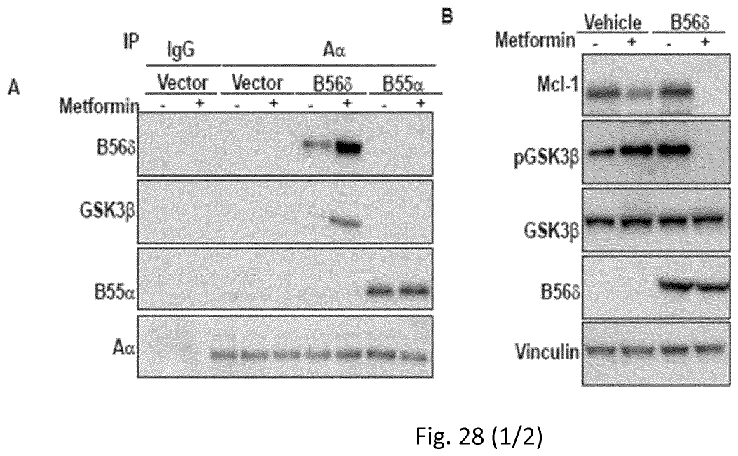

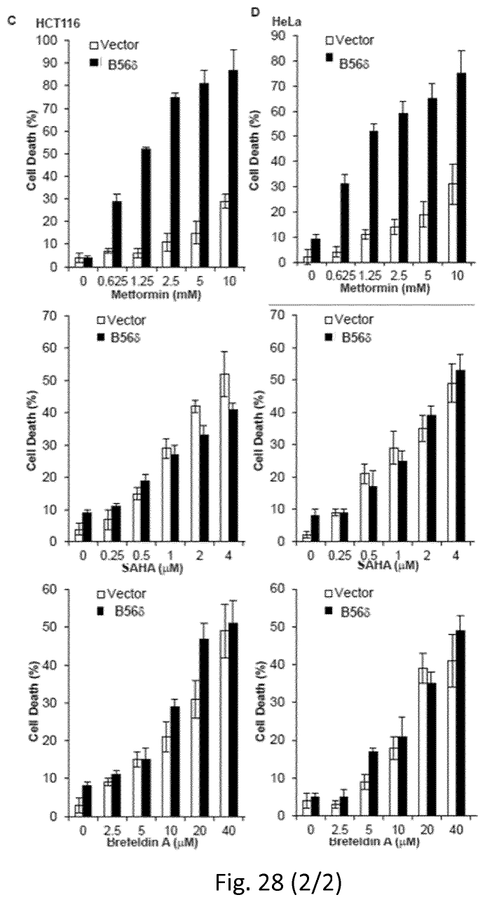

FIG. 28. Analysis of the role of the B56C regulatory subunit. (A) Immunoprecipitation analysis of PP2A Aα from cell lysates derived from HCT116 cells overexpressing either vector, B56δ or B55α construct and cultured for 24 hours in nutrient-rich DMEM in the absence or presence of metformin (5 mM). (B) Immunoblotting analysis of lysates derived from HCT116 cells overexpressing either vector or B56δ construct and cultured in nutrient-rich DMEM and treated for 24 with either vehicle or metformin (5 mM). (C) Percentage of cell death of HCT116 (A) and HeLa cells overexpressing either vehicle or B56δ and treated for 24 hours with the indicated concentrations of metformin, SAHA or Brefeldin A.

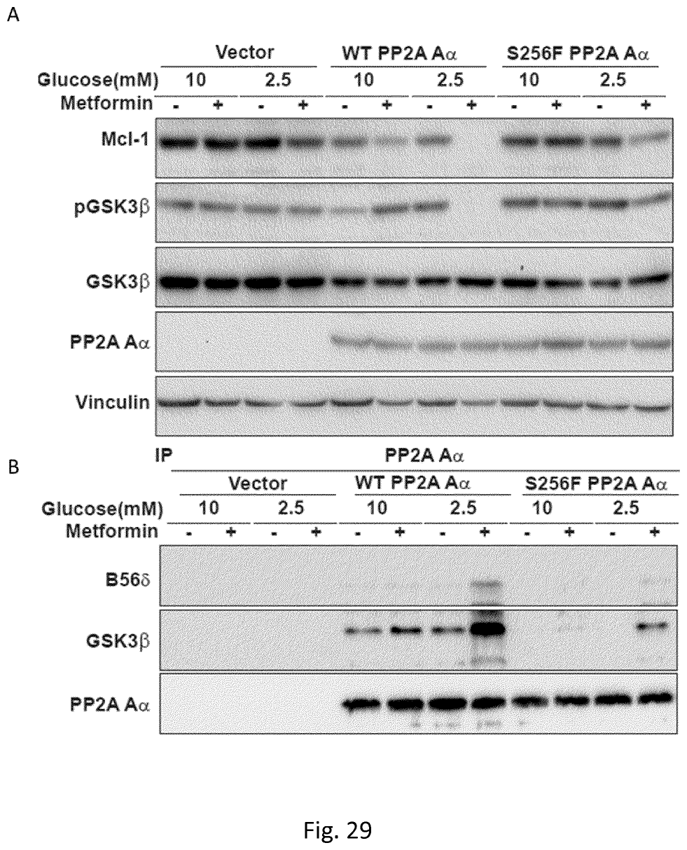

FIG. 29. Enhanced recruitment of B56δ to PP2A complex mediates metformin-low glucose cytotoxicity

(A) Immunoblotting analysis of total cell lysates used for immunoprecipitation (B). Immunoprecipitation analysis of PP2A Aα from cell lysates derived from PP2A-ablated HCT116 cells and reconstituted with vector, wild type PP2A Aα or PP2A Aα mutant S256F and cultured for 24 hours in DMEM (replenished every 6 hours) containing either 10 mM or 2.5 mM glucose (Normal or Low glucose respectively) in the absence or presence of metformin (5 mM).

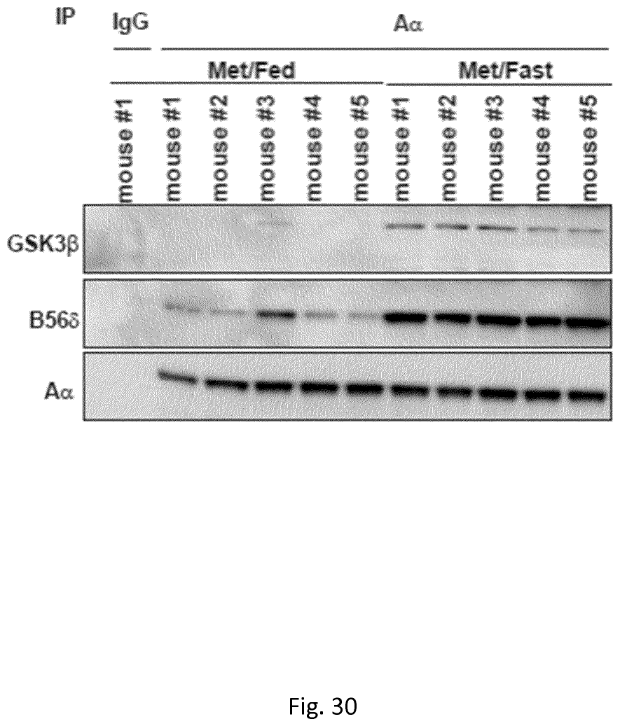

FIG. 30. Immunoprecipitation analysis of PP2A Aα from tumor lysates used in FIG. 19C.

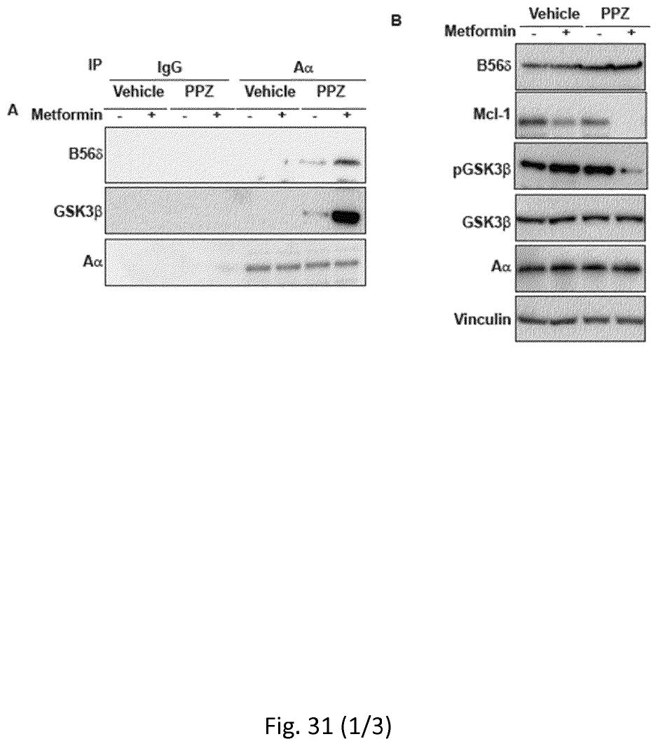

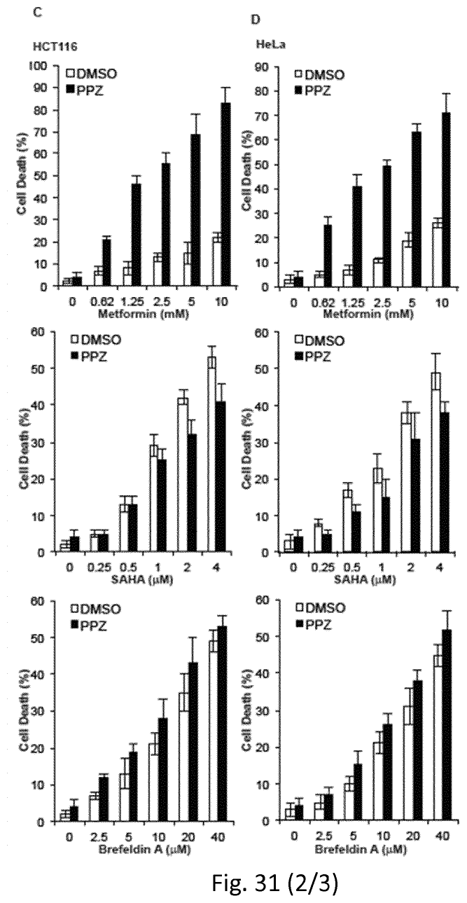

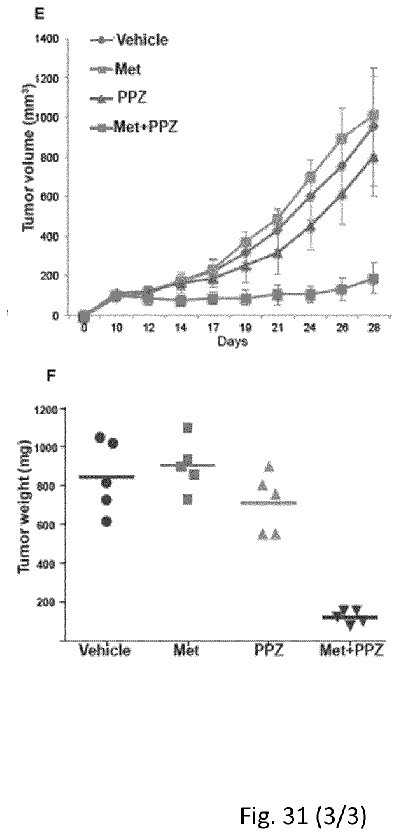

FIG. 31. PPZ synergizes with metformin and recapitulates the molecular effects of low glucose (A) Immunoprecipitation analysis of PP2A Aα from cell lysates derived from HCT116 cells treated with either vehicle or PPZ (10 μM) and cultured for 24 hours in nutrient-rich DMEM in the absence or presence of metformin (5 mM). Treatment with PPZ started 1 hour before metformin treatment. (B) Immunoblotting analysis of total cell lysates used for immunoprecipitation in (A). (C, D) Percentage of cell death of HCT116 (C) and HeLa (D) cells treated for 24 hours with the indicated concentrations of metformin in the absence or presence of PPZ (10 μM). (E) In vivo growth of HCT116 xenograft tumors as measured in mice treated with either dextrose water vehicle, metformin (200 mg/kg administered daily by oral gavage, PPZ (5 mg/kg administered daily by intra-peritoneal injection) or a combination of metformin and PPZ. Error bars indicate SEM. (n=5 per group). (F) Weight of tumors isolated from mice in different groups in (E).

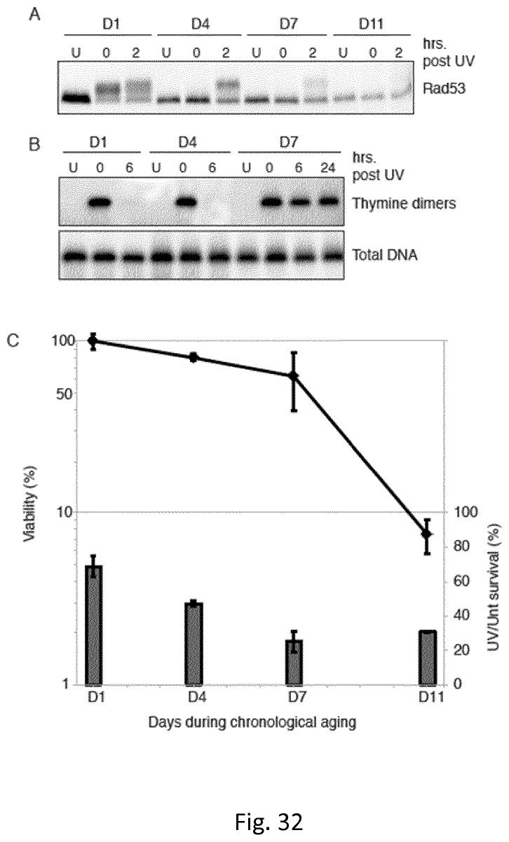

FIG. 32. Readouts to monitor chronological lifespan in yeast.

A. Yeast cells were subjected to chronological aging growth conditions as described in Materials and Methods. Aliquots of cells were removed at Days 1, 4, 7 and 11 and proteins were extracted from untreated, 0 and 2 hours post UV treatment (40J/m²). Western blot analysis was performed and Rad53 was detected.

B. Samples were harvested from untreated, 0, 6 or 24 hours post UV (40 J/m²) at Days 1, 4 and 7. Genomic DNA was prepared and subjected to Southern Blot analysis. Filters were probed with an antibody that detects thymine dimers (top panel). As a loading control, blots were stripped and re-probed with an anti-single stranded DNA antibody (bottom panel).

C. Viability of cells undergoing chronological aging was monitored at Days 1, 4, 7 and 11. The ratio of UV-treated to untreated cells is depicted. Mean values+/−St Dev on n=3 replicates are shown.

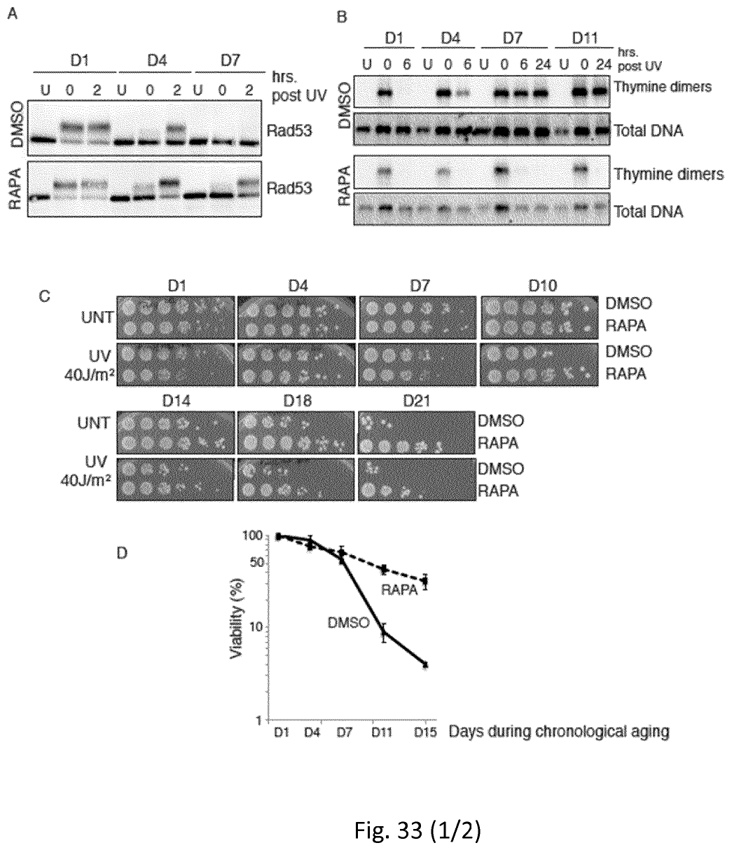

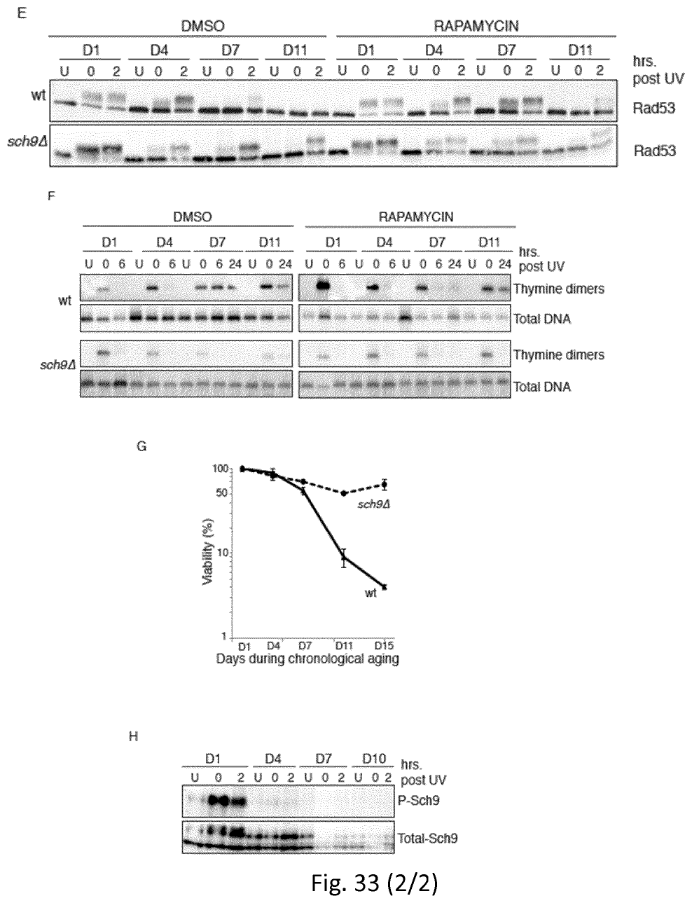

FIG. 33. Rapamycin-mediated inhibition of Tor1 and deletion of SCH9 improve DDR, NER and extend lifespan

A. DMSO or Rapamycin (2 ng/ml) were added to cells at Day 0 and CLS kinetic time course was carried similar to that described in FIG. 32. Western blot analysis on protein samples prepared from untreated, 0 and 2 hours post UV (40 J/m²) was performed using anti-Rad53 antibodies. B. Genomic DNA was prepared from untreated, 0, 6 and 24 hours after UV samples. Thymine dimers removal and total DNA content were detected with anti-dT dimer and anti-ss DNA antibodies respectively. C. Viability of DMSO- and Rapamycin-treated cells was monitored using spot assay analysis. Cells were harvested from the original cultures, serially-diluted and spotted on rich media. Plates were then subjected to 0 or 40 J/m² UV. After incubation at room temperature for 3 days, plates were scanned. D. Viability curve representing DMSO and Rapamycin-treated wt cells at Days 1, 4, 7, 10 and 15. Mean values+/−St Dev on three replicates are shown. E. wt and sch9 cells−/+Rapamycin were subjected to CLS kinetic time course and proteins were extracted from untreated, 0 and 2 hours post UV (40 J/m²). Rad53 phosphorylation was monitored by Western blot analysis. F. Thymine dimer removal was assessed in wt and sch9 cells−/+Rapamycin using same procedure as that described in B. G. Viability curve representing wt and sch9 cells undergoing CLS in control conditions (i.e. DMSO only) spanning Days 1, 4, 7, 11 and 15. Mean values+/−St Dev on three replicates are shown. Inventors note that FIGS. 33D and G share the same reference (wt in DMSO), as they come from a single CLS kinetic here presented in two parts to emphasize the effects of Torc1 inhibition by Rapamycin and of SCH9 ablation, respectively. H. Untreated, 0 and 2 hr post UV protein samples were probed for the status of Sch9 phosphorylation using antibodies specific for p-Sch9 (above panel) and total Sch9 protein levels (bottom panel).

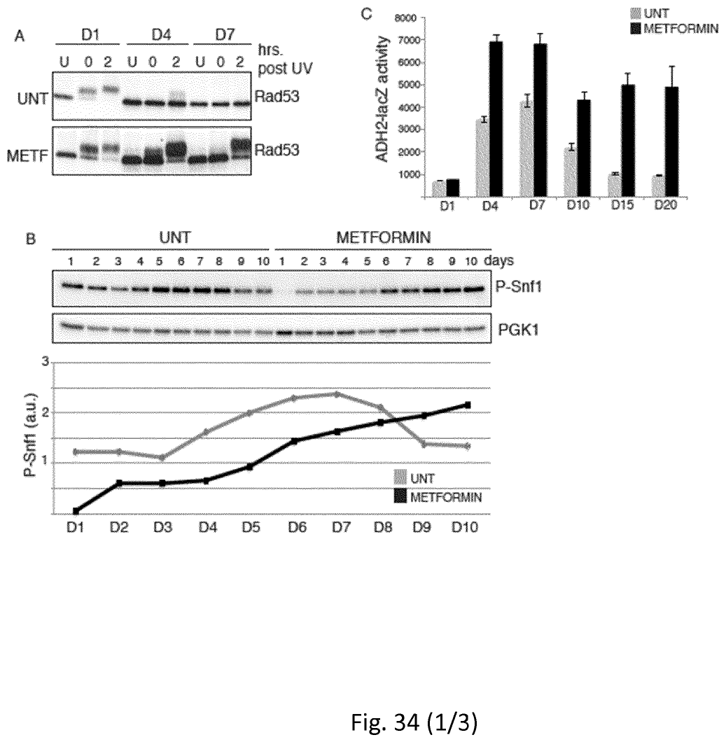

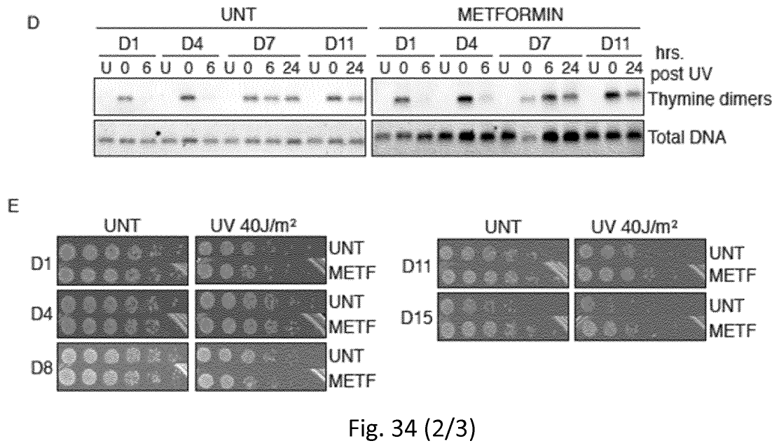

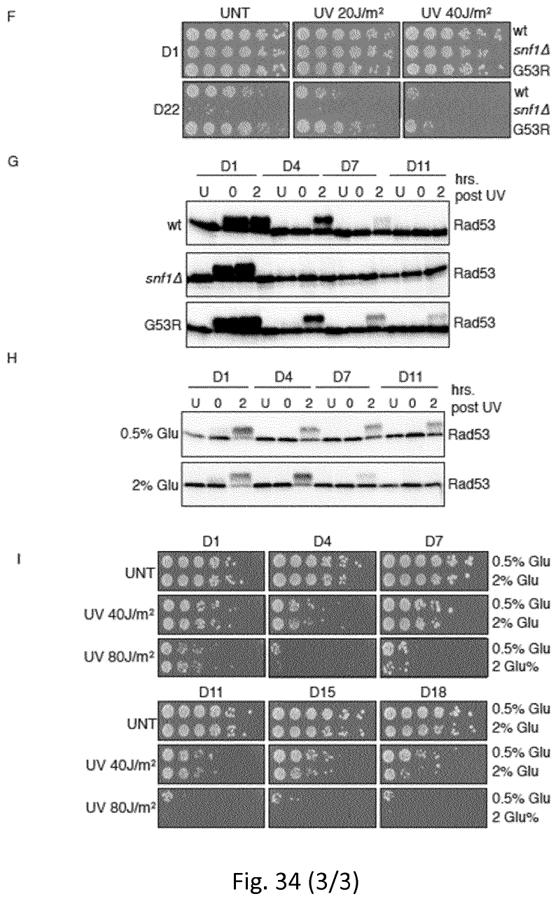

FIG. 34. Metformin and constitutively-active Snf1 improve DDR and promote longevity but not NER

A. Rad53 phosphorylation was monitored in untreated and Metformin (80 mM)-treated cells undergoing CLS at Days 1, 4 and 7.

B. Phosphorylation of Snf1 (T172) was detected throughout CLS (daily from 1-10) in untreated and Metformin-treated cells. Western blot filter was stripped and reprobed with anti-PGK as an equal loading control (top panel). Quantification of phospho-Snf1 levels relative to PGK is represented graphically (bottom panel).

C. Snf1 kinase activity was also monitored using ADH2-lacZ expression. lacZ expression was measured using P3-galactosidase assay as described in Materials and Methods in untreated (grey) and metformin-treated (black) cells at Days 1, 4, 7, 10, 15 and 20.

D. Thymine dimers and single-stranded DNAs were detected in samples harvested from untreated and metformin-treated cells at Days 1, 4, 7 and 11 that represent untreated, 0, 6 and 24 hours post UV (40 J/m²).

E. Viability of untreated and metformin-treated cells undergoing CLS was monitored using spot assay analysis at Days 1, 4, 8, 11 and 15−/+UV (40 J/m²).

F. Cell viability of snf1 and cells expressing constitutively-active Snf1 (referred to as G53R) at Days 1 and 22. UV treatment of 0, 20 and 40 J/m² was applied.

G. DDR activation was assessed using Rad53 phosphorylation in snf1 cells and hyperactive SNF1-G53R allele relative to wt cells undergoing CLS.

H. Cells were grown in 2% or 0.5% glucose and subjected to CLS kinetics. Protein extracts were prepared as described above and Rad53 phosphorylation was assessed by Western blot analysis.

I. Viability of cells grown in 2% or 0.5% glucose was monitored using spot assay analysis at Days 1, 4, 7, 11, 15 and 18.0, 40 or 80 J/m² UV treatment was applied.

FIG. 35. Deletion of positive regulators of PP2A, Rrd1 and Tip41, ameliorates DDR, NER and extends lifespan.

A. RRD1 and TIP41 encode positive regulators of the Serine/Threonine phosphatase PP2A (left panel). A pharmacogenomic screen identified tip41 and rrd1 as resistant to rapamycin (5 ng/ml) and metformin (80 mM) treatment (right panel).

B-D. wt, tip41 and rrd1 cells−/+Rapamycin were subjected to CLS kinetic time course and protein extracts were prepared from each strain at Days 1, 4, 7 and 11. Western blot analysis was performed to assess Rad53 phosphorylation in untreated, 0 and 2 hours post UV (40J/m²) (B). Thymine dimers removal and total DNA loading control were assessed in all 3 strains−/+Rapamycin at Days 1, 4, 7 and 11 in untreated, 0, 6 and 24 hours post UV (40 J/m²) (C). Viability of wt, tip41 and rrd1−/+Rapamycin was monitored using spot assay analysis. Results of Days 1 (young cells) and 27 (old cells) in 0 and 40 J/m² are presented (D).

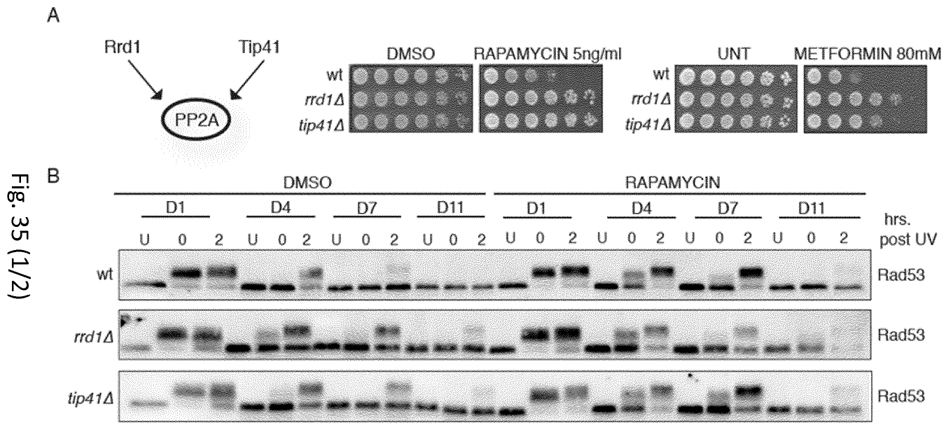

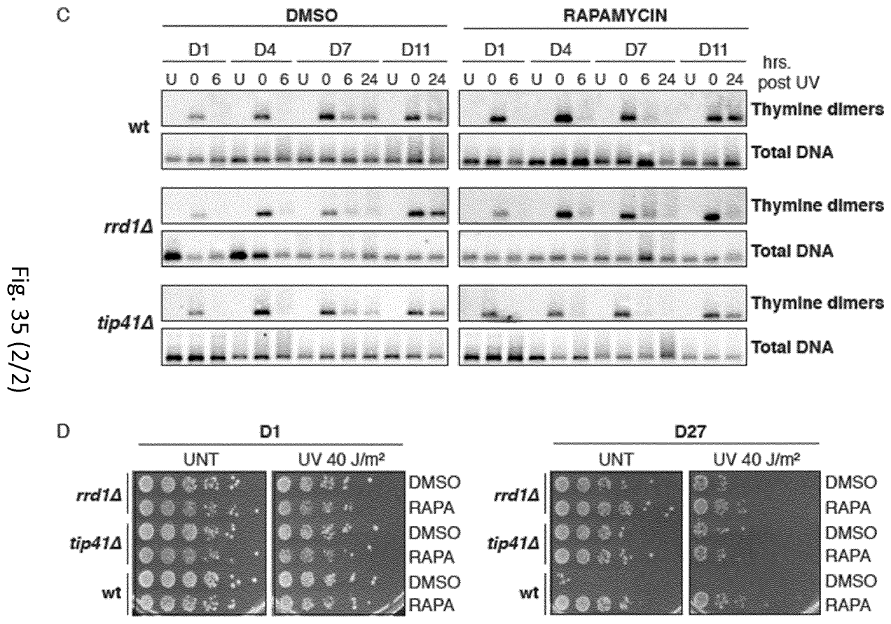

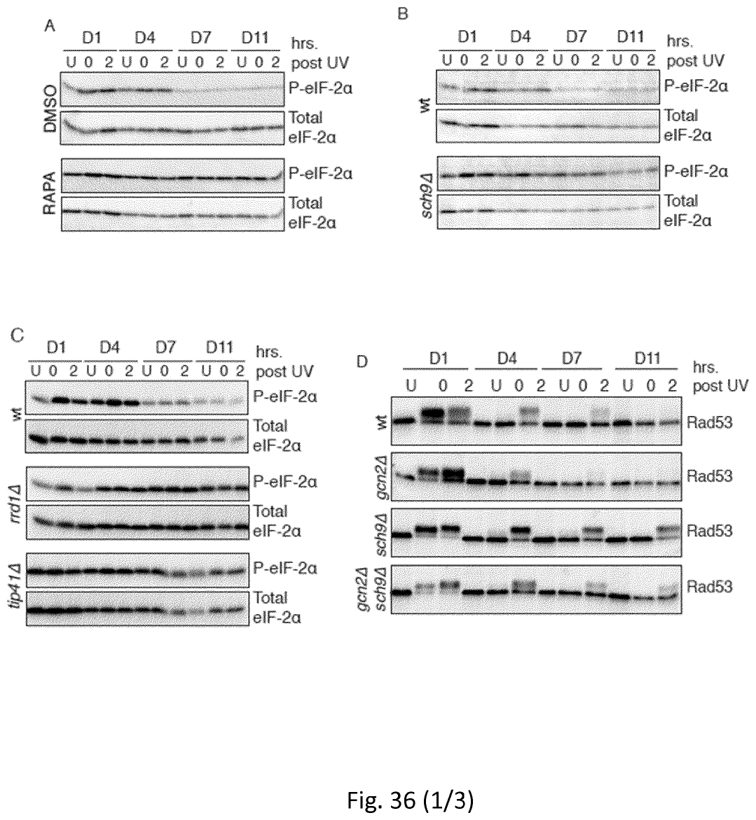

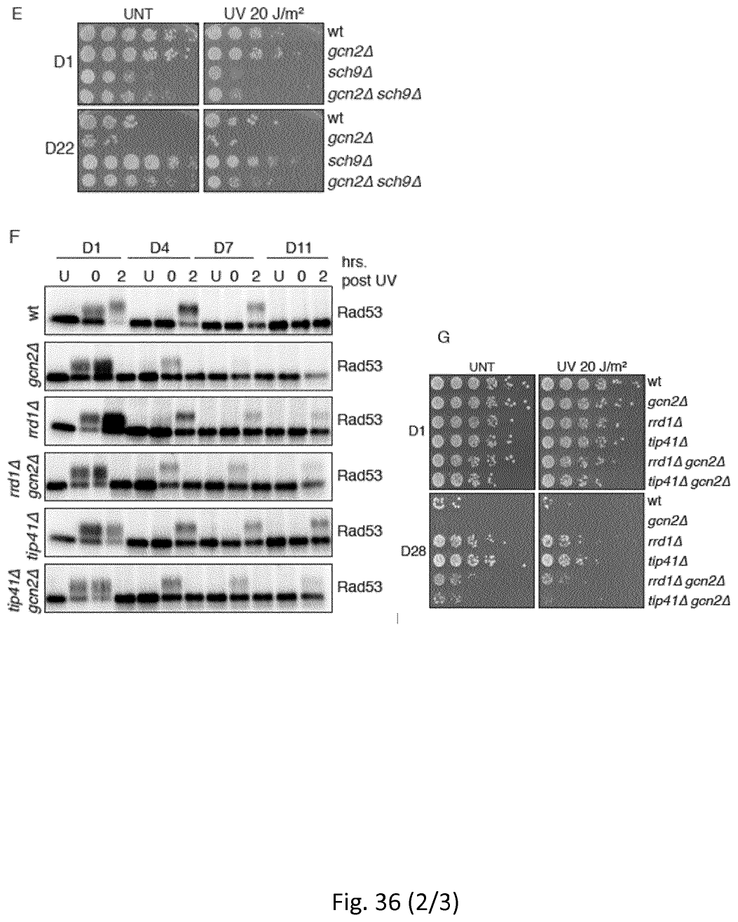

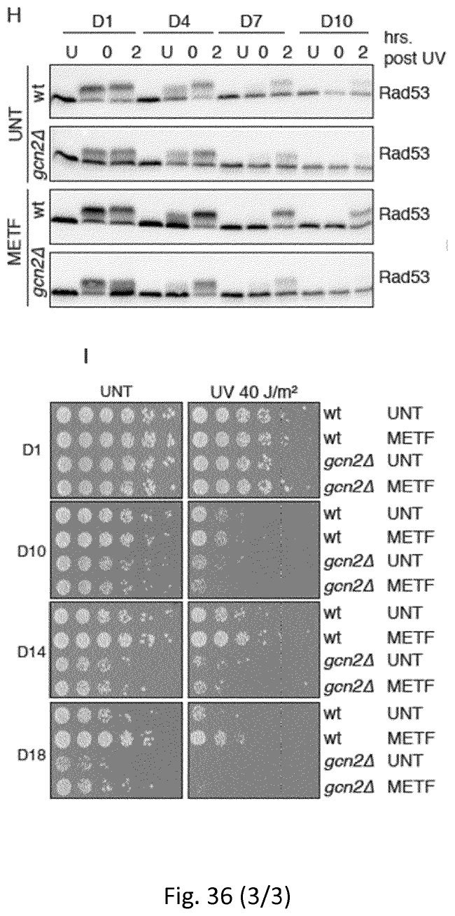

FIG. 36. Gcn2 kinase contribution to pro-longevity conditions.

A-C. Phosphorylation of eIF2α at S51 is assessed using an antibody specific for this site. Western blot analysis was performed on protein samples extracted from DMSO and Rapamycin-treated cells throughout CLS using anti-S51 eIF2α antibody (top panel). Filters were stripped and reprobed with an anti-total eIF2α antibody (bottom panel). The same procedure was repeated for wt and sch9 cells (B) as well as for wt rrd1 and tip41 cells (C).

D-E. Role of Gcn2 kinase during CLS was assessed. Deletion of GCN2 in wt and sch9 cells during CLS aging kinetics resulted in less efficient Rad53 activation as assessed by Western Blot analysis (D). Viability of gcn2 and sch9 gcn2 relative to wt and sch9 cells respectively at Days 1 and 22, in untreated and 20 J/m² UV (E)

F-G. CLS kinetic time course to assess the contribution of Gcn2 in the extended lifespan of tip41 and rrd1 cells. gcn2tip41 and gcn2rrd1 cells were compared to tip41 and rrd1 cells respectively. Western blot analysis on Rad53 is shown in F, while viability by spot assays at Days 1 and 28−/+20 J/m² is depicted in G.

H-I. Rad53 phosphorylation was analyzed in wt and gcn2 cells undergoing CLS without (left panels) or with (right panels) 80 mM Metformin. (H). Viability of gcn2−/+Metformin relative to wt, evaluated by serial dilution and spot assay−/+40J/m² UV (I).

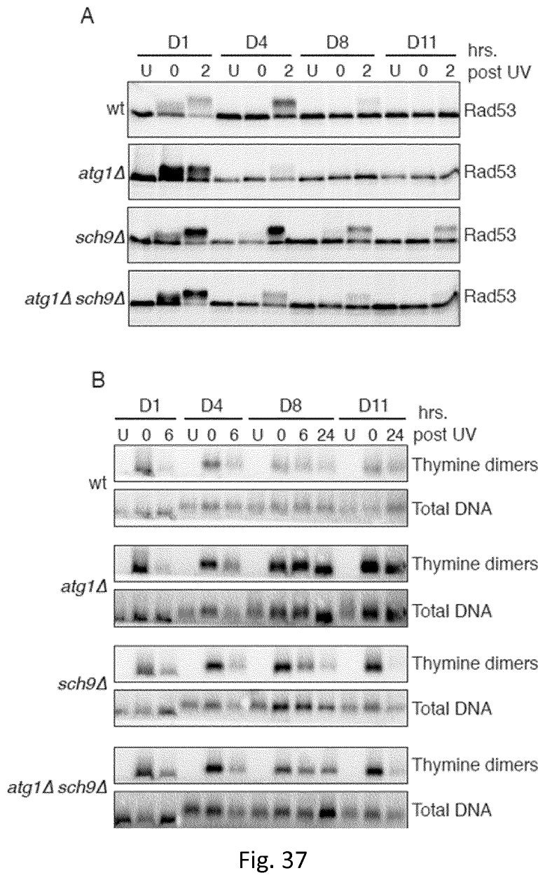

FIG. 37. Absence of autophagy affects DDR and NER efficiency (in wt and sch9 cells)

A. wt cells, atg1 (autophagy defective) and sch9 single mutants and atg1sch9 double mutant were subjected to CLS kinetic. Proteins were extracted at Days 1, 4, 8 and 11 before and after UV treatment (40J/m²) and Rad53 phosphorylation was assessed by Western Blot analysis with EL7 antibodies.

B. NER efficiency was investigated using dT dimers removal as a readout (upper panels). Hybridization with anti-ssDNA antibodies was performed on stripped membranes as loading control (lower panels).

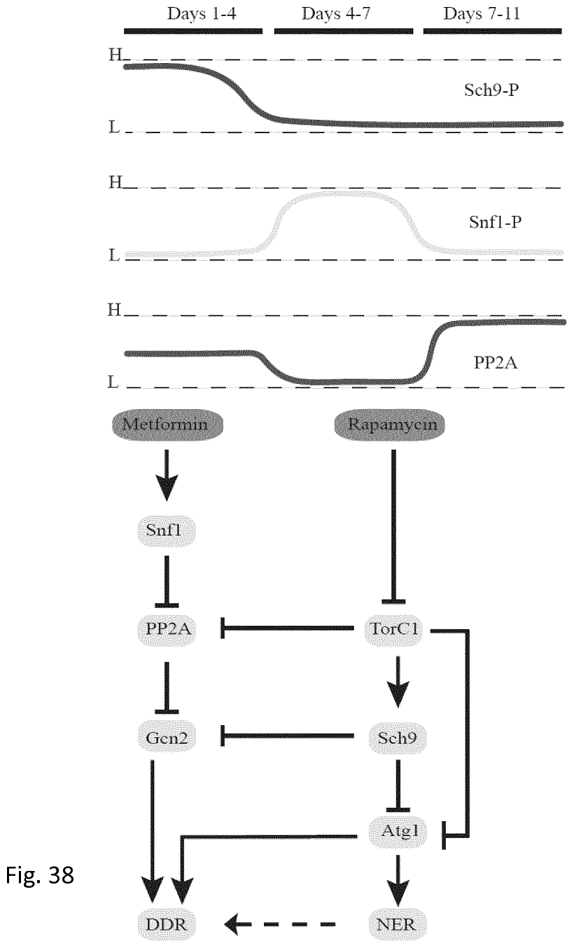

FIG. 38. Model. Top panel: Representation of relative activities of Torc1 (measured using Sch9 phosphorylation as a readout), Snf1 and PP2A during chronological aging. Activity of the Torc1 and Snf1 kinases and of PP2A phosphatase all fluctuate between a low (L) and high (H) level in the different stages of aging, and reciprocally influence each other.

Bottom panel: Cross-talks between metformin- and rapamycin-targeted pathways, affecting NER and DDR.

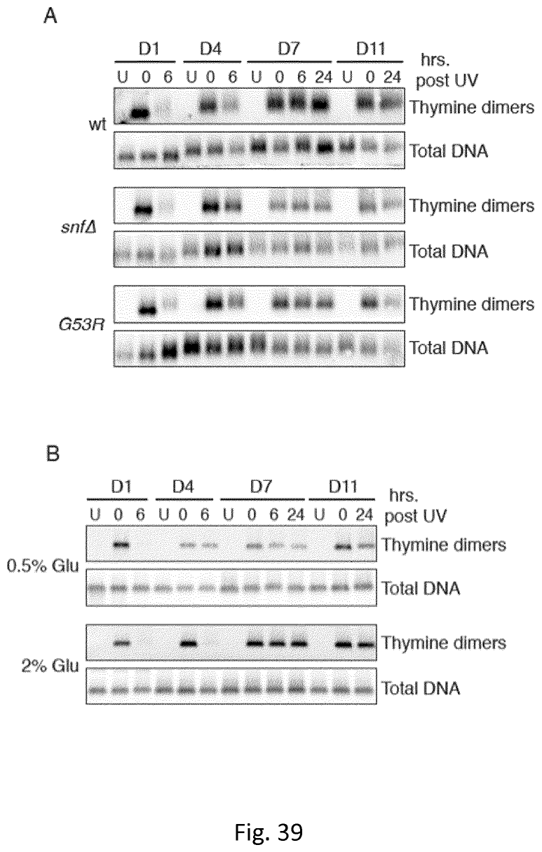

FIG. 39. Activation of the SNF1 pathway by either hyperactive Snf1-G53R or growth in low glucose does not improve NER during CLS

A: Genomic DNA was extracted before and after UV exposure (40J/m²) at Days 1, 4, 7 and 11 from cells of the indicated relevant genotypes. After Southern Blot transfer, membranes were incubated with anti-thymine dimers antibody (upper panels), stripped and re-probed with anti-ssDNA antibody as loading control (lower panels).

B: wt cells were subjected to CLS kinetic in SC medium containing 0.5% glucose (top part) or 2% glucose (bottom part). Total DNA was extracted before and after UV treatment and analysis of NER efficiency was performed with anti-TD (upper panels) and anti-ssDNA (lower panels) antibodies, as described in A.

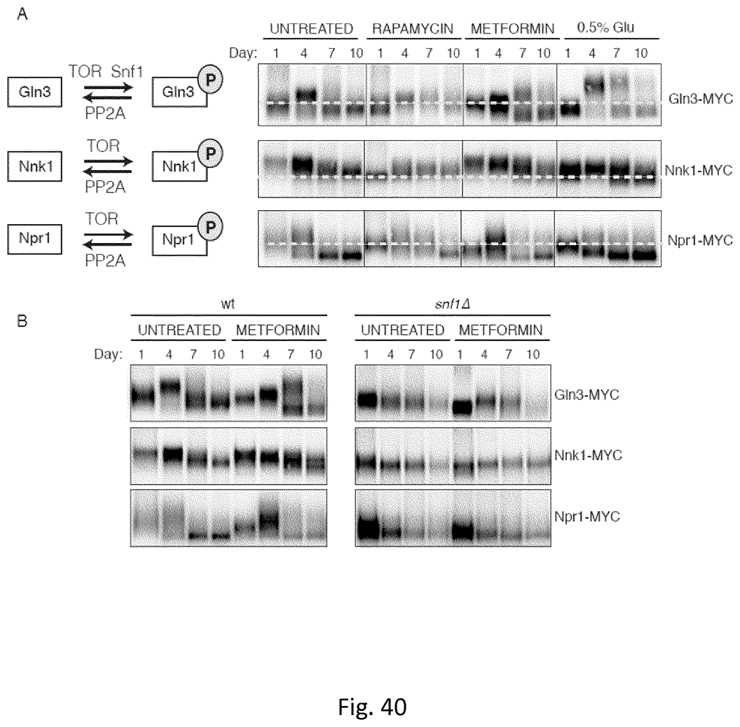

FIG. 40. PP2A activity during CLS using phosphorylation status of the targets Gln3, Nnk1 and Np1.

A. Gln3-Myc, Nnk1-Myc and Npr1-Myc phosphorylation was monitored at Days 1, 4, 7 and 10 in untreated, Metformin, Rapamycin and low glucose (0.5%) treatments.

B. Targets above were assessed in snf1 cells−/+Metformin treatment to monitor the contribution of Snf1 to their phosphorylation during CLS.

FIG. 41. Evidence of phospho-eIF2α independent manner to improve DDR and extend lifespan.

A. Rapamycin treatment rescues the DDR defect of gcn2 cells undergoing CLS. Rad53 phosphorylation is used as a measure of checkpoint activation in wt (top) and gcn2 (bottom) strains.

B. Viability using spot assay analysis is performed on wt and gcn2 cells−/+Rapamycin. Results are presented for young (Day 1) and old cells (Day 23)−/+UV (40 J/m²).

C-E. Monitoring eIF2α phosphorylation as described in FIG. 36, in the Metformin/Snf1 branch. Cells grown in 80 mM Metformin (C) or in low glucose (E), and strains carrying either the snf1A or the hyperactive SNF1-G53R allele (D) are all assessed for the status of eIF2α phosphorylation (top panel) as well as total eIF2α protein as a loading control (bottom panel).

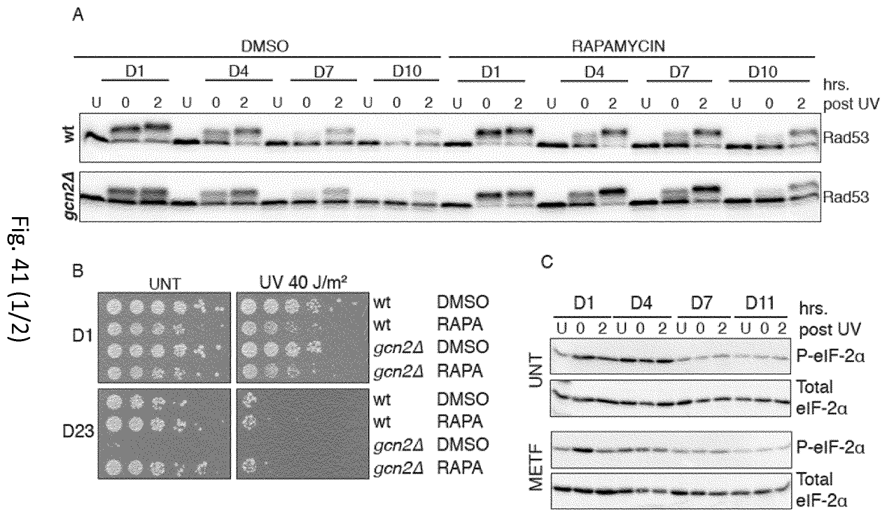

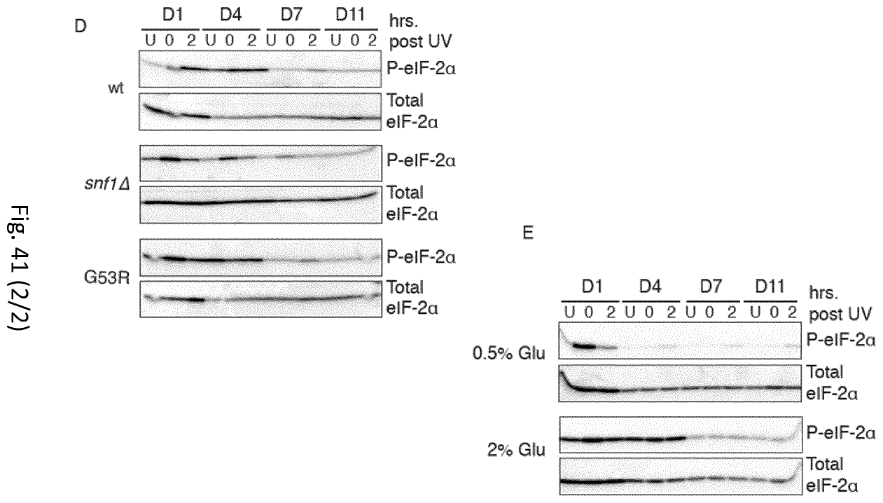

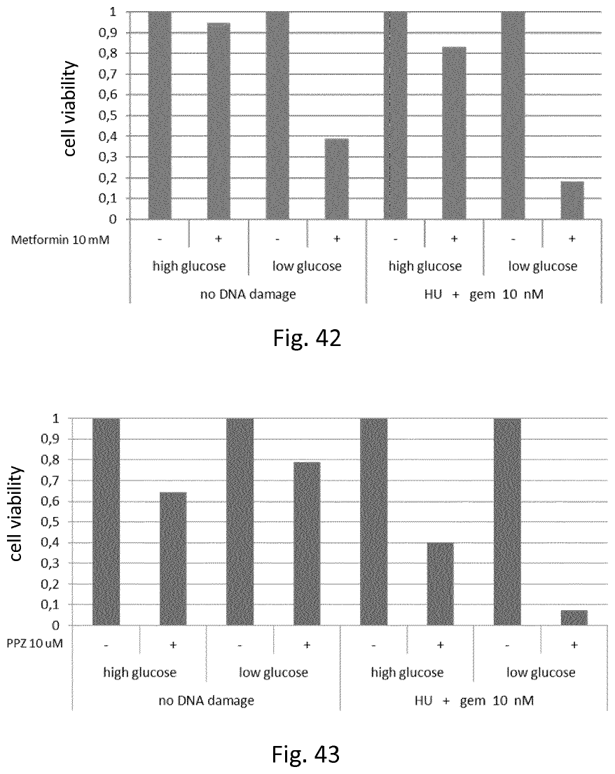

FIG. 42. Metformin enhances the effect of DNA damaging agents.

HeLa cells were seeded in 6-well plates (200,00 cells/well), and left untreated, or treated with DNA damaging agents (hydroxyurea 10 mM+gemcitabine 10 nM), in high (10 mM) or low (2.5 mM) glucose, and treated with metformin (10 mM) as indicated. After 24 hrs treatment, cells are allowed to grow for further 24 hrs (replacing the medium with normal medium), before being harvested and counted with trypan blue to measure viable cells. As observed, not only metformin (as described in other parts of this application) cooperates with low glucose in inducing cell death, but also potentiates the effect of DNA damaging agents in low glucose.

FIG. 43. Perphenazine enhances the effect of DNA damaging agents.

HeLa cells were seeded in 6-well plates (200,00 cells/well), and left untreated, or treated with DNA damaging agents (hydroxyurea 10 mM+gemcitabine 10 nM), in high (10 mM) or low (2.5 mM) glucose, and treated with perphenazine (PPZ) as indicated. After 24 hrs treatment, cells are allowed to grow for further 24 hrs (replacing the medium with normal medium), before being harvested and counted with trypan blue to measure viable cells. PPZ (that activates PP2A, as also shown in other parts of this application where it is shown its ability to cooperate with metformin) cooperates with DNA damage, and this cooperation is further increased in low glucose conditions.

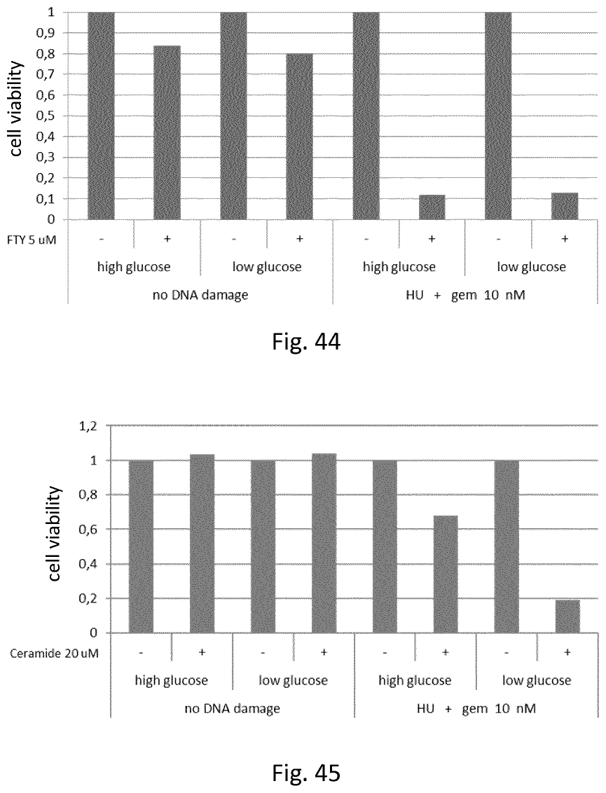

FIG. 44. FTY-720 enhances the effect of DNA damaging agents.

HeLa cells were seeded in 6-well plates (200,00 cells/well), and left untreated, or treated with DNA damaging agents (hydroxyurea 10 mM+gemcitabine 10 nM), in high (10 mM) or low (2.5 mM) glucose, and treated with FTY-720 (FTY) as indicated. After 24 hrs treatment, cells are allowed to grow for further 24 hrs (replacing the medium with normal medium), before being harvested and counted with trypan blue to measure viable cells. FTY-720 (a sphingosine analog that activates PP2A through multiple mechanisms, including suppression of the PP2A inhibitor SET) strongly cooperates with DNA damage.

FIG. 45. Ceramide enhances the effect of DNA damaging agents.

HeLa cells were seeded in 6-well plates (200,00 cells/well), and left untreated, or treated with DNA damaging agents (hydroxyurea 10 mM+gemcitabine 10 nM), in high (10 mM) or low (2.5 mM) glucose, and treated with Ceramide as indicated. After 24 hrs treatment, cells are allowed to grow for further 24 hrs (replacing the medium with normal medium), before being harvested and counted with trypan blue to measure viable cells. Ceramide (a known modulator of PP2A activity, that emerged as an important mediator from the yeast studies) strongly cooperates with DNA damage, especially in low glucose conditions.

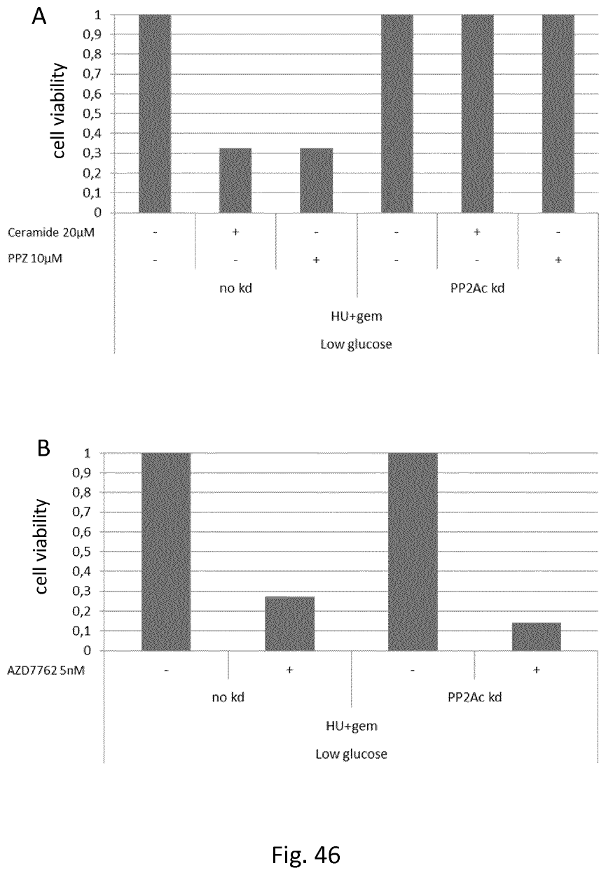

FIG. 46. PP2A mediates the cooperative effect of several drugs with DNA damaging agents.

A) HeLa cells transduced by means of a lentiviral inducible construct (pLKO-Tet-On) carrying shRNA targeting sequences against the catalytic subunit c of PP2A (“PP2Ac-kd”) or control («no kd») were treated with doxycyclin to induce the transcription of shRNAs. 72 hrs post knockdown induction, cells are treated with DNA damaging agents (Hydroxyurea+gemcitabine), ceramide, or perphenazine (PPZ) as indicated for further 24 hours, then medium is replaced with fresh complete DMEM, cells are allowed to grow for further 24 hrs before being harvested and counted with trypan blue. B) Same as in (a), but AZD7762 (an inhibitor of the phosphorylation and activation of the checkpoint kinases involved in the modulation of DDR in mammals—chk1 and chk2) was used in combination with DNA damaging agents. PP2Ac knockdown is able to completely rescue the cooperative reduction of cell viability observed by co-treatment of DNA damaging agents with known PP2A activators (ceramide, PPZ), showing that PP2A activity is required for the efficacy of the drug combination. Strikingly, PP2A knockdown is not protective against AZD7762 in combination with DNA damaging agents, in strong agreement with our hypothesis that PP2A acts upstream of the regulation of the DDR, while agents such as AZD7762, that act downstream, are not affected by PP2A down-modulation.

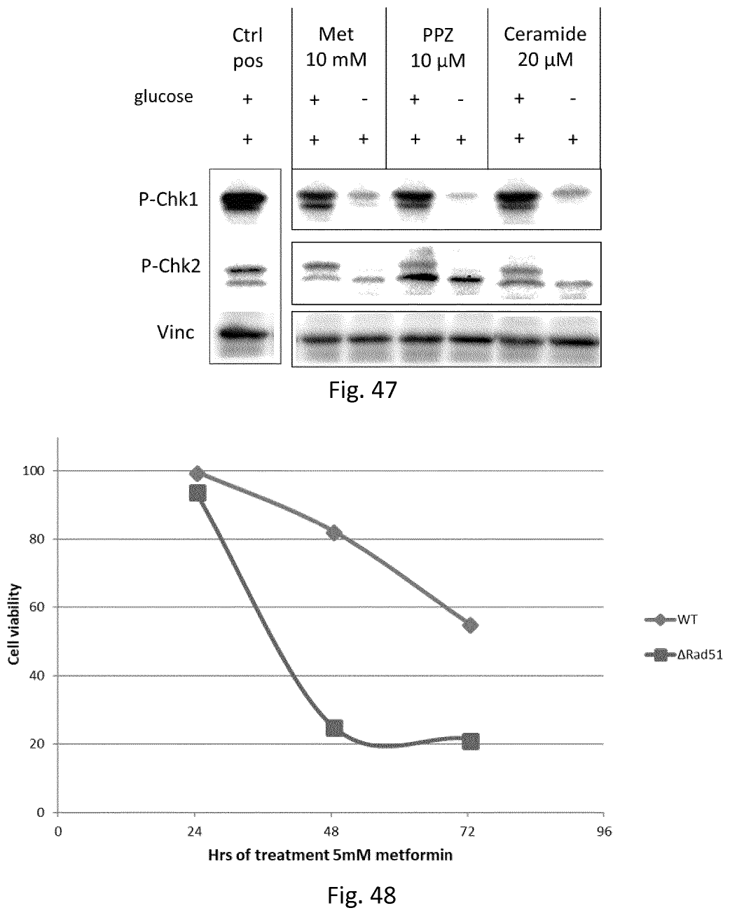

FIG. 47. PP2A activation counteracts the DNA damage response.

Western blot analysis of phosphorylated Chk1 and Chk2 (P-Chk1 and P-Chk2) from Hct116 cells treated with DNA damaging agents (hydroxyurea and gemcitabine) in combination with the indicated drugs (Met=Metformin, PPZ=Perphenazine, Ceramide), in the presence of high or low glucose conditions. Vinculin (Vinc) is used as loading control. Similar results were obtained in HeLa cells. Activation of PP2A triggers a reduction of Chk1/Chk2 phosphorylation (activation) in high glucose condition, that is dramatically amplified in low glucose condition, consistently with the greater antitumor effect observed in low glucose.

FIG. 48. Genetic impairment of the DDR is synthetic lethal to treatments able to activate PP2A

Knockdown of RAD51 in Bx-PC3 cells dramatically sensitizes them to PP2A-inducing treatment. Bx-PC3 cells, WT and transduced with a lentiviral construct (pLKO.1) carrying shRNA targeting sequences against RAD51, were treated with metformin in combination with low glucose for 24, 48 and 72 hrs. Cell viability is measured with Cell Titer Glo assay.

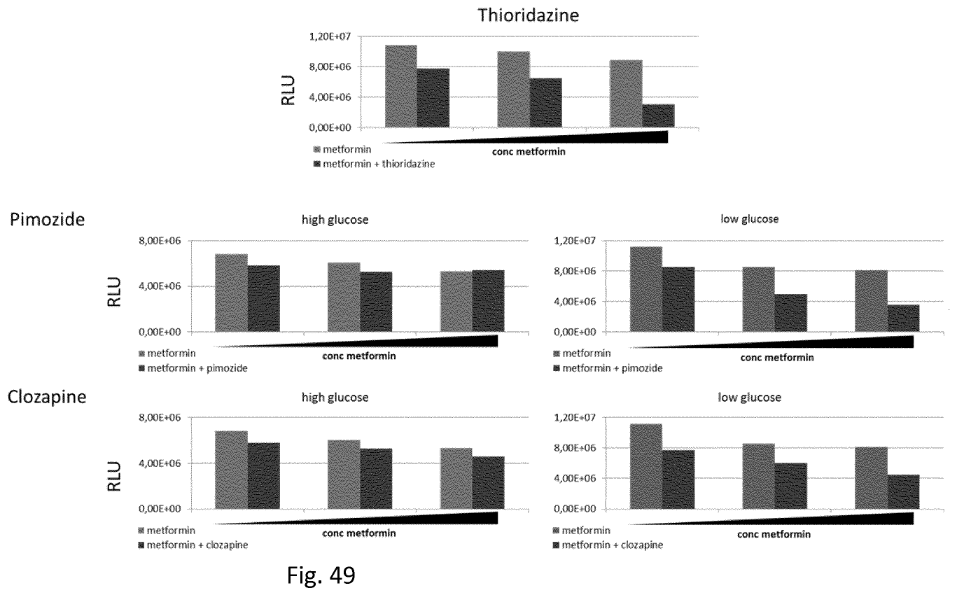

FIG. 49. Characterization of the activity of small molecules known as PP2A activators.

A panel of known PP2A activators were tested in combination with metformin. The assay was performed in 96-well plates (n=4) in HeLa cells, treating cells as described before, in high (10 mM) and low (2.5 mM) glucose conditions. Viability was measured by CellTiter Glo. Parallel assays (cell count with Trypan blue) confirmed the results. Perphenazine and thioridazine were the only drugs able to cooperate with metformin in reducing tumor cell viability in high glucose conditions, while 7 other compounds cooperated with metformin under low glucose conditions (see also table 1B).

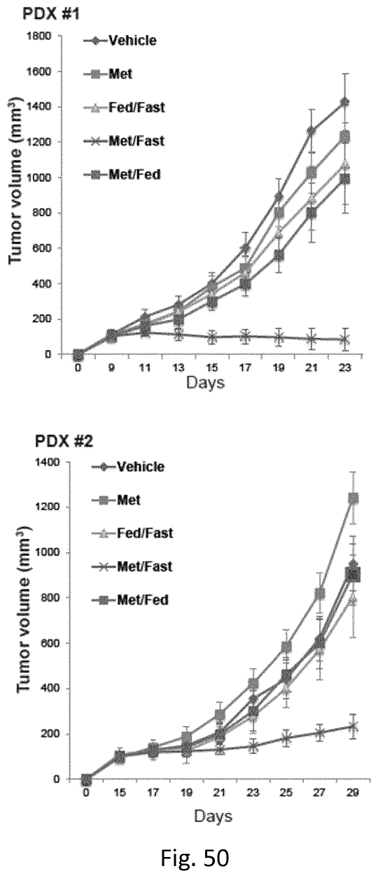

FIG. 50. Combination of Metformin with Intermittent fasting works also in PDX models. In vivo growth of PDX models (patient-derived xenografts) derived from tumor samples isolated from two melanoma patients. Upon establishment of tumors, mice were kept on 24-hour feeding/fasting cycles and treated with either vehicle or metformin (200 mg/kg) administered by oral gavage every 48 hours either during feeding or fasting cycles. Tumor volume was calculated as (length×width×width)/2. Error bars indicate SEM. (n=5 per group).

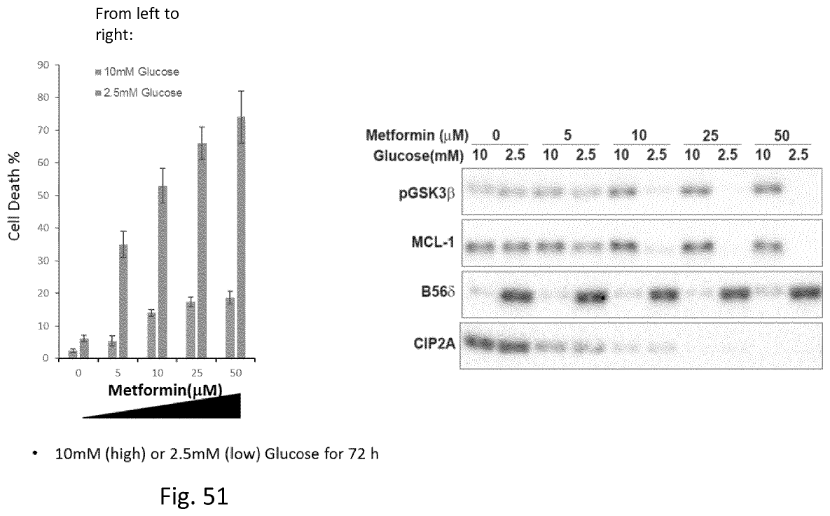

FIG. 51. Combination of Metformin with low glucose occurs at very low doses of metformin

(Left panel) Percentage of cell death of HCT116 cells cultured for 72 hours in DMEM containing either 10 mM glucose (Normal glucose) or 2.5 mM glucose (Low glucose) in the presence or absence of the indicated concentrations of metformin.

(Right panel) Immunoblotting analysis of lysates derived from HCT116 cells cultured as in the left panel.

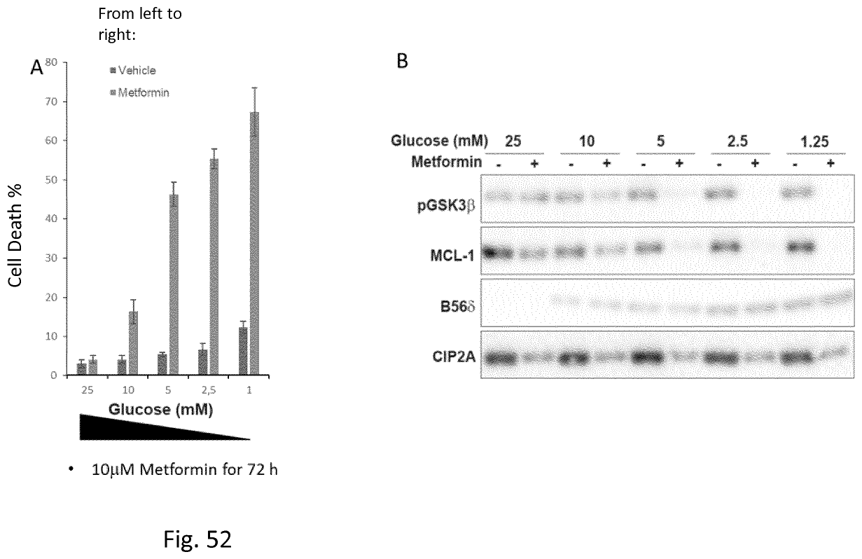

FIG. 52. Treatment with ultra-low doses of metformin (for longer durations) synergizes with low glucose through the PP2A-GSK3ß-Mcl1 axis

(A) Percentage of cell death of HCT116 cells cultured for 72 hours in DMEM as indicated. (B) Immunoblotting analysis of lysates derived from HCT116 cells cultured as in A.

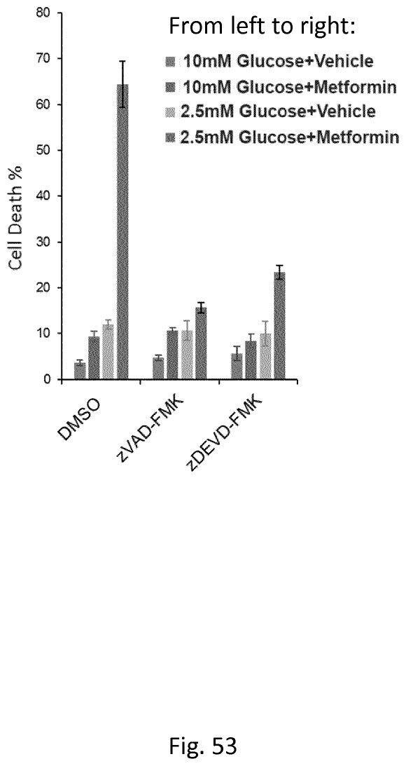

FIG. 53. Cell death by Metformin/low glucose is mediated by caspases

Percentage of cell death of HCT116 cells treated with either DMSO or the caspase inhibitors zVAD-FMK (25 mM), zDEVD-FMK (25 mM) and cultured for 24 hours in DMEM containing either 10 mM or 2.5 mM glucose (Normal or Low glucose respectively) in the absence or presence of metformin (5 mM). Treatment with the inhibitors started 1 hour before metformin treatment.

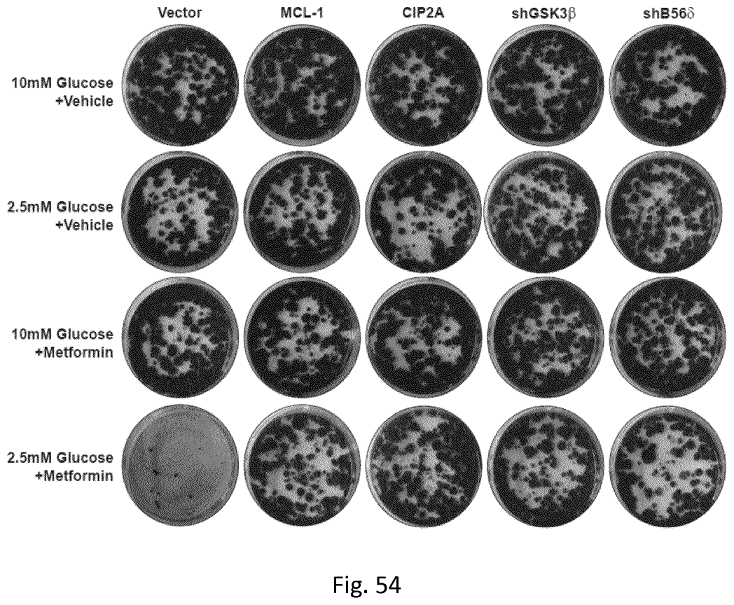

FIG. 54. Modulation of different components of the PP2A-GSK33-Mcl1 axis imparts long-term rescue against Metformin/low glucose in clonogenic survival assay

500 HCT116 cells from different conditions were plated for four weeks in DMEM containing either 10 mM or 2.5 mM glucose (Normal or Low glucose respectively) in the absence or presence of metformin (5 mM). Mcl1-CIP2A: overexpression; shGSK3β, shB560: knockdown

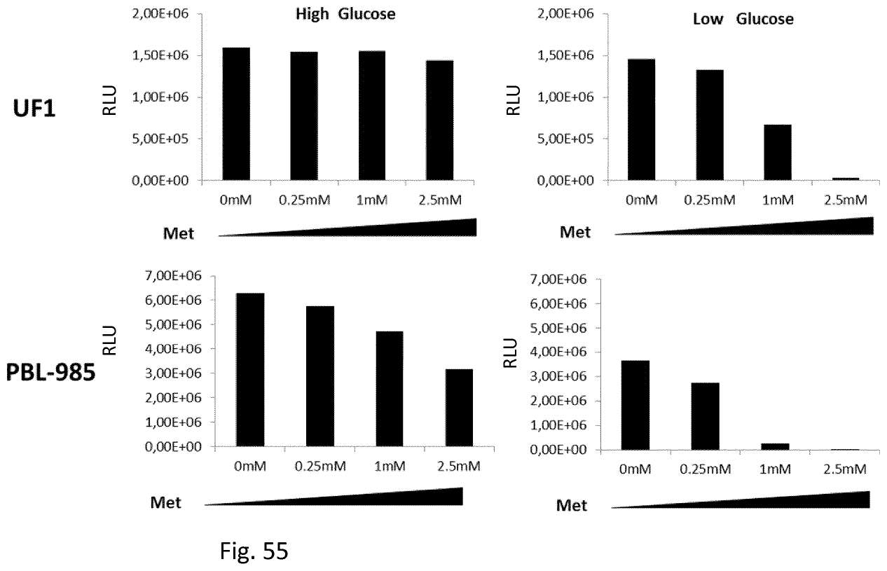

FIG. 55. Cell death by Metformin/low glucose in AML cell lines

Several AML cell lines were incubated in high (10 mM) or low (0 mM) glucose for 72h, in the absence or in the presence of the indicated concentrations of metformin. Cell viability was measured by the Cell Titer Glo assay. Examples of the results obtained are shown in the graphs.

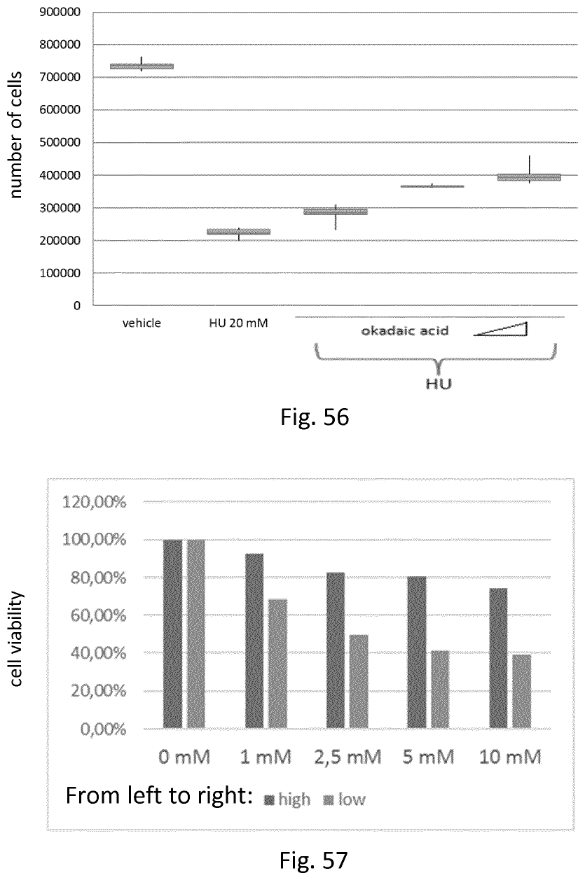

FIG. 56. PP2A inhibition partially protects from DNA damaging agents.

HeLa cells were seeded in 6-well plates (200,00 cells/well), serum starved for 24 hrs to synchronize, then treated in high concentration of HU (20 mM) for 12 hrs in the presence or in the absence of increasing concentrations of okadaic acid (from 0.01 nM to 1 nM). After treatment, cells are allowed to grow for further 24 hrs (replacing the medium with normal medium), before being harvested and counted with trypan blue to measure viable cells.

Okadaic acid inactivation of PP2A results in a better survival of cells treated with HU.

FIG. 57. BON-1 neuroendocrine tumor cells show reduction in cell viability upon combination of metformin with low glucose. Bon-1 cells were treated with the indicated concentrations of metformin, either in high (10 mM) or low (2.5 mM) glucose. The assay as performed as described in the other examples.

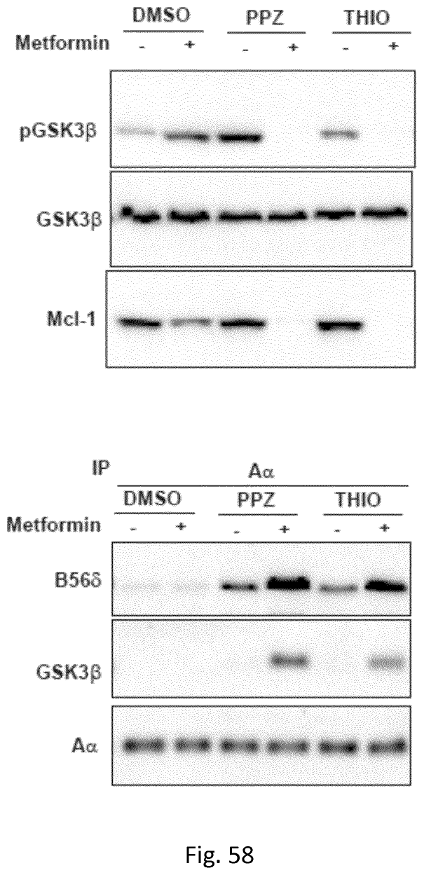

FIG. 58. Thioridazine treatment increase B56δ incorporation in PP2A holoenzyme. Immunoprecipitation and total cell lysate analysis of PP2A Aalpha from cell lysates derived from HCT116 cells treated with either DMSO, 10 uM PERPHENAZINE (PPZ) or 10 uM THIORIDAZINE (Thio) and cultured for 24 hours in high glucose DMEM in the absence or presence of metformin (5 mM).

### DETAILED DESCRIPTION OF THE INVENTION

### Example 1: PP2A Controls Genome Integrity by Integrating Nutrient Sensing and Metabolic Pathways with the DNA Damage Response

Material and Methods

S. cerevisiae Strains, Growth Conditions, Drug Sensitivity Assay

All the strains used in this study are listed in Table 2 and are W303 derivatives with the wild type RAD5 locus. The MATa deletion mutant array and the SGA MATα query strain (S288C) were purchased from OpenBiosystems. Deletion, MYC-tagged, PK-tagged strains were obtained by one-step PCR targeting method (Wach et al., 1994). Unless otherwise stated, yeast strains were grown in yeast extract/peptone with 2% glucose (YPD). YPD agar plates were supplemented with adenine. Cells were synchronized in G1 with α-factor to a final concentration of 3 μg/ml. For drug sensitivity assay, cells were grown overnight. Serial 1:5 dilutions of stationary cultures were made and one drop of each dilution was pin-spotted onto agar plates, containing drugs. Plates were incubated for 2-3 days at 28° C. For liquid drug sensitivity assay, yeast strains were grown in SD liquid medium at the initial concentration of 10⁵ cell/ml in microtiter wells. Cultures were either left untreated (control-solvent) or were treated with the drug of interest. The absorbance (OD₅₉₅) of untreated and treated cultures was measured after 12-18 hours. 3 independent repeats were performed. For ceramide experiments, inventors noticed a rapid response (few minutes for PP2A activity, within one hour for DDR activity).

Chemicals

Hydroxyurea (Sigma H8627), Rapamycin (Sigma R0395), Metformin (Sigma D150959), Caffeine (Sigma C8960), Wortmannin (Sigma W1628), Okadayc acid (LC laboratories O-2220), C₂ ceramide (Sigma A7191), dihydroceramide C₂ (Sigma C7980), Myriocin (Sigma M1177), Paraquat dichloride hydrate (Sigma 36541), Mersalyl acid or in its sodium salt form (Sigma M9784, Pubchem 23690449), Tert-butyl hydroperoxide (Aldrich 416665), Terbinafine hydrochloride (Sigma T8826), Fluonazole (Sigma F8929), Cerulenin (Sigma C2389), Syringomycin E (kindly provided by Jon Takemoto, Utah State University), Fumonisin B1 (Enzo BML-SL220), L-Ethionine (Sigma E1260), Cycloleucine (Aldrich A48105)

Synthetic Genetic Array Screening

Synthetic genetic array (SGA) was carried out as described (Tong et al., 2001; Tong et al., 2004). Shortly, congenic irc21Δ (6 replicates) and wt (4 replicates) query strains were crossed with the haploid viable library (Tong, Evangelista et al. 2001). Colony sizes were quantified with the Colony Grid Analyzer (version 1.1.7) (Collins et al., 2010), and normalized to the intra-dish 80-percentile. A 1σ separation of normalized wt and irc21 colonies sizes was used to call candidate hits.

Tetrad Dissection and Random Spore Analysis

Standard procedures were used for tetrad dissection and random spore analysis (Abdullah and Borts, 2001; Tong and Boone, 2006).

Protein Extraction and Western Blot Analysis

Crude protein extracts were prepared following TCA based protocol and analyzed by SDS-PAGE as previously described (Pellicioli et al., 1999).

Anti-Rad53 (EL7 antibody described in (Fiorani et al., 2008) produced by IFOM monoclonal facility), anti-Mrel1 (clone 263++, described in (Ira et al., 2004) produced by IFOM monoclonal facility), anti-γH2AX (Abcam, ab15083), anti-H2A (Active motif, 39235), anti-c-myc (clone 9E10, produced by IFOM monoclonal facility), anti-PK (V5-TAG, MCA 1360 Bio-Rad) antibodies were used as primary antibodies during western blot procedure. Anti-mouse (Bio-Rad, 170-6510) and anti-rabbit (Bio-Rad, 170-6515) antibodies coupled with horseradish peroxidase enzyme were used as secondary antibodies. Detection was done through electrogenerated chemiluminescence (ECL, GE-Healthcare).

Fluorescence-Activated Cell Sorter (FACS) Analysis

Cell cycle analysis was conducted as previously described (Pellicioli et al., 1999).

Budding Index Analysis

After sonication, cells were fixed by the addition of 3.7% formaldehyde and 0.9% NaCl. Cells were examined under a light microscope, by counting 200 cells per time point.

Measurement of Intracellular Oxidation

ROS measurement was conducted as previously described (Rand and Grant, 2006).

Measurement of Oxygen Consumption

Respiration of log-phase S. cerevisiae cells was measured by polarographic analysis using a Clark's type oxygen electrode (Hansatech Instrument Ltd, Pentney UK) according to standard procedures, upon addition of dinitrophenol as uncoupling agent of respiration/ATP synthesis.

Search for Suppressors of Mec1-100 Sensitivity to HU

To search for suppressor mutations of the HU-sensitivity of mec1-100 mutant, 1×10⁶ mec1-100 cells were plated on YEPD in the presence of 25 mM HU. Survivors were crossed to wt cells to identify by tetrad analysis that the suppression events were due to single-gene mutations. Subsequent genetic analyses allowed grouping the single-gene suppression events in 4 classes. The class that showed the most efficient suppression was chosen and the mutations altering open reading frames within the reference S. cerevisiae genome were identified by next-generation Illumina sequencing (IGA technology services). To confirm that the tap42-G360R mutation was responsible for the suppression, a URA3 gene was integrated downstream of the tap42-G360R stop codon and the resulting strain was crossed to wt cells to verify by tetrad dissection that the suppression of the mec1-100 HU sensitivity co-segregated with the URA3 allele.

Metabolic Analysis

Yeast cells were grown to logarithmic phase (1×10⁷ cells/mL) in YPD medium in 6 replicates. 5×10⁸ cells per replicate were harvested by centrifugation, washed in water, snap-frozen in liquid nitrogen and stored at −80° C. Metabolite extraction and Ultrahigh Performance Liquid Chromatography-Tandem Mass Spectroscopy analysis of 484 metabolites were performed by Metabolon, Inc. (Durham, N.C.) as previously described (Chaudhri et al., 2013). Missing metabolite raw intensity values were filled in with the lowest detectable intensity of the respective metabolite, and all raw intensities were normalized to the median intensity of the respective replicate. Fold changes and significant alterations were calculated with the LIMMA method implemented in MultiExperiment Viewer (MeV) software (version 4.9.0) (Saeed et al., 2003) using an adjusted p value of 0.05 and a minimum fold-change of 1.3.

Statistics and Data Visualization

Interactome correlation analysis of irc21Δ was performed with published genome-wide SGA scores (Costanzo et al., 2010). Unsupervised hierarchical clustering and heatmap representation by SGA scores and genome-wide SGA score Pearson correlation values were done in MeV. Unsupervised hierarchical clustering of metabolome samples by Pearson correlation coefficient and heatmap representation were performed in R using the pheatmap library (version 1.0.8). Significances of the intersections of metabolite alterations were calculated by chi-squared test. Heatmaps for visualization of altered metabolite classes were generated with MeV.

TrueMass Ceramide Panel

Metabolites were isolated from cell pellets by sequential chloroform/methanol extraction and aqueous potassium chloride liquid-liquid extraction. The chloroform/methanol solution contained internal standards (Cer12:0, Cer19:0, dhCer12:0, hexCer12:0, [Avanti Polar Lipids, Alabaster, Ala.]) The organic layer was evaporated in a stream of nitrogen, reconstituted and subjected to a solid phase extraction clean up step on silica [Si, 100 mg, Supelco, Bellefonte, Pa.]. The ceramide fraction was eluted, evaporated in a stream of nitrogen, reconstituted and an aliquot was injected onto an AB Sciex 4000 QTRAP (Sciex, Foster City, Calif.)/Acquity (Waters, Milford, Mass.) LC-MS/MS system equipped with a reversed phase UHPLC column [Zorbax Eclipse Plus C8, 2.1×150 mm, 1.8 μm, Agilent Technologies] using a gradient of 2 mM ammonium formate/0.2% formic acid in water and 1 mM ammonium formate/0.2% formic acid in Acetonitrile:Isopropanol (60:40). The mass spectrometer was operated in MRM mode using positive electrospray ionization. The peak areas of the analyte fragment ions were measured against the peak area of the respective fragment ions of the corresponding internal standards. For the purposes of this panel, the fragment ion m/z 264 was used for ceramides with a sphingosine backbone and the m/z 266 fragment was used for analytes with a sphinganine backbone. Quantitation was based on a series of five calibration standard samples that were included in each run. Calibration standards contained 26 reference compounds. For analytes for which calibration standards were not commercially available, a surrogate analyte from the same compound class was used for quantitation (e.g. quantitation of CER 22:1 is based on CER 20:0 calibration standards). A total of 56 analytes covering ceramides, dihydroceramides, hexosylceramides and lactosylceramides with different fatty acid composition (14:0, 16:0, 18:0, 18:1, 20:0, 20:1, 22:0, 22:1, 24:0, 24:1, 26:0, 26:1) were determined.

Results

Irc21 Influences the Response to Replication Stress.

Out of the class of rad53 suppressors partially rescuing the HU sensitivity of the dominant-negative rad53-D339A and the kinase deficient rad53-K227A mutations (Bermejo et al., 2011; Fay et al., 1997), inventors identified irc21A. The suppression was validated in the W303 (Thomas and Rothstein, 1989) genetic background (FIGS. 1A and B). irc21Δ also rescued the HU sensitivity of mec1A sml1Δ, rad53Δ sml1Δ, mec1Δ rad53Δ sml1Δ, and chk1Δ rad53Δ sml1Δ strains (FIG. 1C). Sml1 inhibits ribonucleotide reductase (Chabes et al., 1999) and its ablation bypasses the essential functions of MEC1 and RAD53 by increasing dNTP levels (Desany et al., 1998; Huang et al., 1998; Zhao et al., 1998). IRC21 and SML1 double ablation caused an additive suppression in a rad53-K227A background; thus, unlikely, Irc21 influences dNTP pools (FIG. 8A). irc21Δ mutants were hypersensitive to high HU doses (FIG. 8B), suggesting that the irc21Δ suppression does not depend on dNTP levels or intrinsic HU resistance.

Inventors addressed whether IRC21 deletion influenced the Mec1-dependent Rad53 phosphorylation and dephosphorylation (Sanchez et al., 1996) during checkpoint activation and deactivation (FIG. 1D). sml1Δ, mec1Δ sml1Δ, irc21Δ sml1Δ and mec1Δ irc21Δ sml1Δ mutants were released from G1 into 0.2M HU to activate Mec1 and released into fresh medium without HU to allow cell cycle checkpoint deactivation and recovery (Pellicioli et al., 1999). In sml1Δ cells, Rad53 phosphorylation was obvious in HU and decreased during recovery. In mec1Δ sml1Δ mutants Rad53 phosphorylation was barely detectable, while in irc21Δ sml1 cells it was evident during HU treatment and persisted longer during recovery, compared to sml1Δ cells. irc21Δ restored Rad53 phosphorylation in a mec1Δ sml1Δ background. The persistence of Rad53 phosphorylation in mec1Δ irc21Δ sml1Δ mutants recovering from HU correlated with the inability to efficiently complete S phase (FIG. 8C). Inventors addressed whether irc21A-mediated rescue of Rad53 phosphorylation in mec1Δ cells was dependent on Tel1, which phosphorylates Mrel1 and shares overlapping functions with Mec1 (Usui et al., 2001). Rad53 was not phosphorylated in mec1Δ tel1Δ irc21Δ sml1Δ cells exposed to HU (FIG. 1E), suggesting that the irc21A-mediated rescue of Rad53 phosphorylation in mec1Δ cells depends on Tel1. irc21Δ suppression mechanism was not due to Tel1 hyperactivation as it is sufficient to ablate MEC1 to observe Tel1 activity (FIG. 1E).

Dun1 is phosphorylated by Rad53 (Bashkirov et al., 2003) and controls dNTP pools by inactivating Sml1 (Zhao and Rothstein, 2002). irc21Δ did not rescue the HU sensitivity of dun1A mutants (FIG. 8D), further suggesting that Irc21 does not cause an increase in dNTP pools. Moreover, IRC21 deletion failed to fully phosphorylate Dun1 in mec1 or rad53 mutants (FIG. 8E). Hence, the Tel1-mediated Rad53 phosphorylation in sml1Δ mec1Δ mutants is somewhat suboptimal as it prevents Rad53 from phosphorylating Dun1. This is consistent with the notion that certain phospho-isoforms of Rad53 are unable to phosphorylate Dun1 (Lee et al., 2003). Inventors monitored the capability of irc21Δ cells to recover from the HU-induced cell cycle block. irc21Δ mutants failed to efficiently recover from the HU treatment as visualized by FACS profile and by the delayed dephosphorylation of Rad53 and Dun1 (FIG. 8F).

Irc21 Affects Mitochondrial Functions and Lipid Biosynthesis.

Irc21 contains a unique domain: the NADH-cytochrome b₅ reductase domain (CBR); CBRs are involved in mitochondrial functions and lipid biosynthesis (Gene Ontology—PANTHER classification system) (FIG. 2A). Inventors therefore measured the oxygen consumption rate of logarithmically growing cells and found that irc21Δ mutants showed a higher respiration rate compared to wild type cells (FIG. 2B). During respiration, mitochondria generate reactive oxygen species (ROS). Using the oxidant-sensing probe 2′,7′-dichlorodihydrofluorescein diacetate (DCFH-DA) to measure ROS levels (Davidson et al., 1996), inventors found that exponentially growing irc21Δ cells accumulated more ROS than wt cells (FIG. 2C), according with previous findings (Neklesa and Davis, 2008). Inventors tested the oxidative stress resistance of irc21Δ cells using paraquat (1,1′-dimethyl-4,4′-bipyridinium dichloride) a redox cycler that stimulates superoxide production (Cocheme and Murphy, 2009). irc21Δ cells were hypersensitive to paraquat (FIG. 2D). Since respiration deficiency causes paraquat resistance (Blaszczynski et al., 1985), the higher respiration rates of irc21 cells may account for paraquat sensitivity. Mersalyl is a mercurial diuretic that affects mitochondrial functions and inhibits the NADH-cytochrome b₅ reductase (Bemardi and Azzone, 1981). irc21Δ cells were resistant to mersalyl, in accordance with the putative Irc21 CBR activity (FIG. 2D). tert-butyl hydroperoxide (t-BOOH, an organic peroxide) produces ROS and damages a variety of cellular constituents, including lipids, causing lipid peroxidation, which results in the oxidative degeneration of cellular polyunsaturated fatty acids (Girotti, 1998). irc21Δ cells were resistant to t-BOOH (FIG. 2D). The cytochrome b₅-dependent electron transport system is also involved in lipid metabolic processes, such as cholesterol/ergosterol biosynthesis and the desaturation and elongation of fatty acids (Aoyama et al., 1981; Osumi et al., 1979; Poklepovich et al., 2012; Tamura et al., 1976). In particular, Heme is required for the enzymatic activities of Erg3p (sterol C5-6 desaturase), Erg5p (sterol C22-23 desaturase), and Erg11p (sterol 14α-demethylase) (Mallory et al., 2005). Inventors analyzed the sensitivity of irc211A cells to the ergosterol-depleting agents fluconazole and terbinafine (FIG. 2D); Cyb5-dependent Erg11 is the target of fluconazole, while Cyb5-independent Erg1 (squalene epoxidase) is inhibited by terbinafine (Kontoyiannis, 2000; Lamb et al., 1999; Petranyi et al., 1984). irc21Δ cells were specifically sensitive to fluconazole and not to terbinafine. Since Cyb5 contributes to Erg11 activation (Lamb et al., 1999), IRC21 ablation could exacerbate the inhibitory effect of fluconazole on ergosterol synthesis. Next, inventors tested Irc21 involvement in fatty acids biosynthesis by analyzing irc21Δ sensitivity to cerulenin, an inhibitor of fatty acid synthase (FAS), that prevents the synthesis of medium and long chain fatty acids (MCFA and LCFA) and of very long chain fatty acids (VLCFA) (Awaya et al., 1975; Kvam et al., 2005; Rossler et al., 2003). VLCFAs participate to the formation of sphingolipids (SL), ceramides, inositolglycerophospholipids (IGP), and the phosphatidylinositol moiety of GPI anchored proteins (Dickson, 1998; Kvam et al., 2005). irc21 mutants were hypersensitive to cerulenin (FIG. 2D), thus implying that Irc21 affects fatty acid synthesis. Hence, the sensitivity/resistance profile suggests that Irc21 influences CBR-dependent processes.

Irc21Δ and PP2A Mutants Genetically Interact and Exhibit Similar Interactome Profiles.

Inventors compared the irc21Δ interactome with the interactomes of 3884 mutant array strains (Costanzo et al., Science, 2010) by calculating the correlation (R) value of their interaction scores with the 1712 query mutants. Among the top 10 array strains with SGA interactomes most similar to irc21Δ mutants, inventors identified deletions in the RRD1 (R=0.39), TIP41 (R=0.33), SAP185 (R=0.19) and RRD2 (R=0.17) genes (FIGS. 3A and 9A), which encode PP2A and PP2A-like regulators (Luke et al., 1996; Van Hoof et al., 2005) (FIG. 3E). The interactome correlation between irc21Δ and rrd1Δ was quantitatively similar to the one between the two PP2A/PP2A-like activators rrd1Δ and tip41A (R=0.37). The second hit with an irc21Δ interactome correlation similar to rrd1Δ was imp2′A (R=0.38) (FIG. 9A), in which the ORF next to RRD1 is disrupted; one possibility is that imp2′A impairs RRD1 expression and thus PP2A-like functionality. This is consistent with the high similarity of rrd1Δ and imp2′Δ interaction profiles (R=0.69). Inventors also identified deletions in the genes encoding Bck2, involved in the protein kinase C signaling pathway (Lee et al., 1993), Rrm3, a replicative DNA helicase targeted by the Mec1-Rad53 pathway (Rossi et al., 2015; Torres et al., 2004), Rnh201, a ribonuclease H2 involved in Okazaki fragment processing (Qiu et al., 1999) and the two phosphatases Ptp2 and Pph3 (Guan et al., 1992; O'Neill et al., 2007). Inventors compared the interactomes of irc21Δ and other PP2A and PP2A-like deletion mutants, by calculating the pairwise interactome correlation scores (R), and performed hierarchical clustering of R-values (FIG. 3B). The analysis revealed 2 clusters, the first containing pph21Δ, ppm1Δ and rrd2Δ (loosely associated with rts1Δ) and the second containing irc21Δ, rrd1Δ and tip41Δ (loosely associated with sap155Δ). Overall, these observations suggest that irc21Δ affects both PP2A and PP2A-like phosphatase activities (FIG. 3E). The signatures of genetic interactions shared between irc21Δ and either rrd1Δ or tip41Δ (Costanzo et al., 2010) (FIG. 3C) showed that common interactors were associated with PP2A/PP2A-like regulated processes, including mitosis, checkpoint recovery and adaptation, and mitochondrial maintenance. In addition, other PP2A mutant strains, including sap185Δ, rrd2Δ, rts1Δ, pph21Δ, and pph22Δ shared interactions with the irc21Δ/rrd1Δ/tip41Δ signatures to varying degrees (FIG. 3C).

Inventors conducted an independent synthetic genetic array (SGA) analysis mating the irc21Δ query strain to the haploid deletion library containing deletions of ˜4700 non-essential genes (FIG. 3D) (Tong et al., 2001). Inventors found 42 negative and 12 positive interactions, causing, respectively, synthetic growth defects or suppression of the mild slow growth phenotype of irc21l mutants (FIG. 9B). In addition, inventors identified 5 high confidence epistatic interactors of IRC21, by filtering potential epistatic interactors from our dataset, with the positive IRC21 interactors previously reported in a genome-wide high-throughput E-MAP screen (Costanzo et al., 2010) (FIG. 9C).

Several PP2A/PP2A-like components displayed negative interactions with irc21Δ (significance: ptc1Δ, rts1Δ; trend: tip41Δ, ppm1Δ, rrd1Δ, rrd2Δ) (FIGS. 3D and E). Some of these interactions were confirmed by random spore analysis and/or tetrad dissection (FIGS. 3D, 9D and E). The largest categories of irc21Δ negative genetic interactors (FIG. 9B) were metabolic (oxidative stress, TCA cycle, lipids), and chromatin/checkpoint pathways, which have been related to PP2A (Heideker et al., 2007; Hughes Hallett et al., 2014; Madeira et al., 2015; Rossetto et al., 2012). In accordance with previous observations (FIG. 2D—Cerulenin sensitivity assay), irc21Δ displayed negative genetic interaction with the deletion in FEN1 (FIG. 9B), encoding the fatty acid elongase, required for the biosynthesis of ceramide (Oh et al., 1997), a PP2A/PP2A-like activators (Nickels and Broach, 1996); moreover, ceramide hydroxylase Scs7, that contains a cytochrome b₅ domain like Irc21 (Mitchell and Martin, 1997), was identified among the top 5 high confidence Irc21 epistatic interactors (FIG. 9C). Rescuing interactors (FIG. 9B) were involved in phospholipid (PGC1), sterol (NSG2) and respiratory (RG12, TRX3) metabolism, mitochondrial localization/inheritance (JSN1), cell morphology (MGAJ, DFG5), nuclear membrane (MLP2), and genome integrity (RAD51); Epistatic interactors (FIG. 9C) were involved in spindle and organelle positioning (DYN3, NIP100), mitochondrial localization/inheritance (MMRI) and ion transport (PMR1).

Overall, the IRC21 interactome analysis supports a function for Irc21 in mitochondrial and lipid metabolism and in influencing PP2A activity, nuclear morphology and genome integrity.

Irc21 is Involved in the TORC1-PP2A Regulatory Axis.

Inventors next characterized the role of Irc21 in regulating PP2A/PP2A-like activities, which are negatively regulated by the TORC1 pathway (Di Como and Arndt, 1996). Inventors found that tor1Δ and tco89Δ, defective in TORC1 components, were positive interactors of irc21Δ (FIGS. 3C and D). TORC1 controls through phosphorylation two main downstream effectors, Tap42 and Sch9 (Huber et al., 2009; Jiang and Broach, 1999; Urban et al., 2007) (FIG. 4A). Sch9 influences ribosome biogenesis, translation initiation and G0 events (Pedruzzi et al., 2003; Urban et al., 2007; Wei and Zheng, 2009). Tap42 regulates PP2A and PP2A-like phosphatases, which control the phosphorylation state of Msn2/Msn4, involved in environmental stress response, Rtg1/3, implicated in the retrograde pathway, and Npr1 and Gln3, connected with the amino acid synthesis and nitrogen assimilation pathways (Crespo et al., 2002; Di Como and Arndt, 1996; Santhanam et al., 2004).

To probe the Tap42/PP2A signaling branch in irc21A mutants, inventors selected four PP2A targets that exhibit clear modifications following TORC1 inhibition through rapamycin treatment: Gln3, Nnk1, Npr1 and Rtg3 (Beck and Hall, 1999; Cox et al., 2004; Hughes Hallett et al., 2014; Schmidt et al., 1998) (FIG. 4A). As expected, rapamycin led to substantial dephosphorylation of the four PP2A targets (Hughes Hallett et al., 2014); in contrast, all four targets remained hyper-phosphorylated in irc21Δ, rrd1Δ and tip41Δ cells, even in the presence of rapamycin (FIG. 4A, left panel). Thus, Irc21 participates in the activation of the PP2A/PP2A-like pathways like Rrd1 and Tip41. To discern whether Irc21 activates PP2A directly, or by inhibiting TORC1, inventors monitored the TORC1-Sch9 branch: Sch9 was normally dephosphorylated after rapamycin treatment in all the mutant strains (FIG. 4A, right panel). Hence, Irc21 is specifically involved in the activation of the PP2A/PP2A-like sub-pathways, which are also regulated by TORC1. Accordingly, irc21Δ mutants are resistant to treatments with a variety of TORC1 inhibitors, such as rapamycin, caffeine, metformin and wortmannin (FIG. 4B), in analogy to certain PP2A mutants (Jacinto et al., 2001; Rempola et al., 2000; Zheng and Jiang, 2005).

PP2A Influences the Checkpoint Response.

Altogether, the previous observations led inventors to test the hypothesis that, similarly to Irc21, PP2A and PP2A-like, control the Rad53-mediated response to replication stress. Ablation of the PP2A positive regulators RRD1 and TIP41 mimicked irc21Δ in suppressing the HU sensitivity of rad53-D339A, rad53-K227A, rad53A sml1Δ and mec1Δ sml1Δ mutant alleles (FIGS. 10A and B, 4C and E). Moreover, the deletions of either RRD1 or TIP41 in a mec1Δ sml1Δ background were able to rescue the crippled Rad53 phosphorylation (FIG. 4D), similarly to irc21Δ (FIG. 1D). rrd1Δ and tip41Δ also exhibited a delayed Rad53 deactivation, following recovery from HU, similarly to irc21Δ mutants.

Inventors performed a screen to find spontaneous extragenic suppressors of mec1-100 (Paciotti et al., 2001) cells lethality on HU 25 mM and inventors identified a mutation in TAP42. tap42-G360R rescued the HU sensitivity of mec1Δ and rad53A cells (FIGS. 4E and F) and abolished the defective Rad53 phosphorylation in HU-treated mec1Δ sml1Δ cells, mimicking the phenotype of irc21Δ, rrd1Δ and tip41Δ (FIG. 4G).

A gain-of-function mutation could account for the above results and for the semi-dominance behavior of the tap42-G360R allele. To test whether the mutation caused a constitutive inhibition of PP2A, inventors analyzed rapamycin and metformin sensitivity of tap42-G360R cells. As expected, and similarly to irc21Δ, rrd1Δ and tip41Δ, tap42-G360R mutants were partially resistant to both drugs (FIG. 10C). Secondly, after rapamycin treatment, tap42-G360R cells showed a defective Gln3, Nnk1 and Npr1 dephosphorylation, which is mediated by PP2A (FIG. 10D) (Hughes Hallett et al., 2014). Hence, the hyperactive Tap42 allele resembles the absence of PP2A activators.

Since PP2A appeared to target Rad53, inventors predicted that chemical compounds acting on PP2A activity should also affect rad53 HU sensitivity. Low doses of okadaic acid (OA) cause a selective inhibition of PP2A (Zhang et al., 1994), and partially rescued the HU sensitivity of rad53K227A mutants (FIG. 4H).

Irc21 and PP2A Metabolic Signatures.

PP2A is a master metabolic regulator and is regulated by metabolic stimuli (Di Como and Arndt, 1996; Oaks and Ogretmen, 2014). To characterize the relationship between PP2A and Irc21, inventors compared the global mass spectrometry metabolic profile of wt, irc21Δ and rrd1Δ cells during logarithmic growth in rich media. Unsupervised clustering by metabolite fold changes clearly grouped the replicates of irc21Δ and rrd1Δ by genotype, but also revealed a degree of similarity between both mutants (FIG. 5A). From a total of 484 examined compounds, 172 and 111 were significantly changed in irc21Δ and rrd1Δ cells, respectively (LIMMA, p_{adj}=0.05) (FIG. 5B). The two mutants shared a significant amount of metabolite alterations (79, p=5×10⁻¹¹). Importantly, nearly all of these were co-regulations (72/79), suggesting that these 72 alterations define a metabolic, shared Irc21-PP2A signature (FIG. 5B). As expected for low PP2A activity (Staschke et al., 2010; Wong et al., 2015), the PP2A signature was characterized by a reduction of amino acid biosynthesis intermediates and dipeptides (FIG. 5C, top panel). It also featured elevated levels of multiple lipids and lipid intermediates (long chain fatty acids, sterol biosynthesis intermediates, lyso-phospholipids, carnitine conjugates, sphingolipid precursors) and a shifted composition of phospholipids to shorter fatty acid chain length (less than C18). Low PP2A activity correlated with high GlcNAc (N-Acetylglucosamine) biosynthesis intermediates, high deoxy-nucleosides (but normal deoxy-nucleotides) and high levels of the methyl donor SAM.

The specific metabolite alterations in irc21Δ that are not shared by rrd1Δ represent PP2A-independent functions of Irc21. In particular, inventors found that several metabolites related to CBR function were altered in irc21Δ cells. Accumulation of TCA cycle intermediates (aconitate, α-ketoglutarate, fumarate, malate) and reduction in late glycolysis intermediates and Ac-CoA were indicative of altered mitochondrial activity, in accordance with the notion that Cytb5 participates in mitochondrial electron transport chain (FIG. 2A, 5C bottom panel); in addition, the levels of several amino acids derived from glycolysis (Gly, Val) and TCA (Gln, Thr, Lys) and their derivatives were reduced, while urea cycle products accumulated (Ornithine, urea). Purine and pyrimidine ribonucleosides and ribonucleotides as well as CTP-dependent phospholipid precursors were also reduced, whereas the nucleotide synthesis substrate PRPP increased. Cytb5 is also involved in fatty acid metabolism (FIG. 2A) and, accordingly, inventors found that while fatty acid accumulation was common to irc21Δ and rrd1Δ, very long chain fatty acids (VLCFAs), which are used in the synthesis of ceramides, accumulated specifically in irc21Δ cells. Accumulation of VLCFAs and the genetic interaction with the VLCFA synthesis enzyme FEN1 suggest that irc21Δ mutants inefficiently condense sphingolipids and VLCFAs into ceramides, and, thus, fail to promote PP2A activation, that depends on ceramide levels.

Inventors therefore performed a quantitative mass spectrometry analysis of ceramides and related lipid metabolites in wt and irc21Δ cells. Dihydroceramides (DHC) were more abundant than phytoceramides (PHC) in wt cells (0.35 pmol/mg and 0.01 pmol/mg respectively) (FIG. 11A). Furthermore, deletion of IRC21 significantly reduced the level of DHC, (FIGS. 5D, 11A-C). In contrast, the levels of DHC precursors, 3-ketodihydrosphingosine (3-keto-DHS), dihydrosphingosine (DHS), dihydrosphingosine-1-P (DHS-1-P) and VLCFA-CoA increased in irc21Δ cells (FIGS. 5C-D, 11A-C). These observations confirmed that IRC21 deletion impairs ceramide biosynthesis and the defective step corresponds to the DHS-DHC conversion (FIGS. 5E and 11C). According to this scenario, inventors found that irc21Δ mutants were resistant to Myriocin (FIG. 5F), an inhibitor of ceramide synthesis acting on serine palmitoyltransferase (SPT), the first enzyme in the sphingolipid biosynthesis pathway (FIG. 11C) (Huang et al., 2012). Moreover, irc21Δ cells were resistant to syringomycin E (SRE) (FIG. 5G) (Julmanop et al., 1993; Takemoto et al., 1993). SRE resistance has been used as a readout reflecting a defect in sphingolipid biosynthesis (Cliften et al., 1996; Stock et al., 2000; Taguchi et al., 1994). Addition of exogenous DHC restored irc21Δ sensitivity to SRE (FIG. 11D). Fumonisin B1, a ceramide synthase inhibitor, pheno-copies irc21Δ mutants, by causing an increase in DHS and PHS levels and a concomitant decrease of ceramide and therefore DHC (Wu et al., 1995). Accordingly, inventors found that irc21Δ mutants were resistant to fumonisin B1 (FIG. 5H). Inventors conclude that, Irc21 promotes the condensation reaction leading to the formation of DHC.

Ceramides, SAM-Mediated Methylation and TORC1 Inhibition Attenuate the Checkpoint Response by Promoting PP2A Activation.

irc21Δ partially rescues the HU sensitivity of checkpoint mutants and Rad53 phosphorylation in HU-treated mec1Δ sml1Δ. Moreover, PP2A activity is defective in irc21Δ, as well as in rrd1Δ, tip41Δ and tap42-G360R mutants, and that attenuated PP2A activity is beneficial for checkpoint mutants exposed to replication stress. Since our observations also involve Irc21 in ceramide synthesis, inventors investigated the possibility to abrogate irc21Δ phenotypes by exogenously providing ceramide. It is known that the cell-permeable ceramide analog C₂-ceramide induces a dose dependent activation of PP2A in yeast (Jiang, 2006; Nickels and Broach, 1996). Inventors first analyzed the effect of exogenous ceramide on the rescue of rad53K227A HU sensitivity by irc21Δ, rrd1Δ and tip41Δ. Intriguingly, the combination of HU treatment with sphingolipid was able to suppress the rescue in all mutants (FIG. 5I). Inventors then asked whether ceramide was also able to suppress the IRC21A-dependent rescue of Rad53 phosphorylation in mec1Δ cells. Cells were arrested in G1 and released in HU with or without ceramide for 3 hours. Ceramide-mediated PP2A activation abolished Rad53 phosphorylation, specifically in irc21Δ mec1Δ cells, and reduced it in wt and irc21Δ cells (FIG. 5J). In addition, ceramide caused Rad53 dephosphorylation during recovery from HU treatment in irc21Δ mec1Δ mutants (FIG. 11E). Sur2 and Scs7 are both required for ceramide hydroxylation and are members of the cytochrome b₅-dependent enzyme family (FIG. 11C) (Haak et al., 1997). Scs7p contains a cytochrome b₅-like domain (Dunn et al., 1998), while cytochrome b₅ may function to transfer electrons to Sur2 (Haak et al., 1997). Both SCS7 and SUR2 deletions partially rescued mec1Δ sml1Δ HU sensitivities (FIG. 6A). Thus, defective cytochrome b₅-dependent enzymes, involved in ceramide biosynthesis, have beneficial consequences for checkpoint mutants exposed to replication stress. SAM levels are critical for methylation and activation of PP2A; in this process, Ppm1 methylates the C terminus of PP2A catalytic subunit (Sutter et al., 2013; Wei et al., 2001; Wu et al., 2000). Accordingly, PPM1 ablation partially impaired dephosphorylation of PP2A targets (FIG. 12A). Inventors found that irc21Δ and rrd1Δ mutants accumulate high levels of SAM (FIG. 5C); accordingly, irc21Δ and rrd1Δ mutants were hypersensitive to SAM limitation caused by ethionine (toxic analogue of methionine) or cycloleucine (inhibitor of methionine adenosyl transferase) (FIG. 6C). In addition, inventors discovered a negative interaction between IRC21 and the PP2A methyltransferase PPM1 (FIG. 3D). Inventors confirmed that irc21Δ mutants are synthetic sick with ppm1Δ cells (FIG. 6B). These results suggest that Irc21 and Ppm1 positively regulate PP2A through different mechanisms. Interestingly PPM1 ablation, like IRC21 deletion rescued the HU sensitivity of mec1Δ sml1Δ mutants (FIG. 6D) and partially recovered the Rad53 defective phosphorylation in HU-treated mec1Δ cells.

Rapamycin inhibits TORC1 that represses PP2A complexes. Both rapamycin and ceramide have been showed to promote PP2A activity (Loewith et al., 2002; Nickels and Broach, 1996).

Inventors tested whether rapamycin and ceramide treatments could modulate the HU induced-DDR response. Cells were released from G1 in the presence of HU alone or combined with rapamycin and ceramide (FIG. 6E). After 5 minute-treatment, when Rad53 was still unphosphorylated, the concomitant presence of rapamycin and ceramide caused PP2A hyperactivation, as indicated by the Nnk1 phosphorylation status. At 60 minutes, Rad53 phosphorylation was evident in HU-treated cells, partial in HU+rapamycin and HU+ceramide and abolished in HU+rapamycin and ceramide. Inventors obtained analogous results by treating exponentially growing cells with HU, in combination with rapamycin and/or ceramide (FIG. 12B), thus ruling out the possibility that the effect of rapamycin and ceramide on Rad53 phosphorylation was due to a delayed entry into S phase.

Altogether these observations suggest that TORC1, Irc21 and Ppm1 influence the HU-induced DDR by regulating the activity of PP2A, and that ceramide levels, as well as SAM levels, are crucial for Mec1 and Rad53 activation.

Discussion

Activation of the Mec1^{ATR}-mediated DNA damage response requires multiple post-translational modifications that integrate chromosomal signals and mechanical stimuli (Awasthi et al., 2016). Deactivation of the Mec1^{ATR} pathway promotes cell cycle recovery or adaptation (Bartek and Lukas, 2007; Clemenson and Marsolier-Kergoat, 2009). A fine-tuning of the Mec1^{ATR} cascade is required to prevent deleterious consequences of unscheduled checkpoint activation (Bastos de Oliveira et al., 2015; Harrison and Haber, 2006). Mec1 and ATR regulate nuclear and non-nuclear pathways (Hilton et al., 2015; Kumar et al., 2014; Matsuoka et al., 2007). In yeast, several phosphatases have been involved in DDR silencing, including PP2C (Ptc2/Ptc3) and PP4 (Pph3-Psy2) required for DSB recovery, and PP1 (Glc7) which promotes HU recovery (Bazzi et al., 2010; Keogh et al., 2006; Leroy et al., 2003; O'Neill et al., 2007). PP2A has been genetically linked to the RAD53-MEC1 pathway, but ruled out as one of the main phosphatases implicated in checkpoint control (Hustedt et al., 2015). In mammals, PP2A shows activity towards γH2AX, ATM, p53, Chk1 and Chk2 (Chen et al., 2015; Dozier et al., 2004; Goodarzi et al., 2004). Here, inventors demonstrate that PP2A inactivation is beneficial when the Mec1-Rad53 axis is defective. Moreover, PP2A/PP2A-like act in a network with Irc21 and TORC1 to integrate metabolic signals with phosphorylation and dephosphorylation events outside and inside the nucleus and to attenuate the Mec1^{ATR} cascade in cells experiencing replication stress.

IRC21 ablation rescues mec1Δ, rad53A, chk1Δ sensitivity to low doses of HU and it is able to promote HU-induced Rad53 phosphorylation when Mec1 is absent, through a process mediated by Tel1. Irc21 was previously connected to the DDR, but the mechanism remained unclear (Guenole et al., 2013).

Irc21 is an uncharacterized protein that consists of a cytochrome b5 domain; in accordance, irc21Δ mutants influence the respiration rate and ROS levels, display resistance to mersalyl, and show a metabolic profile altered in CBR-related functions. Interestingly, Irc21 localization is mainly cytoplasmic (Guenole et al., 2013; Huh et al., 2003), although a fraction has been detected in the nucleus (Guenole et al., 2013), in mitochondria and vacuoles (CYCLoPs (Koh et al., 2015)). A key question is how does Irc21 influence the checkpoint response.

Inventors show that Irc21 positively regulates PP2A/PP2A-like activities. IRC21 ablation causes resistance to TORC1 inhibitors, but does not influence the TORC1-Sch9 axis, suggesting that Irc21 unlikely acts upstream of TORC1. The functional relationship between PP2A and TORC1 is rather complex and still controversial (Duvel et al., 2003). According to the traditional view, TORC1 negatively regulates PP2A/PP2A-like through Tap42 phophorylation (Di Como and Arndt, 1996), and, in the meantime, PP2A stimulates TORC1 through Npr2 phosphorylation (Laxman et al., 2014). Intriguingly, irc21Δ mutants exhibit a negative genetic interaction with PP2A/PP2A-like activators (Rrd1, Rrd2, Tip41, Saps and Ppm1) and positive genetic interactions with TORC1 components (Tco89 and Tor1) (FIG. 7). Inventors speculate that IRC21 ablation ameliorates TORC1-defective mutants by limiting PP2A/PP2A-like activities.

Hence, inventors propose that Irc21 may stimulate PP2A and therefore attenuate the DDR. Indeed, genetic and pharmacological inactivation of PP2A ameliorates the defective response to replication stress of checkpoint mutants. In addition, exogenous ceramide causes Rad53 dephosphorylation during recovery from HU treatment in mec1Δ irc21Δ sml1Δ mutants, and abolishes irc21 rescue of Rad53 phosphorylation in mec1Δ irc21Δ sml1Δ cells during HU treatment.

The next key question is how does Irc21 regulate PP2A activity. Among all metabolic alterations in irc21 mutants, elevated ROS are a potential contributor to PP2A suppression; indeed, ROS accumulation causes PP2A inactivation (Nakahata and Morishita, 2014; Shimura et al., 2016). Inventors excluded this hypothesis since ROS scavengers did not affect the capability of irc21Δ to rescue the HU sensitivity of checkpoint mutants.

IRC21 ablation causes alterations in the sphingolipid metabolism associated with a reduction of DHC and an accumulation of DHC precursors (3-keto-DHS, DHS, DHS-1-P and VLCFA-CoA). Hence, irc21Δ mutants are deficient in ceramide biosynthesis and the defective step corresponds to the DHS-DHC conversion. Inventors note that i) the hydroxylation of sphingolipid long chain bases and ceramides requires Sur2 and Scs7, two members of the cytochrome b₅-dependent enzyme family (Haak et al., 1997) Mitchell and Martin, 1997), ii) Irc21 is a cytochrome b₅-like enzyme and iii) Scs7 was identified among the top 5 high confidence Irc21 epistatic interactors, iiii) absence of either Scs7 or Sur2 ameliorates the replication stress-resistance of checkpoint mutants. Although these observations may suggest a role for Irc21 in ceramide hydroxylation, this is unlikely, as a defect in ceramide hydroxylation would not lead to diminished DHC levels (see FIG. 5C).

DHC is produced by the condensation reaction of DHS with VLCFAs; the reaction is catalyzed by the ceramide synthase (Lag1, Lac1, Lip1) and a defect in this reaction would cause accumulation of DHS and reduction in the levels of DHC, as inventors observe in the absence of Irc21. Accordingly, irc21Δ mutants are resistant to fumonisin B1, a ceramide synthase inhibitor (Wu et al., 1995). Hence inventors favor the hypothesis that Irc21 promotes the condensation reaction leading to the formation of DHC. One possibility is that Irc21 facilitates the activity of the ceramide synthase; intriguingly, LAC1 transcription is regulated by Rox1, a heme-dependent anaerobic repressor (Kolaczkowski et al., 2004). Another possibility is that Irc21 counteracts the activity of the Ydc1 and Ypc1 ceramidases that hydrolyze ceramides into sphingosine and fatty acid. Notably, Ydc1 or Ypc1 overepressions phenocopy irc21 mutants in accumulating DHS with a concomitants reduction in DHC (Mao et al., 2000a; Mao et al., 2000b). Interestingly, Irc21 binds cardiolipin (CL), a mitochondrial phospholipid (Gallego et al., 2010) that is known to activate ceramidases (El Bawab et al., 2001).

Ceramides activate PP2A in yeast and mammals (Dobrowsky et al., 1993; Nickels and Broach, 1996). Hence, Irc21 might directly stimulate PP2A and therefore attenuate the DDR by contributing to the production of ceramides. Interestingly, rapamycin and ceramide treatments cause a synergistic stimulation of PP2A activity, according to the view that TORC1 and Irc21 regulate PP2A activity in a negative and positive way, respectively (FIG. 7). Isc1, involved in the hydrolysis of complex sphingolipids to ceramides, has been also connected to the HU-induced replication stress (Matmati et al., 2013; Tripathi et al., 2011); ISC1 ablation confers HU sensitivity, and this phenotype is suppressed by Cdc55 overexpression, thus suggesting that Isc1 provides protection from HU through a mechanism requiring PP2A (Matmati et al., 2013).

irc21Δ mutants exhibit alterations in glucose homeostasis and glycolytic pathways, grow poorly in low glucose conditions and are synthetic sick in combination with mutations in SNF^{AMPK}(FIG. 5C). Given that Snf1^{AMPK} cross-talks at several levels with the PP2A and TORC1, it is possible that, in irc21 mutants, Snf1 activity may influence, at least in part, PP2A and DDR activities. However, this is unlikely as inventors found that IRC21 ablation rescues the HU sensitivity of checkpoint defective mutants in a SNF1 independent manner.

PP2A activity is stimulated by the SAM-Ppm1 axis (Laxman et al., 2014). The synthetic sickness between ppm1 and irc21 mutants may therefore result from the simultaneous ablation of two independent positive regulatory pathways leading to PP2A activation (FIG. 7). In this scenario, the low PP2A activity observed in ppm1 mutants would depend on the lack of PP2A methylation (Wu et al., 2000), while, in irc21 mutants, may result from limiting ceramide levels. Intriguingly, inventors show that irc21 mutants display elevated SAM levels, raising the possibility that Ppm1-mediated PP2A methylation may facilitate basal PP2A activity in the absence of ceramide-dependent PP2A activation; Accordingly, irc21 mutants are particularly sensitive to treatments that limit SAM availability. Alternatively, since low glucose levels cause increased SAM levels (Ogawa et al., 2016), irc21 SAM levels may reflect glucose homeostasis defects.

Inventors propose that DDR is attenuated by PP2A/PP2A-like, which are negatively regulated by the TORC1-Tap42 axis and positively regulated by the Irc21-Ceramide and SAM-Ppm1 pathways (FIG. 7).-

Nutrients not only supply energy and building blocks for cellular growth, but also exert crucial regulatory functions. Inventors show that PP2A represents a central hub in mediating a crosstalk between nutrients sensing, cell metabolism and the DDR. In fact, PP2A controls the phosphorylation status of several targets involved both in cell metabolism and DDR and it integrates the following nutritional pathways. i) Nitrogen and carbon metabolism: together with Sch9^{S6K}, PP2A is one of the two crucial effectors of TORC1, which is activated by nitrogen and carbon metabolites and promotes anabolic processes (Hughes Hallett et al., 2014; Loewith and Hall, 2011; Orlova et al., 2006; Ramachandran and Herman, 2011) ii) Methionine metabolism: PP2A responds to S-adenosylmethionine levels, which depends on the availability of methionine (Sutter et al., 2013). iii) Sphingolipid metabolism: PP2A/PP2A-like are CAPP (Janssens and Goris, 2001; Nickels and Broach, 1996). Here inventors show that nutritional pathways impinge on the DDR by regulating PP2A, thus demonstrating a key role of PP2A in transducing metabolic signals to checkpoint kinases.

Besides linking ceramide levels with PP2A activity and DDR attenuation, our observations identify synergic combinations between ceramides, TORC1 inhibitors and SAM. Further characterization of the links between DDR, nutrient sensing pathways and cell metabolism may have relevant implications for exploiting novel therapeutic options as well as for repositioning/combining known drugs.

### Example 2: Combination of Hypoglycemia and Metformin Impairs Tumor Metabolic Plasticity and Growth by Modulating PP2A-GSK3B3-MCL-1 Axis

Experimental Procedures

Reagents

Antibodies were purchased from the indicated sources and used at a dilution of 1:1000 unless otherwise described: anti-MCL-1 (Santa Cruz Biotechnology); anti-AMPK, anti-AMPK, anti-pACC, anti-ACC, anti-pGSK3P, anti-GSK3β, anti-pERK (Cell Signaling Technology); anti-BCL-2 and anti-Bcl-xL (BD Biosciences); anti-Vinculin (SIGMA, dilution of 1:10000). Drugs were purchased from the following sources: Metformin (Sigma Aldrich), GSK31 inhibitor xii, GSK3P inhibitor viii, U0126, PD98059, SP600125 and SB 202190 (Selleck Chemicals).

Tissue Culture

HCT116, HeLa, MCF7, SK-MEL28 and A-549 cell lines were grown in Dulbecco's modified Eagle's medium (DMEM) supplemented with 10% fetal bovine serum and 2 mM L-glutamine unless otherwise indicated. Other cell lines were grown in RPMI medium supplemented with 10% fetal bovine serum and 2 mM L-glutamine unless otherwise indicated. For starvation experiments, cells were washed three times with PBS pH 7.2 and then incubated in the indicated starvation conditions. All cultures were maintained in a humidified tissue culture incubator at 37° C. in 5% CO2.

Immunoblotting

Whole cell lysates were prepared by directly lysing cells growing in culturing dishes or collected cell pellets in lysis buffer (40 mM Hepes pH 7.5, 120 mM NaCl, 1 mM EDTA, 10 mM pyrophosphate, 10 mM glycerophosphate, 50 mM NaF, 0.5 mM orthovanadate, and EDTA-free protease inhibitors (Roche) containing 0.3% CHAPS). Lysates were prepared from frozen tumors using GentleMACS dissociator. Lysates were cleared by centrifugation at 13000 g for 15 min. at 4° C., quantified using BioRad DC protein assay reagent followed by mixing 1:1 with 4% SDS, 100 mM Tris.Cl pH 6.8, 20% glycerol, 0.1% bromophenol blue and 5% β-mercaptoethanol added immediately before use and heating at 94° C. for 7 min. Equal amounts of proteins were then electrophoresed on 8-15% SDS-PAGE gels. Gels were run at 100 V (stacking gel)/150 V (separation gel) on Protean III apparatus (BioRad). Gels were transferred onto nitrocellulose and probed with the appropriate primary antibody for a variable incubation time depending on the experimental design, followed by the corresponding secondary antibodies diluted 1:5000-10000. The proteins were visualized by enhanced chemiluminescence (ECL) using ChemiDoc apparatus (BioRad) according to the manufacturer's instructions.

RNA Interference

shRNA pLKO.1 lentiviral constructs were purchased from Open Biosystems. Target sequences are as follows:

| | | |
|---|---|---|
| Scrambled: | GTGGACTCTTGAAAGTACTAT | (SEQ ID NO: 1) |
| GSK3β #1: | GCTGAGCTGTTACTAGGACAA | (SEQ ID NO: 2) |
| GSK3β #2: | CACTGGTCACGTTTGGAAAGA | (SEQ ID NO: 3) |
| PP2A #1: | CAACAATTGCCCTAGCACTTG | (SEQ ID NO: 4) |
| PP2A #2: | GACAACAGCACCTTGCAGAGT | (SEQ ID NO: 5) |
| B55δ #1: | AGTCTGACTGAGCCGGTAATTC | (SEQ ID NO: 6) |
| B56δ #2: | CACATCTCCAGCTCGTGTATGC | (SEQ ID NO: 7) |
| PP2A Aα #1: | TTGCCAATGTCCGCTTCAATGC | (SEQ ID NO: 8) |
| PP2A Aα #2: | CTACGCTCTTCTGCATCAATGC | (SEQ ID NO: 9) |

Lentiviral Transduction

The pLKO.1 vectors and package plasmids were co-transfected into packaging HEK293T cells and the viral supernatants were collected, supplemented with polybrene (8 ug/mL) and used to infect target cells in four 2-hour cycles of transduction over two consecutive days.

Quantification of Cell Proliferation

CellTiter Glo Luminescent Cell Viability Assay (Promega) was used according to manufacturer's protocol. Briefly, cells were plated in 96 well plates, treated 24h later with different doses of drugs in total volume of 100 μl. 24h later, 100 μl of CellTiter Glo reagent was added to the cells and incubated for 15 min at 37° C. and luminescence was measured using a Promega plate reader.

Quantification of Cell Death

Cells were harvested by trypsinization, washed in PBS (pH 7.2), and then stained with propidium iodide (10 mg/ml) added immediately prior to analysis. Cell fluorescence was then measured on a flow cytometer (FACSCalibur; Becton Dickinson, CA) and analyzed using CellQuest software.

Lactate Production Assay

Lactate production was measured using Lactate Assay Kit (Sigma Aldrich) according to manufacturer's instructions.

Oxygen Consumption Assay

Oxygen Consumption Rate was measured using Oxygen Consumption Rate Assay Kit (MitoXpress® Xtra HS Method). (Cayman Chemical) according to manufacturer's instructions.

Xenografts

CD1 nude mice received single subcutaneous flank injections of 5×10⁶ HCT-116 cells or 1×10⁵ patient-derived melanoma cells suspended in 200 μl saline. After the tumors were established, mice were randomized in different groups. Mice were kept on the feeding/fasting protocols described. Fasting cycles were achieved by complete removal of food while allowing free access to water. Metformin was administered via oral gavage at 200 mg/kg dissolved in water. Tumor growth was monitored by bi-dimensional measurements using a caliper. Experiments have been done in accordance with the Italian Laws (D.L.vo 116/92 and following additions), which enforces EU 86/609 Directive (Council Directive 86/609/EEC of 24 Nov. 1986 on the approximation of laws, regulations and administrative provisions of the Member States regarding the protection of animals used for experimental and other scientific purposes). Mice have been housed accordingly to the guidelines set out in Commission Recommendation 2007/526/EC—Jun. 18, 2007 on guidelines for the accommodation and care of animals used for experimental and other scientific purposes. Note that nude mice show a strain-specific decline in glucose levels upon fasting, of a higher degree as compared to other commonly used mouse strains (C57B6).

Immunohistochemistry

Formalin fixed paraffin embedded samples of tumors were cut 5 μm thick on polarized glass; unmasking for both antigen was made with Citrate for 30′ at 99° C.; anti-Mcl1 and anti-pGSK3β antibodies were used at 1:200 and 1:50 concentration respectively for two hours. LSAB 2 System-AP (DAKO) and Vulcan Fast Red Chromogen Kit 2 (Biocare Medical) were used as visualization system according to company working procedure. After hematoxilin and eosin review, the positivity of tumor cells was scored using a scoring system evaluating the staining pattern (homogeneous—i.e. low power reproduce high power- or heterogeneous scoring respectively 0.1), the intensity of staining in the most reactive area (absent/weak/moderate/strong scoring respectively 0, 1, 2 or 3) and the percentage of most reactive cells/total cancer cells (≤10%; more than 10% but ≤50%; and >50% scoring respectively 0, 1 or 2).

Results

Cancer Cells Exhibit Metabolic Plasticity

Our initial observations showed that metformin exerts only weak anti-proliferative effects on an array of cancer cell lines representative of different cancer types as well as patient-derived melanoma cells when cells are kept in nutrient-rich conditions (FIG. 20A). Treatment with metformin was associated with a dose and time-dependent increase in glucose consumption and lactate production indicating a switch towards increased glycolysis (FIG. 20B-20E). Conversely, culturing cells in low glucose conditions induced a rapid increase in oxygen consumption (FIGS. 20F and 20G), suggesting a shift towards increased oxidative phosphorylation (OXPHOS). Taken together, these results suggest that cancer cells possess the capacity to shuffle between OXPHOS and glycolysis to circumvent the inhibition of either process, which may contribute to explaining both; the weak anti-proliferative effect of OXPHOS inhibition by metformin as well as the fact that targeting glycolysis alone has not provided the expected therapeutic benefit given the well-established glucose bias of tumors. The present findings are therefore in line with previous reports that hinted to the ability of tumor cells to adapt and switch among metabolic pathways. Specifically, several recent reports showed separately in different cell systems and models that inhibition of either glycolysis or OXPHOS triggers a compensatory increase in the other pathway (Chance, 2005; Hao et al., 2010; Jose et al., 2011; Lee and Yoon, 2015). Inventors hypothesized therefore that simultaneous targeting of those pathways may be a more rational approach to target tumor metabolism.

Hypoglycemia-Metformin Combination Effectively Restrains Tumor Growth.

Given the observed metabolic plasticity of cancer cells and to device an effective in vivo metabolic approach to target tumors, inventors aimed to simultaneously target parallel metabolic pathways. Particularly, inventors sought to examine the effect of a combination of fasting-induced hypoglycemia and metformin. To this end, mice bearing HCT116 xenografts were distributed into five groups as schematized in FIG. 13; the first two groups were kept on ad libitum feeding while the three other groups were subjected to 24-hour cycles of feeding-fasting. Fasting in those groups was achieved by complete withdrawal of food while allowing free access to water. To test the effect of metformin alone in the absence of intermittent fasting on tumor growth, one of the two groups on ad lib feeding received vehicle (Vehicle group), while the other received metformin (Met group). To examine the effect of intermittent cycles of fasting on the anti-neoplastic effects of metformin, vehicle or metformin was administered in the three other groups kept on feeding/fasting cycle. The first of those three groups (Fed/Fast group) received vehicle every 48h to assess the effect of fasting-feeding cycles alone on tumor growth. The two last groups received metformin every 48 hours administered either while the mice were fasted (Met/Fast group) or fed (Met/Fed group). In those three groups exposed to fasting-feeding cycles, all mice were fasted at the same time for 24 hours (6 pm-6 pm of the following day) and vehicle or metformin was administered (9 am of next day) as shown in FIG. 13A. In this way, metformin was administered following a period of 15 hours of either fasting (Met/Fast) or feeding (Met/Fed) and was allowed the 9 ensuing hours to act before the fasting or feeding cycle was terminated. Of note, the half-life of metformin in mice is around 2.7 hours (Jee et al., 2007) and it does not bind to plasma proteins or accumulate in the plasma (Greenblatt et al., 1977). As expected, fasting cycles resulted in a strong drop in blood glucose levels, which returned back to almost normal during the following cycle of feeding (FIG. 13B). Administration of metformin also reduced blood glucose levels but to a much less extent than that induced by fasting (FIG. 13B). Notably, it has been shown that while metformin can dramatically lower the high glucose levels in type II diabetes, it has relatively modest effects when administered to subjects with normal glucose levels at baseline (Bonanni et al., 2012)(Pollak, 2012; Sambol et al., 1996). As per design of the experiment, metformin was administered during the hypoglycemia periods in Met/Fast group (FIG. 13C) or near the normoglycemia periods in Met/Fed group (FIG. 13D). This design thus allows us to assess not only the gross effect of intermittent fasting on metformin's anti-neoplastic activities, but also the specific effect of the timing of metformin administration during the fasting-feeding cycles while tumor microenvironments are exposed to different nutritional conditions. Tumor growth was monitored in all groups. Our results show that in this setting, metformin alone did not exert any significant tumor restraining effect. Strikingly however, tumor growth was dramatically impeded in the group receiving metformin while fasting (Met/Fast) as compared to all other experimental groups (FIGS. 13E and 13F) indicating that the anti-proliferative effects of metformin were highly enhanced when it was administered during the hypoglycemia periods of a schedule of intermittent fasting.

Low Glucose Levels Sensitize Cancer Cells to Metformin

Fasting reduces the blood levels of glucose but it also results in a decrease in circulating growth factors and nutrients (Lee and Longo, 2011). To examine which of these factors contribute to the observed sensitization of tumor cells to metformin, HCT116 and HeLa cells were cultured under glucose, serum or amino acid deprivation conditions in the presence or absence of metformin. Cells cultured under nutrient-rich conditions with or without metformin served as control. In agreement with recent reports (Birsoy et al., 2014; Choi and Lim, 2014; Zhuang et al., 2014), deprivation of glucose, but not serum or amino acids, markedly sensitized cells to metformin as cells cultured under low glucose conditions showed marked cell death upon metformin treatment while cells deprived of glucose alone or treated with metformin alone for the same time periods did not show comparable levels of cell death (FIGS. 14A and 14B). Moreover, cells sequentially treated with metformin and low glucose did not show the same magnitude of cell death observed in cells treated simultaneously with combination of both (FIG. 21A). These results suggest that lowering glucose levels in tumor microenvironment upon fasting-induced hypoglycemia sensitizes tumor cells to metformin. The synergistic effect between metformin treatment and glucose deprivation was dependent on both metformin concentration and glucose levels (FIGS. 14C-14E) and was observed in several cancer cell lines as well as patient-derived melanoma cells (FIG. 21B), suggesting it is a general phenomenon not confined only to HCT116 and HeLa cells. This effect also seemed to be specific to metformin as glucose deprivation did not sensitize cells to other cytotoxic agents such as SAHA and Brefeldin A (FIG. 14F), confirming the specificity of this effect rather than predisposition to cytotoxicity per se.

Synergistic Cytotoxicity of Low Glucose/Metformin Combination is AMPK-Independent

Next, inventors analyzed the molecular mechanisms that mediate the synergistic cytotoxicity between metformin and glucose deprivation. The activation of the AMP-activated protein kinase (AMPK) is the most widely accepted mechanism to explain the anti-cancer effects of metformin (Cantó and Auwerx, 2011). We, therefore initially aimed to examine whether AMPK contributes to the observed synergistic cytotoxicity of metformin and low glucose combination. Immunoblotting analysis of lysates derived from HCT116 or HeLa cells cultured either under normal or low glucose conditions in the presence or absence of metformin showed that while in HCT116 cells, AMPK phosphorylation was slightly enhanced by the metformin-low glucose combination, in HeLa cells the combination almost completely abolished AMPK phosphorylation (FIG. 22A). Since HCT116 and HeLa cells showed the same synergistic cytotoxicity in the case of the metformin-low glucose combination, differential AMPK phosphorylation detected in the two cell lines initially suggested that AMPK may not be mediating the observed phenotype. Importantly, depletion of AMPK in HCT116 (FIGS. 22B-22D) or expression of a constitutively active form of AMPK in HeLa cells (FIGS. 22E-22G) failed to modulate the synergistic cytotoxicity observed in both cell lines to any significant extent. In both cases, phosphorylation of acetyl CoA carboxylase (ACC), a known downstream target of AMPK was used as a control to verify AMPK activity (FIGS. 22B and 22E). Taken together, these results indicate that the observed synergistic cytotoxicity of the metformin/low glucose combination is AMPK-independent.

Activation of GSK3β Mediates the Synergistic Cytotoxicity of Low Glucose/Metformin Combination

Inventors next aimed to identify the signaling pathway(s) mediating the observed synergistic cytotoxicity between metformin and low glucose. To this end, inventors used pharmacological inhibitors of several major signaling pathways. Screening of a battery of kinase inhibitors showed that HCT116 and HeLa cells treated with GSK3β inhibitors were resistant to cell death triggered by low glucose/metformin combination (FIG. 15A). Glycogen Synthase Kinase 3 (GSK3) is a Ser/Thr kinase that is known to play crucial roles in the regulation of a wide variety of signaling pathways that control protein synthesis, cell proliferation, differentiation, motility and apoptosis and is involved in the pathogenesis of several diseases (Cohen and Frame, 2001; Frame and Cohen, 2001; Jope and Johnson, 2004). GSK3β activity is regulated by diverse stimuli and signaling pathways. Phosphorylation of its N-terminus serine 9 residue inhibits its activity and it is thus commonly used as a marker for the inactive kinase form. Immunoblotting analysis of lysates derived from HCT116 or HeLa cells cultured under either normal or low glucose conditions in the presence or absence of metformin revealed an almost completely abolished GSK3β phosphorylation (and thus hyperactivation) in the condition of low glucose-metformin combination (FIG. 15B). Notably, low glucose/metformin-induced GSK3β dephosphorylation was consistently observed in both HCT116 and HeLa cells and was specifically induced only by the combination, while either metformin or low glucose alone—if anything—slightly increased the level of GSK3β phosphorylation. Phosphorylation of ERK, similarly to what observed for AMPK, did not show a consistent pattern of phosphorylation between the two cell lines, further confirming the specificity of modulation of GSK3β phosphorylation (FIG. 16B). The effect on GSK33 phosphorylation was also dose-dependent on both metformin and glucose (FIGS. 15C and 15D). Furthermore, a combination of metformin and 2-Deoxy-Glucose (2-DG), a glucose analog that inhibits glycolysis via its actions on hexokinase, the rate limiting step of glycolysis, resulted in a similar dramatic reduction in GSK3β phosphorylation (FIG. 23A), which correlated with synergistic cytotoxicity of this combination (FIG. 23B), further confirming the synergistic cytotoxicity between metformin and inhibition of glycolysis even in cells cultured in glucose-rich conditions and suggesting a role for GSK33 in mediating this synergism. Importantly, GSK33-depleted HCT116, HeLa and patient-derived melanoma cells GaLa1949 and LuCa1973 cells cultured under low glucose conditions proliferated almost normally and did not show cell death upon metformin treatment (FIGS. 15E-16G and 24A) confirming the essential role of GSK30 in mediating the synergistic cytotoxicity between low glucose and metformin.

GSK3β-Dependent Decline in MCL-1 Levels Mediates the Synergistic Cytotoxicity of Low Glucose/Metformin Combination

Among the downstream targets modulated by GSK3β, it has been shown that GSK3β phosphorylates and subsequently enhances the proteasomal degradation of MCL-1, an anti-apoptotic member of the BCL-2 family of proteins (Ding et al., 2007a; Inuzuka et al., 2011; Maurer et al., 2006; Ren et al., 2013a). GSK3β-mediated MCL-1 degradation has been shown to be an essential event in mediating cell death triggered by GSK30 activation (Magiera et al., 2012; Maurer et al., 2006; Morel et al., 2009; Ren et al., 2013b; Wang et al., 2012).

Inventors therefore sought to explore whether MCL-1 modulation plays a role in mediating cell death induced by the low glucose and metformin combination. Immunoblotting analysis of lysates derived from HCT116 and HeLa cells cultured under different nutrient deprivation conditions in the presence or absence of metformin showed that metformin treatment of cells cultured in low glucose (but not upon serum or amino acids starvation) resulted in a marked reduction in MCL-1 protein levels. The levels of the other members of the BCL-2 proteins BCL-2 and Bcl-xL were not affected by the various combinations, confirming the specificity of MCL-1 modulation (FIGS. 16A and 16B). The decline in MCL-1 levels was consistent with GSK30 activation in those cells as described before (FIGS. 16A and 16B) and was also dependent on both metformin and glucose concentrations (FIGS. 16C and 16D). To test whether the observed decline in MCL-1 levels was indeed a result of GSK33 activation, GSK3β-depleted or control cells were cultured in normal or low glucose levels in the presence or absence of metformin. Immunoblotting results showed that unlike control cells expressing scrambled shRNA, metformin-low glucose combination did not result in reduction in MCL-1 levels in cells depleted of GSK3β, indicating that the decline in MCL-1 levels is mediated by GSK3β (FIG. 16E). Consistently, cells treated with pharmacological inhibitor of GSK3β did not show the decline in MCL-1 levels observed in control cells upon treatment with metformin and low glucose combination (FIG. 24B).

Finally, inventors tested whether GSK3β-mediated decline in MCL-1 level contributes to the synergistic cytotoxicity between metformin and low glucose. HCT116, HeLa and patient-derived melanoma cells GaLa1949 and LuCa1973 overexpressing MCL-1 (or as control BCL-2 and Bcl-xL) were cultured in a medium containing normal or low glucose in the presence or absence of metformin. Cells expressing MCL-1 were more resistant to cell death observed in control and BCL-2 or Bcl-xL-expressing cells upon treatment with metformin in low glucose conditions (FIGS. 16F, 16G and 25A).

PP2A Acts Upstream of GSK3β to Mediate the Synergistic Cytotoxicity of Low Glucose/Metformin Combination

Protein phosphatase 2A (PP2A) is a major serine-threonine phosphatase in mammalian cells that has been shown to act as a tumor suppressor through its ability to regulate a number of major molecular switches involved in tumorigenesis. Among those molecular switches, PP2A has been shown to regulate GSK-3β activity by removing phosphorylation at serine 9 as well as other regulatory residues (Bennecib et al., 2000; Kapfhamer et al., 2010; Kumar et al., 2012; Lin et al., 2007a, 2007b; Mitra et al., 2012; Wang et al., 2015). To examine whether modulation of GSK3β phosphorylation by PP2A contributes to the synergistic cytotoxicity of metformin and low glucose, control or PP2A-depleted HCT116, HeLa, and patient-derived melanoma cells GaLa1949 and LuCa1973 cells were cultured in a medium containing normal or low glucose in the presence or absence of metformin. Unlike control cells, PP2A-depleted cells did not show decline in GSK3β phosphorylation or MCL-1 levels in the metformin/low glucose combination (FIG. 17A) and consistently were more resistant to cell death readily observed in the control cells (FIGS. 17B, 18C and 25B).

B56δ Upregulation and CIP2A Inhibition Mediate Modulation of GSK3β-MCL-1 Axis by Low Glucose/Metformin Combination

As described earlier, GSK3β dephosphorylation by PP2A was triggered only by the combination while either metformin or low glucose alone—if anything—did the opposite and increased slightly GSK3β phosphorylation. This ruled out the possibility that the effect could simply be an additive sum of two weaker effects combined together and raised the intriguing question as why this happens only in the case of the two treatments combined but not by either treatment alone. To answer this question and as our results implicate PP2A to be the key upstream regulator of the cytotoxicity of the combination, inventors sought to get deeper mechanistic insight into the modulation of PP2A by metformin, low glucose and the combination of both.

PP2A is a trimeric protein complex consisting of a catalytic subunit (PP2Ac or C), a scaffold subunit (PR65 or A), and one of several alternative regulatory B subunits (Janssens and Goris, 2001). Such variability in PP2A composition results in numerous PP2A holoenzymes, each with unique substrate specificities and different signaling functions in a wide variety of physiological processes and sometimes even in seemingly opposing ways (Sents et al., 2013). The determinants governing PP2A trimer assembly are significantly dependent on the regulatory B-type subunit. The specific B subunit incorporated into the complex modulates substrate specificity, subcellular targeting, and fine-tuning of phosphatase activity (Sents et al., 2013). The activity of PP2A holoenzyme is also regulated by upstream inhibitors, among which cancerous inhibitor of protein phosphatase 2A (CIP2A) is an endogenous PP2A inhibitor that is found overexpressed in several types of cancer and has been shown to contribute to malignant transformation through inhibition of PP2A and therefore evading tumor suppressor functions exerted by PP2A (Junttila et al., 2007; Sangodkar et al., 2016).

Immunoblotting analysis of lysates derived from HCT116 and HeLa cells treated with metformin, low glucose and the combination of both showed that metformin treatment led to reduction in the level of CIP2A while low glucose treatment specifically upregulated the levels of B56δ regulatory subunit (FIG. 18A). Cells treated with metformin/low glucose combination therefore showed reduced CIP2A and enhanced B56δ levels simultaneously. Interestingly GSK3β is an established substrate of PP2A complex containing B56δ (Haesen et al., 2016; Houge et al., 2015; Louis et al., 2011; Bennecib et al., 2000; Kapfhamer et al., 2010; Kumar et al., 2012; Lin et al., 2007a, 2007b; Mitra et al., 2012; Wang et al., 2015).

These results therefore suggested a model in which PP2A is activated by metformin through inhibition of its suppressor CIP2A and when combined with low glucose-induced B56δ upregulation, the combination favors the formation of an active PP2A complex containing B56δ subunit which then targets GSK3β for dephosphorylation, ultimately leading to MCL-1 reduction and cell death.

To test this hypothesis, inventors first overexpressed CIP2A to examine whether metformin-induced downregulation of CIP2A contributes to the modulation of PP2A-GSK33-Mcl-1 axis and the synergistic cytotoxicity by the combination. Our results show that cells overexpressing CIP2A did not show the decline in phosphorylated GSK3β and Mcl-1 levels and the induction of cell death observed in control cells expressing vector upon treatment with metformin/low glucose combination (FIGS. 18B, 18C and 26A).

Furthermore, depletion of CIP2A using shRNA was sufficient to recapitulate the effect of metformin in the combination as a combination of CIP2A depletion and low glucose treatment triggered a decline in phosphorylated GSK3β and MCL-1 levels and evoked cell death similar to what is observed in the case of metformin/low glucose combination (FIGS. 26B and 26C). These results thus indicate that metformin-induced downregulation of CIP2A mediates PP2A activation and can be attributed for—at least in a big part—sensitization of cells to low glucose conditions. Next, inventors aimed to examine the contribution of low glucose-induced upregulation of B56δ subunit to the synergetic cytotoxicity of low glucose/metformin combination. Initially, inventors examined the effect of B56δ depletion. As shown in FIG. 18D, combination of low glucose and metformin did not result in reduction in the levels of phosphorylated GSK3β and MCL-1 in B56δ-depleted cells, unlike control cells or cells depleted of another B regulatory subunit B55α (FIG. 18D). Consistently, B56δ-depleted cells were more resistant to low glucose/metformin combination and showed markedly less cell death compared to control cells (FIGS. 18E and 27), confirming that B56δ is required for GSK3β dephosphorylation, MCL-1 reduction and cytotoxicity in response to the combination.

Finally, overexpression of B56δ and subsequent enrichment of B56δ-containing PP2A holoenzyme synergized with metformin to induce GSK3β dephosphorylation, MCL-1 downregulation and cell death, thus mimicking the effect of low glucose in the combination (FIG. 28A-28D).

Formation of an Active PP2A Holoenzyme Containing B56δ Mediates Cytotoxicity of Low Glucose/Metformin Combination

Taken together, our results showed that metformin-elicited CIP2A downregulation as well as low glucose-induced B56δ upregulation mediate modulation GSK3β-MCL-1 axis by the combination and suggested that PP2A activation following CIP2A downregulation together with B56δ upregulation mediate the cytotoxicity of the combination though the formation of an active PP2A holoenzyme incorporating B56δ subunit with high substrate specificity towards GSK3β. To further test this model, inventors aimed to examine the composition of PP2A holoenzyme under different conditions. To this end, scaffold PP2A Aα subunit was immunoprecipitated from cells treated with either low glucose, metformin or a combination of both. Immunoprecipitation analysis showed specifically enhanced recruitment of B56δ subunit and GSK3β to the PP2A holoenzyme in cells treated with low glucose/metformin combination indicating that the combination indeed elicits the formation of a B56δ-containing PP2A holoenzyme that shows higher substrate affinity toward GSK3β (FIG. 18F). Finally, to further confirm these observations, inventors made use of a PP2A Aα subunit mutant (S256Z) that shows defective binding to B56δ (Reference). PP2A Au-ablated cells were reconstituted with either an empty vector, wild type PP2A Aα or S256Z mutant. Our results show that metformin/low glucose combination failed to induce GSK3β dephosphorylation and MCL-1 downregulation in PP2A Aα ablated cells reconstituted with an empty vector. Reconstitution of those cells with wild type PP2A Aα restored GSK3β dephosphorylation and MCL-1 downregulation upon treatment with the combination, which correlated with enhanced binding between PP2A Aα and B56δ as described earlier. Conversely, metformin/low glucose treatment failed to induce similar B56δ recruitment in cells reconstituted with the PP2A Aα mutant (S256Z) deficient for binding B56δ and consistently did not result in similar GSK30 dephosphorylation, MCL-1 reduction and cell death observed in WT PP2A Au-reconstituted cells. (FIGS. 28A and 28B).

Tumors Depleted of GSK3β or Overexpressing MCL-1 are Resistant to Metformin Administered During Fasting

Our in vitro results indicate that the combination of metformin and low glucose conditions exerts synergistic cytotoxic effects on cancer cells through the dephosphorylation, and thus activation, of GSK33 by an induced PP2A holoenzyme containing the B56δ subunit. Active GSK3β in turn, leads to diminished pro-survival MCL-1 levels, and ultimately to cell death. Inventors then aimed to examine whether this mechanistic model accounts for the tumor-restraining effect of metformin-hypoglycemia in vivo. Immunohistochemistry analysis of tumor tissues derived from the in vivo experiment in FIG. 13 showed that the levels of MCL-1 and phosphorylated GSK3β in the tissues derived from mice treated with metformin while fasting (Met/Fast group) were markedly lower compared to the other experimental groups (FIGS. 19A and 19B). Furthermore, immunoblotting analysis of tumor lysates prepared from Met/Fed against Met/Fast mice showed that administration of metformin to hypoglycemic (fasting) mice resulted in decrease in GSK3β phosphorylation and MCL-1 levels and conversely increase in the B56δ levels (FIG. 19C) and the recruitment of B56δ and GSK3β to PP2A holoenzyme (FIG. 30). AMPK phosphorylation however, did not seem to vary greatly among the two groups (FIG. 19C). Taken together, these results indicate that metformin-hypoglycemia combination elicited molecular events in tumors similar to those observed in vitro in cancer cells treated with metformin-low glucose combination. Finally, to further confirm the involvement of GSK3 f activation and subsequent reduction in MCL-1 levels in restraining tumor growth by metformin-hypoglycemia combination in vivo, mice were inoculated with either control, GSK3β-depleted or MCL-1-overexpressing HCT116 cells. Upon establishment of tumors, mice were kept on 24-hour feeding/fasting cycles with half the mice from each group receiving metformin every 48 hours either during feeding or during fasting cycle as previously explained (see FIG. 13). Monitoring tumor growth showed that unlike control tumors (in which metformin markedly inhibited growth when administered in hypoglycemic mice during fasting (but not during feeding cycles), tumor-derived from GSK3β-depleted or MCL-1-overexpressing cells grew similarly in both conditions and metformin-hypoglycemia combination failed to exert similar growth inhibitory effect on tumor growth (FIGS. 19D and 19E). Collectively, these results further highlight the crucial role of modulation of the PP2A-GSK33-MCL-1 axis in mediating the synergistic anti-proliferative effect of metformin-low glucose in vitro and similarly metformin-hypoglycemia in vivo as an approach to tackle tumor metabolic plasticity according to the model depicted in FIG. 19F.

PP2A Inducer Perphenazine Synergizes with Metformin In Vitro and In Vivo

Inventors finally aimed to exploit the molecular insight gained by analyzing the PP2A-GSK3β-MCL-1 axis in response to hypoglycemia/metformin combination to attempt a pharmacological approach that could mimic the effect of this combination with more clinical feasibilities. Inventors made use of perphenazine (PPZ), an FDA-approved anti-psychotic medication that has been shown to induce PP2A activity (Gutierrez et al., 2014; Research, 2014; Tsuji et al., 2016) and that our observations showed that it enhances the assembly of PP2A holoenzyme containing the B56δ subunit and the therefore recruitment of GSK3β (FIG. 31A),

Our results show that similar to hypoglycemia-metformin combination, treatment with a combination of PPZ with metformin diminished the phosphorylated GSK3β and MCL-1 levels (FIG. 31B). Consistently, PPZ sensitized HCT116 and HeLa cells to metformin specifically but not to other cytotoxic agents such as SAHA and Brefeldin A (FIGS. 31C and 31D). PPZ also synergized with metformin in impeding xenograft tumor growth in vivo (FIGS. 31E and 31F). These results suggest that modulation of PP2A-GSK3β-MCL-1 axis can be exploited pharmacologically to enhance the tumor-restraining effects of metformin.

Discussion

Despite the big leap in our understanding of the differences between metabolism in normal versus cancer cells, this understanding has not so far been translated into clinically feasible approaches for therapeutic intervention in cancer. Particularly, given the bias of tumor cells towards increased uptake of glucose and switch to aerobic glycolysis, it is puzzling that many agents designed to inhibit these processes failed to provide the expected therapeutic benefits when tested clinically (Rodriguez-enriquez et al., 2009). In the light of our observations and other emerging reports, a major obstacle for tackling cancer metabolism is the metabolic plasticity of cancer cells demonstrated by their ability to shuffle among different metabolic pathways and thus circumvent the inhibition of a single pathway. Inventors therefore hypothesized that a more rational approach would be to aim to simultaneously inhibit alternative metabolic pathways.

In the present study, inventors exploited intermittent fasting (IF) as a clinically feasible, safe and effective approach to lower glucose availability and explored the potential synergistic effect of a combination of IF with the OXPHOS inhibitor metformin on tumor growth. In glucose-starving tumor cells, OXPHOS becomes critical for survival, which may render those cells particularly sensitive to metformin. Alternatively, fasting-induced glucose limitation may serve to impede the compensatory increase in glycolysis upon OXPHOS inhibition by metformin.

Our results show that the tumor-restraining effect of metformin was dramatically enhanced when it was administered in mice subjected to alternating cycles of fasting/feeding specifically during the periods of fasting-induced hypoglycemia. Interestingly, our results therefore indicate that not only intermittent fasting, but also the timing of metformin administration during the fasting/feeding cycles dictates the sensitivity of tumors to metformin. Notably, in our experimental setting (nude mice), fasting induced a reduction in blood glucose levels by 40-60%. However, the reduction in glucose levels within the tumor microenvironment is likely to be greater, considering the considerable uptake and use of glucose by tumor cells, leading to quicker and more profound glucose depletion in the hypovascularized tumor microenvironment. This could contribute to the observation that while the fasting protocol was well-tolerated with no gross clinical signs of toxicity on mice in whole, the effect on tumor tissues was detrimental when combined with metformin treatment.

Mechanistic analysis of the molecular events triggered by the low glucose-metformin combination initially ruled out a central role for AMPK in mediating cell death in response to the combination. The unexpected independency on AMPK was intriguing given the well-established role of AMPK in mediating the response to either metformin or low glucose individually and indicated that the combination specifically triggers signaling events that are distinguished from those triggered in response to each of the two treatments alone and thus cannot simply be attributed to the sum of the effects of the two individual treatments.

Further mechanistic investigation showed that cell death in response to low glucose/metformin combination was a consequence of a tightly orchestrated signaling process mediated by specific modulation of the PP2A-GSK3β-MCL-1 axis. Importantly, genetic manipulation of this pathway (through overexpression of MCL-1 or depletion of either PP2A or GSK3β) abrogated the synergism and rendered tumor cells resistant to a combination of low glucose and metformin in vitro and fasting-induced hypoglycemia and metformin in vivo. As initially deduced from the independency on AMPK, the specific modulation of GSK3β phosphorylation and MCL-1 levels only in the case of the combination, but neither by metformin or low glucose alone, indicated once again that the combination triggers molecular events that are distinguished from those triggered by either of the treatments alone. Since modulation of PP2A seemed to be an early response to energetic stress triggered by the combination in our model, inventors carried out mechanistic analysis of the PP2A complex assembly and activity in order to get further insight into the specificity of the combination.

PP2A is an important and ubiquitously expressed serine threonine phosphatase that regulates the function of many crucial molecules through mediating their dephosphorylating. PP2A thereby plays important roles in diverse cellular processes including cell cycle progression, DNA replication, gene transcription and protein translation (Westermarck and Hahn, 2008). In tumorigenesis, PP2A has been established as a tumor suppressor and the inactivation of PP2A has become widely accepted as an important step towards full-blown transformation (Eichhorn et al., 2009; Janssens et al., 2005; Mumby, 2007; Perrotti and Neviani, 2008). Indeed, genetic and/or functional inactivation of different PP2A subunits and, therefore, loss of its phosphatase activity have been found in several types of tumors (mutations of PP2A subunits have been found at high frequency in lung, colorectal and breast cancers: Perrotti and Neviani 2013).

Our findings thus establish a novel role for PP2A as an early response sensor of the energetic stress triggered in our model by simultaneous inhibition of alternative metabolic pathways via a combination of metformin and low glucose. Interestingly, in line with our findings, a recent study showed that simultaneous targeting of multiple metabolic through inhibition of nutrient uptake triggered PP2A activation (Kim et al., 2016).

Structurally, PP2A holoenzyme is a heterotrimeric complex of a catalytic C subunit, a scaffolding A subunit, and one of several regulatory B-type subunits. The substrate specificity and activity of PP2A are highly regulated by the type of the regulatory B-type subunit incorporated in the complex. It is thus essential when analyzing the role of PP2A in any biological context, to identify which of the many alternative PP2A complexes is/are involved. Additionally, PP2A activity is regulated by upstream inhibitors among which, CIP2A is an important endogenous PP2A inhibitor in cancer cells.

Delving deep into the molecular mechanisms, our results showed that on the one hand, treatment with metformin diminished the levels of PP2A inhibitor CIP2A in cancer cells. On the other hand, culturing cancer cells in low glucose triggered an increase in the levels of PP2A regulatory subunit B56δ. Taken together, only cells treated with metformin and low glucose combination exhibit a simultaneous decline in CIP2A together with an enrichment in B56δ subunit levels leading to the formation of an active PP2A complex containing the B56δ subunit, which subsequently targets GSK3β for dephosphorylation and ultimately leading to reduction in MCL-1 levels and cell death. This detailed molecular insight explains the specific synergistic cytotoxicity of the combination. Importantly, analysis of tumor samples from our in vivo model indicated that the same molecular model accounts for the tumor-restraining effect of the combination of metformin and fasting-induced hypoglycemia.

The PP2A inhibitor CIP2A is an oncoprotein, originally identified as a binding partner of the PP2A A subunit. CIP2A is specifically overexpressed in numerous types of tumors while is barely detectable in normal cells, making it a potential therapeutic target. CIP2A overexpression has been shown to correlate with poor prognosis in lung cancer, breast cancer, pancreatic cancer, bladder cancer, osteosarcoma, esophageal cancer, gastric cancer, ovarian cancer, cervical cancer, prostate cancer, HCC and colorectal cancer (Haesen et al., 2014; Seshacharyulu et al., 2013). Inhibition of PP2A and thus evading its tumor suppressor actions accounts for a big part of tumorigenic potential of CIP2A. It is not known exactly how CIP2A inhibits PP2A activity however it has been proposed that CIP2A interacts with the A subunit and impedes binding of B subunits to PP2A complexes (Junttila et al., 2007; Khanna et al., 2013; Sangodkar et al., 2016). According to this model, it is possible that metformin-induced downregulation of CIP2A frees PP2A A and C subunits from the inhibitory interaction with CIP2A, which when combined with low-glucose B56δ upregulation, allows the formation of an active complex of PP2A A, C and B56δ subunits. PP2A regulatory subunit B56δ plays a role in tumorigenesis through its established function in the regulation of GSK3β dephosphorylation (Haesen et al., 2016; Houge et al., 2015; Louis et al., 2011; Bennecib et al., 2000; Kapfhamer et al., 2010; Kumar et al., 2012; Lin et al., 2007a, 2007b; Mitra et al., 2012; Wang et al., 2015) and possibly other substrates. Besides its role in cancer, B56δ dysregulation has also been associated with neurological disorders. Mice lacking B56δ develop ataxia and tauopathy (Louis et al., 2011) and mutations in B56δ encoding gene Ppp2r5d that render B56δ deficient for binding PP2A A and C subunits have also been recently identified in patients with intellectual disability (Houge et al., 2015).

Downstream of the PP2A complex, our results also establish a crucial role for GSK3β in mediating the observed synergistic cytotoxicity. GSK3β has been shown to play both tumor suppressor and promoter roles in cancer. The modulation of cell death by GSK3β contributes to its dual role in tumorigenesis. GSK3β has been shown to regulate several targets promoting both cell death and survival. GSK3β has been shown to mediate cell death in response to a wide variety of conditions including DNA damage, hypoxia, endoplasmic reticulum stress, heat shock and growth factor withdrawal (Beurel and Jope, 2006; Bijur and Jope, 2000; Hongisto et al., 2003; Jacobs et al., 2012; King et al., 2001; Loberg et al., 2002; Pap and Cooper, 1998, 2002; Somervaille et al., 2001; Song et al., 2002)

Phosphorylation and subsequent degradation of MCL-1 has been shown to play an essential role in mediating GSK3β-induced cell death in response to certain stimuli such as UV irradiation, anticancer drug treatment and inhibition of growth factor pathways (Magiera et al., 2012; Maurer et al., 2006; Morel et al., 2009; Ren et al., 2013b; Wang et al., 2012). Furthermore, the levels of MCL-1 correlate with phosphorylated GSK3β levels (the inactive form of GSK3β) in multiple cancer cell lines and primary human cancer samples (Ding et al., 2007b).

MCL-1 is a pro-survival member of the BCL-2 family that is upregulated in several types of tumors and contributes to drug resistance and relapse in those tumors (Aichberger et al., 2005; Akgul, 2009; Boisvert-Adamo et al., 2009; Boisvert-adamo et al., 2009; Cho-Vega et al., 2004; Elgendy, 2017, 2017; Gores and Kaufmann, 2012; Gores et al., 2012; Jiang et al., 2008; Khoury et al., 2003; Oyesanya et al., 2012; Quinn et al., 2011; Robillard et al., 2005; Warr and Shore, 2008; Wuilleme-Toumi et al., 2005). MCL-1 plays a key role in the regulation of apoptosis and its tumor-promoting properties have been largely attributed to its anti-apoptotic functions. However, recent reports suggest that MCL-1 might also be involved in other cellular processes that may contribute to its tumorigenic potential. Besides the crucial roles of MCL-1 in apoptosis, apoptosis-independent functions of MCL-1 in the regulation of autophagy and cellular energetics are emerging (Elgendy and Minucci, 2015; Elgendy et al., 2014; Germain et al., 2011; Perciavalle et al., 2012). The short half-life of MCL-1 makes it a particularly potential mediator for the regulation of critical processes such as changes in cellular energetics that require prompt coordination of cellular responses. While metformin may exhibit single-agent activity in some contexts, there is generally more interest in exploring its potential use in combinatorial therapy. Many combinations have been proposed but few have been thoroughly examined. Collectively, our findings suggest that the combination of metformin with intermittent fasting or PP2A inducers may prove efficacious in targeting cancer cells and warrants further clinical evaluation Additionally, our results predict that the functional/genetic loss of PP2A will lead to loss of synergism in treatment, and suggest a potential strategy for stratification of patients.

Targeting PP2A has emerged as a promising therapeutic strategy in cancer potentially capable of overcoming drug-resistance induced in patients by continuous exposure to kinase inhibitors. Although phosphatases remain generally difficult to target by small molecules, PP2A activators such as phenothiazines, forskolin, 1,9-dideoxy-forskolin and FTY720 effectively have been shown to impede leukemogenesis in both in vitro and in vivo models (Perrotti and Neviani, 2008). Importantly, the drug exploited in this study (perphenazine) is approved for clinical use as an anti-psychotic: inventors therefore suggest to exploit its PP2A inducing activity for repurposing it as an anti-cancer agent (Gutierrez et al., 2014; Research, 2014; Tsuji et al., 2016).

Finally, the effects of metformin described here occur at doses that can be achieved clinically, and therefore our model appears to be immediately amenable to validation in clinical studies.

### Example 3: DDR and NER During Chronological Aging

Materials and Methods

Strains and Growth Conditions:

Yeast strains were derived from W303 (Thomas and Rothstein, 1989), but RAD5+ background and are listed in Table 1A.

TABLE 1A
Strains used herein

| Strain | Relevant genotype | Source |
|---|---|---|
| SY 2080 | W303-1a ade2-1 trp1-1 leu2-3,112 his3-11,15 | H. Klein |
| | ura3 can1-100 RAD5+ GAL PSI+ | |
| CY11668 | W303-1a sch9::KanMX6 | This study |
| CY14953 | W303-1a atg1::KanMX6 | This study |
| CY11823 | W303-1a snf1::NatMX6 | This study |
| CY12239 | W303-1a SNF1-G53R | This study |
| CY15050 | W303-1a pBGM18 [URA3, ADH2-lacZ] | This study |
| CY11697 | W303-1a rrd1::NAT | This study |
| CY11900 | W303-1a tip41::KAN | This study |
| CY14101 | W303-1a GLN3::MYC13-KanMX6 | This study |
| CY14103 | W303-1a NNK1::MYC13-KanMX6 | This study |
| CY14105 | W303-1a NPR::MYC13-KanMX6 | This study |
| CY14167 | W303-1a GLN3::MYC13-KanMX6 snf1::HPH | This study |
| CY14169 | W303-1a NNK1::MYC13-KanMX6 snf1::HPH | This study |
| CY14171 | W303-1a NPR1::MYC13-KanMX6 snf1::HPH | This study |
| SY2081 | W303-1α ade2-1 trp1-1 leu2-3,112 his3-11,15 | H. Klein |
| | ura3 can1-100 GAL PSI+ | |
| CY14598 | W303-1α sch9::KanMX6 | This study |
| CY14595 | W303-1α gcn2::KanMX6 | This study |
| CY14597 | W303-1α gcn2::KanMX6 sch9::KanMX6 | This study |
| CY12015 | W303-1a gcn2::KanMX6 | This study |
| CY13855 | W303-1a gcn2::KanMX6 rrd1::Nat | This study |
| CY13857 | W303-1a gcn2::KanMX6 tip41::Nat | This study |
| CY15106 | W303-1a atg1::KanMX6 sch9::KanMX6 | This study |

Deletion and MYC-tagging were obtained using one-step PCR-targeting method (Longtine et al., 1998). The SNF1-G53R allele was constructed through the ‘delitto perfetto’ strategy (Storici and Resnick, 2006). Strain CY14595 was obtained by tetrad dissection of the diploid generated by SY2081 and CY12015 mating. Strains CY14598 and CY14597 were derived from tetrad dissection of the mating between CY11668 and CY14595.

All CLS experiments were performed according to the protocol described (Hu et al., 2013). Rapamycin (Sigma-Aldrich) was used at a final concentration of 2 ng/ml while metformin (Sigma-Aldrich) at 80 mM. Both drugs were supplemented at the initial overnight culture stage (Day 0), prior to cells entering stationary phase. In experiments including rapamycin-treated cultures, DMSO (0.04%) was used in the untreated cultures. For each chronological aging experiment, cells were set up in synthetic complete medium (SDC) and allowed to grow overnight at 28° C. to reach exponentially growing conditions the next day (day 1) until the end of kinetics (day 10/11 for DDR and NER analysis, day 21-28 for viability). At each examined day, small aliquots of cells were removed, exposed to 40 J/m² and further incubated in their exhausted medium. Samples were taken at untreated (prior to UV), 0 and 2 hours (for protein extraction) or 0, 6 and 24 hours (for genomic DNA extraction) after UV treatment.

Western Blot Analysis:

Protein extracts were prepared using TCA extraction as described (Chiolo et al., 2005). Protein samples were loaded on 10% SDS-PAGE, followed by western blot analysis using different antibodies: anti-Rad53 (EL7 antibody (Fiorani et al., 2008) produced by IFOM monoclonal facility), anti-phospho Thr 210 of Snf1 (Cell Signalling), anti-phospho-eIF2α (Cell Signalling), anti-total eIF2α (a gift from Dr. Tom Dever), anti Myc (clone 9E10) antibody and anti-PGK (Life Technologies-Novex) antibody. For Sch9, protein samples were run on 7.5% SDS-PAGE gels, subjected to Western Blot analysis with anti-phospho and total Sch9 antibodies, kindly provided by Dr. Maria E. Cardenas (Kingsbury et al., 2014). PP2A targets: Gln3-myc, Npr1-myc, Nnk1-myc were all separated on NuPage precast 3-8% SDS gels. PonceauS staining was used as a loading control.

Thymine Dimer Repair Assay:

Cells were fixed in one volume of ice-cold 100% ethanol and incubated on ice for 5′. Genomic DNAs were then prepared following standard procedure. Equal amounts of DNA were loaded on 0.8% agarose gel, transferred by Southern blot onto nitrocellulose membrane (Amersham Protran 0.45μ) and cross-linked by baking 1 hour at 80° C.

Filters were then probed with an antibody that specifically recognizes thymine dimers (ab10347, Abcam), stripped and reprobed with an antibody that specifically recognizes ssDNAs (MAB3034, Millipore) as a control for total DNA amount loaded on the gel.

Chronological Lifespan Analysis:

Lifespan Viability Assays were Carried as Follows: Aliquots of Cells were Removed Throughout the CLS kinetics, serially diluted and spotted on YPD plates. The plates were then exposed to 40 J/m² (or 20 J/m² in some cases), incubated at 25° C. and scanned after 3 days. In FIGS. 33, 34 and 36 individual spot assay rows were cut out for space reasons, but the compared strains were always from the same plates.

For viability curves (FIG. 32C, 33D, 33G), cells were taken from the original flasks at the indicated days, counted, serially diluted and spread on YPD plates. Plates were UV-flashed in the range spanning 20-80 J/m² to analyze sensitivity of cells to UV damage, or left untreated as a control. They were incubated at room temperature in the dark and after 3 days, colony-forming units (c.f.u.) were counted. To analyze cell survival in aging, the ratio between viable colonies grown on plates (multiplied by the dilution factor) and total cells present in the flasks was calculated and results were normalized to the Day1 values. To analyze UV sensitivity in aging, the number of colonies counted on UV-treated plates was divided by the number of colonies counted on untreated plates at each day. Values are average+/−st dev on n=3 replicates.

Inventors note that the variety in the kinetics of aging is due to the nature of biological variability inventors consistently observed during CLS. Inventors emphasize that the pattern of phenotypes is consistent.

lacZ Activity Assay:

The ADH2-lacZ plasmid (YCpBGM18) was a gift from Ted Young (Young et al., 2000) and transformed into SY2080 to produce strain CY15050. URA+ colonies were set up to grow in CLS synthetic media as described above, except lacking Uracil, in untreated or Metformin-treated (80 mM) conditions. CLS kinetic time course was performed and samples (˜1×10⁸ cells) were harvested at Days 1, 4, 7, 10, 15 and 20, washed in dH₂O+PMSF and pellets were frozen at −80° C. lacZ expression was assayed using a protocol described by Hepworth et al (Hepworth et al, 1995). Frozen pellets were resuspended in 200 μl Z buffer and cells were broken by vortexing with 500 μm diameter glass beads for 10×1 min pulses at 4° C. Z buffer (0.81 μl) was added and the samples were adjusted to 0.05% SDS and 2% chloroform. Aliquots of the extracts were then added to Z buffer to give a final volume of 0.5 ml. The samples were prewarmed at 28° C. for 2 minutes before addition of 0.1 ml of ONPG (O-Nitrophenyl-β-galactoside) solution (4 mg/ml of 0.1M NaPO4 [pH7]). After incubation at 28° C., the reactions were stopped by addition of 0.25 ml Na₂CO₃. The samples were centrifuged briefly and the optical density at 420 nm of the supernatant was recorded. β galactosidase activity is given as nanomoles of ONPG cleaved per minute per milligram of protein at 28° C. Protein concentration of the extract was determined by the Bradford assay. All the reported values are the average activities obtained from n=3 replicates representing each day in both −/+Metformin.

### Example 4

Experimental Procedures

Reagents

Antibodies were purchased from the indicated sources and used at a dilution of 1:1000 unless otherwise described: anti-pCHK1, anti-pCHK2, (Cell Signaling Technology); anti-Vinculin (SIGMA, dilution of 1:10000). Small molecule compounds were purchased from the following sources: Metformin, perphenazine, FTY-720, ceramide C2, thioridazine, fluphenazine, thiethylperazine, pimozide, clozapine, loratadine, promethazine, haloperidol (Sigma Aldrich).

Tissue Culture

HCT116 and HeLa cell lines were grown in Dulbecco's modified Eagle's medium (DMEM) supplemented with 10% fetal bovine serum and 2 mM L-glutamine unless otherwise indicated. Bx-PC3 cells were grown in RPMI medium supplemented with 10% fetal bovine serum and 2 mM L-glutamine unless otherwise indicated. For starvation experiments, cells were washed three times with PBS pH 7.2 and then incubated in the indicated starvation conditions. All cultures were maintained in a humidified tissue culture incubator at 37° C. in 5% C02.

Immunoblotting

Whole cell lysates were prepared by directly lysing cells growing in culturing dishes or collected cell pellets in lysis buffer (40 mM Hepes pH 7.5, 120 mM NaCl, 1 mM EDTA, 10 mM pyrophosphate, 10 mM glycerophosphate, 50 mM NaF, 0.5 mM orthovanadate, and EDTA-free protease inhibitors (Roche) containing 0.3% CHAPS). Lysates were cleared by centrifugation at 13000 g for 15 min. at 4° C., quantified using BioRad DC protein assay reagent followed by mixing 1:1 with 4% SDS, 100 mM Tris.Cl pH 6.8, 20% glycerol, 0.1% bromophenol blue and 5% (3-mercaptoethanol added immediately before use and heating at 94° C. for 7 min. Equal amounts of proteins were then electrophoresed on 8-15% SDS-PAGE gels. Gels were run at 100 V (stacking gel)/150 V (separation gel) on Protean III apparatus (BioRad). Gels were transferred onto nitrocellulose and probed with the appropriate primary antibody for a variable incubation time depending on the experimental design, followed by the corresponding secondary antibodies diluted 1:5000-10000. The proteins were visualized by enhanced chemiluminescence (ECL) using ChemiDoc apparatus (BioRad) according to the manufacturer's instructions.

RNA Interference

shRNA pLKO-Tet-On and pLKO.1 lentiviral constructs were purchased from Open Biosystems. Target sequences are as follows:

| | | |
|---|---|---|
| Scrambled: | GTGGACTCTTGAAAGTACTAT | (SEQ ID NO: 1) |
| PP2A Cα #1: | ACCGGAATGTAGTAACGATTT | (SEQ ID NO: 10) |
| PP2A Cα #2: | GGCAAATCACCAGATACAAA | (SEQ ID NO: 11) |
| PP2A Cα #3: | TGGAACTTGACGATACTCTAA | (SEQ ID NO: 12) |
| RAD51 #1: | GCTGAAGCTATGTTCGCCATT | (SEQ ID NO: 13) |

Lentiviral Transduction

The pLKO vectors and package plasmids were co-transfected into packaging HEK293T cells and the viral supernatants were collected, supplemented with polybrene (8 ug/mL) and used to infect target cells in four 2-hour cycles of transduction over two consecutive days.

Quantification of Cell Proliferation

CellTiter Glo Luminescent Cell Viability Assay (Promega) was used according to manufacturer's protocol. Briefly, cells were plated in 96 well plates, treated 24h later with different doses of drugs in total volume of 100 μl. 24h later, 100 μl of CellTiter Glo reagent was added to the cells and incubated for 15 min at 37° C. and luminescence was measured using a Promega plate reader.

Quantification of Cell Viability

Cells were harvested by trypsinization, washed in PBS (pH 7.2), and then stained with trypan blue solution 04% v/v (Sigma Aldrich) added immediately prior to analysis. Cells ware then counted on a TC30 automated cell counter (Biorad).

Results

the Modulation of the DNA Damage Response by the Signaling Pathways Regulated by PP2A and Nutrient Availability in Tumor Cells

To parallel the studies performed in yeast, showing a clear involvement of PP2A in regulating the DDR, and to integrate them with the data obtained in mammalian cells (metformin/low glucose impinging on a PP2A-regulated signaling pathway), inventors performed studies in tumor cells where they:
combined metformin with DNA damaging agents (mainly a combination of hydroxyurea/gemcitabine), in conditions where those drugs have a minimal impact on cell viability;
directly triggered PP2A activation through compounds known to modulate PP2A directly or indirectly (ceramide);
studied the relevance of glucose concentrations in conjunction with DNA damaging agents;
evaluated the relevance of PP2A in the observed response, by knocking down PP2A expression;
studied the activation of the DDR by evaluating phosphorylation status of factors involved in the DDR (Chk1 and Chk2).

Inventors then observed that:
several modulators of PP2A cooperate with DNA damaging agents in inducing cell death of tumor cells;
while this effect is present also in normal medium, it is amplified at various extent in low glucose conditions, consistently with the different modalities of PP2A activation by the different compounds;
the cooperation depends on PP2A, as shown by the knockdown experiments;
the cooperations correlates with inhibition of the activation of at least two key components of the DDR (Chk1 and Chk2).

As a parallel to the experiments described above, treatment with okadaic acid (FIG. 56) inhibiting PP2A protects HeLa cells treated with high concentrations of hydroxyurea (HU), counteracting the activity of DNA damaging agents by inhibiting Pp2A and presumably enhancing the efficiency of DDR.

All experiments have been repeated at least 3 times with similar results.

### Example 5

Genetic Impairment of the DDR is Synthetic Lethal to Treatments Able to Activate PP2A

Inventors reasoned that—given the proposed role of PP2A in the regulation of DDR− tumor cells that are already defective for any component of the DDR itself should be more sensitive than cells that do not present this defect to treatments that activate PP2A, such as the metformin/low glucose treatment.

To demonstrate this concept in a stringent way, rather than comparing tumor cell lines from different sources, inventors engineered a pancreatic cancer cell line (Bx-PC3 pancreatic cancer cells) to down-regulate RAD51, a key component of the DDR. They then examined the response of the knocked-down cells compared to the parental ones to metformin-low glucose treatment, known from present studies to activate PP2A.

Indeed, knockdown of RAD51 in Bx-PC3 cells dramatically sensitizes them to PP2A-inducing treatment. They surmise therefore that tumors carrying defects in the DDR (that can be scored for instance by the presence of inactivating mutations in components of the DDR, such as RAD51, or BRCA1/2, or other DDR factors) can be especially suited for treatments activating PP2A, and therefore analysis of DDR markers can be a diagnostic tool to stratify patients for treatment (FIG. 48).

All experiments have been repeated at least 3 times with similar results.

### Example 6

Characterization of the Activity of Small Molecules Known as PP2A Activators

In the past years, several small molecules were shown to have the ability to modulate PP2A, acting either directly on the holo-enzyme, or indirectly on regulatory factors. Inventors have therefore tested several small molecules for their ability to cooperate with metformin treatment. The assay was performed in 96-well plates (n=4) in HeLa cells, treating cells as described before, in high (10 mM) and low (2.5 mM) glucose conditions. Viability was measured by CellTiter Glo. Parallel assays (cell count with Trypan blue) confirmed the results.

Perphenazine and thioridazine were the only drugs able to cooperate with metformin in reducing tumor cell viability in high glucose conditions, while 7 other compounds cooperated with metformin only under low glucose conditions (see table 1B below). Inventors therefore speculated that—while perphenazine and thioridazione, as shown herein for perphenazine, can activate PP2A and recruit actively the B56δ subunit to the PP2A holo-complex—other drugs can activate PP2A, but require low-glucose conditions to increase the amount of the PP2A-B560 containing holoenzyme.

TABLE 1B

| name | Active in high glucose | Active in low glucose |
|---|---|---|
| PERPHENAZINE | + | + |
| THIORIDAZINE | + | + |
| FLUPHENAZINE | − | + |
| THIETHYLPERAZINE | − | + |
| PIMOZIDE | − | + |
| CLOZAPINE | − | + |
| LORATADINE | − | + |
| PROMETHAZINE | − | + |
| HALOPERIDOL | − | + |

In fact, (see FIG. 58) inventors performed immunoprecipitation and total cell lysate analysis of PP2A Aalpha from cell lysates derived from HCT116 cells treated with either DMSO, 10 uM PERPHENAZINE (PPZ) or 10 uM THIORIDAZINE (Thio) and cultured for 24 hours in high glucose DMEM in the absence or presence of metformin (5 mM).

### Example 7

Additional Data on the Combination of Metformin/Low Glucose

Inventors performed additional experiments to characterize present main findings, with the following results, summarized in FIGS. 50-55:
The metformin-intermittent fasting combination (in vitro: metformin-low glucose) also works on PDX models of tumor. So, the data initially observed in cell lines can be reproduced in the in vivo model considered the golden standard;
Low doses of metformin can achieve similar effects to those observed with higher doses shown herein (with an extended duration of treatment). This finding erases the concerns linked to the possibility that the doses of metformin used in some in vitro experiments cannot be reached in the patients. Inventors also show that low doses of metformin/low glucose trigger the same biochemical pathways that inventors have studied at higher doses;
To better characterize the induction of cell death by metformin/low glucose, inventors show that it can be greatly inhibited by caspase inhibitors, linking the observed effect to induction of caspase-mediated cell death;
Inventors have already herein shown that knockdown of several components of the identified PP2A signaling pathway abrogates the response to metformin-low glucose. Here, it is shown that this finding can be reproduced also in long-term clonogenic assays, complementing short-term assays that inventors have shown before.

Inventors have observed the cooperation metformin-low glucose in a comprehensive list of cell lines/primary tumors (see below), as an indication that it can be widely used for tumor treatment in solid tumors and hematological malignancies.

Summary of Cell Lines Analyzed and Responsive to Metformin/Low Glucose Treatment:

Solid Tumors
Colon cancer: HCT116
Cervical cancer: HeLa
Breast cancer: MCF7
Ovarian cancer: COLO704
Melanoma: SK-MEL28, GaLa 1949, LuCa 1970
Lung cancer: A549
Pancreatic cancer: Bx-PC3
Neuroendocrine tumors: BON-1 (see FIG. 57)

AMLs
Eol1, PBL-985, HL-60, ML-2, PL-21, UF1, THP1, MOLM-14 cell lines

TABLE 2
Yeast strains herein used

| Name | Genotype | Reference |
|---|---|---|
| SY2080 | MAT a, ade2-1, ura3-1, his3-11,15, leu2-3,112, trp1-1, can1-100, | H. Klein, Lab collection |
| | GAL, PSI+, RAD5+ | |
| CY9145 | MAT a, ade2-1, ura3-1, his3-11,15, leu2-3,112, trp1-1, can1-100, | This study |
| | GAL, PSI+, RAD5+, irc21::KNMX6 | |
| CY11846 | MAT a, ADE2+, ura3-1, his3-11, leu2-3,112, trp1-1, can1-100, | This study |
| | GAL, PSI+, RAD5+, GAL1-rad53-D339A::URA | |
| CY11845 | MAT a, ADE2+, ura3-1, his3-11, leu2-3,112, trp1-1, can1-100, | This study |
| | GAL, PSI+, RAD5+, GAL1-rad53-D339A::URA, irc21::NAT | |
| CY11864 | MAT a, ADE2+, ura3-1, his3-11, leu2-3,12, trp1-1, can1-100, | This study |
| | GAL, PSI+, RAD5+, GAL1-rad53-D339A::URA, rrd1::NAT | |
| CY12137 | MAT a, ADE2+, ura3-1, his3-11, leu2-3,112, trp1-1, can1-100, | This study |
| | GAL, PSI+, RAD5+, GAL1-rad53-D339A::URA, tip41::NAT | |
| CY10043 | MAT a, ade2-1, ura3-1, his3-11,15, leu2-3,112, trp1-1, can1-100, | Lab collection |
| | GAL, PS1+, RAD5+, rad53K227A::KANMX4 | |
| CY12352 | MAT a, ade2-1, ura3-1, his3-11,15, leu2-3,112, trp1-1, can1-100, | This study |
| | GAL, PSI+, RAD5+, rad53K227A::KANMX4, irc21::NAT | |
| CY13431 | MAT a, ade2-1, ura3-1, his3-11,15, leu2-3,112, trp1-1, can1-100, | This study |
| | GAL, PSI+, RAD5+, rad53K227A::KANMX4, rrd1::NAT | |
| CY13429 | MAT a, ade2-1, ura3-1, his3-11,15, leu2-3,112, trp1-1, can1-100, | This study |
| | GAL, PSI+, RAD5+, rad53K227A::KANMX4, tip41::NAT | |
| CY13156 | MAT a, ade2-1, ura3-1, his3-11,15, leu2-3,112, trp1-1, GAL, PS1+, | Lab collection |
| | RAD5+, rad53K227A::KANMX4, sml1::TRP1 | |
| CY12336 | MAT a, ade2-1, ura3-1, his3-11,15, leu2-3,112, trp1-1, GAL, PSI+, | This study |
| | RAD5+, rad53K227A::KANMX4, sml1::TRP1 irc21::NAT | |
| CY8998 | MAT a, ade2-1, ura3-1, his3-11,15, leu2-3,112, trp1-1, can1-100, | Lab collection |
| | GAL, PSI+, RAD5+, sml1::TRP1 | |
| CY13439 | MAT a, ade2-1, ura3-1, his3-11,15, leu2-3,112, trp1-1, can1-100, | Lab collection |
| | GAL, PSI+, RAD5+, sml1::HIS3 | |
| CY14094 | MAT a, ade2-1, ura3-1, his3-11,15, leu2-3,112, trp1-1, can1-100, | Lab collection |
| | GAL, PSI+, RAD5+, sml1::HPH | |
| CY14095 | MAT a, ade2-1, ura3-1, his3-11,15, leu2-3,112, trp1-1, can1-100, | Lab collection |
| | GAL, PSI+, RAD5+, sml1::HPH, mec1::NAT | |
| CY14097 | MAT a, ade2-1, ura3-1, his3-11,15, leu2-3,112, trp1-1, can1-100, | Lab collection |
| | GAL, PSI+, RAD5+, sml1::HPH, rad53::NAT | |
| CY13441 | MAT a, ade2-1, ura3-1, his3-11,15, leu2-3,112, trp1-1, can1-100, | This study |
| | GAL, PSI+, RAD5+, sml1::HIS3, irc21::KANMX6 | |
| CY13766 | MAT a, ade2-1, ura3-1, his3-11,15, leu2-3,112, trp1-1, can1-100, | This study |
| | GAL, PSI+, RAD5+, sml1::HIS3, rrd1::NAT | |
| CY13770 | MAT a, ade2-1, ura3-1, his3-11,15, leu2-3,112, trp1-1, can1-100, | This study |
| | GAL, PSI+, RAD5+, sml1::HIS3, tip41::KANMX6 | |
| CY14528 | MAT a, ade2-1, ura3-1, his3-11,15, leu2-3,112, trp1-1, can1-100, | This study |
| | GAL, PSI+, RAD5+, sml1::KANMX6, tap42-G360R-URA3 | |
| CY14499 | MAT a, ade2-1, ura3-1, his3-11,15, leu2-3,112, trp1-1, can1-100, | This study |
| | GAL, PSI+, RAD5+, sml1::HPH, mec1::NAT, irc21::KANMX6 | |
| CY13797 | MAT a, ade2-1, ura3-1, his3-11,15, leu2-3,112, trp1-1, GAL, PSI+, | This study |
| | RAD5+, sml1::TRP1, mec1::URA3, rrd1::KANMX6 | |
| CY13772 | MAT a, ade2-1, ura3-1, his3-11,15, leu2-3,112, trp1-1, can1-100, | This study |
| | GAL, PSI+, RAD5+, sml1::TRP1, mec1::URA3, tip41::KANMX6 | |
| CY14532 | MAT a, ade2-1, ura3-1, his3-11,15, leu2-3,112, trp1-1, can1-100, | This study |
| | GAL, PSI+, RAD5+, sml1::KAN, mec1::NAT, tap42-G360R-URA3 | |
| CY14501 | MAT a, ade2-1 ura3-1, his3-11,15 leu2-3,112, trp1-1, can1-100, | This study |
| | GAL, PSI+, RAD5+, sml1::HPH, rad53::NAT, irc21::KANMX6 | |
| CY14063 | MAT a, add2-1, ura3-1, his3-11,15, leu2-3,112, trp1-1, can1-100, | This study |
| | GAL, PSI+, RAD5+, sml1::TRP1, rad53-NAT, rrd1-KANMX6 | |
| CY14061 | MAT a, ade2-1, ura3-1, his3-11,15, leu2-3,112, trp1-1, can1-100, | This study |
| | GAL, PSI+, RAD5+, sml1::HIS, rad53::NAT, tip41::KANMX6 | |
| CY14530 | MAT a, ade2-1, ura3-1, his3-11,15, leu2-3,112, trp1-1, can1-100, | This study |
| | GAL, PSI+, RAD5+, sml1::KANMX6, rad53::NAT, tap42-G360R-URA3 | |
| CY14503 | MAT a, ade2-1, ura3-1, his3-11,15, leu2-3,112, trp1-1, can1-100, | This study |
| | GAL, PSI+, RAD5+, sml1::HPH, mec1::NAT rad53::KANMX6 | |
| CY13988 | MAT a, ade2-1, ura3-1, his3-11,15, leu2-3,112, trp1-1, GAL, PSI+, | This study |
| | RAD5+, sml1::TRP1, mec1::URA3, irc21::NAT, rad53::KANMX6 | |
| CY14070 | MAT a, ade2-1, ura3-1, his3-11,15, leu2-3,112, trp1-1, can1-100, | This study |
| | GAL, PSI+, RAD5+, sml1::HIS3, chk1::HPH | |
| CY14072 | MAT a, ade2-1, ura3-1, his3-11,15, leu2-3,112, trp1-1, can1-100, | This study |
| | GAL, PSI+, RAD5+, sml1::HIS3, chk1::HPH, irc21::KAN | |
| CY14505 | MAT a, ade2-1, ura3-1, his3-11,15, leu2-3,112, trp1-1, can1-100, | This study |
| | GAL, PSI+, RAD5+, sml1::HIS3, chk1::HPH, rad53::KAN | |
| CY13996 | MAT a, ade2-1, ura3-1, his3-11,15, leu2-3,112, trp1-1, GAL, PSI+, | This study |
| | RAD5+, sml1::TRP1, chk1::HIS3, irc21::NAT, rad53::KANMX4 | |
| CY13795 | MAT a, ade2-1, ura3-1, his3-11,15, leu2-3,112, trp1-1, GAL, PSI+, | This study |
| | RAD5+, sml1::TRP1, mec1::URA3, irc21::NAT, tel1::KAN | |
| CY13768 | MAT a, ade2-1, ura3-1, his3-11,15, leu2-3,112, trp1-1, can1-100, | This study |
| | GAL, PSI+, RAD5+, tel1::HIS3, sml1::KANMX6 | |
| CY13774 | MAT a, ade2-1, ura3-1, his3-11,15, leu2-3,112, trp1-1, can1-100, | This study |
| | GAL, PSI+, RAD5+, tel1::HIS3, sml1::KANMX6, irc211::NAT | |
| CY10263 | MAT a, ade2-1, ura3-1, his3-11,15, leu2-3,112, trp1-1, can1-100, | Lab collection |
| | GAL, PSI+, RAD5+, sml1::TRP1, mec1::KANMX6, tel1::HIS3 | |
| CY12161 | MAT a, ade2-1, ura3-1, his3-11,15, leu2-3,112, trp1-1 can1-100, | Lab collection |
| | GAL, PSI+, RAD5+, dun1::HIS3 | |
| CY13875 | MAT a, ade2-1, ura3-1, his3-11,15, leu2-3,112, trp1-1, can1-100, | This study |
| | GAL, PSI+, RAD5+ dun1::HIS3, irc21:NAT | |
| CY13863 | MAT a, ade2-1, ura3-1, his3-11,15, leu2-3,112, trp1-1, can1-100, | This study |
| | GAL, PSI+, RAD5+, sml1::TRP1, Dun1-PK::HIS3 | |
| CY13873 | MAT a, ade2-1, ura3-1, his3-11,15, leu2-3,112, trp1-1, can1-100, | This study |
| | GAL, PSI+, RAD5+, sml1::TRP1, Dun1-PK::HIS3, irc21::NAT | |
| CY14507 | MAT a, ade2-1, ura3-1, his3-11,15, leu2-3,112, trp1-1, can1-100, | This study |
| | GAL, PSI+, RAD5+, sml1::TRP1, Dun1-PK::HIS3, mec1::KAN | |
| CY13867 | MAT a, ade2-1, ura3-1, his3-11,15, leu2-3,112, trp1-1, can1-100, | This study |
| | GAL, PSI+, RAD5+, sml1::TRP1, Dun1-PK::HIS3, rad53::NAT | |
| CY14509 | MAT a, ade2-1, ura3-1, his3-11,15, leu2-3,112, trp1-1, can1-100, | This study |
| | GAL, PSI+, RAD5+, sml1::TRP1, Dun1-PK::HIS3, irc21::NAT, | |
| | mec1::KANMX6 | |
| CY14511 | MAT a, ade2-1, ura3-1, his3-11,15, leu2-3,112, trp1-1, can1-100, | This study |
| | GAL, PSI+, RAD5+, sml1::TRP1, Dun1-PK::HI3S, irc21::NAT, | |
| | rad53::KANMX6 | |
| CY13824 | MAT a, ade2-1, ura3-1, his3-11,15, leu2-3,112, trp1-1, can1-100, | M. P. Longhese, this study |
| | GAL, PSI+, RAD5+, hta1::KANMX4, hta2-S129A-URA3 | |
| CY13828 | MAT a, ade2-1, ura3-1, his3-11,15, leu2-3,112, trp1-1, can1-100, | M. P. Longhese, this study |
| | GAL, PSI+, RAD5+, hta1::KANMX4, hta2-S129A-URA3, irc21::NAT | |
| CY10984 | MAT a, ade2-1, ura3-1, his3-11,15, leu2-3,112, trp1-1, can1-100, | This study |
| | GAL, PSI+, RAD5+, ptc1::KANMX6 | |
| CY9143 | MAT a, ade2-1, ura3-1, his3-11,15, leu2-3,112, trp1-1, can1-100, | This study |
| | GAL, PSI+, RAD5+, rts1::KANMX6 | |
| CY11914 | MAT a, ade2-1, ura3-1, his3-11,15, leu2-3,112, trp1-1, can1-100, | This study |
| | GAL, PSI+, BAD5+, sap190::KANMX6 | |
| CY11697 | MAT a, ade2-1, ura3-1, his3-11,15, leu2-3,112, trp1-1, can1-100, | This study |
| | GAL, PSI+, BAD5+, rrd1::NAT | |
| CY11900 | MAT a, ade2-1, ura3-1, his3-11,15, leu2-3,112, trp1-1, can1-100, | This study |
| | GAL, PSI+, RAD5+, tip41::KANMX6 | |
| CY14523 | MAT a, ade2-1, ura3-1, his3-11,15, leu2-3,112, trp1-1, can1-100, | M. P. Longhese this study |
| | GAL, PSI+, RAD5+, tap42-G360R-URA3 | |
| CY10816 | MAT alpha, ade2-1, ura3-1, his3-11,15, leu2-3,112, trp1-1, can1- | This study |
| | 100, GAL, PSI+, RAD5+, irc21::NAT | |
| CY12323 | MAT alpha, ade2-1, ura3-1, his3-11,15, leu2-3,112, trp1-1, can1-100, | This study |
| | GAL, PSI+, RAD5+, irc21::HPH | |
| CY10363 | MAT alpha, can1::STE2pr-Sp_his5, lyp1, ura3Δ0, leu2Δ0, his3Δ1, | C. Boone, Lab collection |
| | met15Δ0 | |
| CY10678 | MAT alpha, can1::STE2pr-Sp_his5, lyp1, ura3Δ0, leu2Δ0, his3Δ1, | This study |
| | met15Δ0, irc21::NAT | |
| CY14101 | MAT a, ade2-1, ura3-1, his3-11,15, leu2-3,112, trp1-1, can1-100, | This study |
| | GAL, PSI+, RAD5+, GLN3::GLN3-MYC-KANMX6 | |
| CY14134 | MAT a, ade2-1, uria3-1, his3-11,15, leu2-3,112, trp1-1, can1-100, | This study |
| | GAL, PSI+, RAD5+, GLN3::GLN3-MYC-KANMX6, irc21::NAT | |
| CY14136 | MAT a, ade2-1, ura3-1, his3-11,15, leu2-3,112, trp1-1, can1-100, | This study |
| | GAL, PSI+, RAD5+, GLN3::GLN3-MYC-KANMX6, rrd1::NAT | |
| CY14138 | MAT a, ade1-1, ura3-1, his3-11,15, leu2-3,112, trp1-1, can1-100, | This study |
| | GAL, PSI+, RAD5+, GLN3::GLN3-MYC-KANMX6, tip41::NAT | |
| CY14524 | MAT a, ade2-1, ura3-1, his3-11,15, leu2-3,112, trp1-1, can1-100, | M. P. Longhese, this study |
| | GAL, PSI+, RAD5+, GLN3::GLN3-MYC-KANMX6, tap42-G360R-URA3 | |
| CY14103 | MAT a, ade2-1, ura3-1, his3-11,15, leu2-3,112, trp1-1, can1-100, | This study |
| | GAL, PSI+, RAD5+, NNK1::NNK1-MYC-KANMX6 | |
| CY14140 | MAT a, ade2-1, ura3-1, his3-11,15, leu2-3,112, trp1-1, can1-100, | This study |
| | GAL, PSI+, RAD5+, NNK1::NNK1-MYC-KANMX6, irc21::NAT | |
| CY14142 | MAT a, ade2-1, ura3-1, his3-11,15, leu2-3,112, trp1-1, can1-100, | This study |
| | GAL, PSI+, RAD5+, NNK1::NNK1-MYC-KANMX6, rrd1::NAT | |
| CY14144 | MAT a, ade2-1, ura3-1, his3-11,15, leu2-3,112, trp1-1, can1-100, | This study |
| | GAL, PSI+, RAD5+, NNK1::NNK1-MYC-KANMX6, tip41::NAT | |
| CY14525 | MAT a, ade2-1, ura3-1, his3-11,15, leu2-3,112, trp1-1, can1-100, | M. P. Longhese, this study |
| | GAL, PSI+, RAD5+, NNK1::NNK1-MYC-KANMX6, tap42-G360R-URA3 | |
| CY14105 | MAT a, ade2-1, ura3-1, his3-11,15, leu2-3,112, trp1-1, can1-100, | This study |
| | GAL, PSI+, RAD5+, NPR1::NPR1-MYC-KANMX6 | |
| CY14146 | MAT s, ade2-1, ura3-1, his3-11,15, leu2-3,112, trp1-1, can1-100, | This study |
| | GAL, PSI+, RAD5+, NPR1::NPR1-MYC-KANMX6, irc21::NAT | |
| CY14146 | MAT a, ade2-1, ura3-1, his3-11,15, leu2-3,112, trp1-1, can1-100, | This study |
| | GAL, PSI+, RAD5+, NPR1::NPR1-MYC-KANMX6, rrd1::NAT | |
| CY14150 | MAT a, ade2-1, ura3-1, his3-11,15, leu2-3,112, trp1-1, can1-100, | This study |
| | GAL, PSI+, RAD5+, NPR1::NPR1-MYC-KANMX6, tip41::NAT | |
| CY14526 | MAT a, ade2-1, ura3-1, his3-11,15, leu2-3,112, trp1-1, can1-100, | M. P. Longhese, this study |
| | GAL, PSI+, RAD5+, NPR1::NPR1-MYC-KANMX6, tap42-G360R-URA3 | |
| CY14069 | MAT a, ade2-1, ura3-1, his3-11,15, leu2-3,112, trp1-1, can1-100, | This study |
| | GAL, PSI+, RAD5+, RTG3::RTG3-MYC-KANMX6 | |
| CY14107 | MAT a, ade2-1, ura3-1, his3-11,15, leu2-3,112, trp1-1, can1-100, | This study |
| | GAL, PSI+, RAD5+, RTG3::RTG3-MYC-KANMX6, irc21::NAT | |
| CY14109 | MAT a, ade2-1, ura3-1, his3-11,15, leu2-3,112, trp1-1, can1-100, | This study |
| | GAL, PSI+, RAD5+, RTG3::RTG3-MYC-KANMX6, rrd1::NAT | |
| CY14111 | MAT a, ade2-1, ura3-1, his3-11,15, leu2-3,112, trp1-1, can1-100, | This study |
| | GAL, PSI+, RAD5+, RTG3::RTG3-MYC-KANMX6, tip41::NAT | |
| CY11685 | MAT a, ade2-1, ura3-1, his3-11,15, leu2-3,112, trp1-1, can1-100, | Lab collection |
| | GAL, PSI+, RAD5+, SCH9::SCH9-PK-HIS3 | |
| CY11779 | MAT a, ade2-1, ura3-1, his3-11,15, leu2-3,112, trp1-1, can1-100, | This study |
| | GAL, PSI+, RAD5+, SCH9::SCH9-PK-HIS3, irc21::KANMX6 | |
| CY12013 | MAT a, ade2-1, ura3-1, his3-11,15, leu2-3,112, trp1-1, can1-100, | This study |
| | GAL, PSI+, RAD5+, SCH9::SCH9-PK-HIS3, rrd1::NAT | |
| CY14497 | MAT a, ade2-1, ura3-1, his3-11,15, leu2-3,112, trp1-1, can1-100, | This study |
| | GAL, PSI+, RAD5+, SCH9::SCH9-PK-HIS3, tip41::NAT | |
| CY13998 | MAT a, ade2-1, ura3-1, his3-11,15, leu2-3,112, trp1-1, can1-100, | This study |
| | GAL, PSI+, RAD5+, ppm1::KANMX6 | |
| CY14000 | MAT a, ade2-1, ura3-1, his3-11,15, leu2-3,112, trp1-1, can1-100, | This study |
| | GAL, PSI+, RAD5+, irc21::NAT, ppm1::KANMX6 | |
| CY14617 | MAT a, ade2-1, ura3-1, his3-11,15, leu2-3,112, trp1-1, can1-100, | This study |
| | GAL, PSI+, RAD5+, rad53K227A::KANMX4, ppm1::HPH | |
| CY14631 | MAT a, ade2-1, ura3-1, his3-11,15, leu2-3,112, trp1-1, can1-100, | This study |
| | GAL, PSI+, RAD5+, sml1::HPH, ppm1::KANMX6 | |
| CY14619 | MAT a, ade2-1, ura3-1, his3-11,15, leu2-3,112, trp1-1, can1-100, | This study |
| | GAL, PSI+, RAD5+, sml1::HPH, mec1::NAT, ppm1::KANMX6 | |
| CY14621 | MAT a, ade2-1, ura3-1, his3-11,15, leu2-3,112, trp1-1, can1-100, | This study |
| | GAL, PSI+, RAD5+, GLN3::GLN3-MYC-KANMX6, ppm1::HPH | |
| CY14623 | MAT a, ade2-1, ura3-1, hls3.11,15, leu2-3,112, trp1-1, can1-100, | This study |
| | GAL, PSI+, RAD5+, NNK1::NNK1-MYC-KANMX6, ppm1::HPH | |
| CY14625 | MAT a, ade2-1, ura3-1, his3-11,15, leu2-3,112 trp1-1, can1-100, | This study |
| | GAL, PSI+, RAD5+, NPR1::NPR1-MYC-KANX6, ppm1::HPH | |
| CY14627 | MAT a, ade2-1, ura3-1, his3-11,15, leu2-3,112, trp1-1, can1-100, | This study |
| | GAL, PSI+, RAD5+, RTG3::RTG3-MYC-KANMX6, ppm1::HPH | |
| CY14197 | MAT a, ade1-1, uram3-1, his3-11,15, leu2-3,112, trp1-1, can1-100, | This study |
| | GAL, PSI+, RAD5+, scs7::NAT | |
| CY14195 | MAT a, ade2-1, ura3-1, his3-11,15, leu2-3,112, trp1-1, can1-100, | This study |
| | GAL, PSI+, RAD5+, sur2::NAT | |
| CY14199 | MAT a, ade2-1, ura3-1, his3-11,15, leu2-3,112, trp1-1, can1-100, | This study |
| | GAL, PSI+, RAD5+, sur2::NAT, scs7::HPH | |
| CY14203 | MAT a, ade2-1, ura3-1, his3-11,15, leu2-3,112, trp1-1, can1-100, | This study |
| | GAL, PSI+, RAD5+, rad53K227A::KANMX4, sur2::NAT | |
| CY14205 | MAT a, ade2-1, ura3-1, his3-11,15, leu2-3,112, trp1-1, can1-100, | This study |
| | GAL, PSI+, RAD5+, rad53K227A::KANMX4, scs7::NAT | |
| CY14207 | MAT a, ade2-1, ura3-1, his3-11,15, leu2-3,112, trp1-1, can1-100, | This study |
| | GAL, PSI+, RAD5+, rad53K227A::KANMX4, sur2::NAT, scs7::HPH | |
| CY14633 | MAT a, ade2-1, ura3-1, his3-11,15, leu2-3,112 trp1-1, can1-100, | This study |
| | GAL, PSI+, RAD5+, sml1::HPH, scs7::KAN | |
| CY14635 | MAT a, ade2-1, ura3-1, his3-11,15, leu2-3,112, trp1-1, can1-100, | This study |
| | GAL, PSI+, RAD5+, sml1::HPH, sur2::KAN | |
| CY14637 | MAT a, ade2-1, ura3-1, his3-11,15, leu2-3,112, trp1-1, can1-100, | This study |
| | GAL, PSI+, RAD5+, sml1::HPH, scs7::KAN, mec1::NAT | |
| CY14639 | MAT a, ade2-1, ura3-1, his3-11,15, leu2-3,112, trp1-1, can1-100, | This study |
| | GAL, PSI+, RAD5+, sml1::HPH, sur2::KAN, mec1::NAT | |

TABLE 3
Primer sequences herein used.

| | | | | SEQ |
|---|---|---|---|---|
| | | Primer | | ID |
| Target | | Name | Primer Sequence | NO: |
| IRC21 | for- | I21MXF | ACGAATAAGCAGAATATAACATAT | 14 |
| dele- | ward | | TAGCAGGTGCTTAGATTACACTCA | |
| tion | | | TAGAGATACATGGAGGCCCAGAAT | |
| | | | ACCCT | |
| | re- | I21MXR | AGTGTTTTTATATCCTATGTAAGT | 15 |
| | verse | | CTTCAAACTTTTTTTTTATCTCTG | |
| | | | GTAACCTCAGTATAGCGACCAGCA | |
| | | | TTCAC | |
| RRD1 | fo- | RRD1F1 | AAAGAACGCACATATGAACAAGCA | 16 |
| dele- | rward | | TTAAACGAGCAAAGAACGGATCCC | |
| tion | | | CGGGTTAATTAA | |
| | re- | RRD1R1 | TCATAATGCTTGTCATACACATTT | 17 |
| | verse | | ATATGTTTAATTAATAGAATTCGA | |
| | | | GCTCGTTTAAAC | |
| TIP41 | for- | TIP41F1 | ACCTAAGGGCAGCTTTAGACACAA | 18 |
| dele- | ward | | CAGCTCCCCAGAAAAACGGATCCC | |
| tion | | | CGGGTTAATTAA | |
| | re- | TIP41R1 | CGTGTATGTATTTGTACGTATTGT | 19 |
| | verse | | TTTGTATATTTGATTGGAATTCGA | |
| | | | GCTCGTTTAAAC | |
| SML1 | for- | SML1F1 | TCTCACTAACCTCTCTTCAACTGC | 20 |
| dele- | ward | | TCAATAATTTCCCGCTCGGATCCC | |
| tion | | | CGGGTTAATTAA | |
| | re- | SML1R1 | GGGAAATGGAAAGAGAAAAGAAAA | 21 |
| | verse | | GAGTATGAAAGGAACTGAATTCGA | |
| | | | GCTCGTTTAAAC | |
| MEC1 | for- | MEC1MXF | AAGTGAGGCTGGACAACAAGAACG | 22 |
| dele- | ward | | ACATACACCGCGTAAAGGCCCACA | |
| tion | | | AGACTGCACATGGAGGCCCAGAAT | |
| | | | ACCCT | |
| | re- | MEC1MXR | AGTGATGGTTAGATCAAGAGGAAG | 23 |
| | verse | | TTCGTCTGTTGCCGAAAATGGTGG | |
| | | | AAAGTCGCAGTATAGCGACCAGCA | |
| | | | TTCAC | |
| RAD53 | for- | RAD53MXF | AGCTTTAAAAGAGAGAATAGTGAG | 24 |
| dele- | ward | | AAAAGATAGTGTTACACAACATCA | |
| tion | | | ACTAAAAACATGGAGGCCCAGAAT | |
| | | | ACCCT | |
| | re- | RAD53MXR | CTACCATCTTCTCTCTTAAAAAGG | 25 |
| | verse | | GGCAGCATTTTCTATGGGTATTTG | |
| | | | TCCTTGGCAGTATAGCGACCAGCA | |
| | | | TTCAC | |
| CHK1 | for- | CHK1F1 | GTATATCATAAGTTGCTGTATATG | 26 |
| dele- | ward | | GGCAGCACGTATTACTCGGATCCC | |
| tion | | | CGGGTTAATTAA | |
| | re- | CHK1R1 | TGATCAGTGCATCTTAACCCTTCT | 27 |
| | verse | | TTTGTCTCCATTTTTTGAATTCGA | |
| | | | GCTCGTTTAAAC | |
| PTC1 | for- | PTC1F1 | ATTTAGGCACTGCATTTATCTTTT | 28 |
| dele- | ward | | AAAAATCATTATACGGATCCCCGG | |
| tion | | | GTTAATTAA | |
| | re- | PTC1R1 | GTCTATGCATAATTTTTGCGCGGT | 29 |
| | verse | | TTATAACGGATCCTTCGAATTCGA | |
| | | | GCTCGTTTAAAC | |
| TEL1 | for- | TEL1F1 | AAGCCTTCAAAGAAAAAGGGAAAT | 30 |
| dele- | ward | | CAGTGTAACATAGACGCGGATCCC | |
| tion | | | CGGGTTAATTAA | |
| | re- | TEL1R1 | TATAAACAAAAAAAAGAAGTATAA | 31 |
| | verse | | AGCATCTGCATAGCAAGAATTCGA | |
| | | | GCTCGTTTAAAC | |
| RTS1 | for- | RTS1F1 | ATCATAGGCACGTGCTATTTTCGA | 32 |
| dele- | ward | | ACATCCACTTTCAATCGGATCCCC | |
| tion | | | GGGTTAATTAA | |
| | re- | RTS1R1 | AAACTTCCTCACTTCTTCGAGCTT | 33 |
| | verse | | GTAATGAATTGCTGTTGAATTCGA | |
| | | | GCTCGTTTAAAC | |
| SAP190 | for- | SP190F1 | CATTTCTTCATTTACTTAACTGCG | 34 |
| dele- | ward | | AGAAGATTATAATAGCCGGATCCC | |
| tion | | | CGGGTTAATTAA | |
| | re- | SP190R1 | TGAATAAAGGGTGAAAATGTGACA | 35 |
| | verse | | ATGTGAATGTTTTAGTGAATTCGA | |
| | | | GCTCGTTTAAAC | |
| SUR2 | for- | SUR2F1 | TTCTAGTCCGAAGAGGGTGTATAC | 36 |
| dele- | ward | | GAAAAGAAAATATACGCGGATCCC | |
| tion | | | CGGGTTAATTAA | |
| | re- | SUR2R1 | TGCCTTTACCCAGCAATTGAACGG | 37 |
| | verse | | GAGGTATGCAAAAGGGGAATTCGA | |
| | | | GCTCGTTTAAAC | |
| SCS7 | for- | SCS7F1 | CAGGCACTAAAAGCGGTGGTAAGC | 38 |
| dele- | ward | | TAAAACTAGTACGAAGCGGATCCC | |
| tion | | | CGGGTTAATTAA | |
| | re- | SCS7R1 | TTTTCCTAGGTTGACAATTTTGGA | 39 |
| | verse | | CGAGGCTGACCAATAAGAATTCGA | |
| | | | GCTCGTTTAAAC | |
| PPM1 | for- | PPM1F1 | TCCGCATAAACTAGATGATAAAGA | 40 |
| dele- | ward | | GTACAAACAAGTCGCCCGGATCCC | |
| tion | | | CGGGTTAATTAA | |
| | re- | PPM1R1 | AGCATATTAAGATCAAATTAGTTG | 41 |
| | verse | | AGGCTGTAAATAAAAAGAATTCGA | |
| | | | GCTCGTTTAAAC | |
| Gln3- | for- | GLN3F2 | AGCAATTGCTGACGAATTGGATTG | 42 |
| tag- | ward | | GTTAAAATTTGGTATACGGATCCC | |
| ging | | | CGGGTTAATTAA | |
| | re- | GLN3R1 | TTATTAACATAATAAGAATAATGA | 43 |
| | verse | | TAATGATAATACGCGGGAATTCGA | |
| | | | GCTCGTTTAAAC | |
| Nnk1- | for- | NNK1F2 | AATGAACCTAAGCGAGGCCATTCA | 44 |
| tag- | ward | | CGATAATAATGGCTCACGGATCCC | |
| ging | | | CGGGTTAATTAA | |
| | re- | NNK1R1 | TATGTATTTTTTCAATGCAATCAA | 45 |
| | verse | | TATCATTAATCATAAGGAATTCGA | |
| | | | GCTCGTTTAAAC | |
| Npr1- | for- | NPR1F2 | TGCAGGCCTAGAAAAGAAAAAGAA | 46 |
| tag- | ward | | AAAGCAAAATAATCAACGGATCCC | |
| ging | | | CGGGTTAATTAA | |
| | re- | NPR1R1 | TACAAATGCTTGGAAAAGAAATAA | 47 |
| | verse | | AAGTGGGGACGCTTATGAATTCGA | |
| | | | GCTCGTTTAAAC | |
| Rtg3- | for- | RTG3F2 | CTCTAATCCAGCTGACTATCTTTT | 48 |
| tag- | ward | | AGAATTTGGTTCGGGGCGGATCCC | |
| ging | | | CGGGTTAATTAA | |
| | re- | RTG3R1 | TTTTTCAAATTTAATTTTTTCCCG | 49 |
| | verse | | CTAATAAGACCATAAAGAATTCGA | |
| | | | GCTCGTTTAAAC | |

TABLE 4
Chemical structures of the herein mentioned molecules.

| Name | Structure |
|---|---|
| Caffeine | 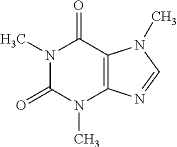 |
| Cardiolipin | 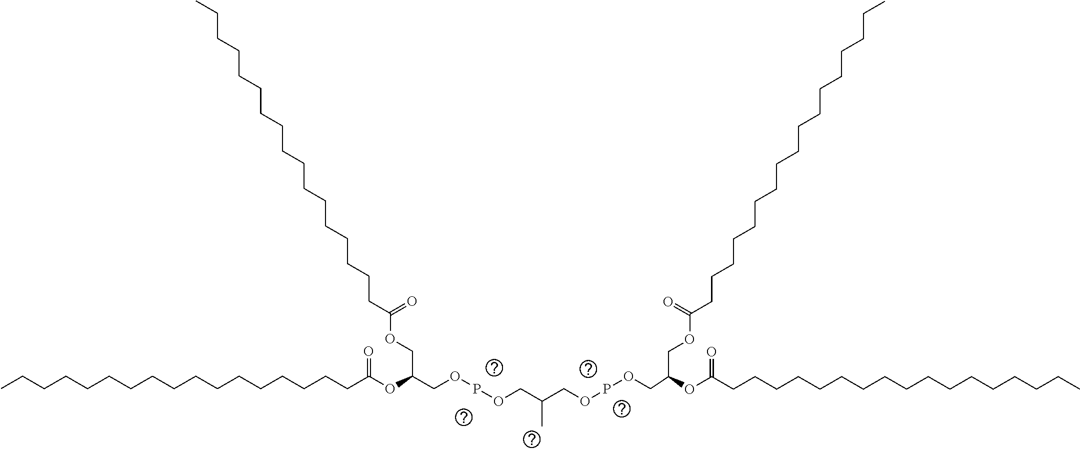 |
| Clozapine | 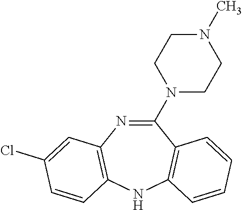 [SMILES: CN1CCN(C2=Nc3cc(Cl)ccc3Nc3ccccc32)CC1] |
| FLUPHENAZINE | 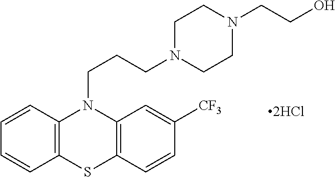 [SMILES: OCCN1CCN(CCCN2c3ccccc3Sc3ccc(C(F)(F)F)cc32)CC1] |
| FTY720 | 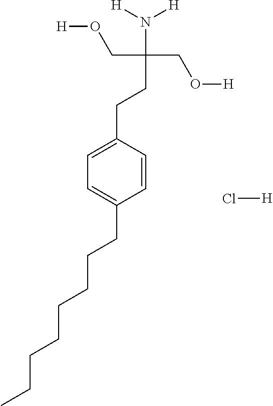 [SMILES: CCCCCCCCc1ccc(CCC(N)(CO)CO)cc1 ; Cl] |
| FUMONISIN B1 | 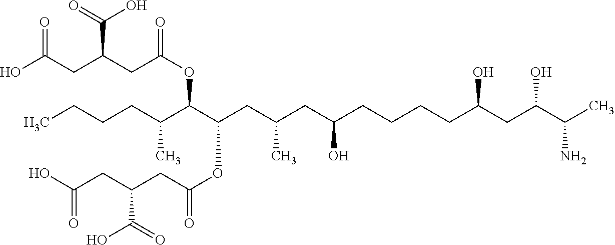 [SMILES: CCCC[C@@H](C)[C@@H](OC(=O)C[C@@H](CC(=O)O)C(=O)O)[C@H](C[C@@H](C)C[C@H](O)CCCC[C@@H](O)C[C@H](O)[C@H](C)N)OC(=O)C[C@@H](CC(=O)O)C(=O)O] |
| HALOPERIDOL | 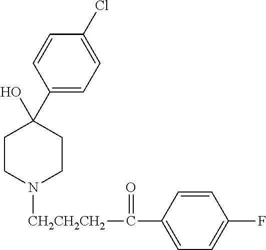 |
| LORATADINE | 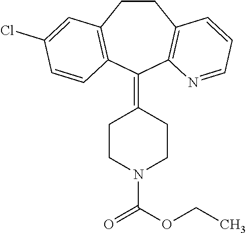 [SMILES: CCOC(=O)N1CCC(=C2c3ccc(Cl)cc3CCc3cccnc32)CC1] |
| MERSALYL ACID | 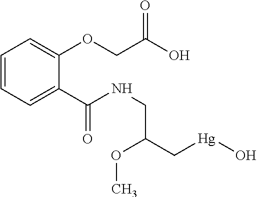 |
| METFORMIN HCl | 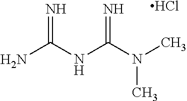 |
| MYRIOCIN | 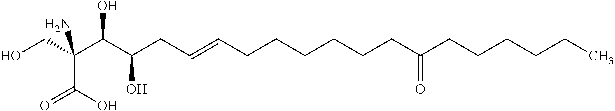 [SMILES: CCCCCCC(=O)CCCCCC/C=C/C[C@@H](O)[C@H](O)[C@@](N)(CO)C(=O)O] |
| OKADAIC ACID | 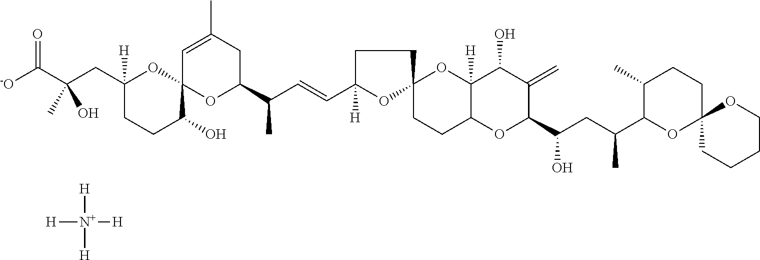 |
| PERPHENAZINE HCl | 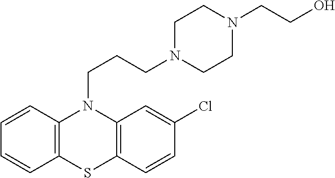 [SMILES: OCCN1CCN(CCCN2c3ccccc3Sc3ccc(Cl)cc32)CC1] |
| PIMOZIDE | 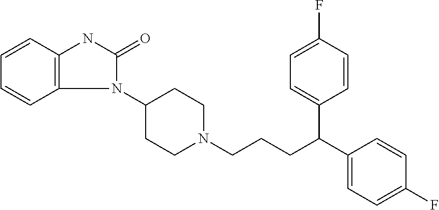 [SMILES: O=c1[nH]c2ccccc2n1C1CCN(CCCC(c2ccc(F)cc2)c2ccc(F)cc2)CC1] |
| PROMETHAZINE HCl | 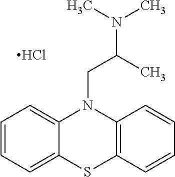 [SMILES: CC(CN1c2ccccc2Sc2ccccc21)N(C)C ; Cl] |
| RAPAMYCIN | 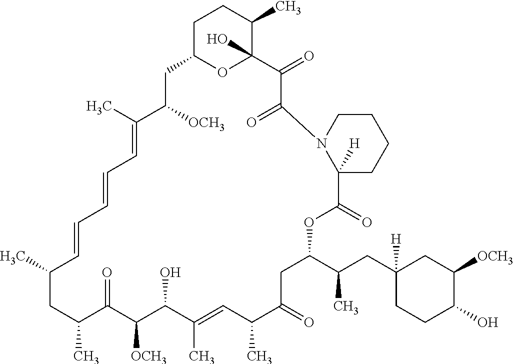 |
| S-ADENOSYL METHIONINE | 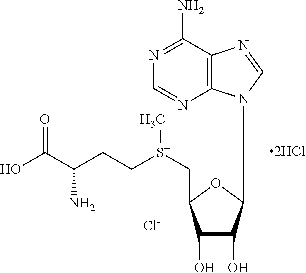 |
| THIETHYLPERAZINE MALEATE | 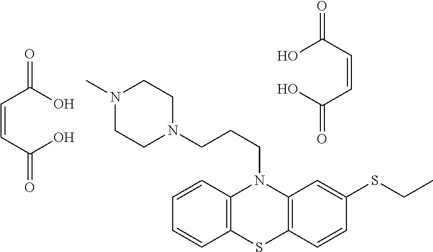 |
| THIORIDAZINE HCl | 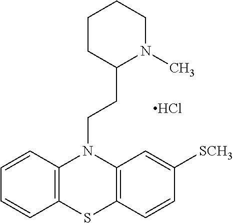 [SMILES: CSc1ccc2c(c1)N(CCC1CCCCN1C)c1ccccc1S2 ; Cl] |
| WORTMANNIN | 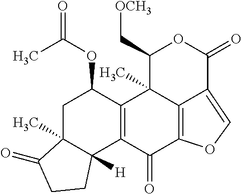 [SMILES: COC[C@H]1OC(=O)c2coc3c2[C@@]1(C)C1=C(C3=O)[C@@H]2CCC(=O)[C@@]2(C)C[C@H]1OC(C)=O] |
| N-Acetyl-D- sphingosine CERAMIDE C2 PubChem CID: 5497136 | 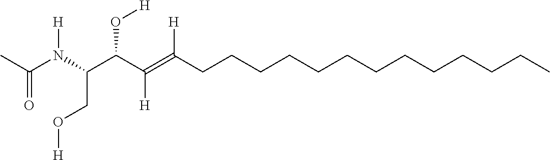 [SMILES: CCCCCCCCCCCCC/C=C/[C@@H](O)[C@H](CO)NC(C)=O] |
| C6-Ceramide | 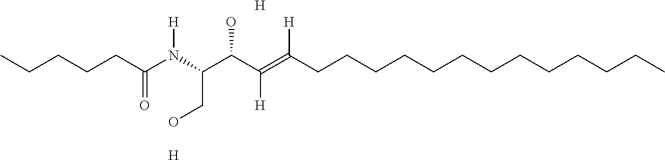 |
| D-e-MAPP | 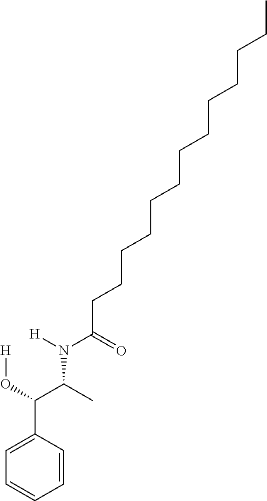 [SMILES: CCCCCCCCCCCCCC(=O)N[C@H](C)[C@@H](O)c1ccccc1] |
| D-NMAPPD (B13) | 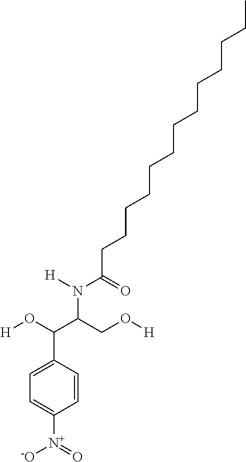 |

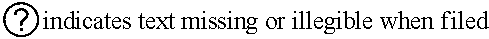

TABLE 5
NCBI Accession numbers of PP2A subunits.

| Name | Gene | Isoform | Accession number | version |
|---|---|---|---|---|
| PP2A sub A alpha | PPP2R1A | | NP_055040 | NP_055040.2 |
| PP2A sub A beta | PPP2R1B | A | NP_002707 | NP_002707.3 |
| | | B | NP_859050 | NP_859050.1 |
| | | C | NP_859051 | NP_859051.1 |
| | | D | NP_001171033 | NP_001171033.1 |
| | | E | NP_001171034 | NP_001171034.1 |
| PP2A sub C alpha | PPP2CA | 1 | NP_002706 | NP_002706.1 |
| | | 2 | NP_001341948 | NP_001341948.1 |
| PP2A sub C beta | PPP2CB | | NP_001009552 | NP_001009552.1 |
| PP2A sub B56delta | PPP2R5D | 1 | NP_006236 | NP_006236.1 |
| | | 2 | NP_851307 | NP_851307 |
| | | 3 | NP_851308 | NP_851308.1 |
| | | 4 | NP_001257405 | NP_001257405.1 |

| Standard name | Systematic name |
|---|---|
| IRC21 | YMR073C |
| RRD1 | YIL153W |
| TIP41 | YPR040W |
| PTC1 | YDL006W |
| SAP190 | YKR028W |
| PPM1 | YDR435C |
| TCO89 | YPL180W |
| TOR1 | YJR066W |

Further accession numbers of PP2A and PP2A-like may be found in Düvel K1, Broach J R. (2011). The role of phosphatases in TOR signaling in yeast. Curr Top Microbiol Immunol. 279:19-38, incorporated by reference and any ortholog thereof, preferably human ortholog.

TOR is a highly conserved protein kinase that is important in both fundamental and clinical biology. In fundamental biology, TOR is a nutrient-sensitive, central controller of cell growth and aging. In clinical biology, TOR is implicated in many diseases and is the target of the drug rapamycin. In the present invention TOR is any TOR as described in Loewith, R., and Hall, M. N. (2011). Target of rapamycin (TOR) in nutrient signaling and growth control. Genetics 189, 1177-1201, incorporated by reference and any ortholog thereof, preferably human ortholog.

### REFERENCES

Alvaro, D., et al. (2007). PLoS Genet 3, e228.
Aoyama, Y., et al. (1981). Biochim Biophys Acta 663, 194-202.
Awasthi, P., et al. (2016). J Cell Sci 129, 1285.
Awaya, J., et al. (1975). Biochim Biophys Acta 409, 267-273.
Bartek, J., and Lukas, J. (2007). Current opinion in cell biology 19, 238-245.
Bashkirov, V. I., et al. (2003). Molecular and cellular biology 23, 1441-1452.
Bastos de Oliveira, F. M., et al. (2015). Molecular cell 57, 1124-1132.
Bazzi, M., et al. (2010). Molecular and cellular biology 30, 131-145.
Beck, T., and Hall, M. N. (1999). Nature 402, 689-692.
Bermejo, R., et al. (2011). Cell 146, 233-246.
Bernardi, P., and Azzone, G. F. (1981). J Biol Chem 256, 7187-7192.
Blaszczynski, M., et al. (1985). Acta Microbiol Pol 34, 243-254.
Chabes, A., et al. (1999). The Journal of biological chemistry 274, 36679-36683.
Chen, L., et al. (2015). J Cell Sci 128, 421.
Clemenson, C., and Marsolier-Kergoat, M. C. (2009). DNA repair 8, 1101-1109.
Cliften, P., et al. (1996). Microbiology 142 (Pt 3), 477-484.
Cocheme, H. M., and Murphy, M. P. (2009). Methods Enzymol 456, 395-417.
Costanzo, M., et al. (2010). Science 327, 425-431.
Cox, K. H., et al. (2004). The Journal of biological chemistry 279, 10270-10278.
Crespo, J. L., et al. (2002). Proceedings of the National Academy of Sciences of the United States of America 99, 6784-6789.
Davidson, J. F., et al. (1996). Proceedings of the National Academy of Sciences of the United States of America 93, 5116-5121.
Desany, B. A., et al. (1998). Genes & development 12, 2956-2970.
Di Como, C. J., and Arndt, K. T. (1996). Genes & development 10, 1904-1916.
Dickson, R. C. (1998). Annual review of biochemistry 67, 27-48.
Dobrowsky, R. T., et al. (1993). The Journal of biological chemistry 268, 15523-15530.
Dozier, C., et al. (2004). Cell 96, 509-517.
Dunn, T. M., et al. (1998). Yeast 14, 311-321.
Duvel, K., et al. (2003). Molecular cell 11, 1467-1478.
El Bawab, S., et al. (2001). The Journal of biological chemistry 276, 16758-16766.
Fay, D. S., et al. (1997). Curr Genet 31, 97-105.
Freeman, A. K., and Monteiro, A. N. (2010). Cell Commun Signal 8, 27.
Gallego, O., et al. (2010). Mol Syst Biol 6, 430.
Girotti, A. W. (1998). J Lipid Res 39, 1529-1542.
Gonzalez, A., et al. (2009). Molecular and cellular biology 29, 2876-2888.
Goodarzi, A. A., et al. (2004). The EMBO journal 23, 4451-4461.
Guan, K., et al. (1992). The Journal of biological chemistry 267, 10024-10030.
Guenole, A., et al. (2013). Molecular cell 49, 346-358.
Haak, D., et al. (1997). The Journal of biological chemistry 272, 29704-29710.
Harrison, J. C., and Haber, J. E. (2006). Annual review of genetics 40, 209-235.
Healy, A. M., et al. (1991). Molecular and cellular biology 11, 5767-5780.
Heideker, J., et al. (2007). Cell cycle 6, 3058-3064.
Hilton, B. A., et al. (2015). Molecular cell 60, 35-46.
Huang, M., et al. (1998). Cell 94, 595-605.
Huang, X., et al. (2012). PLoS Genet 8, e1002493.
Huber, A., et al. (2009). Genes & development 23, 1929-1943.
Hughes Hallett, et al. (2014). Genetics 198, 773-786.
Huh, W. K., et al. (2003). Nature 425, 686-691.
Hustedt, N., et al. (2015). Molecular cell 57, 273-289.
Jacinto, E., et al. (2001). Molecular cell 8, 1017-1026.
Janssens, V., and Goris, J. (2001). The Biochemical journal 353, 417-439.
Jiang, Y. (2006). Microbiol Mol Biol Rev 70, 440-449.
Jiang, Y., and Broach, J. R. (1999). The EMBO journal 18, 2782-2792.
Jordens, J., et al. (2006). The Journal of biological chemistry 281, 6349-6357.
Julmanop, C., et al. (1993). J Gen Microbiol 139, 2323-2327.
Keogh, M. C., et al. (2006). Nature 439, 497-501.
Koh, J. L., et al. (2015). G3 (Bethesda) 5, 1223-1232.
Kolaczkowski, et al. (2004). Eukaryotic cell 3, 880-892.
Kontoyiannis, D. P. (2000). J Antimicrob Chemother 46, 191-197.
Kumar, A., et al. (2014). Cell 158, 633-646.
Kvam, E., et al. (2005). Molecular biology of the cell 16, 3987-3998.
Lamb, D. C., et al. (1999). FEBS letters 462, 283-288.
Laxman, S., et al. (2014). Sci Signal 7, ra120.
Lee, K. S., et al. (1993). Molecular and cellular biology 13, 5843-5853.
Lee, S. J., et al. (2003). Molecular and cellular biology 23, 6300-6314.
Lee, W., et al. (2005). PLoS Genet 1, e24.
Leroy, C., et al. (2003). Mol Cell 11, 827-835.
Loewith, R., and Hall, M. N. (2011). Genetics 189, 1177-1201.
Loewith, R., et al. (2002). Molecular cell 10, 457-468.
Luke, M. M., et al. (1996). Molecular and cellular biology 16, 2744-2755.
Madeira, J. B., et al. (2015). Molecular and cellular biology 35, 737-746.
Mallory, J. C., et al. (2005). Molecular and cellular biology 25, 1669-1679.
Mao, C., et al. (2000a). The Journal of biological chemistry 275, 6876-6884.
Mao, C., et al. (2000b). The Journal of biological chemistry 275, 31369-31378.
Matmati, N., et al. (2013). The Journal of biological chemistry 288, 17272-17284.
Matsuoka, S., et al. (2007). Science 316, 1160-1166.
Mitchell, A. G., and Martin, C. E. (1997). The Journal of biological chemistry 272, 28281-28288.
Nakahata, S., and Morishita, K. (2014). Blood 124, 2163-2165.
Neklesa, T. K., and Davis, R. W. (2008). Proceedings of the National Academy of Sciences of the United
States of America 105, 15166-15171.
Nickels, J. T., and Broach, J. R. (1996). Genes & development 10, 382-394.
O'Neill, B. M., et al. (2007). Proceedings of the National Academy of Sciences of the United States of
America 104, 9290-9295.
Oaks, J., and Ogretmen, B. (2014). Front Oncol 4, 388.
Ogawa, T., et al. (2016). Proceedings of the National Academy of Sciences of the United States of America.
Oh, C. S., et al. (1997). The Journal of biological chemistry 272, 17376-17384.
Orlova, M., et al. (2006). Eukaryotic cell 5, 1831-1837.
Osumi, T., et al. (1979). J Biochem 85, 819-826.
Paciotti, V., et al. (2001). Molecular and cellular biology 21, 3913-3925.
Pedruzzi, I., et al. (2003). Molecular cell 12, 1607-1613.
Pellicioli, A., et al. (1999). EMBO J 18, 6561-6572.
Petranyi, G., et al. (1984). Science 224, 1239-1241.
Poklepovich, T. J., et al. (2012). Steroids 77, 1313-1320.
Qiu, J., et al. (1999). Molecular and cellular biology 19, 8361-8371.
Ramachandran, V., and Herman, P. K. (2011). Genetics 187, 441-454.
Rempola, B., et al. (2000). Molecular & general genetics: MGG 262, 1081-1092.
Ronne, H., et al. (1991). Molecular and cellular biology 11, 4876-4884.
Rossetto, D., et al. (2012). Epigenetics 7, 1098-1108.
Rossi, S. E., et al. (2015). Cell Rep 13, 80-92.
Rossler, H., et al. (2003). Mol Genet Genomics 269, 290-298.
Sanchez, Y., et al. (1996). Science 271, 357-360.
Santhanam, A., et al. (2004). Eukaryotic cell 3, 1261-1271.
Schmidt, A., et al. (1998). The EMBO journal 17, 6924-6931.
Shimura, T., et al. (2016). Oncotarget 7, 3559-3570.
Shu, Y., et al. (1997). Mol Cell Biol 17, 3242-3253.
Slater, M. L. (1973). J Bacteriol 113, 263-270.
Sneddon, A. A., et al. (1990). The EMBO journal 9, 4339-4346.
Sogo, J. M., et al. (2002). Science 297, 599-602.
Staschke, K. A., et al. (2010). The Journal of biological chemistry 285, 16893-16911.
Stock, S. D., et al. (2000). Antimicrob Agents Chemother 44, 1174-1180.
Sun, Z., et al. (1996). Genes & development 10, 395-406.
Sutter, B. M., et al. (2013). Cell 154, 403-415.
Sutton, A., et al. (1991). Molecular and cellular biology 11, 2133-2148.
Taguchi, N., et al. (1994). Microbiology 140 (Pt 2), 353-359.
Takemoto, J. Y., et al. (1993). FEMS Microbiol Lett 114, 339-342.
Tamura, Y., et al. (1976). Archives of biochemistry and biophysics 175, 284-294.
Thomas, B. J., and Rothstein, R. (1989). Cell 56, 619-630.
Tong, A. H., et al. (2001). Science 294, 2364-2368.
Torres, J. Z., et al. (2004). Molecular and cellular biology 24, 3198-3212.
Tripathi, K., et al. (2011). Genetics 189, 533-547.
Urban, J., et al. (2007). Molecular cell 26, 663-674.
Usui, T., et al. (2001). Molecular cell 7, 1255-1266.
Van Hoof, C., et al. (2005). The Biochemical journal 386, 93-102.
van Zyl, W., et al. (1992). IMolecular and cellular biology 12, 4946-4959.
Wei, H., et al. (2001). The Journal of biological chemistry 276, 1570-1577.
Wei, Y., and Zheng, X. F. (2009). Cell cycle 8, 4085-4090.
Wong, P. M., et al. (2015). Nat Commun 6, 8048.
Wu, J., et al. (2000). The EMBO journal 19, 5672-5681.
Wu, W. I., et al. (1995). The Journal of biological chemistry 270, 13171-13178.
Zhang, Z., et al. (1994). The Journal of biological chemistry 269, 16997-17000.
Zhao, X., et al. (1998). Molecular cell 2, 329-340.
Zhao, X., and Rothstein, R. (2002). Proc Natl Acad Sci USA 99, 3746-3751.
Zheng, Y., and Jiang, Y. (2005). Molecular biology of the cell 16, 2119-2127.
Aichberger, K. J., et al. (2005). Blood 105, 3303-3311.
Akgul, C. (2009). Cell. Mol. Life Sci. 66, 1326-1336.
Anisimov, V. N. (2014). Ann. Transl. Med. 2, 60.
Articles, R. (2007). Alternate-day fasting and chronic disease prevention: a review of human and animal trials 1-3 INTRODUCTION. 7-13.
Bennecib, M., et al. (2000). FEBS Lett. 485, 87-93.
Beurel, E., and Jope, R. S. (2006). Prog. Neurobiol. 79, 173-189.
Bijur, G. N., and Jope, R. S. (2000). J. Neurochem. 75, 2401-2408.
Birsoy, K., et al. (2014). Nature 508 VN-, 108-112.
Boisvert-Adamo, K., et al. (2009). Mol. Cancer Res. MCR 7, 549-556.
Boisvert-adamo, K., et al. (2009). Mcl-1 Is Required for Melanoma Cell Resistance to Anoikis Mcl-1 Is
Required for Melanoma Cell Resistance to Anoikis. 549-556.
Bonanni, B., et al. (2012). Dual Effect of Metformin on Breast Cancer Proliferation in a Randomized Presurgical Trial. 30, 2593-2600.
Cantó, C., and Auwerx, J. (2011). Physiology 26, 214-224.
Chance, B. (2005). Cancer Biol. Ther. 4, 125-126.
Choi, Y. W., and Lim, I. K. (2014). Cancer Lett. 346, 300-308.
Cho-Vega, J. H., et al. (2004). Hum. Pathol. 35, 1095-1100.
Cohen, D. H., and LeRoith, D. (2012). Cancer 19, 27-45.
Cohen, P., and Frame, S. (2001). Mol. Cell Biol. 2, 769-776.
DeCensi, A., et al. (2014). Breast Cancer Res. Treat. 148, 81-90.
Ding, Q., et al. (2007a). Cancer Res. 67, 4564-4571.
Ding, Q., He, X., Xia, W., Hsu, J., Chen, C., Li, L., Lee, D., Yang, J., Xie, X., Liu, J., et al. (2007b). Myeloid Cell Leukemia-1 Inversely Correlates with Glycogen Synthase Kinase-3 B Activity and Associates with Poor Prognosis in Human Breast Cancer. 4564-4572.
Dougan, S., et al. (2005). BMJ 330, 1303-1304.
Dowling, R. J. O., Niraula, S., Stambolic, V., and Goodwin, P. J. (2012). Metformin in cancer: Translational challenges. J. Mol. Endocrinol. 48.
Eichhorn, P. J. A., et al. (2009). Biochim. Biophys. Acta—Rev. Cancer 1795, 1-15.
Elgendy, M. (2017). Mol. Cell. Oncol. 0, e1285385.
Elgendy, M., and Minucci, S. (2015). Autophagy 11, 581-582.
Elgendy, M., et al. (2014). Nat. Commun. 5, 5637.
Frame, S., and Cohen, P. (2001). Biochem. J. 359, 1-16.
Gandini, S., et al. (2014). Cancer Prev. Res. (Phila. Pa.) 7, 867-885.
Garg, S. K., et al. (2014). Diabetes Obes. Metab. 16, 97-110.
Gatenby, R. A., and Gillies, R. J. (2004). Nat. Rev. Cancer 4, 891-899.
Germain, M., et al. (2011). EMBO J. 30, 395-407.
Gores, G. J., and Kaufmann, S. H. (2012). Genes Dev. 26, 305-311.
Gores, G. J., Kaufmann, S. H., Glaser, S. P., Lee, E. F., and Trounson, E. (2012). leukemia and solid tumors
Selectively targeting Mcl-1 for the treatment of acute myelogenous leukemia and solid tumors. 305-311.
Greenblatt, D. J., et al. (1977). J. Clin. Pharmacol. 29, 490-494.
Gutierrez, A., et al. (2014). J. Clin. Invest. 124, 644-655.
Haesen, D., Sents, W., Lemaire, K., Hoorne, Y., and Janssens, V. (2014). The Basic Biology of PP2A in
Hematologic Cells and Malignancies. Front. Oncol. 4.
Haesen, D., et al. (2016). Cancer Res. 76, 5719-5731.
Hao, W., et al. (2010). J. Biol. Chem. 285, 12647-12654.
Hongisto, V., et al. (2003). Mol. Cell. Biol. 23, 6027-6036.
Houge, G., et al. (2015). J. Clin. Invest. 125, 3051-3062.
Inuzuka, H., et al. (2011). Nature 471, 104-109.
Jacobs, K. M., et al. (2012). Int. J. Cell Biol. 2012.
Janssens, V., and Goris, J. (2001). Biochem. J. 353, 417-439.
Janssens, V., et al. (2005). Curr. Opin. Genet. Dev. 15, 34-41.
Jee, H. U., et al. (2007). J. Biol. Chem. 282, 20794-20798.
Jiang, C. C., et al. (2008). Cancer Res. 68, 6708-6717.
Jope, R. S., and Johnson, G. V. W. (2004). Trends Biochem. Sci. 29, 95-102.
Jose, C., et al. (2011). Biochim. Biophys. Acta—Bioenerg. 1807, 552-561.
Junttila, M. R., et al. (2007). Cell 130, 51-62.
Kapfhamer, D., et al. (2010). J Neurosci 30, 8830-8840.
Kasznicki, J., et al. (2014). Ann. Transl. Med. 2, 57.
Khanna, A., et al. (2013). Cancer Res. 73, 6548-6553.
Khoury, J. D., et al. (2003). J. Pathol. 199, 90-97.
Kim, S. M., et al. (2016). J. Clin. Invest. 126, 4088-4102.
King, T. D., et al. (2001). Brain Res 919, 106-114.
Kumar, A., et al. (2012). Carcinogenesis 33, 1726-1735.
Laplante, M., and Sabatini, D. M. (2012). Cell 149, 274-293.
Lee, C., and Longo, V. D. (2011). Oncogene 30, 3305-3316.
Lee, M., and Yoon, J.-H. (2015). World J. Biol. Chem. 6, 148-161.
Lee, C., et al. (2012). Sci. Transl. Med. 4, 124ra27.
Lin, C., Chen, C., Chiang, C., Jan, M., Huang, W., and Lin, Y. (2007a). GSK-3P3 acts downstream of
PP2A and the PI 3-kinase-Akt pathway, and upstream of caspase-2 in ceramide-induced mitochondrial
apoptosis. 2935-2943.
Lin, C.-F., et al. (2007b). J. Cell Sci. 120, 2935-2943.
Loberg, R. D., et al. (2002). J. Biol. Chem. 277, 41667-41673.
Longo, V. D., and Mattson, M. P. (2014). Cell Metab. 19, 181-192.
Louis, J. V., et al. (2011). Proc. Natl. Acad. Sci. 108, 6957-6962.
Magiera, M. M., et al. (2012). Cell Death Differ. 20, 281-292.
Martin, M. J., et al. (2012). Cancer Discov. 2, 344-355.
Maurer, U., C et al. (2006). Mol. Cell 21, 749-760.
Mitra, A., et al. (2012). Oncogene 31, 4472-4483.
Morel, C., Carlson, S. M., White, F. M., and Davis, R. J. (2009). Mcl-1 Integrates the Opposing Actions of Signaling Pathways That Mediate Survival and Apoptosis □. 29, 3845-3852.
Mumby, M. (2007). Cell 130, 21-24.
Oyesanya, R. A., Dasgupta, S., Dent, P., and Grant, S. (2012). Targeting Mcl-1 for the therapy of cancer. 20, 1397-1411.
Pap, M., and Cooper, G. M. (1998). Role of Glycogen Synthase Kinase-3 in the Akt Cell Survival Pathway *. 19929-19932.
Pap, M., and Cooper, G. M. (2002). Mol. Cell. Biol. 22, 578-586.
Perciavalle, R. M., et al. (2012). Nat. Cell Biol. 14, 575-583.
Perrotti, D., and Neviani, P. (2008). Cancer Metastasis Rev. 27, 159-168.
Pollak, M. N. (2012). Cancer Discov. 2, 778-790.
Qiu, X., et al. (2010). Cell Metab. 12, 662-667.
Quinn, B. A., et al. (2011). Expert Opin. Investig. Drugs 20, 1397-1411.
Raffaghello, L., et al. (2008). Proc. Natl. Acad. Sci. 105, 8215-8220.
Ren, H., et al. (2013a). Mol. Cancer 12, 146.
Ren, H., et al. (2013b). Cancer Lett. 338, 229-238.
Research, A. A. for C. (2014). Activation of PP2A by Perphenazine Induces Apoptosis in T-ALL. Cancer
Discov. 4, OF14-OF14.
Robillard, N., Gomez, P., Moreau, P., Gouill, S. Le, Harousseau, J., and Amiot, M. (2005). Mcl-1 is
overexpressed in multiple myeloma and associated with relapse and shorter survival. 45, 1248-1252.
Rodríguez-enriquez, S., Marín-hernμndez, A., Gallardo-pørez, J. C., Carreæo-fuentes, L., and Moreno-sμnchez, R. (2009). Review Targeting of cancer energy metabolism. 29-48.
Safdie, M., Dorff, T., Quinn, D., Fontana, L., Wei, M., Cohen, P., and Longo, V. D. (2009). Fasting and cancer treatment in humans: A case series report. 1.
Sambol, N.C., et al. (1996). J. Clin. Pharmacol. 36, 1012-1021.
Sangodkar, J., et al. (2016). FEBS J. 283, 1004-1024.
Sents, W., et al. (2013). FEBS J. 280, 644-661.
Seshacharyulu, P., et al. (2013). Cancer Lett. 335, 9-18.
Somervaille, T. C., et al. (2001). Blood 98, 1374-1381.
Song, L., Sarno, P. De, and Jope, R. S. (2002). Central Role of Glycogen Synthase Kinase-3 [symbol] in Endoplasmic Reticulum Stress-induced Caspase-3 Activation *. 277, 44701-44708.
Suissa, S., and Azoulay, L. (2012). Diabetes Care 35, 2665-2673.
Tsuji, S., et al. (2016). J. Vet. Med. Sci. 78, 1293-1298.
Wang, R., et al. (2012). Leukemia 27, 315-324.
Wang, Y., et al. (2015). Aging 36, 188-200.
Warr, M. R., and Shore, G. C. (2008). Curr. Mol. Med. 8, 138-147.
Westermarck, J., and Hahn, W. C. (2008). Trends Mol. Med. 14, 152-160.
Wuillème-Toumi, S., et al. (2005). Leukemia 19, 1248-1252.
Zhuang, Y., Chan, D. K., Haugrud, A. B., and Miskimins, W. K. (2014). Mechanisms by which low glucose enhances the cytotoxicity of metformin to cancer cells both in vitro and in vivo. PLoS ONE 9.
Alexander, A., et al. (2010). Proc Natl Acad Sci USA 107, 4153-4158.
Alvers, A. L., et al. (2009a). Aging Cell 8, 353-369.
Alvers, A. L., et al. (2009b). Autophagy 5, 847-849.
Andressoo, J. O., et al. (2006). Cell Cycle 5, 2886-2888.
Aris, J. P., et al. (2013). Exp Gerontol 48, 1107-1119.
Bazzi, M., et al. (2010). Mol Cell Biol 30, 131-145.
Bertram, P. G., et al. (2002). Mol Cell Biol 22, 1246-1252.
Bonawitz, N. D., et al. (2007). Cell Metab 5, 265-277.
Braun, K. A., et al. (2014). Sci Signal 7, ra64.
Burtner, C. R., et al. (2009). Cell Cycle 8, 1256-1270.
Chen, Y., and Klionsky, D. J. (2011). J Cell Sci 124, 161-170.
Cherkasova, V., et al. (2010). Mol Cell Biol 30, 2862-2873.
Cherkasova, V. A., and Hinnebusch, A. G. (2003). Genes Dev 17, 859-872.
Conn, C. S., and Qian, S. B. (2013). Sci Signal 6, ra24.
Cosentino, G. P., et al. (2000). Mol Cell Biol 20, 4604-4613.
Daly, M. J. (2012). DNA Repair (Amst) 11, 12-21.
De Haes, W., et al. (2014). Proc Natl Acad Sci USA 111, E2501-2509.
De Virgilio, C. (2012). FEMS Microbiol Rev 36, 306-339.
Dever, T. E., et al. (1992). Cell 68, 585-596.
Diderich, K., et al. (2011). DNA Repair (Amst) 10, 772-780.
Donnelly, N., et al. (2013). Cell Mol Life Sci 70, 3493-3511.
Dotiwala, F., et al. (2013). Proc Natl Acad Sci USA 110, E41-49.
Eapen, V. V., and Haber, J. E. (2013). Autophagy 9, 440-441.
Estruch, F., et al. (1992). Genetics 132, 639-650.
Fabrizio, P., et al. (2004). FEBS Lett 557, 136-142.
Fabrizio, P., et al. (2001). Science 292, 288-290.
Fasolo, J., et al. (2011). Genes Dev 25, 767-778.
Feng, J., et al. (2016). FEMS Yeast Res 16, fow009.
Feng, Z., et al. (2007). Proc Natl Acad Sci USA 104, 16633-16638.
Fiedler, D., et al. (2009). Cell 136, 952-963.
Fiorani, S., et al. (2008). Cell Cycle 7, 493-499.
Fontana, L., et al. (2010). Science 328, 321-326.
Franzke, B., et al. (2015). Mutat Res Rev Mutat Res 766, 48-57.
Freeman, A. K., et al. (2010). Cell Cycle 9, 736-747.
Freeman, A. K., and Monteiro, A. N. (2010). Cell Commun Signal 8, 27.
Galdieri, L., et al. (2010). Omics 14, 629-638.
Gallinetti, J., et al. (2013). Biochem J 449, 1-10.
Ghavidel, A., et al. (2007). Cell 131, 915-926.
Gimeno-Alcaniz, J. V., and Sanz, P. (2003). J Mol Biol 333, 201-209.
Goukassian, D., et al. (2000). Faseb J 14, 1325-1334.
Hardie, D. G. (2014). Cell Metab 20, 939-952.
Harrison, D. E., et al. (2009). Nature 460, 392-395.
Hedbacker, K., and Carlson, M. (2008). Front Biosci 13, 2408-2420.
Hinnebusch, A. G. (2005). Annu Rev Microbiol 59, 407-450.
Hong, S. P., et al. (2003). Proc Natl Acad Sci USA 100, 8839-8843.
Hu, J., et al. (2013). Methods Mol Biol 965, 463-472.
Huber, A., et al. (2009). Genes Dev 23, 1929-1943.
Hughes Hallett, J. E., et al. (2014). Genetics 198, 773-786.
Hussain, S. G., and Ramaiah, K. V. (2007). Biochem Biophys Res Commun 355, 365-370.
Jacinto, E., et al. (2001). Mol Cell 8, 1017-1026.
Kaeberlein, M. (2010). Nature 464, 513-519.
Keogh, M. C., et al. (2006). Nature 439, 497-501.
Kingsbury, J. M., et al. (2014). Genetics 196, 1077-1089.
Klermund, J., et al. (2014). Cell Rep 9, 324-335.
Koga, H., et al. (2011). Ageing Res Rev 10, 205-215.
Krisko, A., and Radman, M. (2013). PLoS Genet 9, e1003810.
Kubota, H., et al. (2003). J Biol Chem 278, 20457-20460.
Leroy, C., et al. (2003). Mol Cell 11, 827-835.
Loewith, R., and Hall, M. N. (2011). Genetics 189, 1177-1201.
Longo, V. D., et al. (2012). Cell Metab 16, 18-31.
Longtine, M. S., et al. (1998). Yeast 14, 953-961.
Lu, J. Y., et al. (2011). Cell 146, 969-979.
Marteijn, J. A., et al. (2014). Nat Rev Mol Cell Biol 15, 465-481.
Martin-Montalvo, A., et al. (2013). Nat Commun 4, 2192.
McCormick, M. A., et al. (2015). Cell Metab 22, 895-906.
Menacho-Marquez, M., et al. (2007). Cell Cycle 6, 2302-2305.
Murguia, J. R., and Serrano, R. (2012). IUBMB Life 64, 971-974.
Neecke, H., et al. (1999). Embo J 18, 4485-4497.
O'Neill, B. M., et al. (2007). Proc Natl Acad Sci USA 104, 9290-9295.
Orlova, M., et al. (2008). Yeast 25, 745-754.
Powers, R. W., 3rd, et al. (2006). Genes Dev 20, 174-184.
Powley, I. R., et al. (2009). Genes Dev 23, 1207-1220.
Ptacek, J., et al. (2005). Nature 438, 679-684.
Qiang, L., et al. (2016). Autophagy 12, 357-368.
Reiling, J. H., and Sabatini, D. M. (2006). Oncogene 25, 6373-6383.
Rempola, B., et al. (2000). Mol Gen Genet 262, 1081-1092.
Reverter-Branchat, G., et al. (2004). J Biol Chem 279, 31983-31989.
Robert, T., et al. (2011). Nature 471, 74-79.
Rohde, J. R., et al. (2004). Mol Cell Biol 24, 8332-8341.
Ruvolo, P., (2016). BBA Clin. 2016 December; 6: 87-99)
Saha, A., et al. (2015). Cancer Prev Res (Phila) 8, 597-606.
Selman, C., et al. (2009). Science 326, 140-144.
Sertic, S., et al. (2012). Cell Cycle 11, 668-674.
Smets, B., et al. (2008). FEMS Yeast Res 8, 1276-1288.
Smith, D. L., Jr., et al. (2007). Aging Cell 6, 649-662.
Steffen, K. K., and Dillin, A. (2016). Cell Metab 23, 1004-1012.
Steffen, K. K., et al. (2008). Cell 133, 292-302.
Steinkraus, K. A., et al. (2008). Annu Rev Cell Dev Biol 24, 29-54.
Storici, F., and Resnick, M. A. (2006). Methods Enzymol 409, 329-345.
Szyjka, S. J., et al. (2008). Genes Dev 22, 1906-1920.
Tate, J. J., et al. (2009). J Biol Chem 284, 2522-2534.
Thomas, B. J., and Rothstein, R. (1989). Genetics 123, 725-738.
Tvegard, T., et al. (2007). Genes Dev 21, 649-654.
Urban, J., et al. (2007). Mol Cell 26, 663-674.
Vaidya, A., et al. (2014). PLoS Genet 10, e1004511.
Viollet, B., et al. Clin Sci (Lond) 122, 253-270.
Vlanti, A., et al. (2013). Aging (Albany N.Y.) 5, 584-585.
Wang, Y., et al. (2014). Oncol Res 22, 193-201.
Weinberger, M., et al. (2010). Aging (Albany N.Y.) 2, 709-726.
Weinberger, M., et al. (2013). Cell Cycle 12, 1189-1200.
Yao, Y., et al. (2015). PLoS Genet 11, e1004968.
Young, E. T., et al. (2000). Gene 245, 299-309.
Yu, G., et al. (2015). Oncotarget 6, 12748-12762.
Zabrocki, P., et al. (2002). Mol Microbiol 43, 835-842.
Zhang, J., et al. (2011). Mol Syst Biol 7, 545.
Zhou, K., et al. (2011). Nat Genet 43, 117-120.

1. (canceled)

2. The method according to claim 26, wherein said modulator modulates the PP2A-GSK3β-MCL-1 axis.

3. The method at least one modulator or combination thereof for use according to claim 2, wherein said modulator is selected from the group consisting of:
a) a small molecule;
b) a polypeptide;
c) an antibody or a fragment thereof;
d) a polynucleotide coding for said antibody or polypeptide or a functional derivative thereof;
e) a polynucleotide, such as antisense construct, antisense oligonucleotide, RNA interference construct or siRNA,
f) a vector comprising or expressing the polynucleotide as defined in d) or e); and
g) a host cell genetically engineered expressing said polypeptide or antibody or comprising the polynucleotide as defined in d) or e).

4. The method according to claim 3 wherein said modulator is selected from the group consisting of: a TORC1 inhibitor, a Ppm1 methyltransferase activator, a TOR inhibitor or wherein said modulator is an intervention and/or an agent that inhibits nutrient uptake (inhibition of nutrient uptake).

5. The method according to claim 7 wherein the ceramide is selected from the group consisting of: N-Acetyl-D-sphingosine c2 ceramide, C6-Ceramide, ceramidase inhibitor, such as D-e-MAPP and D-NMAPPD (B13).

6. The method according to claim 4 wherein the TORC1 inhibitor inhibits the TORC1-Tap42 pathway.

7. The method according to claim 3, wherein the modulator is selected from the group consisting of: metformin, thioridazine, perphenazine, ceramide, Irc21, rapamycin, caffeine, wortmannin, S-adenosyl methionine, FTY-720, fluphenazine, thiethylperazine, pimozide, clozapine, loratadine, promethazine, haloperidol, mersalyl acid, myriocin, fumonisin B1, okadaic acid, cardiolipin, thiethylperazine maleate.

8. The method according to claim 26, wherein said modulator or combination thereof is used in combination with low glucose and/or with at least one DNA damaging agent.

9. The method according to according to claim 26, wherein said DNA damaging agent is an agent selected from the group consisting of: hydroxyurea, gemcitabine, carboplatin, platin-based drug, camptotechin, topoisomerase inhibitors and other chemoterapic drugs or combination thereof.

10. The method according to claim 26, wherein said modulator or combination thereof is used in combination with an inhibitor of glycosidase and/or an inhibitor of amylase.

11. The method according to claim 26, wherein the combination is selected from the group consisting of the combination of: perphenazine and metformin; metformin and thioridazine; metformin and fasting; metformin and intermittent fasting; metformin and fasting mimicking diets; metformin and any form of fasting and at least one compound selected from table 1B such as fluphenazine, thiethylperazine, pimozide, clozapine, loratadine, promethazine, haloperidol; metformin and 2-Deoxy-Glucose; metformin and rapamycin; metformin and amylases and/or glycosidases inhibitors, such as acarbose, quercetin, 5,4′-dihydroxy-3,7-dimethoxyflavone, flavone luteolin, luteolin-7-O-glucoside, eupafolin.

12. The method according to claim 26, wherein the disease characterized by an alteration in the DNA damage response is a cancer and the modulator is an activator of PP2A and/or of PP2A-like phosphatase.

13. The method according to claim 26 wherein the modulator is used in combination with low glucose and/or with at least one DNA damaging agent.

14. The method according to claim 26 wherein the PP2A activator is a compound able to form an active PP2A holoenzyme comprising the regulatory subunit B56∂ or an activator that induces a PP2A holoenzyme that includes the B56c subunit, such as PPZ and Thioridazine, or an activator that needs low glucose (or fasting) and metformin to achieve the formation of an active PP2A holoenzyme that includes B56∂.

15. The method according to 26 wherein said activator of PP2A and/or of PP2A-like phosphatase is selected from the group consisting of: metformin, thioridazine, perphenazine, ceramide, Irc21, a Ppm1 methyltransferase activator, TORC1 inhibitor, rapamycin, caffeine, wortmannin, S-adenosyl methionine, FTY-720, fluphenazine, thiethylperazine, pimozide, clozapine, loratadine, promethazine, haloperidol, and TOR inhibitors.

16. The method according to 12 wherein the cancer presents at least one defect in at least one DDR pathways gene.

17. The method according to 26 wherein the subjects to be treated were previously stratified by analysis of DDR markers.

18-19. (canceled)

20. An in vitro method to identify a subject to be treated with a modulator which is an activator of PP2A and/or of PP2A-like phosphatase or a combination thereof comprising detecting in the genome of said patient a mutation in PP2A and/or a mutation in PP2A-like phosphatase or measuring expression level variation of PP2A and/or PP2A-like phosphatase.

21. The in vitro method according to claim 20 wherein said patient is resistant to treatment with metformin.

22. An in vitro method to identify a subject to be treated with a modulator which is an activator of PP2A and/or of PP2A-like phosphatase or a combination thereof comprising detecting in the genome of said patient at least one mutation in at least one DDR pathways gene.

23-25. (canceled)

26. A method for the prevention and/or treatment of a disease characterized by an alteration in the DNA damage response (DDR) comprising administering to a subject in need thereof a modulator which is an activator of PP2A and/or of PP2A-like phosphatase or a combination thereof.

27. The method according to claim 26, wherein said modulator or combination thereof is administered in combination with a therapeutic agent.
